(19) United States
(12) Patent Application Publication
(10) Pub. No.: US 2014/0335050 A1
(43) Pub. Date: Nov. 13, 2014

(54) METHODS, COMPOSITIONS, AND KITS FOR THE TREATMENT OF CANCER

(71) Applicants: Timothy J. Haggerty, Concord, MA (US); James T. Kurnick, Quincy, MA (US); Ian S. Dunn, Sydney (AU)

(72) Inventors: Timothy J. Haggerty, Concord, MA (US); James T. Kurnick, Quincy, MA (US); Ian S. Dunn, Sydney (AU)

(73) Assignees: The General Hospital Corporation, Boston, MA (US); CytoCure LLC, Beverly, MA (US)

(21) Appl. No.: 14/122,531

(22) PCT Filed: May 25, 2012
(86) PCT No.: PCT/US12/39628

§ 371 Date: Jul. 31, 2014

Related U.S. Application Data
(60) Provisional application No. 61/490,935, filed on May 27, 2011.

(51) Int. Cl. A61K 35/14; A61K 31/4965; A61K 45/06; A61K 38/21; A61K 39/395

(52) U.S. Cl. CPC A61K 35/14; A61K 38/217; A61K 38/215; A61K 39/3955; A61K 35/17; A61K 45/06; A61K 31/4965
USPC 424/85.5; 424/85.6; 424/158.1; 424/174.1; 424/93.7

(57) ABSTRACT

The invention features methods, compositions, and kits for the administration of an HSP90 inhibitor, OBAA, flunarizine, aphidicolin, damnacanthal, dantrolene, or an analog thereof, alone, or in combination with, e.g., a TAA, an antigen-binding scaffold (e.g., an antibody, a soluble T cell receptor, or a chimeric receptor) specific for a TAA, a cell (e.g., a white blood cell that targets a cancer cell), and/or an IFN-β receptor agonist or an IFN-γ receptor agonist, for the treatment of cancer.

### CROSS-REFERENCE TO RELATED APPLICATION

This application claims benefit of priority to U.S. Provisional Application No. 61/490,935, filed May 27, 2011, which is hereby incorporated by reference.

### BACKGROUND OF THE INVENTION

This invention relates to the treatment of cancer.

Over the last several decades, many important breakthroughs have resulted in significant insights in the mechanism of immuno-recognition of tumor cells and their destruction by cytotoxic T-lymphocytes (CTLs). This culminated in improved immunotherapy either by adoptive transfer of activated CTLs or by vaccination with tumor-associated antigens (TAAs). Immunotherapy is especially effective in tumors eliciting a strong immuno-response, such as malignant melanoma.

Tumors develop several mechanisms to evade the immuno-response, including down regulation of TAAs and other molecules that are essential for T cell recognition, such as HLA. Accordingly, there exists a need for compounds and methods that restore TAA expression and sensitize cancer cells to immunotherapy.

### SUMMARY OF THE INVENTION

In one aspect, the invention features a method of treating a cancer in a subject by administering to the subject a composition that includes an HSP90 inhibitor, 3-(4-octadecyl)benzoylacrylic acid (OBAA), flunarizine, aphidicolin, damnacanthal, dantrolene, or an analog thereof. This composition can be administered singly or multiple times.

In a related aspect, the invention features a method of treating cancer in a subject by administering to the subject a first composition that includes an HSP90 inhibitor, OBAA, flunarizine, aphidicolin, damnacanthal, dantrolene, or an analog thereof (e.g., administering the first composition singly or multiple times) and a second composition that includes a cell (e.g., a white blood cell). In this method, the first composition up-regulates expression of one or more tumor associated antigens (TAAs) on a cancer cell and the cell of the second composition interacts with a TAA on the cancer cell. The white blood cell can be a T cell, an NK cell, a LAK cell, monocyte, or a macrophage. Furthermore, the cell can be engineered to express a receptor specific for at least one of the TAAs (e.g., a chimeric T cell receptor optionally including an antibody or antibody fragment specific for a TAA). This cell can be, e.g., autologous or allogeneic to the subject.

In another related aspect, the invention features a method of treating cancer in a subject by administering to the subject a first composition that includes an HSP90 inhibitor, OBAA, flunarizine, aphidicolin, damnacanthal, dantrolene, or an analog thereof (e.g., administering the first composition singly or multiple times) and a second composition that includes an antigen-binding scaffold, e.g., an antibody, soluble T cell receptor, or chimeric receptor, specific for the TAA, or other antibody analog (e.g., single domain antibodies (e.g., shark IgNAR and camelid VHH), protein frameworks including complementary determining regions (e.g., anticalins, affibodies, 4-helix bundle proteins, ankyrin repeat proteins, tetranectins, adnectins, A-domain proteins, lipocalins, immunity protein ImmE7, cytochrome b₅₆₂, amyloid β-protein precursor inhibitor, cellulose binding domain from cellobiohydrolase Cel7A, and carbohydrate binding module CBM4-2, C-type lectins), RNA and DNA aptamers, and molecularly imprinted polymer nanoparticles). In this method, the first composition up-regulates expression of one or more tumor associated antigens (TAAs) on a cancer cell and the antigen-binding scaffold of the second composition interacts with a TAA on the cancer cell.

In any of the foregoing aspects, the invention can also include the administration of a TAA to the subject. The TAA can be administered singly or multiple times to the subject before or after (e.g., 1 to 14 days, 14 to 30 days, or 1 to 6 months before or after) administering any of the foregoing compositions singly or multiple times. The TAA can be a full length TAA protein or peptide fragment. Furthermore, the TAA can be administered with an adjuvant (e.g., GM-CSF, including using Sipuleucel-T treatment) or can be administered loaded onto a cell, e.g., a dendritic cell.

In another related aspect, the invention features a method of treating cancer in a subject by administering to the subject a first composition that includes an HSP90 inhibitor, OBAA, flunarizine, aphidicolin, damnacanthal, dantrolene, or an analog thereof (e.g., administering the first composition singly or multiple times) and a second composition that includes a TAA. The TAA can be administered singly or multiple times to the subject before or after (e.g., 1 to 14 days, 14 to 30 days, or 1 to 6 months before or after) administering any of the foregoing compositions singly or multiple times. The TAA can be a full length TAA protein or peptide fragment. Furthermore, the TAA can be administered with an adjuvant (e.g., GM-CSF, including using Sipuleucel-T treatment) or can be administered loaded onto a cell, e.g., a dendritic cell.

In any of the foregoing aspects, the method can also include administration of an additional anti-cancer therapy, e.g., an immune stimulating molecule, a chemotherapeutic agent, an analgesic, an angiogenesis inhibitor, a steroid, surgical resection, or radiotherapy, to the subject. In one embodiment, the additional anti-cancer therapy is a compound known to increase TAA expression (e.g., an IFN-fl receptor agonist (e.g., IFN-β (e.g., human or variant IFN-β, e.g., IFN-β-1a or IFN-β-1b), an IFN-β mimic, or an IFN-β receptor antibody, or a fragment thereof)), an IFN-γ receptor agonist (e.g., IFN-γ (e.g., human or variant IFN-γ, e.g,. human natural IFN-γ, IFN-γ-1a, IFN-γ-1b, or IFN-γ-1c), an IFN-γ mimic, or an IFN-γ receptor antibody, or a fragment thereof)), a cytotoxic T-lymphocyte antigen-4 (CTLA-4) antagonist (e.g., MDX-010), an antibody or antagonist to PD-1, PD-L1, PD-L2, B7-H3, B7x/B7-H4, BTLA, B7.1, B7.2, or ICOS-L, and/or an agonist (e.g., an antibody agonist) against CD137 (4-1BB), ICOS, OX40, Toll like receptors (e.g., TLR9), or glucocorticoid induced tumor necrosis factor receptor (GITR). An IFN-β receptor agonist may, e.g., include a polypeptide the amino acid sequence of which includes or consists of the amino acid sequence of SEQ ID NO: 1 or SEQ ID NO: 2. An IFN-γ receptor agonist may, e.g., include a polypeptide the amino acid sequence of which includes or consists of the amino acid sequence of SEQ ID NO: 3, SEQ ID NO: 4, SEQ ID NO: 5, or SEQ ID NO: 6.

In any of the foregoing aspects, the method can include administration of an HSP90 inhibitor and an IFN-β receptor agonist (e.g., IFN-β, e.g., human or variant IFN-β, e.g., IFN-β-1a or IFN-β-1b), an IFN-γ receptor agonist (e.g., IFN-γ, e.g., human or variant IFN-γ, e.g,. human natural IFN-γ, IFN-γ-1a, IFN-γ-1b, or IFN-γ-1c), or a CTLA-4 antagonist (e.g., MDX-010).

In some embodiments, the composition that includes the additional anti-cancer therapy, e.g., the IFN-β receptor agonist or IFN-γ receptor agonist, and the composition that includes an HSP90 inhibitor, OBAA, flunarizine, aphidicolin, damnacanthal, dantrolene, or an analog thereof, are administered within 14 days of each other. For example, the composition that includes the additional anti-cancer therapy may be administered between one and seven days, one and three days, or one and 24 hours prior to or following administration of the composition that includes an HSP90 inhibitor, OBAA, flunarizine, aphidicolin, damnacanthal, dantrolene, or an analog thereof. Other dosing intervals are described herein.

In another related aspect, the invention features a method of treating cancer in a subject by administering to the subject a first composition that includes an HSP90 inhibitor, OBAA, flunarizine, aphidicolin, damnacanthal, dantrolene, or an analog thereof (e.g., administering the first composition singly or multiple times) and a second composition that includes an IFN-β receptor agonist (e.g., IFN-β, e.g., human or variant IFN-β, e.g., IFN-β-1a or IFN-β-1b) or an IFN-γ receptor agonist (e.g., IFN-γ, e.g., human or variant IFN-γ, e.g,. human natural IFN-γ, IFN-γ-1a, IFN-γ-1 b, or IFN-γ-1c) (e.g., administering the second composition singly or multiple times). In some embodiments, the first composition includes an HSP90 inhibitor, and the second composition includes IFN-β or IFN-γ. The first and second compositions may be administered within 14 days of each other. For example, the second composition may be administered between one and seven days, one and three days, or one and 24 hours prior to or following administration of the first composition. Other dosing intervals are described herein.

The methods described herein feature treatment of any cancer, including acute lymphoblastic leukemia, acute myeloid leukemia, adrenocortical carcinoma, anal cancer, appendix cancer, astrocytoma, atypical teratoid/rhabdoid tumor, basal cell carcinoma, bile duct cancer, bladder cancer, bone cancer, brain stem glioma, brain tumor, breast cancer, bronchial tumor, Burkitt lymphoma, carcinoid tumor, cervical cancer, chordoma, chronic lymphocytic leukemia, chronic myeloproliferative disorder, colon cancer, colorectal cancer, craniopharyngioma, cutaneous T cell lymphoma, endometrial cancer, ependymoblastoma, ependymoma, esophageal cancer, Ewing sarcoma, extracranial germ cell tumor, extragonadal germ cell tumor, extrahepatic bile duct cancer, eye cancer, gallbladder cancer, gastric cancer, gastroesophageal cancer, gastrointestinal cancer, germ cell tumor, gestational trophoblastic tumor, glioma (e.g., glioblastoma, astrocytoma, or oligodendrocytoma), hairy cell leukemia, head and neck cancer, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, leukemia, lip and oral cavity cancer, liver cancer, lung cancer, malignant teratoma, non-Hodgkin lymphoma, macroglobulinemia, osteosarcoma, medulloblastoma, melanoma, merkel cell carcinoma, mesothelioma, mouth cancer, mycosis fungiodes, myelodysplastic syndrome, multiple myeloma, nasal cavity and paranasal sinus cancer, nasopharyngeal cancer, non-small cell lung cancer, oral cancer, oropharyngeal cancer, osteosarcoma, ovarian cancer, ovarian epithelial cancer, pancreatic cancer, papillomatosis, parathyroid cancer, penile cancer, pharyngeal cancer, pituitary tumor, prostate cancer, rectal cancer, renal cell cancer, retinoblastoma, rhabdomycosarcoma, salivary gland cancer, sarcoma, skin cancer, small intestine cancer, soft tissue sarcoma, testicular cancer, throat cancer, thomoma, thymic carcinoma, thyroid cancer, urethral cancer, uterine cancer, vaginal cancer, and Wilms tumor.

In certain embodiments, the cancer is bladder cancer, brain tumor, breast cancer, colorectal cancer, esophageal cancer, gastric cancer, gastroesophageal cancer, leukemia, lung cancer, melanoma, non-Hodgkin lymphoma, ovarian cancer, pancreatic cancer, prostate cancer, renal cell cancer, thyroid cancer, and uterine cancer.

The TAA of any of the above methods can be (aa) Melan-A/MART-1, (ab) tyrosinase, (ac) gp100/pmel 17, (ad) TRP-1, (ae) TRP-2, (at) an MITF, (ag) MITF-A, (ah) MITF-M, (ai) melanoma GP75, (aj) Annexin I, (ak) Annexin II, (al) adenosine deaminase-binding protein (ADAbp), (am) PGP 9.5, (an) Colorectal associated antigen (CRC)-C017-1A/GA733, (ao) Ab2 BR3E4, (ap) CI17-1A/GA733, (aq) Hsp70, (ar) Hsp90, (as) Hsp96, (at) Hsp 105, (au) Hsp110, (av) HSPPC-96, (aw) stress protein gp96, (ax) gp96-associated cellular peptide, (ay) G250, (az) Dipeptidyl peptidase IV (DPPIV), (ba) Mammaglobin, (be) thyroglobulin, (bd) STn, (be) Carcinoembryonic Antigen (CEA), (bf) CEA epitope CAP-I, (bg) CEA epitope CAP-2, (bh) etv6, (bi) amI1, (bj) Prostate Specific Antigen (PSA), (bk) PSA epitope PSA-1, (bl) PSA epitope PSA-2, (bm) PSA epitope PSA-3, (bn) Ad5-PSA, (ho) prostate-specific membrane antigen (PSMA), (bp) Prostatic Acid Phosphatase (PAP), (bq) Prostate epithelium-derived Ets transcription factor (PDEF), (br) Parathyroid-hormone-related protein (PTH-rP), (bs) EGFR, (bt) PLU1, (bu) Oncofetal antigen-immature laminin receptor (OFA-iLR), (bv) MN/CA IX (CA9), (bw) HP59, (bx) Cytochrome oxidase 1, (by) sp100, (bz) msa, (ca) Ran GTPase activating protein, (cb) a Rab-GAP (Rab GTPase-activating) protein, (cc) PARIS-I, (cd) T cell receptor/CD3-zeta chain, (ce) cTAGE-1, (cf) SCP-1, (cg) Glycolipid antigen-GM2, (ch) GD2 or GD3, (ci) GM3, (cj) FucosylGM1, (ck) Glycoprotein (mucin) antigens-Tn, (cl) Sialyl-Tn, (cm) TF, and (cn) Mucin-I, (co) CA125 (MUC-16), (cp) a MAGE family antigen, (cq) GAGE-1,2, (cr) BAGE, (es) RAGE, (et) LAGE-1, (cu) GnT-V, (cv) EP-CAM/KSA, (cw) CDK4, (cx) a MUC family antigen, (cy) HER2/neu, (cz) ErbB-2/neu, (da) p21ras, (db) RCAS1, (dc) α-fetoprotein, (dd) E-cadherin, (de) α-catenin, (df) β-catenin, (dg) NeuGcGM3, (dh) Fos related antigen, (di) Cyclophilin B, (dj) RCAS1, (dk) S2, (dl) L10a, (dm) Telomerase rt peptide, (dn) cdc27, (do) fodrin, (dp) p120ctn, (dq) PRAME, (dr) GA733/EoCam, (ds) NY-BR-1, (dt) NY-BR-2, (du) NY-BR-3, (dv) NY-BR-4, (dw) NY-BR-5, (dx) NY-BR-6, (dy) NY-BR-7, (dz) NY-ESO-1, (fa) L19H1, (fb) MAZ, (fc) PINCH, (fd) PRAME, (fe) Prp1p/Zer1p, (ff) WT1, (fg) adenomatous polyposis coli protein (APC), (fh) PHF3, (fi) LAGE-1, (fj) SART3, (fk) SCP-1, (fl) SSX-1, (fm) SSX-2, (fn) SSX-4, (fo) TAG-72, (fp) TRAG-3, (fq) MBTAA, (fr) a Smad tumor antigen, (fs) Imp1, (ft) HPV-16 E7, (fu) c-erbB-2, (fv) EBV-encoded nuclear antigen (EBNA)-1, (fw) Herpes simplex thymidine kinase (HSVtk), (fx) alternatively spliced isoform of XAGE-1 (L552S), (fy) TGF beta RII frame shift mutation, (fz) BAX frame shift mutation, (ga) any of the TAAs listed in Table 6, or an (gb) immunogenic fragment thereof. In particular, the TAA may be selected from Table 6 or an immunogenic fragment thereof. The TAA may also include an MHC Class I molecule, an MHC Class II molecule, or an immunogenic fragment thereof.

In any of the foregoing methods, the treating can, e.g., reduce tumor volume, inhibit an increase in tumor volume, stimulate tumor cell lysis or apoptosis, reduce tumor metastasis, reduce the volume of the tumor, reduce the cell number or viability of cells within a mestastasis, or reduce the number of new metastases. In some embodiments, the cancer being treated is melanoma. Desirably, TAAs are selected from the group consisting of Melan-A/Mart-1, Gp100, Tyrosinase, TRP-1, TRP-2, MITF, NY-ESO-1, MAGE, Her2/neu, EphA2, GM2 Ganglioside, GM3 Ganglioside, GD2 ganglioside, GD3 ganglioside, and an immunogenic fragment of any of the above. Treatment of melanoma may include, e.g., administration of a composition including such a TAA, a cell that interacts with the TAA, or an antigen-binding scaffold, e.g., an antibody, soluble T cell receptor, or chimeric receptor, specific for the TAA. A first composition as described herein may further be administered, e.g., containing a compound selected from the group consisting of an HSP90 inhibitor, OBAA, flunarizine, aphidicolin, damnacanthal, dantrolene, and an analog thereof. Desirably, the HSP90 inhibitor is selected from the group consisting of 17-AAG, 17-AEP, 17-DMAG, BIIB021, CCT018159, Celastrol, Gedunin, NVP-AUY922 (aka AUY922), PU-H71, and Radicicol.

In some embodiments, the cancer being treated is a brain tumor. Desirably, TAAs are selected from the group consisting of IL-13Rα 2, Gp100, TRP2, EGFR, GM2 Ganglioside, GM3 Ganglioside, GD2 ganglioside, GD3 ganglioside, CSPG4, EphA2, PRAME, YKL40, hTERT, and an immunogenic fragment of any of the above. Treatment of a brain tumor may include, e.g., administration of a composition including such a TAA, a cell that interacts with the TAA, or an antigen-binding scaffold, e.g., an antibody, soluble T cell receptor, or chimeric receptor, specific for the TAA. A first composition as described herein may further be administered, e.g., containing a compound selected from the group consisting of an HSP90 inhibitor, OBAA, flunarizine, aphidicolin, damnacanthal, dantrolene, and an analog thereof. Desirably, the HSP90 inhibitor is selected from the group consisting of 17-AAG, 17-AEP, 17-DMAG, BIIB021, CCT018159, Celastrol, Gedunin, NVP-AUY922, PU-H71, and Radicicol.

In some embodiments, the cancer being treated is lung cancer. Desirably, TAAs are selected from the group consisting of NY-ESO-1, MAGE, MUC1, TERT, NeuGC-GM3, EGFR, PRAME, EGF, EGFR, STEAP, hTERT, and an immunogenic fragment of any of the above. Treatment of lung cancer may include, e.g., administration of a composition including such a TAA, a cell that interacts with the TAA, or an antigen-binding scaffold, e.g., an antibody, soluble T cell receptor, or chimeric receptor, specific for the TAA. A first composition as described herein may further be administered, e.g., containing a compound selected from the group consisting of an HSP90 inhibitor, OBAA, flunarizine, aphidicolin, damnacanthal, dantrolene, and an analog thereof. Desirably, the HSP90 inhibitor is selected from the group consisting of 17-AAG, 17-AEP, 17-DMAG, BIIB021, CCT018159, Celastrol, Gedunin, NVP-AUY922, PU-H71, and Radicicol.

In some embodiments, the cancer being treated is breast cancer. Desirably, TAAs are selected from the group consisting of BAGE, NY-ESO-1, LAGE, MAGE, Ep-CAM, ErbB2, Estrogen Receptor, Androgen Receptor, Progesterone Receptor, EGFR, EGF, Her2/neu, hTERT, and an immunogenic fragment of any of the above. Treatment of breast cancer may include, e.g., administration of a composition including such a TAA, a cell that interacts with the TAA, or an antigen-binding scaffold, e.g., an antibody, soluble T cell receptor, or chimeric receptor, specific for the TAA. A first composition as described herein may further be administered, e.g., containing a compound selected from the group consisting of an HSP90 inhibitor, OBAA, flunarizine, aphidicolin, damnacanthal, dantrolene, and an analog thereof. Desirably, the HSP90 inhibitor is selected from the group consisting of 17-AAG, 17-AEP, 17-DMAG, BIIB021, CCT018159, Celastrol, Gedunin, NVP-AUY922, PU-H71, and Radicicol.

In some embodiments, the cancer being treated is esophageal, gastric, or gastroesophageal cancer. Desirably, TAAs are selected from the group consisting of MUC1, Her2/neu, EpCAM, EphA2, MAGE, GAGE, NY-ESO-1, CEA, hTERT, and an immunogenic fragment of any of the above. Treatment of esophageal, gastric, or gastroesophageal cancer may include, e.g., administration of a composition including such a TAA, a cell that interacts with the TAA, or an antigen-binding scaffold, e.g., an antibody, soluble T cell receptor, or chimeric receptor, specific for the TAA. A first composition as described herein may further be administered, e.g., containing a compound selected from the group consisting of an HSP90 inhibitor, OBAA, flunarizine, aphidicolin, damnacanthal, dantrolene, and an analog thereof. Desirably, the HSP90 inhibitor is selected from the group consisting of 17-AAG, 17-AEP, 17-DMAG, BIIB021, CCT018159, Celastrol, Gedunin, NVP-AUY922, PU-H71, and Radicicol.

In some embodiments, the cancer being treated is non-Hodgkin lymphoma. Desirably, TAAs are selected from the group consisting of EBV protein LMP1, CD19, CD20, ROR1, ALK, WT1, hTERT, and an immunogenic fragment of any of the above. Treatment of non-Hodgkin lymphoma may include, e.g., administration of a composition including such a TAA, a cell that interacts with the TAA, or an antigen-binding scaffold, e.g., an antibody, soluble T cell receptor, or chimeric receptor, specific for the TAA. A first composition as described herein may further be administered, e.g., containing a compound selected from the group consisting of an HSP90 inhibitor, OBAA, flunarizine, aphidicolin, damnacanthal, dantrolene, and an analog thereof. Desirably, the HSP90 inhibitor is selected from the group consisting of 17-AAG, 17-AEP, 17-DMAG, BIIB021, CCT018159, Celastrol, Gedunin, NVP-AUY922, PU-H71, and Radicicol.

In some embodiments, the cancer being treated is prostate cancer. Desirably, TAAs are selected from the group consisting of PAP (Prostatic Acid Phosphatase), PSA, NY-ESO-1, PSCA, PSMA, ErbB2, Her2/neu, 5T4, STEAP, hTERT, and an immunogenic fragment of any of the above. Treatment of prostate cancer may include, e.g., administration of a composition including such a TAA, a cell that interacts with the TAA, or an antigen-binding scaffold, e.g., an antibody, soluble T cell receptor, or chimeric receptor, specific for the TAA. A first composition as described herein may further be administered, e.g., containing a compound selected from the group consisting of an HSP90 inhibitor, OBAA, flunarizine, aphidicolin, damnacanthal, dantrolene, and an analog thereof. Desirably, the HSP90 inhibitor is selected from the group consisting of 17-AAG, 17-AEP, 17-DMAG, BIIB021, CCT018159, Celastrol, Gedunin, NVP-AUY922, PU-H71, and Radicicol.

In some embodiments, the cancer being treated is renal cell cancer. Desirably, TAAs are selected from the group consisting of 5T4, MUC1, hTERT, CA9, Her2, G250/Carbonic anhydrase IX (CA-IX)/neu, STEAP, FGF-5, and an immunogenic fragment of any of the above. Treatment of renal cell cancer may include, e.g., administration of a composition including such a TAA, a cell that interacts with the TAA, or an antigen-binding scaffold, e.g., an antibody, soluble T cell receptor, or chimeric receptor, specific for the TAA. A first composition as described herein may further be administered, e.g., containing a compound selected from the group consisting of an HSP90 inhibitor, OBAA, flunarizine, aphidicolin, damnacanthal, dantrolene, and an analog thereof. Desirably, the HSP90 inhibitor is selected from the group consisting of 17-AAG, 17-AEP, 17-DMAG, BIIB021, CCT018159, Celastrol, Gedunin, NVP-AUY922, PU-H71, and Radicicol.

In some embodiments, the cancer being treated is bladder cancer. Desirably, TAAs are selected from the group consisting of MAGE, NY-ESO-1, ErbB2, LAGE, PRAME, mannose receptor, Her2/ncu, EpCAM, STEAP, hTERT, and an immunogenic fragment of any of the above. Treatment of bladder cancer may include, e.g., administration of a composition including such a TAA, a cell that interacts with the TAA, or an antigen-binding scaffold, e.g., an antibody, soluble T cell receptor, or chimeric receptor, specific for the TAA. A first composition as described herein may further be administered, e.g., containing a compound selected from the group consisting of an HSP90 inhibitor, OBAA, flunarizine, aphidicolin, damnacanthal, dantrolene, and an analog thereof. Desirably, the HSP90 inhibitor is selected from the group consisting of 17-AAG, 17-AEP, 17-DMAG, BIIB021, CCT018159, Celastrol, Gedunin, NVP-AUY922, PU-H71, and Radicicol.

In some embodiments, the cancer being treated is pancreatic cancer. Desirably, TAAs are selected from the group consisting of MUC1, PSCA, Ep-CAM, Her2/neu, ErbB2, PAP, PSMA, CEA, hTERT, and an immunogenic fragment of any of the above. Treatment of pancreatic cancer may include, e.g., administration of a composition including such a TAA, a cell that interacts with the TAA, or an antigen-binding scaffold, e.g., an antibody, soluble T cell receptor, or chimeric receptor, specific for the TAA. A first composition as described herein may further be administered, e.g., containing a compound selected from the group consisting of an HSP90 inhibitor, OBAA, flunarizine, aphidicolin, damnacanthal, dantrolene, and an analog thereof. Desirably, the HSP90 inhibitor is selected from the group consisting of 17-AAG, 17-AEP, 17-DMAG, BIIB021, CCT018159, Celastrol, Gedunin, NVP-AUY922, PU-H71, and Radicicol.

In some embodiments, the cancer being treated is ovarian cancer. Desirably, TAAs are selected from the group consisting of STEAP, MUC1, Estrogen Receptor, Her2/neu, hTERT, Fralpha, G250, Mesothelin, CEA, ErbB2, and an immunogenic fragment of any of the above. Treatment of ovarian cancer may include, e.g., administration of a composition including such a TAA, a cell that interacts with the TAA, or an antigen-binding scaffold, e.g., an antibody, soluble T cell receptor, or chimeric receptor, specific for the TAA. A first composition as described herein may further be administered, e.g., containing a compound selected from the group consisting of an HSP90 inhibitor, OBAA, flunarizine, aphidicolin, damnacanthal, dantrolene, and an analog thereof. Desirably, the HSP90 inhibitor is selected from the group consisting of 17-AAG, 17-AEP, 17-DMAG, BIIB021, CCT018159, Celastrol, Gedunin, NVP-AUY922, PU-H71, and Radicicol.

In some embodiments, the cancer being treated is colorectal cancer. Desirably, TAAs are selected from the group consisting of MAGE, NY-ESO-2, CEA, 5T4, MUC1, MUC2, ErbB2, FRα, STEAP, hTERT, and an immunogenic fragment of any of the above. Treatment of colorectal cancer may include, e.g., administration of a composition including such a TAA, a cell that interacts with the TAA, or an antigen-binding scaffold, e.g., an antibody, soluble T cell receptor, or chimeric receptor, specific for the TAA. A first composition as described herein may further be administered, e.g., containing a compound selected from the group consisting of an HSP90 inhibitor, OBAA, flunarizine, aphidicolin, damnacanthal, dantrolene, and an analog thereof. Desirably, the HSP90 inhibitor is selected from the group consisting of 17-AAG, 17-AEP, 17-DMAG, BIIB021, CCT018159, Celastrol, Gedunin, NVP-AUY922, PU-H71, and Radicicol.

In some embodiments, the cancer being treated is leukemia. Desirably, TAAs are selected from the group consisting of EBV protein LMP1 and LMP-2, EBV EBNA-1, WT1, Bcr-ab1, NY-ESO-1, Pm1/RARα, CD19, CD20, ROR1, PR1, hTERT, and an immunogenic fragment of any of the above. Treatment of leukemia may include, e.g., administration of a composition including such a TAA, a cell that interacts with the TAA, or an antigen-binding scaffold, e.g., an antibody, soluble T cell receptor, or chimeric receptor, specific for the TAA. A first composition as described herein may further be administered, e.g., containing a compound selected from the group consisting of an HSP90 inhibitor, OBAA, flunarizine, aphidicolin, damnacanthal, dantrolene, and an analog thereof. Desirably, the HSP90 inhibitor is selected from the group consisting of 17-AAG, 17-AEP, 17-DMAG, BIIB021, CCT018159, Celastrol, Gedunin, NVP-AUY922, PU-H71, and Radicicol.

In some embodiments, the cancer being treated is uterine cancer, including cancers of the endometrium and eterine cervix. Desirably, TAAs are selected from the group consisting of HPV E6, HPV E7, HPV L1, ErbB2, RAS p21, Her2/neu, hTERT, and an immunogenic fragment of any of the above. Treatment of uterine cancer may include, e.g., administration of a composition including such a TAA, a cell that interacts with the TAA, or an antigen-binding scaffold, e.g., an antibody, soluble T cell receptor, or chimeric receptor, specific for the TAA. A first composition as described herein may further be administered, e.g., containing a compound selected from the group consisting of an HSP90 inhibitor, OBAA, flunarizine, aphidicolin, damnacanthal, dantrolene, and an analog thereof. Desirably, the HSP90 inhibitor is selected from the group consisting of 17-AAG, 17-AEP, 17-DMAG, BIIB021, CCT018159, Celastrol, Gedunin, NVP-AUY922, PU-H71, and Radicicol.

In some embodiments, the cancer being treated is thyroid cancer. Desirably, TAAs are selected from the group consisting of GAGE 1-6, MAGE-1, MAGE-2, MAGE-3, SSX 1-5, NY-ESO-1, hTERT, WT1, RU2, calcitonin, and an immunogenic fragment of any of the above. Treatment of thyroid cancer may include, e.g., administration of a composition including such a TAA, a cell that interacts with the TAA, or an antigen-binding scaffold, e.g., an antibody, soluble T cell receptor, or chimeric receptor, specific for the TAA. A first composition as described herein may further be administered, e.g., containing a compound selected from the group consisting of an HSP90 inhibitor, OBAA, flunarizine, aphidicolin, damnacanthal, dantrolene, and an analog thereof. Desirably, the HSP90 inhibitor is selected from the group consisting of 17-AAG, 17-AEP, 17-DMAG, BIIB021, CCT018159, Celastrol, Gedunin, NVP-AUY922, PU-H71, and Radicicol.

In another aspect, the invention features a composition including one or more of a first compound including an HSP90 inhibitor, OBAA, flunarizine, aphidicolin, damnacanthal, dantrolene, or an analog thereof, and a second compound including one or more TAAs. The invention also features kits including these compositions and instructions for the administration of these compositions to a subject having cancer or having an increased risk of developing a cancer.

In another aspect, the invention features a composition including an HSP90 inhibitor, OBAA, flunarizine, aphidicolin, damnacanthal, dantrolene, and an analog thereof; and an IFN-β receptor agonist (e.g., IFN-β-1a) or IFN-γ receptor agonist (e.g., IFN-γ-1b). The invention also features kits including these compositions and instructions for the administration of these compositions to a subject having cancer or having an increased risk of developing a cancer.

In another aspect, the invention features a kit including a first composition including an HSP90 inhibitor, OBAA, flunarizine, aphidicolin, damnacanthal, dantrolene, or an analog thereof, and a second composition including a TAA. This kit also includes instructions for the administration of the first and second compositions to a subject having cancer or having an increased risk of developing a cancer.

In another aspect, the invention features a kit including an HSP90 inhibitor, OBAA, flunarizine, aphidicolin, damnacanthal, dantrolene, or an analog thereof, and instructions for administering this agent in combination with a TAA to a subject having cancer or having an increased risk of developing a cancer.

In another aspect, the invention features a kit including a TAA and instructions for administering the TAA with an HSP90 inhibitor, OBAA, flunarizine, aphidicolin, damnacanthal, dantrolene, or an analog thereof, to a subject having cancer or having an increased risk of developing a cancer.

In another aspect, the invention features a kit including a first composition including an HSP90 inhibitor, OBAA, flunarizine, aphidicolin, damnacanthal, dantrolene, or an analog thereof, and a second composition including an IFN-β receptor agonist or IFN-γ receptor agonist. This kit also includes instructions for the administration of the first and second compositions to a subject having cancer or having an increased risk of developing a cancer.

In any of the foregoing aspects, the HSP90 inhibitor can be (a) 17-AAG-nab; (b) 17-AAG; (c) 17-AEP; (d) 17-DMAG; (e) Alvespimycin; (f) Autolytimycin; (g) AUY13387; (h) NVP-AUY922; (i) AT13387; (j) BIIB028; (k) BIIB021; (1) BX-2819; (m) CCT018159; (n) Celastrol; (o) CUDC-305; (p) CUDC-305; (q) Curvularin; (r) Debio 0932; (s) DS-2248; (t) Flavopiridol; (u) Geldamycin; (v) Gedunin; (w) Herbimycin (x) A; (y) Herbimycin B; (z) Herbimycin C; (aa) HSP990; (bb) IPI-493; (cc) IPI-504; (dd) KW 2478; (ee) Lebstatin; (ff) L-783,277; (gg) LL-Z1640-2; (hh) Macbecin I; (ii) Maytansine; (jj) MPC-3100; (kk) MPC-6827; (ll) Mycograb; (mm) NCS-683664; (nn) NXD30001; (oo) NVP-HSP990; (pp) Novobiocin; (qq) PF-04929113; (rr) Pochonin D; (ss) PU-H71; (tt) PU24FC1; (uu) PU-3; (vv) Radicicol; (ww) Reblastatin; (xx) Redicicol; (yy) Rifabutin; (zz) SNX-2112; (aaa) SNX-5422; (bbb) SNX-7081; (ccc) STA-1474; (ddd) STA-9090; (eee) Tanespimycin; (fff) VER49009; (ggg) Xestodecalactone; (hhh) XL888; (iii) Zearalenone, or (jjj) any of the compounds listed in Table 1. In some embodiments, the HSP90 inhibitor is selected from the group consisting of 17-AAG, 17-AEP, 17-DMAG, BIIB021, CCT018159, Celastrol, Gedunin, NVP-AUY922, PU-H71, and Radicicol.

In any of the foregoing aspects, the OBAA analog can be darapladib, varespladib, SB-480848 or selected from Table 2; the flunarizine analog can be cinnarizine amlodipine, aranidipine, azelnidipine, barnidipine, benidipine, cilnidipine, clevidipine, darodipine, efonidipine, felodipine, isradipine, lacidipine, manidipine, lercanidipine, mepirodipine, nicardipine, nifedipine, niludipin, nilvadipine, nimodipine, nisoldipine, nitrendipine, oxodipine, pranidipine, ryodipine, anipamil, devapamil, emopamil, falipamil, gallopamil, norverapamil, verapamil, clentiazem, diltiazem, bepridil, fendiline, lidoflazine, perhexiline, amrinone, anandamide, azimilide, bencyclane, berbamine, bevantolol, canadine, carboxyamidotriazole, caroverine, cinnarizine, conotoxins, dauricine, dimeditiapramine, dotarizine, enpiperate, eperisone, fantofarone, fasudil, fenamic acid, fostedil, gabapentin, lamotrigine, magnesium sulfate, manoalide, mibefradil, monatepil, naftopidil, niguldipine, ochratoxin a, octylonium, osthol, pinaverium, piperidine, pregabalin, prenylamine, risedronic acid, sesamodil, stepholidine, terodiline, tetrahydropalmatine, tetrandrine, tolfenamic acid, tranilast, trox-1, ziconotide, or selected from Table 3; the aphidicolin analog can be selected from table 4; the damnacanthal analog can be selected from Table 5; and the dantrolene compound can be azumolene.

In several embodiments, the HSP90 inhibitor may be selected from Table 1; the flunarizine analog may be cinnarizine; or the HSP90 inhibitor, OBAA, flunarizine, aphidicolin, damnacanthal, dantrolene, or an analog thereof may be selected from Tables 2-5.

In several embodiments, the invention features combinations (e.g., compositions including a combination of agents, methods of administering a combination of agents, and use of a combination of agents for treating cancer, or in the manufacture of a medicament for treating cancer) of an HSP90 inhibitor (A1), OBAA (A2), or an analog thereof (A3), aphidicolin (A4), or an analog thereof (A5), damnacanthal (A6), or an analog thereof (A7), dantrolene (A8), or an analog thereof (A9), with (B) a TAA, (C) an antigen-binding scaffold, e.g., an antibody, soluble T cell receptor, or chimeric receptor, specific for a TAA, (D) a cell (e.g., a cell that interacts with a TAA on a cancer cell), and/or (e) an IFN-β receptor agonist or IFN-γ receptor agonist. One of ordinary skill in the art would appreciate that the present invention features each and every combination of (A), (B), (C), (D), and (E) compositions provided that the combination includes an (A) composition. Based on this classification of compounds, the invention features the following individual exemplary embodiments: A1,B; A1,C; A1,D; A1, E; A2,B; A2,C; A2,D; A2, E; A3,B; A3,C; A3,D; A3, E; A4,B; A4,C; A4,D; A4, E; A5,B; A5,C; A5,D; A5, E; A6,B; A6,C; A6,D; A6, E; A7,B; A7,C; A7,D; A7, E; A8,B; A8,C; A8,D; A8, E; A9,B; A9,C; A9,D, and A9, E. Furthermore, based on the classification of above compounds, the invention features each individual combination of HSP90 inhibitor (A1a-A1jjj) with each TAA (B1aa-B1ga), antigen-binding scaffold, e.g., antibody, soluble T cell receptor, or chimeric receptor, specific for a TAA (C1aa-C1ga), cell that interacts with a TAA (D1aa-D1ga), and IFN-β receptor agonist or IFN-γ receptor agonist (E). For example, the invention features specific embodiments drawn from the following series as if each combination of the series was specifically listed: A1a,B1aa; A1a,B1ab; A1a,B1ac; . . . A1a,B1ga; A1a,B1gb; A1b,B1aa; A1b,B1ab; A1b,B1ac; . . . A1b,B1ga; A1b,B1gb; A1c,B1aa; A1c,B1ab; A1c,B1ac; . . . A1c,B1ga; A1c,B1gb; . . . A1z,B1aa; A1z,B1ab; A1z,B1ac; . . . A1z,B1ga; A1z,B1gb; A1aa,B1aa; A1aa,B1ab; A1aa,B1ac; . . . A1aa,B1ga; A1aa,B1gb; A1bb,B1aa; A1bb,B1 ab; A1bb,B1ac; . . . A1bb,B1ga; A1bb,B1gb; . . . A1zz,B1aa; A1zz,B1ab; A1zz,B1ac; . . . A1zz,B1ga; A1zz,B1gb; A1aaa,B1aa; A1aaa,B1ab; A1aa,B1ac; . . . A1aaa,B1ga; A1aaa,B1gb; . . . A1bbb,B1aa; A1bbb,B1ab; A1bbb,B1ac; . . . A1bbb,B1ga; A1bbb,B1gb; A1ccc,B1aa; A1ccc,B1ab; A1ccc,B1ac; . . . A1ccc,B1ga; A1ccc,B1gb; . . . A1jjj,B1aa; A1jjj,B1 ab; A1jjj,B1ac; . . . A1jjj,B1ga; A1jjj,B1gb;A1a,C1aa; A1a,C1ab; A1a,C1ac; . . . A1a,C1ga; A1a,C1gb; A1b,C1aa; A1b,C1ab; A1b,C1ac; . . . A1b,C1ga; A1b,C1gb; A1c,C1aa; A1c,C1ab; A1c,C1ac; . . . A1c,C1ga; A1c,C1gb; . . . A1z,C1aa; A1z,C1ab; A1z,C1ac; . . . A1z,C1ga; A1z,C1gb; A1aa,C1aa; A1aa,C1ab; A1aa,C1ac; . . . A1aa,C1ga; A1aa,C1gb; A1bb,C1aa; A1bb,C1ab; A1bb,C1ac; . . . A1bb,C1ga; A1bb,C1gb; . . . A1zz,C1aa; A1zz,C1ab; A1zz,C1ac; . . . A1zz,C1ga; A1zz,C1gb; A1aaa,C1aa; A1aaa,C1ab; A1aa,C1ac; . . . A1aaa,C1ga; A1aaa,C1gb; . . . A1bbb,C1aa; A1bbb,C1ab; A1bbb,C1ac; . . . A1bbb,C1ga; A1bbb,C1gb; A1ccc,C1aa; A1ccc,C1ab; A1ccc,C1ac; . . . A1ccc,C1ga; A1ccc,C1gb; . . . A1jjj,C1aa; A1jjj,C1ab; A1jjj,C1ac; . . . A1jjj,C1ga; A1jjj,C1gb; A1a,D1aa; A1a,D1ab; A1a,D1ac; . . . A1a,D1ga; A1a,D1gb; A1b,D1aa; A1b,D1ab; A1b,D1ac; . . . A1b,D1ga; A1b,D1gb; A1c,D1aa; A1c,D1ab; A1c,D1ac; . . . A1c,D1ga; A1c,D1gb; . . . A1z,D1aa; A1z,D1ab; A1z,D1ac; . . . A1z,D1ga; A1z,D1gb; A1aa,D1aa; A1aa,D1ab; A1aa,D1ac; . . . A1aa,D1ga; A1aa,D1gb; A1bb,D1aa; A1bb,D1ab; A1bb,D1ac; . . . A1bb,D1ga; A1bb,D1gb; . . . A1zz,D1aa; A1zz,D1ab; A1zz,D1ac; . . . A1zz,D1ga; A1zz,D1gb; A1aaa,D1aa; A1aaa,D1ab; A1aa,D1ac; . . . A1aaa,D1ga; A1aaa,D1gb; . . . A1bbb,D1aa; A1bbb,D1ab; A1bbb,D1ac; . . . A1bbb,D1ga; A1bbb,D1gb; A1ccc,D1aa; A1ccc,D1ab; A1ccc,D1ac; . . . A1ccc,D1ga; A1ccc,D1gb; . . . A1jjj,D1aa; A1jjj,D1ab; A1jjj,D1ac; . . . A1jjj,D1ga; A1jjj,D1gb. Any of these combinations may further include an IFN-β receptor agonist or IFN-γ receptor agonist (E), e.g., IFN-β-1a, IFN-β-1b, human natural IFN-γ, IFN-γ-1a, IFN-β-1b, or IFN-β-1c.

Furthermore, based on the classification of above compounds, the invention features each individual combination of OBAA (A2), or an analog thereof (A3), aphidicolin (A4), or an analog thereof (A5), damnacanthal (A6), or an analog thereof (A7), dantrolene (A8), or an analog thereof (A9) with each TAA (B1aa-B1gb), antigen-binding scaffold, e.g., antibody, soluble T cell receptor, or chimeric receptor, specific for a TAA (C1aa-C1gb), cell that interacts with a TAA (D1aa-D1gb), and IFN-β receptor agonist or IFN-γ receptor agonist (E). For example, the invention features specific embodiments drawn from the following series as if each combination of the series was specifically listed: A2,B1aa; A2,B1ab; A2,B1ac; . . . A2,B1ga; A2,B1gb; A3,B1aa; A3,B1ab; A3,B1ac; . . . A3,B1ga; A3,B1gb; A4,B1aa; A4,B1ab; A4,B1ac; . . . A4,B1ga; A4,B1gb; A5,B1aa; A5,B1ab; A5,B1ac; . . . A5,B1ga; A5,B1gb; A6,B1aa; A6,B1ab; A6,B1ac; . . . A6,B1ga; A6,B1gb; A7,B1aa; A7,B1ab; A7,B1ac; . . . A7,B1ga; A7,B1gb; A8,B1aa; A8,B1ab; A8,B1ac; . . . A8,B1ga; A8,B1gb; A9,B1aa; A9,B1ab; A9,B1ac; . . . A9,B1ga; A9,B1gb.

By “HSP90 inhibitor” is meant any member of a class of compounds that inhibits a biological activity (e.g., ATP binding activity or protein binding activity) of an HSP90 protein (e.g., through binding to the HSP90 inhibitor). An HSP90 inhibitor may include, e.g., an antigen-binding scaffold, e.g., an antibody, soluble T cell receptor, or chimeric receptor, or a small molecule. Non-limiting examples of HSP90 inhibitors are described herein.

As used herein, “immune response” refers to a cell mediated or humoral (antibody mediated) response known in the art to be a function of the immune system. Stimulating, inducing, or up-regulating an immune response means that either a cell mediated or humoral immune response is increased or triggered. For example, a melanoma TAA (e.g., an epitope of Melan-A/MART-1), an antigen-binding scaffold, e.g., an antibody, soluble T cell receptor, or chimeric receptor, specific for a TAA, or a cell that specifically interacts with a TAA) can be administered and a CTL response to this antigen in a subject with metastatic melanoma elicited.

By “antigen-binding scaffold” is meant any agent that binds a particular antigen, e.g., a TAA. Exemplary antigen-binding scaffolds are antibodies, e.g., intact antibodies and antibody fragments. Antigen-binding scaffolds also include, for example, other soluble receptors, e.g., soluble T cell receptors and chimeric receptors. Antigen-binding scaffolds further include, e.g., RNA and DNA aptamers, and molecularly imprinted nanoparticles. Antigen-binding scaffolds may be naturally-occurring or engineered, e.g., an engineered protein, and may have similar or equivalent binding function to an antibody.

By “antibody” is meant an intact antibody or an antibody fragment.

By “intact antibody” is meant an antibody which comprises an antigen-binding site as well as a CL and at least heavy chain constant domains CH1, CH2, and CH3. The constant domains can be native sequence constant domains (e.g., human native sequence constant domains) or amino acid sequence variants thereof. Preferably, the intact antibody has one or more effector functions.

By “antibody fragment” is meant a portion of an intact antibody, preferably the antigen binding or variable region of the intact antibody. Examples of antibody fragments include Fab, Fab′, F(ab′)2, and Fv fragments; diabodies; linear antibodies (see U.S. Pat. No. 5,641,870, Example 2; Zapata et al., Protein Eng. 8(10): 1057-1062 (1995)); single-chain antibody molecules; and multispecific antibodies formed from antibody fragments.

The expression “linear antibodies” generally refers to the antibodies described in Zapata et al., Protein Eng., 8(10):1057-1062 (1995). Briefly, these antibodies comprise a pair of tandem Fd segments (VH-CH1-VH-CH1) which, together with complementary light chain polypeptides, form a pair of antigen binding regions. Linear antibodies can be bispecific or monospecific.

Papain digestion of antibodies produces two identical antigen-binding fragments, called “Fab” fragments, and a residual “Fe” fragment, a designation reflecting the ability to crystallize readily. The Fab fragment consists of an entire L chain along with the variable region domain of the H chain (VH), and the first constant domain of one heavy chain (CH1). Pepsin treatment of an antibody yields a single large F(ab′)2 fragment which roughly corresponds to two disulfide linked Fab fragments having divalent antigen-binding activity and is still capable of cross-linking antigen. Fab′ fragments differ from Fab fragments by having additional few residues at the carboxy terminus of the CH1 domain including one or more cysteines from the antibody hinge region. Fab′-SH is the designation herein for Fab′ in which the cysteine residue(s) of the constant domains bear a free thiol group. F(ab′)2 antibody fragments originally were produced as pairs of Fab′ fragments which have hinge cysteines between them. Other chemical couplings of antibody fragments are also known.

The Fc fragment comprises the carboxy-terminal portions of both H chains held together by disulfides. The effector functions of antibodies are determined by sequences in the Fc region; this region is also the part recognized by Fc receptors (FcR) found on certain types of cells.

“Fv” consists of a dimer of one heavy- and one light-chain variable region domain in tight, non-covalent association. From the folding of these two domains emanate six hypervariable loops (3 loops each from the H and L chain) that contribute the amino acid residues for antigen binding and confer antigen binding specificity to the antibody. However, even a single variable domain (or half of an Fv comprising only three CDRs specific for an antigen) has the ability to recognize and bind antigen, although often at a lower affinity than the entire binding site.

“Single-chain Fv” also abbreviated as “sFv” or “scFv” are antibody fragments that comprise the VH and VL antibody domains connected into a single polypeptide chain. Preferably, the sFv polypeptide further comprises a polypeptide linker between the VH and VL domains which enables the sFv to form the desired structure for antigen binding. For a review of sFv, see Pluckthun in The Pharmacology of Monoclonal Antibodies, vol. 113, Rosenburg and Moore eds., Springer-Verlag, New York, pp. 269-315 (1994); Borrebaeck 1995.

The term “diabodies” refers to small antibody fragments prepared by constructing sFv fragments (see preceding paragraph) with short linkers (about 5-10 residues) between the VH and VL domains such that inter-chain but not intra-chain pairing of the V domains is achieved, resulting in a bivalent fragment, i.e., fragment having two antigen-binding sites. Bispecific diabodies are heterodimers of two “crossover” sFv fragments in which the VH and VL domains of the two antibodies are present on different polypeptide chains. Diabodies are described more fully in, for example, EP 404,097; WO 93/11161; and Hollinger et al., Proc. Natl. Acad. Sci. USA, 90:6444-6448 (1993).

“Humanized” forms of non-human (e.g., rodent) antibodies are chimeric antibodies that contain minimal sequence derived from the non-human antibody. For the most part, humanized antibodies are human immunoglobulins (recipient antibody) in which residues from a hypervariable region of the recipient are replaced by residues from a hypervariable region of a non-human species (donor antibody) such as mouse, rat, rabbit or non-human primate having the desired antibody specificity, affinity, and capability. In some instances, framework region (FR) residues of the human immunoglobulin are replaced by corresponding non-human residues. Furthermore, humanized antibodies can comprise residues that are not found in the recipient antibody or in the donor antibody. These modifications are made to further refine antibody performance. In general, the humanized antibody will comprise substantially all of at least one, and typically two, variable domains, in which all or substantially all of the hypervariable loops correspond to those of a non-human immunoglobulin and all or substantially all of the FRs are those of a human immunoglobulin sequence. The humanized antibody optionally also will comprise at least a portion of an immunoglobulin constant region (Fc), typically that of a human immunoglobulin. For further details, see Jones et al., Nature 321:522-525 (1986); Riechmann et al., Nature 332:323-329 (1988); and Presta, Curr. Op. Struct. Biol. 2:593-596 (1992).

As used herein, the term “tumor-associated antigen” or “TAA” refers to an antigen capable of expression by a tumor cell, or on cells of the same lineage as the tumor. The TAA in tumor may be expressed in amounts greater than normal relative to a non-tumor (normal) cell counterpart, or may be expressed at similar levels, or at levels less than normal cell counterparts, particularly if the gene encoding the TAA is down-modulated in the tumor cell.

As used herein, an “IFN-β receptor agonist” means a molecule that binds to IFN-α/β receptor (IFNAR), subunits IFNAR-1 or IFNAR-2, and which elicits a response typical of IFN-β. An exemplary response includes increasing TAA expression, i.e., a TAA inducing activity, and/or increasing MHC Class I expression.

As used herein, an “IFN-γ receptor agonist” means a molecule that binds to IFN-γ receptor (IFNGR), subunit IFNGR-1, and which elicits a response typical of IFN-γ. An exemplary response includes increasing either MHC Class I or both MHC Class I and MHC Class II expression.

As used herein, the terms “mimetic” and “mimic” refer to a synthetic chemical compound which has substantially the same structural and/or functional characteristics as the reference molecule. The mimetic can be entirely composed of synthetic, non-natural amino acid analogues, or can be a chimeric molecule including one or more natural peptide amino acids and one or more non-natural amino acid analogs. The mimetic can be any molecule shose shape, structure, charge, hydrophilicity or hydrophobicity matches that of the reference molecule such that receptors or any other partner proteins of the reference molecule are also recognized by the mimetic. The mimetic can also incorporate any number of natural amino acid conservative substitutions as long as such substitutions do not destroy activity. As with polypeptides which are conservative variants, routine testing can be used to determine whether a mimetic has detectable TAA inducing activity.

By a “cancer” is meant is a member of a class of diseases in which a group of cells display uncontrolled growth, aberrant decreases in rate of cell death, or failure to differentiate normally. A cancer may also be a metastatic cancer (spread to other locations in the body). Non-limiting examples of cancer are: acute lymphoblastic leukemia, acute myeloid leukemia, adrenocortical carcinoma, anal cancer, appendix cancer, astrocytoma, atypical teratoid/rhabdoid tumor, basal cell carcinoma, bile duct cancer, bladder cancer, bone cancer, brain stem glioma, brain tumor, breast cancer, bronchial tumor, Burkitt lymphoma, carcinoid tumor, cervical cancer, chordoma, chronic lymphocytic leukemia, chronic myeloproliferative disorders, colon cancer, colorectal cancer, craniopharyngioma, cutaneous T cell lymphoma, endometrial cancer, ependymoblastoma, ependymoma, esophageal cancer, Ewing sarcoma, extracranial germ cell tumor, extragonadal germ cell tumor, extrahepatic bile duct cancer, eye cancer, gallbladder cancer, gastric cancer, gastroesophageal cancer, gastrointestinal cancer, germ cell tumor, gestational trophoblastic tumor, glioma, hairy cell leukemia, head and neck cancer, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, leukemia, lip and oral cavity cancer, liver cancer, lung cancer, malignant teratoma, non-Hodgkin lymphoma, macroglobulinemia, osteosarcoma, medulloblastoma, melanoma, merkel cell carcinoma, mesothelioma, mouth cancer, mycosis fungiodes, myelodysplastic syndrome, multiple myeloma, nasal cavity and paranasal sinus cancer, nasopharyngeal cancer, non-small cell lung cancer, oral cancer, oropharyngeal cancer, osteosarcoma, ovarian cancer, ovarian epithelial cancer, pancreatic cancer, papillomatosis, parathyroid cancer, penile cancer, pharyngeal cancer, pituitary tumor, prostate cancer, rectal cancer, renal cell cancer, retinoblastoma, rhabdomycosarcoma, salivary gland cancer, sarcoma, skin cancer, small intestine cancer, soft tissue sarcoma, testicular cancer, throat cancer, thomoma and thymic carcinoma, thyroid cancer, urethral cancer, uterine cancer, vaginal cancer, and Wilms tumor.

By a “low dosage” or “sub-therapeutic dose” is meant at least 5% less (e.g., at least 10%, 20%, 50%, 80%, 90%, or even 95%) than the lowest standard dosage of a particular compound formulated for a given route of administration for treatment of any human disease or condition (e.g., a cancer). For example, a low dosage of an agent formulated for administration by intramuscular injection will differ from a low dosage of the agent formulated for oral administration.

By a “high dosage” is meant at least 5% (e.g., at least 10%, 20%, 50%, 100%, 200%, or even 300%) more than the highest standard dosage of a particular compound for treatment of any human disease or condition (e.g., a cancer).

By “standard dosage” is meant the dosage of a particular compound that is normally administered to a subject for treatment of a disorder (e.g., a cancer).

By “treating” is meant the application or administration of a composition (e.g., an HSP90 inhibitor, OBAA, flunarizine, aphidicolin, damnacanthal, dantrolene, or an analog thereof, a TAA, an antigen-binding scaffold, e.g., an antibody, soluble T cell receptor, or chimeric receptor, or a cell) to a patient, who has a disease (e.g., cancer) or a predisposition toward a disease, with the purpose to cure, heal, alleviate, relieve, alter, remedy, ameliorate, improve, or affect the disease, the symptoms of disease, or the predisposition toward disease, or to slow the progression of the disease.

By “subject” is meant any animal. Animals that can be treated using the methods, compositions, and kits of the invention include humans, horses, dogs, cats, pigs, goats, rabbits, hamsters, monkeys, guinea pigs, rats, mice, lizards, snakes, sheep, cattle, fish, and birds.

By “an amount sufficient” is meant the amount of a compound, in a combination of the invention, required to treat a cancer in a clinically relevant manner. A sufficient amount of an active compound used to practice the present invention for therapeutic treatment of a cancer varies depending upon the manner of administration, the age, body weight, and general health of the patient. Ultimately, the prescribers will decide the appropriate amount and dosage regimen. Additionally, an effective amount may be that amount of compound in the combination of the invention that is safe and efficacious in the treatment of a patient having a cancer, over each agent alone as determined and approved by a regulatory authority (such as the U.S. Food and Drug Administration).

By “more effective” is meant that a treatment exhibits greater efficacy, or is less toxic, safer, more convenient, or less expensive than another treatment with which it is being compared. Efficacy may be measured by a skilled practitioner using any standard method that is appropriate for a given indication. Efficacy may also mean greater or enhanced killing of cancer cells in a subject.

By a “synergistic” effect is meant a therapeutic effect observed following administration of two or more agents that is greater than the sum of the therapeutic effects observed following the administration of each single agent. By “synergistic increase” is meant the combination of two or more agents that results in an increase in cancer cell death in a subject that is greater than the sum of the cancer cell death observed following the administration of each individual agent. By “synergistic decrease” is meant the combination of two or more agents that results in a decrease in one or more symptoms of a cancer that is greater than the sum of the decrease in one or more symptoms of the cancer observed following the administration of each individual agent. In another example of synergy, a therapeutic effect is observed for the combination of two or more agents, wherein one or more of the agents is present at a dose that is normally non-therapeutic. In another example of synergy, the combination of two or more agents results in an unexpected decrease in toxicity (i.e., a level of toxicity that is less than the sum of the toxicity observed following administration of the single agents).

As used herein, an “anti-cancer” therapy means any treatment that inhibits, decreases, retards, slows, reduces or prevents tumor, cancer or neoplastic growth, metastasis, proliferation or survival, in vitro or in vivo. Particular non-limiting examples of anti-cancer therapy include chemotherapy, immunotherapy, radiotherapy (ionizing or chemical), local thermal (hyperthermia) therapy, and surgical resection. Any treatment having an anti-cell proliferative activity or effect can be used in combination with the HSP90 inhibitor, OBAA, flunarizine, aphidicolin, damnacanthal, dantrolene, or an analog thereof, in accordance with the invention.

The term “pharmaceutically acceptable salt” represents those salts which are, within the scope of sound medical judgement, suitable for use in contact with the tissues of humans and lower animals without undue toxicity, irritation, allergic response and the like, and are commensurate with a reasonable benefit/risk ratio. Pharmaceutically acceptable salts are well known in the art. The salts can be prepared in situ during the final isolation and purification of the compounds of the invention, or separately by reacting the free base function with a suitable organic acid. Representative acid addition salts include acetate, adipate, alginate, ascorbate, aspartate, benzenesulfonate, benzoate, bisulfate, borate, butyrate, camphorate, camphersulfonate, citrate, cyclopentanepropionate, digluconate, dodecylsulfate, ethanesulfonate, fumarate, glucoheptonate, glycerophosphate, hemisulfate, heptonate, hexanoate, hydrobromide, hydrochloride, hydroiodide, 2-hydroxy-ethanesulfonate, isethionate, lactobionate, lactate, laurate, lauryl sulfate, malate, maleate, malonate, mesylate, methanesulfonate, 2-naphthalenesulfonate, nicotinate, nitrate, oleate, oxalate, palmitate, pamoate, pectinate, persulfate, 3-phenylpropionate, phosphate, picrate, pivalate, propionate, stearate, succinate, sulfate, tartrate, thiocyanate, toluenesulfonate, undecanoate, valerate salts, and the like. Representative alkali or alkaline earth metal salts include sodium, lithium, potassium, calcium, magnesium, and the like, as well as nontoxic ammonium, quaternary ammonium, and amine cations, including, but not limited to ammonium, tetramethylammonium, tetraethylammonium, methylamine, dimethylamine, trimethylamine, triethylamine, ethylamine, and the like.

Compounds useful in the invention include those described herein in any of their pharmaceutically acceptable forms, including isomers such as diastereomers and enantiomers, salts, esters, amides, thioesters, solvates, and polymorphs thereof, as well as racemic mixtures and pure isomers of the compounds described herein.

Other features and advantages of the invention will be apparent from the following detailed description, and from the claims.

### BRIEF DESCRIPTION OF THE DRAWINGS

FIG. 1 is a pair of graphs showing the level of IL-2 in supernatants from co-culture of 5×10⁴ melanoma tumor cell line stimulator tumor cells (MU89) and 2.5×10⁴ responder T cells (J-TCR-M1). The mean and standard deviation of untreated negative control cells (n=16) and positive control cells (n=16) treated for three days with 5000 U/ml IFN-β were plotted. The small dash dotted line represents the average and the large dash dotted line represent plus and minus one standard deviation from the mean for the replicates.

FIG. 2 is a graph showing IL-2 levels produced with a fixed number (2×10⁴) of responder T cells (J-TCR-M1) while varying the number of untreated stimulator tumor cells (MU89). Number of tumor cells on x-axis plotted in log₂. Mean and standard deviation of three replicates are shown.

FIG. 3 is a graph showing primary screen results. 480 compounds from the Known Bioactive Library are shown as circles, triangles represent IFN-β treated positive control, and squares indicate untreated controls. Each point corresponds to a well that has been normalized to the average of the untreated controls for that plate. Dark circles are considered hits and the IL-2 level relative to untreated controls is listed next to these points.

FIG. 4 is a graph showing IL-2 expression from the secondary screen for nonspecific T cell activation. Bars indicate the incubation of 2.5×10⁴ T cells alone with the indicated compounds. The concentration of compounds used in this assay are; 17-AAG 10 μg/ml, OBAA 25 μg/ml, aphidicolin 4 μg/ml, flunarizine 8 μg/ml, dantrolene 2 μg/ml, glyburide 4 μg/ml, and PMA 2 μg/ml.

FIG. 5 is a series of graphs showing IL-2 expression in experiments including the hits in a repeat of the tumor T cell co-culture IL-2 ELISA. Note that the x-axes (drug concentrations) and y-axes (IL-2 production levels) cover different ranges to best indicate the activities of individual compounds. Data from a single determination, representative of at least three confirmatory experiments, are shown.

FIG. 6 is a series of graphs showing EGFP detection of in cells treated with the indicated hits showing ability to upregulate a cell line containing a Melan-A/MART-1 promoter-driven EGFP reporter. Note that the x-axes (drug concentrations) and y-axes (EGFP fluorescent measurement) cover different ranges to best indicate the activities of individual compounds. Data from a single determination, representative of at least three confirmatory experiments, is shown.

FIG. 7 is a series of graphs showing intracellular staining and flow histograms used to generate data for Table 10. All cells were stained with an antibody to gp100. Thin line represents untreated cells, bold line represents cells treated for three days with 17-AAG (1 μg/ml). Numbers represent geometric mean of flow histograms.

FIG. 8 is a series of graphs showing flow histograms for three melanoma cell lines. Upper panels show level of Class I MHC for untreated control cells (dark line) and seven day IFN-β treated cells (grey line). Lower panels show level of Class I MHC for 17-AEP three day treated cells (dark line) and IFN-β seven day and 17-AEP three day treated cells (grey line). Con=untreated control, IFN=IFN-β 5000 Units/ml, AEP=17-AEP 1 μg/ml, A+I=17-AEP and IFN-β combination treatment.

FIG. 9 is a series of graphs showing MCH class I expression. Treatment of MU89 tumor cells with IFN-β and HSP90 inhibitors increases IL-2 secretion by HLA-A2 reactive Jurkat T cells. Two seperate experiments are shown. For each experiment, cells were stained for MHC Class I levels, and the geometric mean of flow histograms is graphed in the top two graphs. The same cells were used in a co-culture experiment to assay HLA-A2 levels using a HLA-A2 reactive Jurkat T cell. The results of an IL-2 ELISA are plotted in the graphs on the bottom of the figure. The average and standard deviation of three replicates is shown.

FIG. 10 is a set of four graphs showing the effect of Hsp90 inhibition on MU89 growth. A WST assay was used to assess cell numbers in control and Hsp90-inhibitor treated tumors. WST levels were assayed at time zero and after 3 days. Cells were treated with the indicated Hsp90 inhibitors at the doses indicated. Percent growth was calculated as described in Methods and is plotted on the left y-axis. Data represent the average and standard deviation of triplicate wells. The level of Melan-A/MART-1 (geometric mean), as assayed by intracellular staining and flow cytometry, is shown for comparison on the right y-axis. The data for Melan-A/MART-1 staining are from one representative experiment.

FIG. 11 is a set of four graphs showing the kinetics of Melan-A/MART-1 increase. The flow cytometry data show the effect of four Hsp90 inhibitors on the MU89 MART::EGFP cell line at the indicated doses as assessed over time. The same number of cells per well were plated in each well of a 24 well plate and drug was added on day zero. Each day cells were collected and assayed for that time point. Control untreated cells are shown for comparison. The data are from one representative experiment.

FIG. 12 is a set of four graphs showing the effect of transient exposure to HSP90 inhibitors on Melan-A/MART-1 promoter driven EGFP expression. In order to determine the requirement for continued IIsp90 inhibitor exposure to achieve enhanced promoter activity, the A375 MART::EGFP cell line was exposed to four different Hsp90 inhibitors for the times indicated. In each case the measurement of EGFP-fluorescence was assayed on day 3. At the times indicated, media with the Hsp90 inhibitor was removed and replaced with media without drug. The data are from one representative experiment.

FIGS. 13A and 13B are a Western blot and a protein gel electrophoresis characterizing Hsp90 inhibition of MAPK signal transduction pathway and Melanoma Associated Antigens. A. Western blot performed on extracts of the MU89 cell line treated as indicated for three days. Extracts were probed with antibodies to BRAF, Melan-A/MART-1, TRP-2, or beta-Actin. 30 μg of total protein was loaded in each lane of the gel. 1.0 μg/ml of 17-AAG, 1.0 μg/ml of 17-AEP, or 2.5 μg/ml CCT018159 were used to treat the cells. B. Protein gel electrophoresis was performed using 30 μg of total protein extracts prepared from the indicated cell lines. Cells were untreated (control) or treated with 0.15 μg/ml of BIIB021 for three days. After transfer Western blots were probed with antibodies to phosphorylated MEK (p-MEK) or β-Actin.

FIGS. 14A and 14B are a pair of charts showing increased T-cell recognition of Hsp90 inhibitor-treated tumor cells. A. Tumor cells were co-cultured with the Jurkat T cell line expressing the Melan-A/MART-1 specific T cell receptor and IL-2 secretion was measured by ELISA. B. Tumor cells were co-cultured with CD8+ isolated from PBLs and infected with the either a vector control or a Melan-A/MART-1 reactive TCR (about 20% positive infection determined by tetramer staining and flow cytometry). The treated MU89 tumor cells used for co-culture in both experiments (A and B) are the same. Cells were treated for three days with IFN-beta (5000 U/ml), 17-AEP (0.5 μg/ml), CCT018159 (5 μg/ml), or PU-H71 (0.15 μg/ml) for three days in a flask before being collected and counted. A 25:10 ratio of tumor cells to T cells were mixed for co-culture (5×10⁴ of tumor cells and 2×10⁴ T cells).

FIG. 15 is a series of histograms showing the effect of Hsp90 Inhibitors on Melan-A/MART-1 promoter. Data shown are flow cytometry-generated histograms of EGFP production in reporter cell lines with EGFP linked to the Melan-A/MART-1 promoter. In each histogram, the thin line curve represents the untreated control, and bold line is Hsp90 inhibitor treated cells. In each case, the reporter cells were treated for three days prior to assessing EGFP-related fluorescence. Data are from one representative experiment. The first and third column are low antigen A375 cells and the second and fourth column are high antigen-expressing MM96L+ cells. Doses of Hsp90 inhibitor used are listed in Table 16.

FIG. 16 is a series of dose response curves for various Hsp90 inhibitors. As in FIG. 15, reporter cells expressing EGFP-linked to the Melan-AMART-1 promoter were treated with a series of Hsp90 inhibitors. Filled diamonds: A375 antigen low Melan-A/MART-1 promoter EGFP reporter cell line. Open circles: MM96L+, high antigen-expressing Melan-A/MART-1 promoter EGFP reporter cell line. Data are from one representative experiment.

FIG. 17 is a series of Western blots that show changes in protein levels and state of phosphorylation after treatment with an Hsp90 inhibitor. A primary Anti BRAF (H145) antibody was used at a 1:5000 dilution. A secondary Goat anti rabbit antibody was used at a dilution of 1:5000. 30 ug total protein was added to each well. Samples were collected from cells that were 3-day treated with indicated amounts of 17-AEP. These data confirm other studies showing that Hsp90 inhibitor 17-AEP reduces levels of BRAF in both A375 and MU89 tumor cells, and that downstream effects of this BRAF decrease leads to decreased levels of both Phosphorylated MEK and Phosphorylated ERK that are induced by BRAF in untreated cells, but which are no longer phosphorylated after Hsp90 inhibitor treatment.

FIG. 18: Luciferase assay of MART-promoter activation following treatment of tumor cells with an Hsp90 inhibitor. We utilized a MART 233 bp minimal promoter to drive synthesis of firefly luciferase. Y-axis represents firefly relative to Ubc driven renilla luciferase control. MU89 melanoma cells were transfected and after one day exposed to the indicated HSP90 inhibitors for 3 days before assaying for luciferase activity. Number and standard deviation are from 2 replicates. These data are further proof that there is induction of increased promoter activity for Melan-A/MART-1 as a further demonstration that there is a true increase in antigen expression in Hsp90-treated cells.

FIG. 19 is a graph showing that the Hsp90 inhibitor, PU-H71 enhances Class I MHC on a variety of tumor types. The greatest levels of MHC induction are seen on the melanoma (MU89), cervical carcinoma (HeLa) and B cell lymphoma (RAJI), while lower levels of induction are seen on the Breast carcinoma (MCF7), osteosarcoma (U2OS) and glioma (U118).

FIG. 20 is a graph showing T Cell recognition is enhanced by treatment with IFN-beta or Hsp90 inhibitors.

FIG. 21 is a graph showing the response of MHC Class I and Class II to treatment with IFN-beta, IFN-gamma, PU-H71, or PU-H71 in combination with IFN-beta or IFN-gamma.

FIG. 22 is a graph showing the effect of iHsp90 on Class I after IFN-beta pre-treatment.

FIG. 23 is a graph showing the effect of iHsp90 on Class II after IFN-gamma pre-treatment.

### DETAILED DESCRIPTION

We discovered that administration of an HSP90 inhibitor, OBAA, flunarizine, aphidicolin, damnacanthal, dantrolene, or an analog thereof, increases expression of tumor associated antigens (TAAs) on cancer cells. Furthermore, we have determined that this increased expression sensitizes cancer cells to an anti-cancer immune response.

Accordingly, the invention features methods and compositions for the administration of an HSP90 inhibitor, OBAA, flunarizine, aphidicolin, damnacanthal, dantrolene, or an analog thereof, alone, or in combination with a TAA, antigen-binding scaffold (e.g., an antibody, soluble T cell receptor, or chimeric receptor), a cell (e.g., a white blood cell that targets a cancer cell), and/or an IFN-β receptor agonist or IFN-γ receptor agonist, for the treatment of cancer. The invention also features a composition including an HSP90 inhibitor, OBAA, flunarizine, aphidicolin, damnacanthal, dantrolene, or an analog thereof, alone, or in combination with a TAA, an antigen-binding scaffold (e.g., an antibody, soluble T cell receptor, or chimeric receptor), cell (e.g., a white blood cell that targets a cancer cell), and/or an IFN-β receptor agonist or IFN-γ receptor agonist, for use in the treatment of cancer. Additionally, the invention features the use of an HSP90 inhibitor, OBAA, flunarizine, aphidicolin, damnacanthal, dantrolene, or an analog thereof, alone, or in combination with a TAA, antigen-binding scaffold (e.g., antibody, soluble T cell receptor, or chimeric receptor), cell (e.g., a white blood cell that targets a cancer cell), and/or an IFN-β receptor agonist or IFN-γ receptor agonist, in the manufacture of a medicament for the treatment of cancer.

The invention also provides methods of increasing TAA expression on a cell (e.g., a tumor cell), e.g., for the treatment of cancer. These methods include administering to a subject having a tumor an amount of an HSP90 inhibitor, OBAA, flunarizine, aphidicolin, damnacanthal, dantrolene, or an analog thereof, sufficient to increase tumor associated antigen expression on a tumor cell. An immune-enhancing agent (e.g., lymphocytes or an antibody or antibody-expressing cells specific for a TAA expressed by the tumor), and/or an IFN-β receptor agonist or IFN-γ receptor agonist, can be administered prior to, substantially contemporaneously with, or following, administration of the HSP90 inhibitor, OBAA, flunarizine, aphidicolin, damnacanthal, dantrolene, or an analog thereof, and/or TAA.

The invention further provides methods of inhibiting silencing of a TAA, e.g., for the treatment of cancer. In one embodiment, a method includes administering to a subject with a tumor an amount of an HSP90 inhibitor, OBAA, flunarizine, aphidicolin, damnacanthal, dantrolene, or an analog thereof, to inhibit silencing of the TAA. In one aspect, the subject has been administered a TAA prior to, substantially contemporaneously with, or following HSP90 inhibitor, OBAA, flunarizine, aphidicolin, damnacanthal, dantrolene, or an analog thereof, administration. In addition, or alternatively, an IFN-β receptor agonist or IFN-γ receptor agonist may be administered prior to, substantially contemporaneously with, or following HSP90 inhibitor, OBAA, flunarizine, aphidicolin, damnacanthal, dantrolene, or an analog thereof, administration.

One mechanism by which TAA silencing occurs is through suppression or inhibition of TAA gene expression at the transcriptional level, which may occur by what is referred to in the art as gene silencing, or by a mechanism in which the gene promoter is inhibited. Gene silencing is believed to occur through chromatin remodeling or proteins that bind DNA, and that directly or indirectly inhibit transcription of the gene. Promoter based inhibition can also occur by positive or negative influences on transcription factors required for gene transcription. An additional mechanism by which TAA silencing occurs is through increased TAA protein degradation or reduced TAA protein stability. The invention includes inhibiting, reversing and reducing TAA silencing, regardless of the biological mechanism.

The invention is described in greater detail below.

### HSP90 Inhibitors

The methods, compositions, and kits of the invention may employ an HSP90 inhibitor that inhibits the biological activity (e.g., ATP binding or protein binding activity) of an HSP90 protein. An HSP90 inhibitor may be an antibody or a small compound. A variety of compounds that inhibit the activity of an HSP90 protein are known in the art.

Non-limiting examples of HSP90 inhibitors are 17-AAG-nab; 17-AAG; 17-AEP; 17-DMAG; Alvespimycin; Autolytimycin; AUY13387; NVP-AUY922; AT13387; BIIB028; BIIB021; BX-2819; CCT018159; Celastrol; CUDC-305; CUDC-305; Curvularin; Debio 0932; DS-2248; Flavopiridol; Geldamycin; Gedunin; Herbimycin A; Herbimycin B; Herbimycin C; HSP990; IPI-493; IPI-504; KW 2478; Lebstatin; L-783,277; LL-Z1640-2; Macbecin I; Maytansine; MPC-3100; MPC-6827; Mycograb; NCS-683664; NXD30001; NVP-HSP990; Novobiocin; PF-04929113; Pochonin D; PU-H71; PU24FC1; PU-3; Radicicol; Reblastatin; Redicicol; Rifabutin; SNX-2112; SNX-5422; SNX-7081; STA-1474; STA-9090; Tanespimycin; VER49009; Xestodecalactone; XL888; and Zearalenone. Additional examples of HSP90 inhibitors are described in Xiao et al. (Mini Reviews Med Chem. 2006; 6(10):1137-1143); Chiosis et al. (Bioorg Med Chem. 2002 November; 10(mi11):3555-3564); Aherne et al. (Methods Mol Med. 2003; 85:149-161); Janin (Drug Discovery Today 2010; 16(9/10):342-353); Janin (J Med Chem. 2005; 48(24):7503-7512); and Rowlands et al. (Anal Biochem. 2004 April 15; 327(2):176-183); U.S. Pat. Nos. 7,160,885; 7,799,781; 7,820,658; 7,544,672 (e.g., compounds of general formula (I)); 7,632,855 (e.g., compounds of general formula (I)); 7,700,625 (e.g., compounds of general formula (I)); 7,767,693 (e.g., compounds of general formula (IA)); 7,834,181; and U.S. Patent Application Publication Nos. 2004/0102458 A1; 2005/0107343 A1; 2005/0113339 A1; 2005/0113340 A1; 2005/0119282 A1; 2005/0209158 A1; 2006/0205705 A1; 2006/0223797 A1 (e.g., compounds of general formula (I)); 2007/0112192 A1 (e.g., compounds of general formula (I)); 2007/0155809 A1; 2007/0191445 A1 (e.g., compounds of general formula (I)); 2007/0253896 A1 (e.g., compounds of general formula (I)); 2007/0265268 A1 (e.g., compounds of general formula (I)); 2008/0004277 A1 (e.g., compounds of general formula (I)); 2008/0027047 A1; 2008/0090880 A1 (e.g., compounds of general formula (I)); 2008/0119507 A1 (e.g., compounds of general formula (I)); 2008/0125446 A1; 2008/0146545 A1 (e.g., compounds of general formula (I)); 2008/0176840 A1; 2008/0214586 A1 (e.g., compounds of general formula (I)); 2008/0234297 A1 (e.g., compounds of general formula (I)); 2008/0234314 A1 (e.g., compounds of general formula (I)); 2008/0269218 A1 (e.g., compounds of general formula (I)); 2009/0054421 A1 (e.g., compounds of general formula (I)); 2009/0054452 A1 (e.g., compounds of general formula (I)); 2009/0163490 A1 (e.g., compounds of general formula (I)); 2009/0197882 A1; 2009/0215777 A1 (e.g., compounds of general formula (I) and (IIa)); 2009/0247524 A1 (e.g., compounds of general formula (I)); 2009/0305998 A1; 2009/0325974 A1 (e.g., compounds of general formula (I)); 2010/0010037 A1 (e.g., compounds of general formula (I)); 2010/0035901 A1 (e.g., compounds of general formula (I)); 2010/0113447 A1; 2010/0240656 A1 (e.g., compounds of general formula (I)); 2010/0249231 A1; 2010/0298331 A1 (e.g., compounds of general formula (I)); 2011/0009397 A1; 2011/0046155 A1 (e.g., compounds of general formula (I)); and 2011/0046387 A1 (e.g., compounds of general formula (I)) (each herein incorporated by reference).

Additional examples of HSP90 inhibitors are 17-AAG analogs set forth in Table 1 below.

TABLE 1
17-AAG analogs

[(3R,5S,6R,7S,10S,11S)-6-hydroxy-5,11-dimethoxy-3,7,9,15-tetramethyl-16,20,22-trioxo-21-(prop-2-
enylamino)-17-azabicyclo[16.3.1]docosa-1(21),8,12,14,18-pentaen-10-yl] carbamate
[6-hydroxy-5,11-dimethoxy-3,7,9,15-tetramethyl-16,20,22-trioxo-21-(prop-2-enylamino)-17-
azabicyclo[16.3.1]docosa-1(21),8,12,14,18-pentaen-10-yl] carbamate
[(3R,5R,6S,7R,8E,10R,11R,12Z,14E)-6-hydroxy-5,11-dimethoxy-3,7,9,15-tetramethyl-16,20,22-trioxo-
21-(prop-2-enylamino)-17-azabicyclo[16.3.1]docosa-1(21),8,12,14,18-pentaen-10-yl] carbamate
[(6S,7R,8E,12Z,14E)-6-hydroxy-5,11-dimethoxy-3,7,9,15-tetramethyl-16,20,22-trioxo-21-
(propylamino)-17-azabicyclo[16.3.1]docosa-1(21),8,12,14,18-pentaen-10-yl] carbamate
[(8E,12E,14Z)-6-hydroxy-5,11-dimethoxy-3,7,9,15-tetramethyl-16,20,22-trioxo-21-(prop-2-enylamino)-
17-azabicyclo[16.3.1]docosa-1(21),8,12,14,18-pentaen-10-yl] carbamate
[(8Z,12Z,14Z)-6-hydroxy-5,11-dimethoxy-3,7,9,15-tetramethyl-16,20,22-trioxo-21-(prop-2-enylamino)-
17-azabicyclo[16.3.1]docosa-1(21),8,12,14,18-pentaen-10-yl] carbamate
[(3R,5S,6R,7S,8E,10S,11S,12E,14E)-6-hydroxy-5,11-dimethoxy-3,7,9,15-tetramethyl-16,20,22-trioxo-
21-(prop-2-enylamino)-17-azabicyclo[16.3.1]docosa-1(21),8,12,14,18-pentaen-10-yl] carbamate
[(3R,5S,6R,7S,8E,10S,11S,12Z,14E)-6-hydroxy-5,11-dimethoxy-3,7,9,15-tetramethyl-16,20,22-trioxo-
21-(prop-2-enylamino)-17-azabicyclo[16.3.1]docosa-1(21),8,12,14,18-pentaen-10-yl] carbamate
[(8E,12Z,14E)-6-hydroxy-5,11-dimethoxy-3,7,9,15-tetramethyl-16,20,22-trioxo-21-(prop-2-enylamino)-
17-azabicyclo[16.3.1]docosa-1(21),8,12,14,18-pentaen-10-yl] carbamate
[(6S,7R,8E,10R,11R,12Z,14E)-6-hydroxy-5,11-dimethoxy-3,7,9,15-tetramethyl-16,20,22-trioxo-21-
(propylamino)-17-azabicyclo[16.3.1]docosa-1(21),8,12,14,18-pentaen-10-yl] carbamate
[(3S,5S,6S,7R,8E,10S,11S,12E,14Z)-6-hydroxy-5,11-dimethoxy-3,7,9,15-tetramethyl-16,20,22-trioxo-
21-(prop-2-enylamino)-17-azabicyclo[16.3.1]docosa-1(21),8,12,14,18-pentaen-10-yl] carbamate
[(3R,5S,6R,7S,8E,10S,11S,12Z,14E)-6-hydroxy-5,11-dimethoxy-3,7,9,15-tetramethyl-16,20,22-trioxo-
21-(propylamino)-17-azabicyclo[16.3.1]docosa-1(21),8,12,14,18-pentaen-10-yl] carbamate
[(3R,5S,6R,7S,8E,10S,11S,12Z,14E)-21-(ethylamino)-6-hydroxy-5,11-dimethoxy-3,7,9,15-tetramethyl-
16,20,22-trioxo-17-azabicyclo[16.3.1]docosa-1(21),8,12,14,18-pentaen-10-yl] carbamate
[(3R,5S,6R,7S,8E,10R,11S,12Z,14E)-6-hydroxy-5,11-dimethoxy-3,7,9,15-tetramethyl-16,20,22-trioxo-
21-(prop-2-enylamino)-17-azabicyclo[16.3.1]docosa-1(21),8,12,14,18-pentaen-10-yl] carbamate
[(3R,5R,6S,7R,8E,10R,11R,12E,14Z)-6-hydroxy-5,11-dimethoxy-3,7,9,15-tetramethyl-16,20,22-trioxo-
21-(prop-2-enylamino)-17-azabicyclo[16.3.1]docosa-1(21),8,12,14,18-pentaen-10-yl] carbamate
[(6S,7R,8E,12E,14Z)-6-hydroxy-5,11-dimethoxy-3,7,9,15-tetramethyl-16,20,22-trioxo-21-
(propylamino)-17-azabicyclo[16.3.1]docosa-1(21),8,12,14,18-pentaen-10-yl] carbamate
[(3R,5S,6R,7S,8E,10S,11S,12E,14Z)-6-hydroxy-5,11-dimethoxy-3,7,9,15-tetramethyl-16,20,22-trioxo-
21-(prop-2-enylamino)-17-azabicyclo[16.3.1]docosa-1(21),8,12,14,18-pentaen-10-yl] carbamate
[(3S,5R,6S,7S,8Z,10S,11S,12E,14E)-6-hydroxy-5,11-dimethoxy-3,7,9,15-tetramethyl-16,20,22-trioxo-
21-(prop-2-enylamino)-17-azabicyclo[16.3.1]docosa-1(21),8,12,14,18-pentaen-10-yl] carbamate
[(3R,5S,6R,7S,8E,10S,11S,12E,14Z)-6-hydroxy-5,11-dimethoxy-3,7,9,15-tetramethyl-16,20,22-trioxo-
21-(propylamino)-17-azabicyclo[16.3.1]docosa-1(21),8,12,14,18-pentaen-10-yl] carbamate
[(6S,7R,8E,10R,11R,12E,14Z)-6-hydroxy-5,11-dimethoxy-3,7,9,15-tetramethyl-16,20,22-trioxo-21-
(propylamino)-17-azabicyclo[16.3.1]docosa-1(21),8,12,14,18-pentaen-10-yl] carbamate
[(3R,5R,6S,7R,8E,10S,11R,12E,14Z)-6-hydroxy-5,11-dimethoxy-3,7,9,15-tetramethyl-16,20,22-trioxo-
21-(prop-2-enylamino)-17-azabicyclo[16.3.1]docosa-1(21),8,12,14,18-pentaen-10-yl] carbamate
[(8Z,12Z,14E)-6-hydroxy-5,11-dimethoxy-3,7,9,15-tetramethyl-16,20,22-trioxo-21-(prop-2-enylamino)-
17-azabicyclo[16.3.1]docosa-1(21),8,12,14,18-pentaen-10-yl] carbamate
[(3R,5S,6R,7S,8Z,10S,11S,12Z,14Z)-6-hydroxy-5,11-dimethoxy-3,7,9,15-tetramethyl-16,20,22-trioxo-
21-(prop-2-enylamino)-17-azabicyclo[16.3.1]docosa-1(21),8,12,14,18-pentaen-10-yl] carbamate
[(3R,5R,6S,7S,8E,10S,11S,12Z,14E)-6-hydroxy-5,11-dimethoxy-3,7,9,15-tetramethyl-16,20,22-trioxo-
21-(prop-2-enylamino)-17-azabicyclo[16.3.1]docosa-1(21),8,12,14,18-pentaen-10-yl] carbamate
[(3S,5R,6R,7R,8Z,10S,11S,12Z,14Z)-6-hydroxy-5,11-dimethoxy-3,7,9,15-tetramethyl-16,20,22-trioxo-
21-(prop-2-enylamino)-17-azabicyclo[16.3.1]docosa-1(21),8,12,14,18-pentaen-10-yl] carbamate
[(3R,5S,6S,7S,8E,10S,11S,12Z,14E)-6-hydroxy-5,11-dimethoxy-3,7,9,15-tetramethyl-16,20,22-trioxo-
21-(prop-2-enylamino)-17-azabicyclo[16.3.1]docosa-1(21),8,12,14,18-pentaen-10-yl] carbamate
[(3S,5S,6S,7R,8E,10S,11S,12Z,14E)-6-hydroxy-5,11-dimethoxy-3,7,9,15-tetramethyl-16,20,22-trioxo-
21-(prop-2-enylamino)-17-azabicyclo[16.3.1]docosa-1(21),8,12,14,18-pentaen-10-yl] carbamate
[(5S,8E,10S,11S,12Z,14E)-6-hydroxy-5,11-dimethoxy-3,7,9,15-tetramethyl-16,20,22-trioxo-21-(prop-2-
enylamino)-17-azabicyclo[16.3.1]docosa-1(21),8,12,14,18-pentaen-10-yl] carbamate
[(3R,5S,6R,7S,8E,10S,11R,12Z,14E)-6-hydroxy-5,11-dimethoxy-3,7,9,15-tetramethyl-16,20,22-trioxo-
21-(prop-2-enylamino)-17-azabicyclo[16.3.1]docosa-1(21),8,12,14,18-pentaen-10-yl] carbamate
[(3S,5S,6R,7S,8E,10R,11R,12E,14Z)-6-hydroxy-5,11-dimethoxy-3,7,9,15-tetramethyl-16,20,22-trioxo-
21-(prop-2-enylamino)-17-azabicyclo[16.3.1]docosa-1(21),8,12,14,18-pentaen-10-yl] carbamate
[(3R,6R,7S,8E,10S,11S,12Z,14E)-6-hydroxy-5,11-dimethoxy-3,7,9,15-tetramethyl-16,20,22-trioxo-21-
(prop-2-enylamino)-17-azabicyclo[16.3.1]docosa-1(21),8,12,14,18-pentaen-10-yl] carbamate
[(3S,5S,6R,7S,8E,10S,11S,12Z,14E)-6-hydroxy-5,11-dimethoxy-3,7,9,15-tetramethyl-16,20,22-trioxo-
21-(prop-2-enylamino)-17-azabicyclo[16.3.1]docosa-1(21),8,12,14,18-pentaen-10-yl] carbamate
[(3R,5S,6R,7S,8E,10S,11S,14Z)-6-hydroxy-5,11-dimethoxy-3,7,9,15-tetramethyl-16,20,22-trioxo-21-
(prop-2-enylamino)-17-azabicyclo[16.3.1]docosa-1(21),8,12,14,18-pentaen-10-yl] carbamate
[(3R,5S,6R,7S,8E,10S,11S,12Z,14E)-6-hydroxy-5,11-dimethoxy-3,7,9,15-tetramethyl-16,20,22-trioxo-
21-(1,1,2,3,3-pentadeuterioprop-2-enylamino)-17-azabicyclo[16.3.1]docosa-1(21),8,12,14,18-pentaen-
10-yl] carbamate
[(3R,5S,6R,7S,8E,10S,11S,12Z,14Z)-6-hydroxy-5,11-dimethoxy-3,7,9,15-tetramethyl-16,20,22-trioxo-
21-(prop-2-enylamino)-17-azabicyclo[16.3.1]docosa-1(21),8,12,14,18-pentaen-10-yl] carbamate
[(3R,5R,6R,7R,8E,10R,11R,14Z)-6-hydroxy-5,11-dimethoxy-3,7,9,15-tetramethyl-16,20,22-trioxo-21-
(prop-2-enylamino)-17-azabicyclo[16.3.1]docosa-1(21),8,12,14,18-pentaen-10-yl] carbamate
[(3R,5S,6R,7S,8E,10S,11S,12Z,14E)-6-hydroxy-5,11-dimethoxy-3,7,9,15-tetramethyl-16,20,22-trioxo-
21-(propan-2-ylamino)-17-azabicyclo[16.3.1]docosa-1(21),8,12,14,18-pentaen-10-yl] carbamate
[(3R,5S,6R,7S,8E,10S,11S,12Z,14E)-6-hydroxy-21-(3-hydroxypropylamino)-5,11-dimethoxy-3,7,9,15-
tetramethyl-16,20,22-trioxo-17-azabicyclo[16.3.1]docosa-1(21),8,12,14,18-pentaen-10-yl] carbamate
[(3R,5S,6R,7S,8E,10S,11S,12Z,14E)-21-(heptylamino)-6-hydroxy-5,11-dimethoxy-3,7,9,15-tetramethyl-
16,20,22-trioxo-17-azabicyclo[16.3.1]docosa-1(21),8,12,14,18-pentaen-10-yl] carbamate
[(3R,5S,6R,7S,8E,10S,11S,12Z,14E)-21-(dodecylamino)-6-hydroxy-5,11-dimethoxy-3,7,9,15-
tetramethyl-16,20,22-trioxo-17-azabicyclo[16.3.1]docosa-1(21),8,12,14,18-pentaen-10-yl] carbamate
[(3R,5S,6R,7S,8E,10S,11S,12E,14Z)-6-hydroxy-5,11-dimethoxy-3,7,9,15-tetramethyl-16,20,22-trioxo-
21-(propan-2-ylamino)-17-azabicyclo[16.3.1]docosa-1(21),8,12,14,18-pentaen-10-yl] carbamate
[(3R,5S,6R,7S,8E,10S,11S,12E,14Z)-6-hydroxy-21-(3-hydroxypropylamino)-5,11-dimethoxy-3,7,9,15-
tetramethyl-16,20,22-trioxo-17-azabicyclo[16.3.1]docosa-1(21),8,12,14,18-pentaen-10-yl] carbamate
[(3R,5S,6R,7S,8Z,10S,11S,12Z,14Z)-6,11-dihydroxy-5-methoxy-3,7,9,15-tetramethyl-16,20,22-trioxo-
21-(prop-2-enylamino)-17-azabicyclo[16.3.1]docosa-1(21),8,12,14,18-pentaen-10-yl] carbamate
[(3R,5S,6R,7S,8E,10S,11S,12Z,14E)-21-(2-fluoroethylamino)-6-hydroxy-5,11-dimethoxy-3,7,9,15-
tetramethyl-16,20,22-trioxo-17-azabicyclo[16.3.1]docosa-1(21),8,12,14,18-pentaen-10-yl] carbamate
[(3R,5S,6R,7S,8E,10S,11S,12Z,14E)-21-(3-aminopropylamino)-6-hydroxy-5,11-dimethoxy-3,7,9,15-
tetramethyl-16,20,22-trioxo-17-azabicyclo[16.3.1]docosa-1(21),8,12,14,18-pentaen-10-yl] carbamate
[(3R,5S,6R,7S,8E,10S,11S,12Z,14E)-6-hydroxy-21-[[(2R)-2-hydroxypropyl]amino]-5,11-dimethoxy-
3,7,9,15-tetramethyl-16,20,22-trioxo-17-azabicyclo[16.3.1]docosa-1(21),8,12,14,18-pentaen-10-yl]
carbamate
[(3R,5S,6R,7S,8E,10S,11S,12Z,14E)-6-hydroxy-21-[[(2S)-2-hydroxypropyl]amino]-5,11-dimethoxy-
3,7,9,15-tetramethyl-16,20,22-trioxo-17-azabicyclo[16.3.1]docosa-1(21),8,12,14,18-pentaen-10-yl]
carbamate
[(3R,5S,6R,7S,8E,10S,11S,12Z,14E)-6-hydroxy-5,11-dimethoxy-3,7,9,15-tetramethyl-16,20,22-trioxo-
21-(prop-2-enylamino)-17-azabicyclo[16.3.1]docosa-1(21),8,12,14,18-pentacn-10-yl] N-ethylcarbamate
[(3R,5S,6S,7S,8E,10S,11S,12Z,14E)-21-(2-fluoroethylamino)-6-hydroxy-5,11-dimethoxy-3,7,9,15-
tetramethyl-16,20,22-trioxo-17-azabicyclo[16.3.1]docosa-1(21),8,12,14,18-pentaen-10-yl] carbamate
[(3R,5S,6R,7S,8E,10S,11S,12Z,14E)-6-hydroxy-21-(2-hydroxyethylamino)-5,11-dimethoxy-3,7,9,15-
tetramethyl-16,20,22-trioxo-17-azabicyclo[16.3.1]docosa-1(21),8,12,14,18-pentaen-10-yl] carbamate
[(3R,5S,6R,7S,8E,10S,11S,12Z,14E)-21-(heptan-2-ylamino)-6-hydroxy-5,11-dimethoxy-3,7,9,15-
tetramethyl-16,20,22-trioxo-17-azabicyclo[16.3.1]docosa-1(21),8,12,14,18-pentaen-10-yl] carbamate
[(3R,5S,6R,7S,8E,10S,11S,12E,14Z)-6-hydroxy-5,11-dimethoxy-3,7,9,15-tetramethyl-16,20,22-trioxo-
21-(prop-2-enylamino)-17-azabicyclo[16.3.1]docosa-1(21),8,12,14,18-pentaen-10-yl] N-ethylcarbamate
[(3R,5S,6S,7S,8E,10S,11S,12E,14Z)-21-(2-fluoroethylamino)-6-hydroxy-5,11-dimethoxy-3,7,9,15-
tetramethyl-16,20,22-trioxo-17-azabicyclo[16.3.1]docosa-1(21),8,12,14,18-pentaen-10-yl] carbamate
[(3R,5S,6R,7S,8E,10S,11S,12E,14Z)-21-(2-fluoroethylamino)-6-hydroxy-5,11-dimethoxy-3,7,9,15-
tetramethyl-16,20,22-trioxo-17-azabicyclo[16.3.1]docosa-1(21),8,12,14,18-pentaen-10-yl] carbamate
[(3R,5S,6R,7S,8E,10S,11S,12E,14Z)-21-(3-aminopropylamino)-6-hydroxy-5,11-dimethoxy-3,7,9,15-
tetramethyl-16,20,22-trioxo-17-azabicyclo[16.3.1]docosa-1(21),8,12,14,18-pentaen-10-yl] carbamate
[(3R,5S,6R,7S,8E,10S,11S,12E,14Z)-6-hydroxy-21-[[(2R)-2-hydroxypropyl]amino]-5,11-dimethoxy-
3,7,9,15-tetramethyl-16,20,22-trioxo-17-azabicyclo[16.3.1]docosa-1(21),8,12,14,18-pentaen-10-yl]
carbamate
[(3R,5S,6R,7S,8E,10S,11S,12E,14Z)-6-hydroxy-21-[[(2S)-2-hydroxypropyl]amino]-5,11-dimethoxy-
3,7,9,15-tetramethyl-16,20,22-trioxo-17-azabicyclo[16.3.1]docosa-1(21),8,12,14,18-pentaen-10-yl]
carbamate
[(3R,5R,6S,7S,8E,10S,11S,12Z,14E)-21-(2-fluoroethylamino)-6-hydroxy-5,11-dimethoxy-3,7,9,15-
tetramethyl-16,20,22-trioxo-17-azabicyclo[16.3.1]docosa-1(21),8,12,14,18-pentaen-10-yl] carbamate
[(3R,5S,6R,7S,8E,10R,12R,13Z,15E)-6-hydroxy-5,12-dimethoxy-3,7,9,16-tetramethyl-17,21,23-trioxo-
22-(prop-2-enylamino)-18-azabicyclo[17.3.1]tricosa-1(22),8,13,15,19-pentaen-10-yl] carbamate
[(3R,5S,6R,7S,8E,10S,11S,12Z,14E)-6-hydroxy-21-(2-hydroxypropylamino)-5,11-dimethoxy-3,7,9,15-
tetramethyl-16,20,22-trioxo-17-azabicyclo[16.3.1]docosa-1(21),8,12,14,18-pentaen-10-yl] carbamate
[(3S,5S,6R,7S,8E,11S,12E,14E)-6-hydroxy-5,11-dimethoxy-3,7,9,15-tetramethyl-16,20,22-trioxo-21-
(prop-2-enylamino)-17-azabicyclo[16.3.1]docosa-1(21),8,12,14,18-pentaen-12-yl] carbamate
[(3R,5S,6R,7S,8E,10S,11S,12Z,14E)-21-(ethenylamino)-6-hydroxy-5,11-dimethoxy-3,7,9,15-
tetramethyl-16,20,22-trioxo-17-azabicyclo[16.3.1]docosa-1(21),8,12,14,18-pentaen-10-yl] carbamate
[(6S,7R,8E,10R,11R,12Z,14E)-6-hydroxy-5,11-dimethoxy-3,7,9,15-tetramethyl-21-(methylamino)-
16,20,22-trioxo-17-azabicyclo[16.3.1]docosa-1(21),8,12,14,18-pentaen-10-yl] carbamate
[(3R,5S,6R,7S,8E,10S,11S,14E)-6-hydroxy-5,11-dimethoxy-3,7,9,15-tetramethyl-16,20,22-trioxo-21-
(prop-2-enylamino)-17-azabicyclo[16.3.1]docosa-1(21),8,14,18-tetraen-10-yl] carbamate
[(3S,5S,6S,7R,8E,10S,11S,14Z)-6-hydroxy-5,11-dimethoxy-3,7,9,15-tetramethyl-16,20,22-trioxo-21-
(prop-2-enylamino)-17-azabicyclo[16.3.1]docosa-1(21),8,14,18-tetraen-10-yl] carbamate
[(3R,5R,6S,7R,8E,10S,11R,14Z)-6-hydroxy-5,11-dimethoxy-3,7,9,15-tetramethyl-16,20,22-trioxo-21-
(prop-2-enylamino)-17-azabicyclo[16.3.1]docosa-1(21),8,14,18-tetraen-10-yl] carbamate
[(6S,7R,8E,10R,11R,12E,14Z)-6-hydroxy-5,11-dimethoxy-3,7,9,15-tetramethyl-21-(methylamino)-
16,20,22-trioxo-17-azabicyclo[16.3.1]docosa-1(21),8,12,14,18-pentaen-10-yl] carbamate
[(8Z,14Z)-6-hydroxy-5,11-dimethoxy-3,7,9,15-tetramethyl-16,20,22-trioxo-21-(prop-2-enylamino)-17-
azabicyclo[16.3.1]docosa-1(21),8,14,18-tetraen-10-yl] carbamate
[(3R,5S,6R,7S,8E,10S,11S,12Z,14E)-6-hydroxy-5,11-dimethoxy-3,7,9,15-tetramethyl-21-
(methylamino)-16,20,22-trioxo-17-azabicyclo[16.3.1]docosa-1(21),8,12,14,18-pentaen-10-yl] carbamate
[21-(cyclopropylamino)-6-hydroxy-5,11-dimethoxy-3,7,9,15-tetramethyl-16,20,22-trioxo-17-
azabicyclo[16.3.1]docosa-1(21),8,12,14,18-pentaen-10-yl] carbamate
[(8E,12E,14Z)-21-(cyclopropylamino)-6-hydroxy-5,11-dimethoxy-3,7,9,15-tetramethyl-16,20,22-trioxo-
17-azabicyclo[16.3.1]docosa-1(21),8,12,14,18-pentaen-10-yl] carbamate
[(8Z,12Z,14Z)-21-(cyclopropylamino)-6-hydroxy-5,11-dimethoxy-3,7,9,15-tetramethyl-16,20,22-trioxo-
17-azabicyclo[16.3.1]docosa-1(21),8,12,14,18-pentaen-10-yl] carbamate
[(3R,5S,6R,7S,8E,10S,11S,12Z,14E)-21-(cyclopropylamino)-6-hydroxy-5,11-dimethoxy-3,7,9,15-
tetramethyl-16,20,22-trioxo-17-azabicyclo[16.3.1]docosa-1(21),8,12,14,18-pentaen-10-yl] carbamate
[(3R,5S,6R,7S,8E,10S,11S,12Z,14E)-6-hydroxy-21-[[(2R)-1-hydroxypropan-2-yl]amino]-5,11-
dimethoxy-3,7,9,15-tetramethyl-16,20,22-trioxo-17-azabicyclo[16.3.1]docosa-1(21),8,12,14,18-pentaen-
10-yl] carbamate
[(3R,5S,6R,7S,8E,10S,11S,12Z,14E)-6-hydroxy-9-(hydroxymethyl)-5,11-dimethoxy-3,7,15-trimethyl-
16,20,22-trioxo-21-(prop-2-enylamino)-17-azabicyclo[16.3.1]docosa-1(21),8,12,14,18-pentaen-10-yl]
carbamate
[(3R,5S,6R,7S,8E,10S,11S,12Z,14E)-6-hydroxy-21-(3-iodopropylamino)-5,11-dimethoxy-3,7,9,15-
tetramethyl-16,20,22-trioxo-17-azabicyclo[16.3.1]docosa-1(21),8,12,14,18-pentaen-10-yl] carbamate
[(3R,5S,6R,7S,8E,10S,11S,12Z,14E)-6-hydroxy-5,11-dimethoxy-21-(2-methoxyethylamino)-3,7,9,15-
tetramethyl-16,20,22-trioxo-17-azabicyclo[16.3.1]docosa-1(21),8,12,14,18-pentaen-10-yl] carbamate
[(3R,5S,6R,7S,8E,10S,11S,12Z,14E)-6-hydroxy-21-[[(2S)-1-hydroxypropan-2-yl]amino]-5,11-
dimethoxy-3,7,9,15-tetramethyl-16,20,22-trioxo-17-azabicyclo[16.3.1]docosa-1(21),8,12,14,18-pentaen-
10-yl] carbamate
3-[[(3R,5S,6R,7S,8E,10S,11S,12Z,14E)-10-carbamoyloxy-6-hydroxy-5,11-dimethoxy-3,7,9,15-
tetramethyl-16,20,22-trioxo-17-azabicyclo[16.3.1]docosa-1(21),8,12,14,18-pentaen-21-
yl]amino]propanoic acid
[(3R,5S,6R,7S,8E,10S,11S,12Z,14E)-21-(2,3-dihydroxypropylamino)-6-hydroxy-5,11-dimethoxy-
3,7,9,15-tetramethyl-16,20,22-trioxo-17-azabicyclo[16.3.1]docosa-1(21),8,12,14,18-pentaen-10-yl]
carbamate
[(3R,5S,6R,7S,8E,10S,11S,12Z,14E)-21-[(2-amino-2-oxoethyl)amino]-6-hydroxy-5,11-dimethoxy-
3,7,9,15-tetramethyl-16,20,22-trioxo-17-azabicyclo[16.3.1]docosa-1(21),8,12,14,18-pentaen-10-yl]
carbamate
[(3R,5S,6R,7S,8E,10S,11S,12Z,14E)-21-[[(2S)-2,3-dihydroxypropyl]amino]-6-hydroxy-5,11-dimethoxy-
3,7,9,15-tetramethyl-16,20,22-trioxo-17-azabicyclo[16.3.1]docosa-1(21),8,12,14,18-pentaen-10-yl]
carbamate
[(3R,5S,6R,7S,8E,10S,11S,12Z,14E)-6-hydroxy-5,11-dimethoxy-3,7,9,15-tetramethyl-21-[2-
(methylamino)ethylamino]-16,20,22-trioxo-17-azabicyclo[16.3.1]docosa-1(21),8,12,14,18-pentaen-10-yl]
carbamate
[(3R,5S,6R,7S,8E,10S,11S,12Z,14E)-21-[(4-amino-4-methylpentyl)amino]-6-hydroxy-5,11-dimethoxy-
3,7,9,15-tetramethyl-16,20,22-trioxo-17-azabicyclo [16.3.1]docosa-1(21),8,12,14,18-pentaen-10-yl]
carbamate
[(3R,5S,6R,7S,8E,10S,11S,12E,14Z)-21-[[(2S)-2,3-dihydroxypropyl]amino]-6-hydroxy-5,11-dimethoxy-
3,7,9,15-tetramethyl-16,20,22-trioxo-17-azabicyclo[16.3.1]docosa-1(21),8,12,14,18-pentaen-10-yl]
carbamate
[(3R,5S,6R,7S,8E,10S,11S,12E,14Z)-6-hydroxy-5,11-dimethoxy-3,7,9,15-tetramethyl-21-[2-
(methylamino)ethylamino]-16,20,22-trioxo-17-azabicyclo[16.3.1]docosa-1(21),8,12,14,18-pentaen-10-yl]
carbamate
[(3R,5S,6R,7S,8E,10S,11S,12E,14Z)-21-[(4-amino-4-methylpentyl)amino]-6-hydroxy-5,11-dimethoxy-
3,7,9,15-tetramethyl-16,20,22-trioxo-17-azabicyclo[16.3.1]docosa-1(21),8,12,14,18-pentaen-10-yl]
carbamate
[(3R,5S,6R,7S,8E,10S,11S,12E,14Z)-6-hydroxy-21-(3-iodopropylamino)-5,11-dimethoxy-3,7,9,15-
tetramethyl-16,20,22-trioxo-17-azabicyclo[16.3.1]docosa-1(21),8,12,14,18-pentaen-10-yl] carbamate
[(3R,5S,6R,7S,8E,10S,11S,12E,14Z)-6-hydroxy-5,11-dimethoxy-21-(2-methoxyethylamino)-3,7,9,15-
tetramethyl-16,20,22-trioxo-17-azabicyclo[16.3.1]docosa-1(21),8,12,14,18-pentaen-10-yl] carbamate
[(3R,5S,6R,7S,8E,10S,11S,12E,14Z)-6-hydroxy-21-[[(2S)-1-hydroxypropan-2-yl]amino]-5,11-
dimethoxy-3,7,9,15-tetramethyl-16,20,22-trioxo-17-azabicyclo[16.3.1]docosa-1(21),8,12,14,18-pentaen-
10-yl] carbamate
3-[[(3R,5S,6R,7S,8E,10S,11S,12E,14Z)-10-carbamoyloxy-6-hydroxy-5,11-dimethoxy-3,7,9,15-
tetramethyl-16,20,22-trioxo-17-azabicyclo[16.3.1]docosa-1(21),8,12,14,18-pentaen-21-
yl]amino]propanoic acid
[(3R,5S,6R,7S,8E,10S,11S,12E,14Z)-21-(2,3-dihydroxypropylamino)-6-hydroxy-5,11-dimethoxy-
3,7,9,15-tetramethyl-16,20,22-trioxo-17-azabicyclo[16.3.1]docosa-1(21),8,12,14,18-pentaen-10-yl]
carbamate
[(3R,5S,6R,7S,8E,10S,11S,12E,14Z)-6-hydroxy-9-(hydroxymethyl)-5,11-dimethoxy-3,7,15-trimethyl-
16,20,22-trioxo-21-(prop-2-enylamino)-17-azabicyclo[16.3.1]docosa-1(21),8,12,14,18-pentaen-10-yl]
carbamate
[(3R,5S,6R,7S,8E,10S,11S,12E,14Z)-21-[(2-amino-2-oxoethyl)amino]-6-hydroxy-5,11-dimethoxy-
3,7,9,15-tetramethyl-16,20,22-trioxo-17-azabicyclo[16.3.1]docosa-1(21),8,12,14,18-pentaen-10-yl]
carbamate
[(3R,5S,6R,7S,8E,10S,11S,12E,14Z)-21-(cyclopropylamino)-6-hydroxy-5,11-dimethoxy-3,7,9,15-
tetramethyl-16,20,22-trioxo-17-azabicyclo[16.3.1]docosa-1(21),8,12,14,18-pentaen-10-yl] carbamate
[(3R,5S,6R,7S,8E,10S,11S,12E,14Z)-6-hydroxy-21-[[(2R)-1-hydroxypropan-2-yl]amino]-5,11-
dimethoxy-3,7,9,15-tetramethyl-16,20,22-trioxo-17-azabicyclo[16.3.1]docosa-1(21),8,12,14,18-pentaen-
10-yl] carbamate
[(8Z,12Z,14E)-21-(cyclopropylamino)-6-hydroxy-5,11-dimethoxy-3,7,9,15-tetramethyl-16,20,22-trioxo-
17-azabicyclo[16.3.1]docosa-1(21),8,12,14,18-pentaen-10-yl] carbamate
[(3R,5S,6R,7S,8E,10S,11S,12Z,14E)-21-(cyclopropylmethylamino)-6-hydroxy-5,11-dimethoxy-3,7,9,15-
tetramethyl-16,20,22-trioxo-17-azabicyclo[16.3.1]docosa-1(21),8,12,14,18-pentaen-10-yl] carbamate
[(3R,5S,6R,7S,8E,10S,11S,12Z,14E)-6-hydroxy-21-(1-hydroxypropan-2-ylamino)-5,11-dimethoxy-
3,7,9,15-tetramethyl-16,20,22-trioxo-17-azabicyclo[16.3.1]docosa-1(21),8,12,14,18-pentaen-10-yl]
carbamate
[(3R,5S,6R,7S,8E,10S,11S,14E)-6-hydroxy-5,11-dimethoxy-3,7,9,15-tetramethyl-16,20,22-trioxo-21-
(propylamino)-17-azabicyclo[16.3.1]docosa-1(21),8,14,18-tetraen-10-yl] carbamate
[(3R,5S,6R,7S,8E,10S,11S,14E)-21-(ethylamino)-6-hydroxy-5,11-dimethoxy-3,7,9,15-tetramethyl-
16,20,22-trioxo-17-azabicyclo[16.3.1]docosa-1(21),8,14,18-tetraen-10-yl] carbamate
[(8E,14E)-21-(ethylamino)-6-hydroxy-5,11-dimethoxy-3,7,9,15-tetramethyl-16,20,22-trioxo-17-
azabicyclo[16.3.1]docosa-1(21),8,14,18-tetraen-10-yl] carbamate
[(3R,5S,6R,7S,8E,10S,11S,12Z,14E)-21-[3-(dimethylamino)propylamino]-6-hydroxy-5,11-dimethoxy-
3,7,9,15-tetramethyl-16,20,22-trioxo-17-azabicyclo[16.3.1]docosa-1(21),8,12,14,18-pentaen-10-yl]
carbamate
[(3R,5S,6R,7S,8E,10S,11S,12Z,14E)-21-(cyclopentylamino)-6-hydroxy-5,11-dimethoxy-3,7,9,15-
tetramethyl-16,20,22-trioxo-17-azabicyclo[16.3.1]docosa-1(21),8,12,14,18-pentaen-10-yl] carbamate
[(3S,5R,6S,7R,8E,10R,11R,12Z,14E)-21-[3-[2-(3-aminopropoxy)ethoxy]propylamino]-6-hydroxy-5,11-
dimethoxy-3,7,9,15-tetramethyl-16,20,22-trioxo-17-azabicyclo[16.3.1]docosa-1(21),8,12,14,18-pentaen-
10-yl] carbamate
[(3R,5S,6R,7S,8E,10S,11S,12Z,14E)-21-[(3-amino-3-methylbutyl)amino]-6-hydroxy-5,11-dimethoxy-
3,7,9,15-tetramethyl-16,20,22-trioxo-17-azabicyclo[16.3.1]docosa-1(21),8,12,14,18-pentaen-10-yl]
carbamate
[(3R,5S,6R,7S,8E,10S,11S,12E,14Z)-21-[(3-amino-3-methylbutyl)amino]-6-hydroxy-5,11-dimethoxy-
3,7,9,15-tetramethyl-16,20,22-trioxo-17-azabicyclo[16.3.1]docosa-1(21),8,12,14,18-pentaen-10-yl]
carbamate
[(3R,5S,6R,7S,8E,10S,11S,12E,14Z)-21-[3-(dimethylamino)propylamino]-6-hydroxy-5,11-dimethoxy-
3,7,9,15-tetramethyl-16,20,22-trioxo-17-azabicyclo[16.3.1]docosa-1(21),8,12,14,18-pentaen-10-yl]
carbamate
[(3R,5S,6R,7S,8E,10S,11S,12E,14Z)-21-(cyclopentylamino)-6-hydroxy-5,11-dimethoxy-3,7,9,15-
tetramethyl-16,20,22-trioxo-17-azabicyclo[16.3.1]docosa-1(21),8,12,14,18-pentaen-10-yl] carbamate
[(3S,5R,6S,7R,8E,10R,11R,12E,14Z)-21-[3-[2-(3-aminopropoxy)ethoxy]propylamino]-6-hydroxy-5,11-
dimethoxy-3,7,9,15-tetramethyl-16,20,22-trioxo-17-azabicyclo[16.3.1]docosa-1(21),8,12,14,18-pentaen-
10-yl] carbamate
[(3R,5S,6R,7S,8E,10S,11S,12Z,14E)-21-[(3-amino-2-hydroxypropyl)amino]-6-hydroxy-5,11-dimethoxy-
3,7,9,15-tetramethyl-16,20,22-trioxo-17-azabicyclo[16.3.1]docosa-1(21),8,12,14,18-pentaen-10-yl]
carbamate
[(3R,5S,6R,7S,8E,10S,11S,12Z,14E)-21-(2-fluoroethylamino)-6-hydroxy-5,11-dimethoxy-3,7,9,15-
tetramethyl-16,20,22-trioxo-17-azabicyclo[16.3.1]docosa-1(21),8,12,14,18-pentaen-10-yl]N-
hydroxycarbamate
[(3R,5S,6R,7S,8E,10S,11S,12Z,14E)-6-hydroxy-5,11-dimethoxy-3,7,9,15-tetramethyl-16,20,22-trioxo-
21-(prop-2-ynylamino)-17-azabicyclo[16.3.1]docosa-1(21),8,12,14,18-pentaen-10-yl] carbamate
[(3R,5S,6R,7S,8E,10S,11S,12E,14Z)-6-hydroxy-5,11-dimethoxy-3,7,9,15-tetramethyl-16,20,22-trioxo-
21-(prop-2-ynylamino)-17-azabicyclo[16.3.1]docosa-1(21),8,12,14,18-pentaen-10-yl] carbamate
[(3R,5S,6R,7S,8E,10S,11S,12Z,14E)-6-hydroxy-5,11-dimethoxy-3,7,9,15-tetramethyl-16,20,22-trioxo-
19,21-bis(propylamino)-17-azabicyclo[16.3.1]docosa-1(21),8,12,14,18-pentaen-10-yl] carbamate
[(3R,5S,6R,7S,8E,10S,11S,14E)-6-hydroxy-5,11-dimethoxy-3,7,9,15-tetramethyl-16,20,22-trioxo-21-
(propan-2-ylamino)-17-azabicyclo[16.3.1]docosa-1(21),8,14,18-tetraen-10-yl] carbamate
[(3R,5S,6R,7S,8E,10S,11S,12Z,14E)-21-(dimethylamino)-6-hydroxy-5,11-dimethoxy-3,7,9,15-
tetramethyl-16,20,22-trioxo-17-azabicyclo[16.3.1]docosa-1(21),8,12,14,18-pentaen-10-yl] carbamate
[(3R,5S,6R,7S,8E,10S,11S,12Z,14E)-21-acetamido-6-hydroxy-5,11-dimethoxy-3,7,9,15-tetramethyl-
16,20,22-trioxo-17-azabicyclo[16.3.1]docosa-1(21),8,12,14,18-pentaen-10-yl] carbamate
[(3R,5S,6R,7S,8E,10S,11S,12Z,14E)-6-hydroxy-5,11-dimethoxy-3,7,9,15-tetramethyl-16,20,22-trioxo-
21-(propanoylamino)-17-azabicyclo[16.3.1]docosa-1(21),8,12,14,18-pentaen-10-yl] carbamate
[(8Z,12Z,14Z)-6-hydroxy-5,7,9,11,15-pentamethyl-16,20,22-trioxo-21-(prop-2-enylamino)-17-
azabicyclo[16.3.1]docosa-1(21),8,12,14,18-pentaen-10-yl] carbamate
[(3R,5S,6R,7S,8E,10S,11S,12E,14Z)-21-acetamido-6-hydroxy-5,11-dimethoxy-3,7,9,15-tetramethyl-
16,20,22-trioxo-17-azabicyclo[16.3.1]docosa-1(21),8,12,14,18-pentaen-10-yl] carbamate
[(3R,5S,6R,7S,8E,10S,11S,12E,14Z)-6-hydroxy-5,11-dimethoxy-3,7,9,15-tetramethyl-16,20,22-trioxo-
21-(propanoylamino)-17-azabicyclo[16.3.1]docosa-1(21),8,12,14,18-pentaen-10-yl] carbamate
[(3R,5R,6S,7R,10R,11R)-21-[2-(dimethylamino)ethylamino]-6-hydroxy-5,11-dimethoxy-3,7,9,15-
tetramethyl-16,20,22-trioxo-17-azabicyclo[16.3.1]docosa-1(21),8,12,14,18-pentaen-10-yl] carbamate
[21[2-(dimethylamino)ethylamino]-6-hydroxy-5,11-dimethoxy-3,7,9,15-tetramethyl-16,20,22-trioxo-17-
azabicyclo[16.3.1]docosa-1(21),8,12,14,18-pentaen-10-yl] carbamate
[(3R,5S,6R,7S,8E,10S,11S,12Z,14E)-21-[2-(dimethylamino)ethylamino]-6-hydroxy-5,11-dimethoxy-
3,7,9,15-tetramethyl-16,20,22-trioxo-17-azabicyclo[16.3.1]docosa-1(21),8,12,14,18-pentaen-10-yl]
carbamate
[(3R,5R,6S,7R,8E,10R,11R,12Z,14E)-21-[2-(dimethylamino)ethylamino]-6-hydroxy-5,11-dimethoxy-
3,7,9,15-tetramethyl-16,20,22-trioxo-17-azabicyclo[16.3.1]docosa-1(21),8,12,14,18-pentaen-10-yl]
carbamate
[(8Z,12Z,14Z)-21-[2-(dimethylamino)ethylamino]-6-hydroxy-5,11-dimethoxy-3,7,9,15-tetramethyl-
16,20,22-trioxo-17-azabicyclo[16.3.1]docosa-1(21),8,12,14,18-pentaen-10-yl] carbamate
[(8E,12Z,14E)-21-[2-(dimethylamino)ethylamino]-6-hydroxy-5,11-dimethoxy-3,7,9,15-tetramethyl-
16,20,22-trioxo-17-azabicyclo[16.3.1]docosa-1(21),8,12,14,18-pentaen-10-yl] carbamate
[(3R,5S,6R,7S,8E,10S,11S,12Z,14E)-21-[4-[[(3R,5S,6R,7S,8E,10S,11S,12Z,14E)-10-carbamoyloxy-6-
hydroxy-5,11-dimethoxy-3,7,9,15-tetramethyl-16,20,22-trioxo-17-azabicyclo[16.3.1]docosa-
1(21),8,12,14,18-pentaen-21-yl]amino]butylamino]-6-hydroxy-5,11-dimethoxy-3,7,9,15-tetramethyl-
16,20,22-trioxo-17-azabicyclo[16.3.1]docosa-1(21),8,12,14,18-pentaen-10-yl] carbamate
[(3R,5S,6R,7S,8E,10S,11S,12Z,14E)-21-(2-acetamidoethylamino)-6-hydroxy-5,11-dimethoxy-3,7,9,15-
tetramethyl-16,20,22-trioxo-17-azabicyclo[16.3.1]docosa-1(21),8,12,14,18-pentaen-10-yl] carbamate
[(3R,5S,6R,7S,8E,10S,11S,12Z,14E)-21-(2-fluoroethylamino)-6-hydroxy-9-(hydroxymethyl)-5,11-
dimethoxy-3,7,15-trimethyl-16,20,22-trioxo-17-azabicyclo[16.3.1]docosa-1(21),8,12,14,18-pentaen-10-
yl] carbamate
[(3R,5S,6R,7S,8E,10S,11S,12Z,14E)-6-hydroxy-5,11-dimethoxy-3,7,9,15-tetramethyl-16,20,22-trioxo-
21-(piperidin-4-ylmethylamino)-17-azabicyclo[16.3.1]docosa-1(21),8,12,14,18-pentaen-10-yl] carbamate
[(3R,5S,6R,7S,8E,10S,11S,12Z,14E)-21-[(2-amino-2-methylpropyl)amino]-6-hydroxy-5,11-dimethoxy-
3,7,9,15-tetramethyl-16,20,22-trioxo-17-azabicyclo[16.3.1]docosa-1(21),8,12,14,18-pentaen-10-yl]
carbamate
[(3R,5S,6R,7S,8E,10S,11S,12Z,14E)-21-[[(E)-4-[[(3R,5S,6R,7S,8E,10S,11S,12Z,14E)-10-
carbamoyloxy-6-hydroxy-5,11-dimethoxy-3,7,9,15-tetramethyl-16,20,22-trioxo-17-
azabicyclo[16.3.1]docosa-1(21),8,12,14,18-pentaen-21-yl]amino]but-2-enyl]amino]-6-hydroxy-5,11-
dimethoxy-3,7,9,15-tetramethyl-16,20,22-trioxo-17-azabicyclo[16.3.1]docosa-1(21),8,12,14,18-pentaen-
10-yl] carbamate
[(3R,5S,6R,7S,8E,10S,11S,12Z,14E)-21-[2-(diethylamino)ethylamino]-6-hydroxy-5,11-dimethoxy-
3,7,9,15-tetramethyl-16,20,22-trioxo-17-azabicyclo[16.3.1]docosa-1(21),8,12,14,18-pentaen-10-yl]
carbamate
[(3R,5S,6R,7S,8E,10S,11S,12E,14Z)-21-[(2-amino-2-methylpropyl)amino]-6-hydroxy-5,11-dimethoxy-
3,7,9,15-tetramethyl-16,20,22-trioxo-17-azabicyclo[16.3.1]docosa-1(21),8,12,14,18-pentaen-10-yl]
carbamate
[(3S,5R,6S,7S,8Z,10S,11S,12E,14E)-21-[2-(dimethylamino)ethylamino]-6-hydroxy-5,11-dimethoxy-
3,7,9,15-tetramethyl-16,20,22-trioxo-17-azabicyclo[16.3.1]docosa-1(21),8,12,14,18-pentaen-10-yl]
carbamate
[(3S,5S,6R,7R,8Z,10R,11R,12E,14E)-21-[2-(dimethylamino)ethylamino]-6-hydroxy-5,11-dimethoxy-
3,7,9,15-tetramethyl-16,20,22-trioxo-17-azabicyclo[16.3.1]docosa-1(21),8,12,14,18-pentaen-10-yl]
carbamate
[(3R,5R,6S,7R,8E,10R,11R,12E,14Z)-21-[2-(dimethylamino)ethylamino]-6-hydroxy-5,11-dimethoxy-
3,7,9,15-tetramethyl-16,20,22-trioxo-17-azabicyclo[16.3.1]docosa-1(21),8,12,14,18-pentaen-10-yl]
carbamate
[(3R,5R,6S,7R,8E,10S,11R,12E,14Z)-21-[2-(dimethylamino)ethylamino]-6-hydroxy-5,11-dimethoxy-
3,7,9,15-tetramethyl-16,20,22-trioxo-17-azabicyclo[16.3.1]docosa-1(21),8,12,14,18-pentaen-10-yl]
carbamate
[(3R,5S,6R,7S,8E,10S,11S,12E,14Z)-21-(2-fluoroethylamino)-6-hydroxy-9-(hydroxymethyl)-5,11-
dimethoxy-3,7,15-trimethyl-16,20,22-trioxo-17-azabicyclo[16.3.1]docosa-1(21),8,12,14,18-pentaen-10-
yl] carbamate
[(3R,5S,6R,7S,8E,10S,11S,12E,14Z)-6-hydroxy-5,11-dimethoxy-3,7,9,15-tetramethyl-16,20,22-trioxo-
21-(piperidin-4-ylmethylamino)-17-azabicyclo[16.3.1]docosa-1(21),8,12,14,18-pentaen-10-yl] carbamate
[(3R,5S,6R,7S,8E,10S,11S,12E,14Z)-21-(2-acetamidoethylamino)-6-hydroxy-5,11-dimethoxy-3,7,9,15-
tetramethyl-16,20,22-trioxo-17-azabicyclo[16.3.1]docosa-1(21),8,12,14,18-pentaen-10-yl] carbamate
[(3R,5S,6R,7S,8E,10S,11S,12E,14Z)-21-[2-(dimethylamino)ethylamino]-6-hydroxy-5,11-dimethoxy-
3,7,9,15-tetramethyl-16,20,22-trioxo-17-azabicyclo[16.3.1]docosa-1(21),8,12,14,18-pentaen-10-yl]
carbamate
[(3R,5S,6R,7S,8E,10S,11S,12Z,14E)-21-[4-[[(3R,5S,6R,7S,8E,10S,11S,12E,14Z)-10-carbamoyloxy-6-
hydroxy-5,11-dimethoxy-3,7,9,15-tetramethyl-16,20,22-trioxo-17-azabicyclo[16.3.1]docosa-
1(21),8,12,14,18-pentaen-21-yl]amino]butylamino]-6-hydroxy-5,11-dimethoxy-3,7,9,15-tetramethyl-
16,20,22-trioxo-17-azabicyclo[16.3.1]docosa-1(21),8,12,14,18-pentaen-10-yl] carbamate
[(3R,5S,6S,7S,105,11R)-21-[2-(dimethylamino)ethylamino]-6-hydroxy-5,11-dimethoxy-3,7,9,15-
tetramethyl-16,20,22-trioxo-17-azabicyclo[16.3.1]docosa-1(21),8,12,14,18-pentaen-10-yl] carbamate
[(3R,5R,6S,75,8E,10S,11S,12Z,14E)-21-[2-(dimethylamino)ethylamino]-6-hydroxy-5,11-dimethoxy-
3,7,9,15-tetramethyl-16,20,22-trioxo-17-azabicyclo[16.3.1]docosa-1(21),8,12,14,18-pentaen-10-yl]
carbamate
[(3S,5R,6R,7R,8Z,10S,11S,12Z,14Z)-21-[2-(dimethylamino)ethylamino]-6-hydroxy-5,11-dimethoxy-
3,7,9,15-tetramethyl-16,20,22-trioxo-17-azabicyclo[16.3.1]docosa-1(21),8,12,14,18-pentaen-10-yl]
carbamate; N,N-dimethylmethanamine
[(3S,5R,6R,7R,8Z,10S,11S,12Z,14Z)-21-[2-(dimethylamino)ethylamino]-6-hydroxy-5,11-dimethoxy-
3,7,9,15-tetramethyl-16,20,22-trioxo-17-azabicyclo[16.3.1]docosa-1(21),8,12,14,18-pentaen-10-yl]
carbamate
[(3S,5R,6S,7R,8E,10S,11R,12E,14E)-21-[2-(dimethylamino)ethylamino]-6-hydroxy-5,11-dimethoxy-
3,7,9,14-tetramethyl-16,20,22-trioxo-17-azabicyclo[16.3.1]docosa-1(21),8,12,14,18-pentaen-10-yl]
carbamate
[(3R,5S,6R,7S,8E,10S,11S,12E,14E)-21-[4-[[(3R,5S,6R,7S,8E,10S,11S,12E,14E)-10-carbamoyloxy-6-
hydroxy-5,11-dimethoxy-3,7,9,15-tetramethyl-16,20,22-trioxo-17-azabicyclo[16.3.1]docosa-
1(21),8,12,14,18-pentaen-21-yl]amino]butylamino]-6-hydroxy-5,11-dimethoxy-3,7,9,15-tetramethyl-
16,20,22-trioxo-17-azabicyclo[16.3.1]docosa-1(21),8,12,14,18-pentaen-10-yl] carbamate
[(3R,5S,6R,7S,8E,10S,11S,12E,14E)-21-[12-[[(3R,5S,6R,7S,8E,10S,11S,12E,14E)-10-carbamoyloxy-6-
hydroxy-5,11-dimethoxy-3,7,9,15-tetramethyl-16,20,22-trioxo-17-azabicyclo[16.3.1]docosa-
1(21),8,12,14,18-pentaen-21-yl]amino]dodecylamino]-6-hydroxy-5,11-dimethoxy-3,7,9,15-tetramethyl-
16,20,22-trioxo-17-azabicyclo[16.3.1]docosa-1(21),8,12,14,18-pentaen-10-yl] carbamate
[(3R,5S,6R,7S,8E,10S,11S,12E,14E)-21-[16-[[(3R,5S,6R,7S,8E,10S,11S,12E,14E)-10-carbamoyloxy-6-
hydroxy-5,11-dimethoxy-3,7,9,15-tetramethyl-16,20,22-trioxo-17-azabicyclo[16.3.1]docosa-
1(21),8,12,14,18-pentaen-21-yl]amino]hexadecylamino]-6-hydroxy-5,11-dimethoxy-3,7,9,15-
tetramethyl-16,20,22-trioxo-17-azabicyclo[16.3.1]docosa-1(21),8,12,14,18-pentaen-10-yl] carbamate
[(3R,5R,6S,7R,8E,10S,11S,12Z,14E)-21-[2-(dimethylamino)ethylamino]-6-hydroxy-5,11-dimethoxy-
3,7,9,15-tetramethyl-16,20,22-trioxo-17-azabicyclo[16.3.1]docosa-1(21),8,12,14,18-pentaen-10-yl]
carbamate
[(3S,5R,6R,7R,8Z,10R,11R,12Z,14Z)-21-[2-(dimethylamino)ethylamino]-6-hydroxy-5,11-dimethoxy-
3,7,9,15-tetramethyl-16,20,22-trioxo-17-azabicyclo[16.3.1]docosa-1(21),8,12,14,18-pentaen-10-yl]
carbamate
[(3R,5S,6R,7S,8E,10S,11S,12Z,14E)-19,21-bis[3-(dimethylamino)propylamino]-6-hydroxy-5,11-
dimethoxy-3,7,9,15-tetramethyl-16,20,22-trioxo-17-azabicyclo[16.3.1]docosa-1(21),8,12,14,18-pentaen-
10-yl] carbamate
[(3R,5S,6R,7S,8E,10S,11S,12E,14Z)-19,21-bis[3-(dimethylamino)propylamino]-6-hydroxy-5,11-
dimethoxy-3,7,9,15-tetramethyl-16,20,22-trioxo-17-azabicyclo[16.3.1]docosa-1(21),8,12,14,18-pentaen-
10-yl] carbamate
[(3R,5S,6R,7S,8E,10S,11S,12Z,14E)-6-hydroxy-19,21-bis(3-hydroxypropylamino)-5,11-dimethoxy-
3,7,9,15-tetramethyl-16,20,22-trioxo-17-azabicyclo[16.3.1]docosa-1(21),8,12,14,18-pentaen-10-yl]
carbamate
[(3R,5S,6R,7S,8E,10S,11S,12Z,14E)-6-hydroxy-9-(hydroxymethyl)-5,11-dimethoxy-3,7,15-trimethyl-
21-(methylamino)-16,20,22-trioxo-17-azabicyclo[16.3.1]docosa-1(21),8,12,14,18-pentaen-10-yl]
carbamate
[(3R,5S,6R,7S,8E,10S,11S,12E,14Z)-6-hydroxy-9-(hydroxymethyl)-5,11-dimethoxy-3,7,15-trimethyl-
21-(methylamino)-16,20,22-trioxo-17-azabicyclo[16.3.1]docosa-1(21),8,12,14,18-pentaen-10-yl]
carbamate
[(3R,5S,6R,7S,8E,10S,11S,12Z,14E)-21-(2-fluoroethylamino)-6-hydroxy-5,11-dimethoxy-3,7,9,15-
tetramethyl-16-methylidene-20,22-dioxo-17-azabicyclo[16.3.1]docosa-1(21),8,12,14,18-pentaen-10-yl]
carbamate
[(3R,5S,6R,7S,8E,10S,11S,14E)-21-(2-fluoroethylamino)-6-hydroxy-5,11-dimethoxy-3,7,9,15-
tetramethyl-16,20,22-trioxo-17-azabicyclo[16.3.1]docosa-1(21),8,14,18-tetraen-10-yl]carbamate
[(3R,5S,6R,7S,8E,10S,11S,14E)-6-hydroxy-21-[[(2R)-2-hydroxypropyl]amino]-5,11-dimethoxy-
3,7,9,15-tetramethyl-16,20,22-trioxo-17-azabicyclo[16.3.1]docosa-1(21),8,14,18-tetraen-10-yl]
carbamate
[(3R,5S,6R,7S,8E,10S,11S,12Z,14E)-21-amino-6-hydroxy-5,11-dimethoxy-3,7,9,15-tetramethyl-
16,20,22-trioxo-17-(2-oxopropyl)-17-azabicyclo[16.3.1]docosa-1(21),8,12,14,18-pentaen-10-yl]
carbamate
[(3R,5S,6R,7S,8E,10S,11S,12E,14Z)-21-amino-6-hydroxy-5,11-dimethoxy-3,7,9,15-tetramethyl-
16,20,22-trioxo-17-(2-oxopropyl)-17-azabicyclo[16.3.1]docosa-1(21),8,12,14,18-pentaen-10-yl]
carbamate
[(3R,5S,6S,7S,8E,10S,11S,12E,14Z)-21-(2-fluoroethylamino)-6-hydroxy-5,11-dimethoxy-3,7,9,15-
tetramethyl-16-methylidene-20,22-dioxo-17-azabicyclo[16.3.1]docosa-1(21),8,12,14,18-pentaen-10-yl]
carbamate
[(3R,5S,6R,7S,8E,10S,11S,14E)-6-hydroxy-21-(2-hydroxyethylamino)-5,11-dimethoxy-3,7,9,15-
tetramethyl-16,20,22-trioxo-17-azabicyclo[16.3.1]docosa-1(21),8,14,18-tetraen-10-yl] carbamate
[(3R,5S,6R,7S,8E,10S,11S,14E)-6-hydroxy-21-(2-hydroxypropylamino)-5,11-dimethoxy-3,7,9,15-
tetramethyl-16,20,22-trioxo-17-azabicyclo[16.3.1]docosa-1(21),8,14,18-tetraen-10-yl] carbamate
[(3R,5S,6R,7S,8E,10S,11S,12Z,14E)-6-hydroxy-5,11-dimethoxy-3,7,9,15-tetramethyl-19,21-
bis(methylamino)-16,20,22-trioxo-17-azabicyclo[16.3.1]docosa-1(21),8,12,14,18-pentaen-10-yl]
carbamate
(21-amino-6-hydroxy-5,11-dimethoxy-3,7,9,15-tetramethyl-16,20,22-trioxo-17-
azabicyclo[16.3.1]docosa-1(21),8,12,14,18-pentaen-10-yl) carbamate
[(3R,5R,6S,7R,8E,10R,11R,12Z,14E)-21-amino-6-hydroxy-5,11-dimethoxy-3,7,9,15-tetramethyl-
16,20,22-trioxo-17-azabicyclo[16.3.1]docosa-1(21),8,12,14,18-pentaen-10-yl] carbamate
[(8E,12Z,14E)-21-amino-6-hydroxy-5,11-dimethoxy-3,7,9,15-tetramethyl-16,20,22-trioxo-17-
azabicyclo[16.3.1]docosa-1(21),8,12,14,18-pentaen-10-yl] carbamate
[(8Z,12Z,14Z)-21-amino-6-hydroxy-5,11-dimethoxy-3,7,9,15-tetramethyl-16,20,22-trioxo-17-
azabicyclo[16.3.1]docosa-1(21),8,12,14,18-pentaen-10-yl] carbamate
[(8Z,12E,14E)-6-hydroxy-5,11-dimethoxy-3,7,9,15-tetramethyl-16,20,22-trioxo-17-
azabicyclo[16.3.1]docosa-1(21),8,12,14,18-pentaen-10-yl] carbamate
[(3R,5R,6S,7S,10S,11R)-21-amino-6-hydroxy-5,11-dimethoxy-3,7,9,15-tetramethyl-16,20,22-trioxo-17-
azabicyclo[16.3.1]docosa-1(21),8,12,14,18-pentaen-10-yl] carbamate
[(3R,5S,6R,7S,8E,10S,11S,12Z,14E)-21-amino-6-hydroxy-5,11-dimethoxy-3,7,9,15-tetramethyl-
16,20,22-trioxo-17-azabicyclo[16.3.1]docosa-1(21),8,12,14,18-pentaen-10-yl] carbamate
[(3R,5S,6R,7S,8E,10S,11S,12Z,14E)-6-hydroxy-5,11-dimethoxy-3,7,9,15-tetramethyl-16,20,22-trioxo-
17-azabicyclo[16.3.1]docosa-1(21),8,12,14,18-pentaen-10-yl] carbamate
ethyl N-[(3R,5S,6R,7S,8E,10S,11S,12Z,14E)-10-carbamoyloxy-6-hydroxy-5,11-dimethoxy-3,7,9,15-
tetramethyl-16,20,22-trioxo-17-azabicyclo[16.3.1]docosa-1(21),8,12,14,18-pentaen-21-yl]carbamate
[(3R,5S,6R,7S,8E,10S,11S,12Z,14E)-6-hydroxy-5,11-dimethoxy-3,7,9,15-tetramethyl-16,20,22-trioxo-
21-prop-2-enyl-17-azabicyclo[16.3.1]docosa-1(21),8,12,14,18-pentaen-10-yl] carbamate
[(3R,5R,6S,7R,8Z,10R,11R,12Z,14Z)-21-amino-6-hydroxy-5,11-dimethoxy-3,7,9,15-tetramethyl-
16,20,22-trioxo-17-azabicyclo[16.3.1]docosa-1(21),8,12,14,18-pentaen-10-yl] carbamate
[(3R,5R,6R,7R,8E,10R,11R,12E,14Z)-21-amino-6-hydroxy-5,11-dimethoxy-3,7,9,15-tetramethyl-
16,20,22-trioxo-17-azabicyclo[16.3.1]docosa-1(21),8,12,14,18-pentaen-10-yl] carbamate
ethyl N-[(3R,5S,6R,7S,8E,10S,11S,12E,14Z)-10-carbamoyloxy-6-hydroxy-5,11-dimethoxy-3,7,9,15-
tetramethyl-16,20,22-trioxo-17-azabicyclo[16.3.1]docosa-1(21),8,12,14,18-pentaen-21-yl]carbamate
[(3R,5S,6R,7S,8E,10S,11S,12E,14Z)-6-hydroxy-5,11-dimethoxy-3,7,9,15-tetramethyl-16,20,22-trioxo-
17-azabicyclo[16.3.1]docosa-1(21),8,12,14,18-pentaen-10-yl] carbamate
[(3R,5S,6R,7S,8E,10S,11S,12E,14Z)-21-amino-6-hydroxy-5,11-dimethoxy-3,7,9,15-tetramethyl-
16,20,22-trioxo-17-azabicyclo[16.3.1]docosa-1(21),8,12,14,18-pentaen-10-yl] carbamate
[(3R,5S,6R,7S,8E,10S,11S,12Z,14E)-21-ethenyl-6-hydroxy-5,11-dimethoxy-3,7,9,15-tetramethyl-
16,20,22-trioxo-17-azabicyclo[16.3.1]docosa-1(21),8,12,14,18-pentaen-10-yl] carbamate
[(3R,5R,6S,7S,8E,10S,11S,12Z,14E)-21-amino-6-hydroxy-5,11-dimethoxy-3,7,9,15-tetramethyl-
16,20,22-trioxo-17-azabicyclo[16.3.1]docosa-1(21),8,12,14,18-pentaen-10-yl] carbamate
[(8E,12Z,14E)-6-hydroxy-5,11-dimethoxy-3,7,9,15-tetramethyl-16,20-dioxo-21-(prop-2-enylamino)-17-
azabicyclo[16.3.1]docosa-1(21),8,12,14,18-pentaen-10-yl] carbamate
[(3R,5S,6R,7S,8E,10S,11S,14E)-6-hydroxy-5,11-dimethoxy-3,7,9,15-tetramethyl-21-(methylamino)-
16,20,22-trioxo-17-azabicyclo[16.3.1]docosa-1(21),8,14,18-tetraen-10-yl] carbamate
[(3R,5S,6R,7S,8E,10S,11S,12Z,14E)-6-hydroxy-5,11-dimethoxy-3,7,9,15-tetramethyl-16,20,22-trioxo-
21-(2-oxopropanoylamino)-17-azabicyclo[16.3.1]docosa-1(21),8,12,14,18-pentaen-10-yl] carbamate
[(3R,5S,6R,7S,8E,10S,11S,12Z,14E)-21-azanyl-6-hydroxy-5,11-dimethoxy-3,7,9,15-tetramethyl-
16,20,22-trioxo-17-azabicyclo[16.3.1]docosa-1(21),8,12,14,18-pentaen-10-yl] carbamate
[(8E,12E,14Z)-6-hydroxy-5,11-dimethoxy-3,7,9,15-tetramethyl-16,20,22-trioxo-17-
azabicyclo[16.3.1]docosa-1(21),8,12,14,18-pentaen-10-yl] carbamate

Several HSP90 inhibitors are commercially available. Standard doses of HSP90 inhibitors are known in the art (e.g., 0.5 mg, 1.0 mg, 2.0 mg, 3.0 mg, 4.0 mg, 5.0 mg, 7.5 mg, 10 mg, 15 mg, 20 mg, 25 mg, 50 mg, 75 mg, 100 mg, 125 mg, 150 mg, 175 mg, 200 mg, 225 mg, 250 mg, 275 mg, 300 mg, 350 mg, 400 mg, 450 mg, 500 mg, 550 mg, 600 mg, 650 mg, 700 mg, 750 mg, 800 mg, 850 mg, 900 mg, or any range between any pair of recited doses) and can range, e.g., from 0.1 mg to 300 mg (e.g., 0.1 mg to 200 mg, 0.1 mg to 150 mg, 0.1 mg to 100 mg, and 1.0 mg to 50 mg) for each individual HSP90 inhibitor.

### 3-(4-octadecyl)benzoylacrylic acid (OBAA)

The methods, compositions, and kits of the invention may employ OBAA or OBAA analog that exhibit phospholipase A2 inhibitory activity. A variety of OBAA analogs are known in the art (e.g., darapladib, varespladib, and SB-480848).

Additional OBAA analogs are listed in Table 2.

TABLE 2
OBAA analogs

(E)-4-(3-octadecan-4-ylphenyl)-4-oxobut-2-enoic acid
(E)-3-(3-acetylphenyl)prop-2-enoic acid
(E)-4-(3-methylphenyl)-4-oxobut-2-enoic acid
4-(3-methylphenyl)-4-oxobut-2-enoic acid
(E)-3-(3-benzoyl-5-ethylphenyl)prop-2-enoic acid
(E)-4-(2-nonylphenyl)-4-oxobut-2-enoic acid
(Z)-4-(4-dodecylphenyl)-4-oxobut-2-enoic acid
(Z)-4-(4-hexadecylphenyl)-3-methyl-4-oxobut-2-enoic acid
(Z)-4-oxo-4-(4-tetradecylphenyl)but-2-enoic acid
(Z)-3-methyl-4-(4-octadecylphenyl)-4-oxobut-2-enoic acid
(Z)-4-(4-dodecylphenyl)-3-methyl-4-oxobut-2-enoic acid
(Z)-4-(4-hexadecylphenyl)-4-oxobut-2-enoic acid
(Z)-3-methyl-4-oxo-4-(4-tetradecylphenyl)but-2-enoic acid
(Z)-4-(4-octadecylphenyl)-4-oxobut-2-enoic acid
(E)-4-(4-dodecylphenyl)-4-oxobut-2-enoic acid
(E)-4-(4-octadecylphenyl)-4-oxobut-2-enoic acid
4-(4-dodecylphenyl)-4-oxobut-2-enoic acid
4-(4-octadecylphenyl)-4-oxobut-2-enoic acid
(Z)-4-(3-benzoylphenyl)-2-methylbut-2-enoic acid
(E)-4-(4-hexylphenyl)-4-oxobut-2-enoic acid
(E)-3-[4-(3-methylbutanoyl)phenyl]prop-2-enoic acid
(E)-3-[4-(3-methyl-2-methylidenebutanoyl)phenyl]prop-2-enoic acid
(Z)-4-(4-hexylphenyl)-4-oxobut-2-enoic acid
(E)-2-methyl-4-(4-octadecylphenyl)-4-oxobut-2-enoic acid
(E)-4-(4-hexadecylphenyl)-2-methyl-4-oxobut-2-enoic acid
(E)-4-(4-dodecylphenyl)-2-methyl-4-oxobut-2-enoic acid
(E)-2-methyl-4-oxo-4-(4-tetradecylphenyl)but-2-enoic acid
(Z)-4-(4-octylphenyl)-4-oxobut-2-enoic acid
(E)-4-oxo-4-(2,3,5,6-tetramethylphenyl)but-2-enoic acid
(E)-4-oxo-4-(4-propan-2-ylphenyl)but-2-enoic acid
4-oxo-4-(2,3,5,6-tetramethylphenyl)but-2-enoic acid
4-oxo-4-(4-propan-2-ylphenyl)but-2-enoic acid
(E)-3-[3-[(E)-3-oxo-3-phenylprop-1-enyl]phenyl]prop-2-enoic acid
(E)-3-methyl-4-oxo-4-phenylbut-2-enoic acid
(Z)-3-methyl-4-oxo-4-phenylbut-2-enoic acid
(E)-3-(3-acetylphenyl)prop-2-enoate
(4,4-dihydroxy-3,5-dimethylcyclohexa-2,5-dien-1-yl)-(3,5-dimethylphenyl)methanone
(Z)-4-(2,5-dimethylphenyl)-4-oxobut-2-enoic acid
(E)-4-(3,4-dimethylphenyl)-4-oxobut-2-enoic acid
(E)-4-(3-methylphenyl)-4-oxobut-2-enoate
(E)-3-[3-[(E)-3-phenylprop-2-enoyl]phenyl]prop-2-enoic acid
(E)-4-(4-cyclohexylphenyl)-4-oxobut-2-enoic acid
(E)-4-oxo-4-(2,4,6-trimethylphenyl)but-2-enoic acid
(E)-4-(2,4-dimethylphenyl)-4-oxobut-2-enoic acid
(E)-4-(2,5-dimethylphenyl)-4-oxobut-2-enoic acid
4-(4-cyclohexylphenyl)-4-oxobut-2-enoic acid
4-(2,5-dimethylphenyl)-4-oxobut-2-enoic acid
4-oxo-4-(2,4,6-trimethylphenyl)but-2-enoic acid
4-(2,4-dimethylphenyl)-4-oxobut-2-enoic acid
(E)-3-methyl-4-(4-methylphenyl)-4-oxobut-2-enoic acid
(Z)-3-methyl-4-(4-methylphenyl)-4-oxobut-2-enoic acid
(E)-4-(4-ethylphenyl)-4-oxobut-2-enoic acid
(E)-6-(3-hexanoylphenyl)hex-5-enoic acid
(Z)-2,3-dimethyl-4-oxo-4-phenylbut-2-enoic acid
2-methylbut-3-enoic acid; (E)-4-oxo-4-phenylbut-2-enoic acid
(E)-3-(4-butanoylphenyl)prop-2-enoic acid
(E)-3-(4-propanoylphenyl)prop-2-enoic acid
(E)-3-[4-(2-methylprop-2-enoyl)phenyl]prop-2-enoic acid
(E)-3-[4-(2-methylidenebutanoyl)phenyl]prop-2-enoic acid
(E)-3-[3-methyl-4-(2-phenylprop-2-enoyl)phenyl]prop-2-enoic acid
(E)-3-(2-acetylphenyl)prop-2-enoic acid
(E)-3-(4-acetylphenyl)prop-2-enoic acid
(E)-3-[4-[2,4-di(propan-2-yl)benzoyl]phenyl]-2-methylprop-2-enoic acid
(E)-4-(2-methylphenyl)-4-oxobut-2-enoic acid
(E)-2-methyl-4-oxo-4-phenylbut-2-enoic acid
(2E)-2-(7-hexadecyl-1-oxo-3,4-dihydronaphthalen-2-ylidene)acetic acid
(2E)-2-(1-oxo-7-tetradecyl-3,4-dihydronaphthalen-2-ylidene)acetic acid
(E)-3-[3-[(2,4,5-trimethyl-3,6-dioxocyclohexa-1,4-dien-1-yl)methyl]phenyl]prop-2-enoic acid
(2E)-2-(7-dodecyl-1-oxo-3,4-dihydronaphthalen-2-ylidene)acetic acid
(E)-4-(4-butylphenyl)-2-methyl-4-oxobut-2-enoic acid
(Z)-6-(3-hexanoylphenyl)hex-5-enoic acid
4-(4-tert-butylphenyl)-4-oxobut-2-enoic acid
(Z)-4-(4-methylphenyl)-4-oxobut-2-enoic acid
(E)-4-(4-methylphenyl)-4-oxobut-2-enoic acid
4-(4-methylphenyl)-4-oxobut-2-enoic acid
(E)-4-(4-tert-butylphenyl)-4-oxobut-2-enoic acid

OBAA and several OBAA analogs are commercially available. Standard doses of OBAA and several OBAA analogs are known in the art and can range from 0.1 mg to 300 mg (e.g., 0.1 mg to 200 mg, 0.1 mg to 150 mg, 0.1 mg to 100 mg, and 1.0 mg to 50 mg).

### Flunarizine

The methods, compositions, and kits of the invention may employ flunarizine or a flunarizine analog that has Ca²⁺-channel blockering activity. A variety of flunarizine analogs are known in the art. Nonlimiting examples of flunarizine analogs are cinnarizine and those described in U.S. Pat. No. 3,773,939 (e.g., compounds of general formula (I)); U.S. Pat. No. 3,940,386 (e.g., compounds of general formula (I)), U.S. Pat. No. 4,008,324; (e.g., compounds of general formula (I)); U.S. Pat. No. 4,068,070 (e.g., compounds of formulas 1-11); U.S. Pat. No. 4,703,048 (e.g., compounds of general formula (I)); U.S. Pat. No. 4,882,331 (e.g., compounds of general formula (I)); U.S. Pat. No. 5,371,088 (e.g., compounds of general formula (I)); and U.S. Patent Application Publication No. 2008/0200474 A1 (each of which is incorporated by reference).

Additional flunarizine analogs are set forth in Table 3 below.

TABLE 3
Flunarizine analogs

1-[bis(4-fluorophenyl)methyl]-4-[(E)-3-phenylprop-2-enyl]piperazine
1-[bis(4-fluorophenyl)methyl]-2,2,3,3,5,5,6,6-octadeuterio-4-[(E)-3-phenylprop-2-enyl]piperazine
1-[bis(4-fluorophenyl)methyl]-4-[(Z)-3-phenylprop-2-enyl]piperazine
1-[bis(4-fluorophenyl)methyl]-4-(3-phenylprop-2-enyl)piperazine
1-[bis(4-fluorophenyl)methyl]-4-[(2E,4E)-5-phenylpenta-2,4-dienyl]piperazine
1-benzyl-4-[bis(4-fluorophenyl)methyl]piperazine
1-[bis(4-fluorophenyl)methyl]-4-(3-phenylpropyl)piperazine
1-[bis(4-fluorophenyl)methyl]-4-[(3,4-difluorophenyl)methyl]piperazine
1-[bis(4-fluorophenyl)methyl]-4-[(2,4-difluorophenyl)methyl]piperazine
1-[bis(4-fluorophenyl)methyl]-4-[(E)-3-phenylprop-2-enyl]piperazine chloride
1-benzhydryl-4-[(3-fluorophenyl)methyl]piperazine
1,4-dibenzyl-2-[2-(2,5-difluorophenyl)ethyl]piperazine
1-[bis(4-fluorophenyl)methyl]-4-[(Z)-3-phenylprop-2-enyl]piperazine hydrochloride
1,2-bis[(4-fluorophenyl)methyl]-4-[(E)-3-phenylprop-2-enyl]piperazine
1-[bis(4-fluorophenyl)methyl]-4-[(E)-3-phenylprop-2-enyl]piperazine hydrochloride
1-[bis(4-fluorophenyl)methyl]-4-[(E)-3-phenylprop-2-enyl]piperazine; hydron; chloride
1-benzhydryl-4-[(2-fluorophenyl)methyl]piperazine
1-benzhydryl-4-[(4-fluorophenyl)methyl]piperazine
1-[bis(4-fluorophenyl)methyl]-4-[(E)-3-phenylprop-2-enyl]piperazine dichloride
1,4-dibenzyl-2-[2-(4-fluorophenyl)ethyl]piperazine
1,4-dibenzyl-2-[2-(3-fluorophenyl)ethyl]piperazine
1,4-dibenzyl-2-[2-(2-fluorophenyl)ethyl]piperazine
1-benzyl-4-[3,3-bis(4-fluorophenyl)prop-2-enyl]piperazine
1-[bis(4-fluorophenyl)methyl]-4-[(E)-3-phenylprop-2-enyl]piperazine dihydrochloride
1-[bis(4-fluorophenyl)methyl]-4-(3-phenylprop-2-enyl)piperazine dihydrochloride
MW: 477.416726 g/mol|MF: C26H28Cl2F2N2
1-[bis(4-fluorophenyl)methyl]-4-[(3-methylphenyl)methyl]piperazine
MW: 392.484146 g/mol|MF: C25H26F2N2
1-[bis(4-fluorophenyl)methyl]-4-[(E)-2-thenylethenyl]piperazine
1-[4-(2-fluorophenyl)cyclohex-3-en-1-yl]-4-[(4-fluorophenyl)methyl]piperazine
1-[4-(4-fluorophenyl)cyclohex-3-en-1-yl]-4-[(4-fluorophenyl)methyl]piperazine
1-benzyl-2,2-bis(4-fluorophenyl)-4-prop-2-enylpiperazine
1-benzhydryl-2,2,3,3,5,5,6,6-octadeuterio-4-[(E)-3-phenylprop-2-enyl]piperazine
1-[bis(4-fluorophenyl)methyl]-4-(2-piperidin-1-ylethyl)piperazine
6-benzhydryl-N-[(4-fluorophenyl)methyl]-N,1-dimethylpiperidin-3-amine
1-[(2,5-difluorophenyl)methyl]-4-[4-(4-fluorophenyl)cyclohexyl]piperazine
1-[bis(4-fluorophenyl)methyl]-4-propan-2-ylpiperazine
1-[1-[4-(3,4-difluorophenyl)phenyl]ethyl]-4-[(E)-3-phenylprop-2-enyl]piperazine
1-[1-[4-(2,4-difluorophenyl)phenyl]ethyl]-4-[(E)-3-phenylprop-2-enyl]piperazine
1-benzhydryl-4-[[3-(trifluoromethyl)phenyl]methyl]piperazine
1-benzyl-4-[4-(4-fluorophenyl)cyclohex-3-en-1-yl]piperazine
1,4-dibenzyl-2-[2-[4-(trifluoromethyl)phenyl]ethyl]piperazine
1,4-dibenzyl-2-[1-(4-fluorophenyl)ethyl]piperazine
1,4-dibenzyl-2-[2-[3-(trifluoromethyl)phenyl]ethyl]piperazine
1-[bis(4-fluorophenyl)methyl]-4-methylpiperazine
1-benzhydryl-4-(1-phenylprop-2-enyl)piperazine
1-benzyl-4-[3,3-bis(4-fluorophenyl)prop-2-enyl]piperazine dihydrochloride
(2S)-1,4-dibenzyl-2-[2-[3-(trifluoromethyl)phenyl]ethyl]piperazine
1-[bis(4-fluorophenyl)methyl]-4-pentylpiperazine
1-benzhydryl-4-[(2E,4E)-5-phenylpenta-2,4-dienyl]piperazine
1-benzyl-4-[2,2-bis(4-fluorophenyl)ethyl]piperazine
(3R,6R)-6-benzhydryl-N-[(4-fluorophenyl)methyl]-N,1-dimethylpiperidin-3-amine
1-[bis(4-fluorophenyl)methyl]-4-butylpiperazine
1-benzhydryl-4-[(Z)-3-phenylprop-2-enyl]piperazine
1-benzhydryl-4-[(E)-3-phenylprop-2-enyl]piperazine
1-benzhydryl-4-benzylpiperazine
1-benzhydryl-4-(3-phenylprop-2-enyl)piperazine
1-benzhydryl-4-(1-phenylethyl)piperazine
1-benzhydryl-4-benzylpiperazine hydrochloride
1-[4,4-bis(4-fluorophenyl)butyl]-4-[(3,4-difluorophenyl)methyl]piperazine
1-[bis(4-fluorophenyl)methyl]-4-[(E)-3-phenylprop-2-enyl]piperazin-4-ium
1-[1-[4-(4-fluorophenyl)phenyl]ethyl]-4-[(E)-3-phenylprop-2-enyl]piperazine
1-[1-[4-(2-fluorophenyl)phenyl]ethyl]-4-[(E)-3-phenylprop-2-enyl]piperazine
1-benzyl-4-[4-(4-fluorophenyl)cyclohexyl]piperazine
1-benzyl-2,2-bis(4-fluorophenyl)-4-prop-2-enylpiperazine dihydrochloride
1-[(E)-3-(4-fluorophenyl)prop-2-enyl]-4-[iodo(diphenyl)methyl]piperazine
1-benzhydryl-4-[(E)-3-phenylprop-2-enyl]piperazine hydrochloride
1,4-dibenzyl-2-(2-phenylethyl)piperazine
1,4-dibenzyl-2-[2-[2-(trifluoromethyl)phenyl]ethyl]piperazine
MW: 438.527770 g/mol|MF: C27H29F3N2
1-[bis(4-fluorophenyl)methyl]-4-tert-butylpiperazine
1-[bis(4-fluorophenyl)methyl]-4-[(E)-3-phenylprop-2-enyl]piperazine; dichloromethane
(2R,3R,5S)-5-benzyl-1,4-dimethyl-2,3-diphenylpiperazine
1-benzyl-4-[2,2-bis(4-fluorophenyl)ethyl]piperazine hydrochloride
1-benzyl-4-[bis(4-fluorophenyl)methylidene]piperidine
1-benzhydryl-4-(4-phenylbutyl)piperazine
1-[(1R)-1,2-diphenylethyl]-4-(4-phenylbutyl)piperazine
1-[(1R)-1,2-diphenylethyl]-4-(3-phenylpropyl)piperazine
1-benzhydryl-4-(1-phenylpropan-2-yl)piperazine
1-[bis(4-fluorophenyl)methyl]-4-[(E)-3-phenylprop-2-enyl]piperazine-1,4-diium
1-[(4-fluorophenyl)methyl]-4-(4-phenylcyclohexyl)piperazine
1-[(3-fluorophenyl)methyl]-4-(4-phenylcyclohexyl)piperazine
1-[(2-fluorophenyl)methyl]-4-(4-phenylcyclohexyl)piperazine
5-benzyl-1,4-dimethyl-2,3-diphenylpiperazine
1,4-dibenzyl-2-phenylpiperazine
1-[bis(4-fluorophenyl)methyl]-4-(3-phenylprop-2-enyl)piperazine-1,4-diium
1-benzhydryl-4-(1-phenylethyl)piperazine hydrochloride
1-[bis(4-fluorophenyl)methyl]-4-prop-2-ynylpiperazine
1-benzyl-4-[(2R)-1-(2-fluorophenyl)propan-2-yl]piperazine
1-benzyl-4-[(2S)-1-(2-fluorophenyl)propan-2-yl]piperazine
1-benzyl-4-[(2R)-1-(4-fluorophenyl)propan-2-yl]piperazine
1-benzyl-4-[(2S)-1-(4-fluorophenyl)propan-2-yl]piperazine
1-benzhydryl-4-[(4-fluorophenyl)methyl]piperazin-4-ium
1-benzhydryl-4-[(2-fluorophenyl)methyl]piperazin-4-ium
1-[bis(4-fluorophenyl)methyl]-4-[(2Z)-2-(6,7,8,9-tetrahydrobenzo[7]annulen-5-ylidene)ethyl]piperazine
1-[1-[4-[4-(difluoromethyl)phenyl]phenyl]ethyl]-4-[(E)-3-phenylprop-2-enyl]piperazine
1-[(E)-3-phenylprop-2-enyl]-4-[1-[4-[4-(trifluoromethyl)phenyl]phenyl]ethyl]piperazine
2-[3-[(4-fluorophenyl)methyl]phenyl]-1-[(E)-3-phenylprop-2-enyl]piperidine
N,N,1-tribenzyl-3-(fluoromethyl)piperidin-3-amine
1-[bis(4-fluorophenyl)methyl]piperidine
1,4-dibenzyl-2-[(E)-2-phenylethenyl]piperazine
1-[bis(4-fluorophenyl)methyl]-4-but-2-ynylpiperazine
2-(4-fluorophenyl)-1-[(4-fluorophenyl)methyl]piperidine
1-benzyl-4-(4,4-diphenylbut-3-enyl)piperazine
3-[4-[bis(2-fluorophenyl)methyl]piperazin-1-yl]propanenitrile
1-[bis(4-fluorophenyl)methyl]-4-(2-pyrrolidin-1-ylethyl)piperazine
1,4-dibenzyl-2,6-bis(fluoromethyl)piperazine
(7Z)-7-benzylidene-2-[bis(4-fluorophenyl)methyl]-3,4,6,8,9,9a-hexahydro-1H-pyrido[1,2-a]pyrazine
N-benzhydryl-2-[1-[(4-fluorophenyl)methyl]piperidin-4-yl]-N-methylethanamine
2-benzyl-7-[bis(4-fluorophenyl)methylidene]-3,4,6,8,9,9a-hexahydro-1H-pyrido[1,2-a]pyrazine
(7E)-7-benzylidene-2-[bis(4-fluorophenyl)methyl]-3,4,6,8,9,9a-hexahydro-1H-pyrido[1,2-a]pyrazine
3-[4-[bis(4-fluorophenyl)methyl]piperazin-1-yl]propanenitrile
1-benzhydryl-4-[(2-fluorophenyl)methyl]piperazine-1,4-diium
1-benzhydryl-4-[(4-fluorophenyl)methyl]piperazine-1,4-diium
MW: 362.482983 g/mol|MF: C24H27FN2+2
1-benzhydryl-4-[(E)-3-phenylprop-2-enyl]piperazine dihydrochloride
1-cyclohexyl-4-[(1R)-1,2-diphenylethyl]piperazine
1-cyclohexyl-4-[(1S)-1,2-diphenylethyl]piperazine
1-benzyl-4-[1-(4-fluorophenyl)propan-2-yl]piperazine
1-benzyl-4-[1-(2-fluorophenyl)propan-2-yl]piperazine
(4-benzhydrylpiperazin-1-yl)-(4-fluorophenyl)methanethione
1-cyclohexyl-4-(1,2-diphenylethyl)piperazine
1-[1-[1-(4-fluorophenyl)ethyl]piperidin-4-yl]-4-[(4-fluorophenyl)methyl]piperazine
10,11-dibenzyl-4-fluoro-11-azaspiro[5.5]undecane
1-[2-(3-fluorophenyl)-1-phenylethyl]piperidine
1-[phenyl-(4-phenylphenyl)methyl]-4-prop-2-enylpiperazine
1-[(3,4-difluorophenyl)methyl]-4-[(4-fluorophenyl)methyl]piperazine
1-[(3,4-difluorophenyl)methyl]-4-[(2-fluorophenyl)methyl]piperazine
1-[1-(4-fluorophenyl)-2-phenylethyl]piperidine
2-benzhydryl-4-benzyl-1-methylpiperazine
2-benzhydryl-1-methyl-4-[[2-(trifluoromethyl)phenyl]methyl]piperazine
1-benzyl-4-[4-[4-(trifluoromethyl)phenyl]cyclohex-3-en-1-yl]piperazine
1-[(4-fluorophenyl)methyl]-4-[4-[4-(trifluoromethyl)phenyl]cyclohex-3-en-1-yl]piperazine
1-benzyl-4-(4-benzylcyclohex-3-en-1-yl)piperazine
1-benzyl-2-[2-(4-fluorophenyl)ethyl]piperidine
2-(4-fluorophenyl)-1-(2-phenylethyl)piperidine
1-benzhydryl-4-[(3-methylphenyl)methyl]piperazine
1-[2-(4-fluorophenyl)ethyl]-2-phenylpiperidine
1,4-dibenzyl-2-[(E)-4-phenylbut-3-enyl]piperazine
1-benzhydryl-4-methanidylpiperazine
1-benzyl-4-(4,4-diphenylbut-3-enyl)piperazine hydrochloride
1-[(4-fluorophenyl)-phenylmethyl]piperidine
1-benzhydryl-4-methylidene-2-phenylpiperidine
1-benzyl-4-[4,4-bis(4-fluorophenyl)butylidene]piperidine
MW: 417.533366 g/mol|MF: C28II29F2N
1-benzyl-4-[2-(3-fluorophenyl)ethyl]piperazine
5-[4-[bis(4-fluorophenyl)methyl]piperazin-1-yl]pentan-1-amine
4-[4-[bis(4-fluorophenyl)methyl]piperazin-1-yl]butan-1-amine
2-[4-[bis(4-fluorophenyl)methyl]piperazin-1-yl]ethanamine
1-benzhydryl-4-(2-phenylpropan-2-yl)piperazine
2-benzyl-1-[(4-fluorophenyl)methyl]piperidine
1,4-dibenzyl-2-(trifluoromethyl)piperazine
2-[4-(3-fluorophenyl)phenyl]-1-[(E)-3-phenylprop-2-enyl]piperidine
4-benzyl-2-[1-(4-fluorophenyl)cyclohexyl]-1-propylpiperazine
1-[4,4-bis(2-fluorophenyl)but-3-enyl]-4-[2-(4-fluorophenyl)-2-(4-propan-2-ylpiperazin-1-
yl)ethyl]piperazine
1-[4,4-bis(4-fluorophenyl)but-3-enyl]-4-[2-(4-fluorophenyl)-2-(4-propan-2-ylpiperazin-1-
yl)ethyl]piperazine
1-[5,5-bis(4-fluorophenyl)pent-4-enyl]-4-[2-(4-fluorophenyl)-2-(4-propan-2-ylpiperazin-1-
yl)ethyl]piperazine
1-[4,4-bis(3-fluorophenyl)but-3-enyl]-4-[2-(4-fluorophenyl)-2-(4-propan-2-ylpiperazin-1-
yl)ethyl]piperazine
4-[[4-[bis(4-fluorophenyl)methyl]piperazin-1-yl]methyl]benzonitrile
1,4-dibenzyl-2-(fluoromethyl)piperazine
1-benzyl-4-[bis(4-fluorophenyl)methyl]piperidine
1-[bis(4-fluorophenyl)methyl]-4-[(2E)-2-(3,4-dihydro-2H-naphthalen-1-ylidene)ethyl]piperazine
(2S)-1-benzyl-4-[(1R)-1-phenylethyl]-2-(2-phenylethyl)piperazine
N,N,N′,N′-tetraethyl-1,2-bis(3-fluorophenyl)ethane-1,2-diamine
(3R,6R)-6-benzhydryl-N-[(4-fluorophenyl)methyl]-N,1-dimethylpiperidin-3-amine dihydrochloride
1-benzyl-4-methylidene-2,6-diphenylpiperidine
1-benzyl-4-[(E)-1,1,1-trifluoro-4-phenylbut-3-en-2-yl]piperazine
N-benzyl-2-[4-[bis(4-fluorophenyl)methylidene]piperidin-1-yl]ethanamine
1-(1-benzhydrylazetidin-3-yl)-4-benzylpiperazine
1-cyclohexyl-4-[(1R)-1,2-diphenylethyl]piperazine hydrochloride
4-[bis(4-fluorophenyl)methyl]-1-aza-4-azoniabicyclo[2.2.2]octane
1-[(3-fluorophenyl)methyl]-4-(3-phenylpropyl)piperazine
1-[(3,4-difluorophenyl)methyl]-4-[(E)-3-phenylprop-2-enyl]piperazine
1-benzhydryl-4-[(E)-3-phenylprop-1-enyl]piperazine
1-benzhydryl-4-[(4-methylphenyl)methyl]piperazine
1-benzhydryl-4-[(4-ethylphenyl)methyl]piperazine
3-[[4-[bis(4-fluorophenyl)methyl]piperazin-1-yl]methyl]benzonitrile
1-[(3,4-difluorophenyl)methyl]-4-(3-phenylprop-2-enyl)piperazine
2-[4-[4,4-bis(4-fluorophenyl)butyl]-2-phenylpiperazin-1-yl]ethanamine
1-(1-benzylpiperidin-4-yl)-4-(4-fluorophenyl)-3,6-dihydro-2H-pyridine
1-benzyl-4-[(2,6-difluorophenyl)methyl]piperazine
1,4-dibenzhydrylpiperazine
1-benzhydryl-4-(3-phenylprop-2-ynyl)piperazine
1,4-bis[(2-fluorophenyl)methyl]piperazine
1,4-bis[(3-fluorophenyl)methyl]piperazine
1,4-bis[(4-fluorophenyl)methyl]piperazine
1-[(2-fluorophenyl)methyl]-4-[(3-fluorophenyl)methyl]piperazine
1-[(2-fluorophenyl)methyl]-4-[(4-fluorophenyl)methyl]piperazine
1-[(3-fluorophenyl)methyl]-4-[(4-fluorophenyl)methyl]piperazine
1-cyclohexyl-4-[(3-fluorophenyl)methyl]piperazine
1-[(2,6-difluorophenyl)methyl]-4-[(E)-3-phenylprop-2-enyl]piperazine
1-benzyl-4-[(3,4-difluorophenyl)methyl]piperazine
1-[(2,4-difluorophenyl)methyl]-4-[(E)-3-phenylprop-2-enyl]piperazine
1-[(2,4-difluorophenyl)methyl]-4-(3-phenylprop-2-enyl)piperazine
1-cyclohexyl-4-[(4-fluorophenyl)methy]piperazine
1-cyclohexyl-4-[(2-fluorophenyl)methyl]piperazine
1-benzyl-4-[(2,4-difluorophenyl)methyl]piperazine
1-[(2,6-difluoropheyl)methyl]-4-(3-phenylprop-2-enyl)piperazine
1-benzyl-4-[(2,5-difluorophenyl)methyl]piperazine
1-[(2,5-difluorophenyl)methyl]-4-[(E)-3-phenylprop-2-enyl]piperazine
1-[(2,5-difluorophenyl)methyl]-4-(3-phenylprop-2-enyl)piperazine
1-[(4-fluorophenyl)methyl]-4-(3-phenylpropyl)piperazine
1-[(2-fluorophenyl)methyl]-4-(3-phenylpropyl)piperazine
N,N,N′,N′-tetraethyl-1,2-bis(4-fluorophenyl)ethane-1,2-diamine
1-(1,2-diphenylethyl)-4-prop-2-enylpiperazine
1-benzyl-1,4-bis(1-phenylpropan-2-yl)piperazin-1-ium
1-benzhydryl-4-prop-2-enylpiperazine
1-benzhydryl-4-(3-phenylprop-1-enyl)piperazine
1-[1-(4-fluorophenyl)ethyl]-4-[1-[1-(4-fluorophenyl)ethyl]piperidin-4-yl]piperazine
(1S,2R)-2-(4-benzylpiperazin-1-yl)-1,2-diphenylethanamine
1-cyclohcxyl-4-[(2-phenylphenyl)methyl]piperazine
2-[[4-[bis(4-fluorophenyl)methyl]piperazin-1-yl]methyl]benzonitrile
1-(3,3-diphenylpropyl)-4-(1-phenylethyl)piperazine
1-(1-phenylethyl)-4-[(2-phenylphenyl)methyl]piperazine
1-methyl-2-phenyl-4-[(2-phenylphenyl)methyl]piperazine
2-(2,4-difluorophenyl)-N,N-diethyl-2-(4-methylpiperazin-1-yl)ethanamine
2-(2,6-difluorophenyl)-N,N-diethyl-2-(4-methylpiperazin-1-yl)ethanamine
6-benzhydryl-N-[(3,4-difluorophenyl)methyl]piperidin-3-amine
1-[deuterio(diphenyl)methyl]-4-(trideuteriomethyl)piperazine
2,2,6,6-tetradeuterio-4-[deuterio(diphenyl)methyl]-1-methylpiperazine
4-benzhydryl-2,2,6,6-tetradeuterio-1-methylpiperazine
2,2,6,6-tetradeuterio-1-[deuterio(diphenyl)methyl]-4-methylpiperazine
1-[deuterio(diphenyl)methyl]-4-methylpiperazine
1-benzhydryl-2,2,6,6-tetradeuterio-4-methylpiperazine
1-benzhydryl-4-(trideuteriomethyl)piperazine
1-benzyl-4-[(2R)-1-[3-(trifluoromethyl)phenyl]propan-2-yl]piperazine
1-benzyl-4-[(2S)-1-[3-(trifluoromethyl)phenyl]propan-2-yl]piperazine
1-[bis(4-fluorophenyl)methyl]piperazin-4-ium
N-methyl-1,2-diphenyl-N-(2-piperidin-1-ylethyl)ethanamine
1-(1,2-diphenylethyl)-4-prop-2-enylpiperazine chloride
1-[(3,4-difluorophenyl)methyl]-4-[(4-ethylphenyl)methyl]piperazine
1-benzyl-4-(2,2-diphenylethyl)piperazine
(3R)-1-benzhydryl-3-fluoropiperidine
(3R)-3-fluoro-1-tritylpiperidine
1-[bis(4-fluorophenyl)methyl]-4-pentylpiperidine
1-[bis(4-fluorophenyl)methyl]-4-methylpiperidine
1-[bis(4-fluorophenyl)methyl]-4-ethylpiperidine
1-benzhydryl-4-methanidylpiperazine; tungsten(2+)
N-[bis(4-fluorophenyl)methyl]-2-piperazin-1-ylethanamine
1-[bis(4-fluorophenyl)methyl]-4-butylpiperidine
4-benzhydryl-2-[(2,3,6-trifluorophenyl)methyl]-3,4,6,7,8,8a-hexahydro-1H-pyrrolo[1,2-a]pyrazine
2-[4-[(4-fluorophenyl)methyl]phenyl]-1-[(E)-3-phenylprop-2-enyl]-3,6-dihydro-2H-pyridine
2-[3-[(4-fluorophenyl)methyl]phenyl]-1-[(E)-3-phenylprop-2-enyl]-3,6-dihydro-2H-pyridine
1-benzyl-4-[3-[1-(4-fluorophenyl)cyclohexyl]propyl]piperazine
1-benzhydryl-4-[(2-methylphenyl)methyl]piperazine
1-benzyl-4-(3,3-diphenylpropyl)piperazine
1-(1,1-diphenylethyl)-4-[(E)-3-phenylprop-2-enyl]piperazine
1-(1-benzhydrylazetidin-3-yl)-4-fluoropiperidine
1-benzyl-4-fluoro-4-[3-(4-fluorophenyl)propyl]piperidine
1-ethyl-4-tritylpiperazine
1-[bis(4-methylphenyl)methyl]-4-[(E)-3-phenylprop-2-enyl]piperazine
N-benzyl-4,4-bis(3-fluorophenyl)-N-(1-phenylethyl)but-3-en-2-amine
(1-benzhydrylpiperidin-2-yl)-phenylmethanamine
1-benzhydryl-4-propylpiperazine
1-benzyl-4-(1-phenylcyclohexyl)piperazine
1-[(4-fluorophenyl)-phenylmethyl]piperidine hydrochloride
1-(4-fluorophenyl)-N-[(4-fluorophenyl)methyl]-1-[1-(2-phenylethyl)piperidin-4-yl]methanamine
1-benzyl-4-[1-[4,4-bis(4-fluorophenyl)butyl]piperidin-4-yl]piperazine
2-benzyl-1-[1-(2-fluorophenyl)ethyl]piperidine
1-[4,4-bis(2-fluorophenyl)but-3-enyl]-4-[2-(4-fluorophenyl)-2-(4-propan-2-ylpiperazin-1-
yl)ethyl]piperazine hydrochloride
1-[4,4-bis(3-fluorophenyl)butyl]-4-[2-(4-fluorophenyl)-2-(4-propan-2-ylpiperazin-1-yl)ethyl]piperazine
1-[4,4-bis(2-fluorophenyl)butyl]-4-[2-(4-fluorophenyl)-2-(4-propan-2-ylpiperazin-1-yl)ethyl]piperazine
1-(4,4-diphenylbut-3-enyl)-4-[2-(4-fluorophenyl)-2-(4-methylpiperazin-1-yl)ethyl]piperazine
1-[4,4-bis(4-fluorophenyl)butyl]-4-[2-(4-fluorophenyl)-2-(4-propan-2-ylpiperazin-1-yl)ethyl]piperazine
3-[4-[bis(4-fluorophenyl)methyl]piperazin-1-yl]propan-1-amine
(2R,3R,5S)-1,4,5-trimethyl-2,3-diphenylpiperazine
1-[bis(4-fluorophenyl)methyl]-4-[(2E)-2-(2,3-dihydroinden-1-ylidene)ethyl]piperazine
1-benzhydryl-4-butylpiperazine
1-[bis(2-fluorophenyl)methyl]piperazine
1-benzhydryl-4-ethylpiperazine
1-cyclohexyl-4-[[3-(2-phenylethyl)phenyl]methyl]piperazine
1,2-bis(4-fluorophenyl)-N,N,N′,N′-tetramethylethane-1,2-diamine
1,2-bis(3-fluorophenyl)-N,N,N,N′-tetramethylethane-1,2-diamine
1-benzyl-4-[(E)-4,4,5,5,5-pentafluoro-1-phenylpent-1-en-3-yl]piperazine
1-cyclohexyl-4-[[4-(2-phenylethyl)phenyl]methyl]piperazine
1,1-bis(4-fluorophenyl)-N-[[1-(2-phenylethyl)piperidin-4-yl]methyl]methanamine
2-(1-benzylpiperidin-4-yl)-N-[bis(4-fluorophenyl)methyl]ethanamine
N[4,4-bis(4-fluorophenyl)butyl]-N′-[(4-fluorophenyl)methyl]-N,N′-dimethylethane-1,2-diamine
(2S,5R)-1-benzhydryl-2,5-dimethyl-4-prop-2-enylpiperazine
1-[(1-benzhydrylazetidin-2-yl)methyl]-4-benzylpiperazine
1-[(1-benzhydrylazetidin-2-yl)methyl]-4-[bis(4-fluorophenyl)methyl]piperazine
N-benzhydryl-2-[1-[(3,4-difluorophenyl)methyl]piperidin-4-yl]ethanamine
1-benzyl-N-[bis(4-fluorophenyl)methyl]piperidin-4-amine
(1-benzylpiperidin-1-ium-4-yl)-[bis(4-fluorophenyl)methyl]azanium
(2R)-3-[4-[bis(4-fluorophenyl)methyl]piperazin-1-yl]-2-methylpropanenitrile
(2S)-3-[4-[bis(4-fluorophenyl)methyl]piperazin-1-yl]-2-methylpropanenitrile
1-[bis(4-fluorophenyl)methyl]piperazine-1,4-diium
1-[(2,4-difluorophenyl)-(3-fluorophenyl)methyl]piperazine
1-[(2,4-difluorophenyl)-phenylmethyl]piperazine
1-[(2-fluorophenyl)-(4-fluorophenyl)methyl]piperazine
1-[(2,4-difluorophenyl)-(4-fluorophenyl)methyl]piperazine
1-[bis(3-fluorophenyl)methyl]piperazine
1-[(2-fluorophenyl)-(3-fluorophenyl)methyl]piperazine
1-[(2,4-difluorophenyl)-(4-phenylphenyl)methyl]piperazine
1-[(3-fluorophenyl)-(4-fluorophenyl)methyl]piperazine
1-[(2,4-difluorophenyl)-(2-fluorophenyl)methyl]piperazine
1-cyclohexyl-4-[(1R)-1,2-diphenylethyl]piperazine dihydrochloride
1-cyclohexyl-4-(1,2-diphenylethyl)piperazine dihydrochloride
1-cyclohexyl-4-[(1S)-1,2-diphenylethyl]piperazine dihydrochloride
2-[4-[4,4-bis(4-fluorophenyl)butyl]-2-phenylpiperazin-1-yl]ethanamine trihydrochloride
4-benzhydryl-1,1-dimethylpiperazin-1-ium
4-benzhydryl-1-methyl-1-propan-2-ylpiperazin-1-ium
4-benzhydryl-1-ethyl-1-propylpiperazin-1-ium
4-benzhydryl-1-ethyl-1-methylpiperazin-1-ium
4-benzhydryl-1-ethyl-1-propan-2-ylpiperazin-1-ium
4-benzhydryl-1,1-diethylpiperazin-1-ium
4-benzhydryl-1-butyl-1-methylpiperazin-1-ium
1-benzyl-4-[1-[3-(trifluoromethyl)phenyl]propan-2-yl]piperazine
1-benzyl-4-(4-phenylcyclohexyl)piperazine
1-(1-benzylpiperidin-4-yl)-4-[(3-fluorophenyl)methyl]piperazine
1-(1-benzylpiperidin-4-yl)-4-[(2-fluorophenyl)methyl]piperazine
1-benzyl-4-[1-[(4-fluorophenyl)methyl]piperidin-4-yl]piperazine
1-benzhydryl-4-(2-methylprop-2-enyl)piperazine
1-(1-benzylpiperidin-4-yl)-4-[(4-fluorophenyl)methyl]piperazine
1-benzyl-4-[(3-fluorophenyl)methyl]piperazine
1-benzyl-4-[(2-fluorophenyl)methyl]piperazine
1-[(4-fluorophenyl)methyl]-4-[(E)-3-phenylprop-2-enyl]piperazine
1-[(4-fluorophenyl)methyl]-4-(3-phenylprop-2-enyl)piperazine
1-[(2-fluorophenyl)methyl]-4-[(E)-3-phenylprop-2-enyl]piperazine
1-[(2-fluorophenyl)methyl]-4-(3-phenylprop-2-enyl)piperazine
1-[(3-fluorophenyl)methyl]-4-[(E)-3-phenylprop-2-enyl]piperazine
1-[(3-fluorophenyl)methyl]-4-(3-phenylprop-2-enyl)piperazine
1-benzyl-4-[(4-fluorophenyl)methyl]piperazine
1,4-bis[(2,6-difluorophenyl)methyl]piperazine
1,4-dimethyl-2,3-diphenylpiperazine
1,4,5-trimethyl-2,3-diphenylpiperazine
1-(1,3-diphenylpropyl)-4-methylpiperazine
1-(1,2-diphenylethyl)-4-methylpiperazine
1-benzyl-1,4-bis(1-phenylpropan-2-yl)piperazin-1-ium bromide
1-[bis(4-fluorophenyl)methyl]piperazine
1-benzhydryl-4-methylpiperazine
2-benzhydryl-6-[(4-fluorophenyl)methyl]-1,6-diazabicyclo[3.3.1]nonane
1-[1-(4-fluorophenyl)ethyl]-4-[1-[1-(4-fluorophenyl)ethyl]piperidin-4-yl]piperazine hydrochloride
1-[1-[1-(4-fluorophenyl)ethyl]piperidin-4-yl]-4-[(4-fluorophenyl)methyl]piperazine hydrochloride
1-benzhydryl-4-[(1E,5E)-1,6-diphenylhexa-1,5-dien-3-yl]piperazine
4-benzhydryl-1,1-bis[(E)-3-phenylprop-2-enyl]piperazin-1-ium
1-[(2,4-difluorophenyl)methyl]-4-(1,2,3,4-tetrahydronaphthalen-2-yl)piperazine
1-(2-phenylethyl)-4-[(2-phenylphenyl)methyl]piperazine
1-[(2-phenylphenyl)methyl]-4-[(E)-3-phenylprop-2-enyl]piperazine
N-benzyl-2-(4-benzylphenyl)-2-(3-fluoropiperazin-1-yl)ethanamine
1-[1-(4-fluorophenyl)-2-[4-[3-[2-(4-fluorophenyl)phenyl]propyl]piperazin-1-yl]ethyl]-4-propan-2-
ylpiperazine
1-[1-(4-fluorophenyl)-2-[4-[3-[2-(3-fluorophenyl)phenyl]propyl]piperazin-1-yl]ethyl]-4-propan-2-
ylpiperazine
1-[1-(4-fluorophenyl)-2-[4-[3-[2-(2-fluorophenyl)phenyl]propyl]piperazin-1-yl]ethyl]-4-propan-2-
ylpiperazine
1-[1-(4-fluorophenyl)-2-[4-[3-(2-fluoro-6-phenylphenyl)propyl]piperazin-1-yl]ethyl]-4-propan-2-
ylpiperazine
1-[1-(4-fluorophenyl)-2-[4-[3-(5-fluoro-2-phenylphenyl)propyl]piperazin-1-yl]ethyl]-4-propan-2-
ylpiperazine
1-[1-(4-fluorophenyl)-2-[4-[3-(4-fluoro-2-phenylphenyl)propyl]piperazin-1-yl]ethyl]-4-propan-2-
ylpiperazine
1-[1-(4-fluorophenyl)-2-[4-[3-(3-fluoro-2-phenylphenyl)propyl]piperazin-1-yl]ethyl]-4-propan-2-
ylpiperazine
N,N-diethyl-2-(2-fluorophenyl)-2-(4-methylpiperazin-1-yl)ethanamine
(2R)-N,N-diethyl-2-(2-fluorophenyl)-2-(4-methylpiperazin-1-yl)ethanamine
(2S)-N,N-diethyl-2-(2-fluorophenyl)-2-(4-methylpiperazin-1-yl)ethanamine
2,2,3,3,5,5,6,6-oetadeuterio-1-[deuterio(diphenyl)methyl]-4-(trideuteriomethyl)piperazine
2,2,3,3,5,5,6,6-octadeuterio-1-[deuterio(diphenyl)methyl]-4-methylpiperazine
2,2,6,6-tetradeuterio-4-[deuterio(diphenyl)methyl]-1-(trideuteriomethyl)piperazine
1-benzhydryl-2,2,3,3,5,5,6,6-oetadeuterio-4-(trideuteriomethyl)piperazine
1-benzhydryl-2,2,3,3,5,5,6,6-oetadeuterio-4-methylpiperazine
1-benzhydryl-2,2,3,3,5,5-hexadeuterio-4-(trideuteriomethyl)piperazine
4-[bis(2,3,4,5,6-pentadeuteriophenyl)methyl]-2,2,6,6-tetradeuterio-1-methylpiperazine
2,2,6,6-tetradeuterio-1-[deuterio(diphenyl)methyl]-4-(trideuteriomethyl)piperazine
1-[deuterio-bis(2,3,4,5,6-pentadeuteriophenyl)methyl]-4-(trideuteriomethyl)piperazine
1-[deuterio-bis(2,3,4,5,6-pentadeuteriophenyl)methyl]-4-methylpiperazine
1-[benzhydryl-2,2,6,6-tetradeuterio-4-(trideuteriomethyl)piperazine
1-[bis(2,3,4,5,6-pentadeuteriophenyl)methyl]-2,2,6,6-tetradeuterio-4-methylpiperazine
1-[bis(2,3,4,5,6-pentadeuteriophenyl)methyl]-4-(trideuteriomethyl)piperazine
(2R)-2-(4-benzylpiperidin-1-yl)-2-(4-fluorophenyl)ethanamine
(2S)-2-(4-benzylpiperidin-1-yl)-2-(4-fluorophenyl)ethanamine
(2R)-2-(4-benzylpiperazin-1-yl)-2-(2-fluorophenyl)ethanamine
(2S)-2-(4-benzylpiperazin-1-yl)-2-(2-fluorophenyl)ethanamine
(2R)-2-(4-benzylpiperazin-1-yl)-2-(4-fluorophenyl)ethanamine
(2S)-2-(4-benzylpiperazin-1-yl)-2-(4-fluorophenyl)ethanamine
(2R)-2-(4-benzylpiperidin-1-yl)-2-(2-fluorophenyl)ethanamine
(2S)-2-(4-benzylpiperidin-1-yl)-2-(2-fluorophenyl)ethanamine
1-[(2R)-1-(2-fluorophenyl)propan-2-yl]-4-[(E)-3-phenylprop-2-enyl]piperazine
1-[(2S)-1-(2-fluorophenyl)propan-2-yl]-4-[(E)-3-phenylprop-2-enyl]piperazine
1-benzhydryl-4-[(Z)-3-phenylprop-2-enyl]piperazin-4-ium
(2R)-2-[4-[(4-fluorophenyl)methyl]piperazin-1-yl]-2-phenylethanamine
(2S)-2-[4-[(4-fluorophenyl)methyl]piperazin-1-yl]-2-phenylethanamine
(2R)-2-[4-[(3-fluorophenyl)methyl]piperazin-1-yl]-2-phenylethanamine
(2S)-2-[4-[(3-fluorophenyl)methyl]piperazin-1-yl]-2-phenylethanamine
(2R)-2-[4-[(2-fluorophenyl)methyl]piperazin-1-yl]-2-phenylethanamine
(2S)-2-[4-[(2-fluorophenyl)methyl]piperazin-1-yl]-2-phenylethanamine
1-[(R)-(4-fluorophenyl)-phenylmethyl]piperazin-4-ium
1-[(S)-(4-fluorophenyl)-phenylmethyl]piperazin-4-ium
1-benzhydryl-4-[(E)-3-phenylprop-2-enyl]piperazin-4-ium
1-benzhydryl-4-benzylpiperazin-4-ium
1-benzyl-4-[(2-phenylphenyl)methyl]piperazine
1-benzhydryl-4-methylpiperazine; hydron; chloride
(2R,5S)-4-benzhydryl-1,1-diethyl-2,5-dimethylpiperazin-1-ium
N-benzyl-1-(2-phenylethyl)-N-(3-phenylpropyl)piperidin-4-amine
3-(4-benzhydrylpiperazin-1-yl)-3-phenylpropan-1-amine
N-benzyl-N-[(2,5-difluorophenyl)methyl]-3-phenylpropan-1-amine
N-[(2,5-difluorophenyl)methyl]-3-phenyl-N-(3-phenylprop-2-enyl)propan-1-amine
1-[1-(2-fluorophenyl)propan-2-yl]-4-(3-phenylprop-2-enyl)piperazine
N,N-bis[(4-fluorophenyl)methyl]-3-phenylpropan-1-amine
N-[(2,5-difluorophenyl)methyl]-N-[(4-fluorophenyl)methyl]-3-phenylpropan-1-amine
N-[(2,5-difluorophenyl)methyl]-3-phenyl-N-(2-phenylethyl)propan-1-amine
1-benzhydryl-4-methylpiperazine chloride
1-[4-[bis(4-fluorophenyl)methyl]piperazin-1-yl]-4-methylpentan-2-amine
1-[bis(4-fluorophenyl)methyl]-4-[(2E)-2-(3,4-dihydro-2H-naphthalen-1-ylidene)ethyl]piperazine
dihydrochloride
2-benzhydryl-4-benzyl-1-methylpiperazine dihydrochloride
2-benzhydryl-1-methyl-4-[[2-(trifluoromethyl)phenyl]methyl]piperazine dihydrochloride
N,N,1-tribenzyl-4-(fluoromethyl)piperidin-4-amine
1,1-bis(4-fluorophenyl)-N-[[1-(1-phenylethyl)piperidin-4-yl]methyl]methanamine
6-benzhydryl-N-[(4-fluorophenyl)methyl]piperidin-3-amine
N-benzhydryl-2-(1-benzylpiperidin-4-yl)-N-methylethanamine
1,5-dibenzyl-4-(4-fluorophenyl)-3,6-dihydro-2H-pyridine
1,3-dibenzyl-4-(4-fluorophenyl)-3,6-dihydro-2H-pyridine
N-[[3,5-bis(trifluoromethyl)phenyl]methyl]-2-phenyl-2-[4-(2-phenylethyl)piperazin-1-yl]ethanamine
1-benzyl-4-[2-(2-fluorophenyl)propyl]piperazine
1,1,4,4-tetramethyl-2,5-diphenylpiperazine-1,4-diium
1-[(1R,2R)-1,2-diphenyl-2-piperidin-1-ylethyl]piperidine
1-[(4-fluorophenyl)methyl]-2-(1-fluoropropyl)-4-phenylpiperidine
1-benzyl-2,3-diphenylpiperazine
N-[(2,5-difluorophenyl)methyl]-2-phenylpiperidin-3-amine
1-[bis(4-fluorophenyl)methyl]-3-methylpiperazine
1-[bis(4-fluorophenyl)methyl]-3,5-dimethylpiperazine
1-benzhydryl-4-(3-methylbutyl)piperazine
1-butyl-4-tritylpiperazine
1-benzhydryl-4-butyl-2,5-dimethylpiperazine
1-[bis(4-fluorophenyl)methyl]-3-butylpiperazine
N-benzyl-3,3-bis(3-fluorophenyl)-N-methylprop-2-en-1-amine
N-benzyl-1-(3-fluorophenyl)-N-(1-phenylethyl)propan-1-amine
1-benzyl-4-(2-fluorophenyl)-4-phenylpiperidine
1,2-bis[(4-fluorophenyl)methyl]piperazine
2-(4-benzylpiperazin-1-yl)-N-[[3,5-bis(trifluoromethyl)phenyl]methyl]-2-phenylethanamine
1-benzyl-4-[1-[4,4-bis(4-fluorophenyl)butyl]piperidin-4-yl]piperazine trihydrochloride
N′-benzyl-N-[4,4-bis(4-fluorophenyl)butyl]-N,N′-dimethylethane-1,2-diamine
N-benzyl-2-(2,3,4-trifluorophenyl)piperidin-3-amine
N-[(2,6-difluorophenyl)methyl]-2-phenylpiperidin-3-amine
2-(4-benzhydrylpiperazin-1-yl)acetonitrile
1-benzyl-4-(5-phenylpentyl)piperazine
2-(4-fluorophenyl)-1-(2-phenylethyl)-4-[3-(trifluoromethyl)phenyl]-3,6-dihydro-2H-pyridine
2-(4-benzhydrylpiperazin-1-yl)-N-benzylethanamine
(2S)-1,4-dibenzyl-2-ethenylpiperazine
1-(4,4-diphenylbutyl)-4-[2-(4-fluorophenyl)-2-(4-methylpiperazin-1-yl)ethyl]piperazine
1-[5,5-bis(4-fluorophenyl)pent-4-enyl]-4-[2-(4-fluorophenyl)-2-(4-propan-2-ylpiperazin-1-
yl)ethyl]piperazine tetrahydrochloride
1-[4,4-bis(3-fluorophenyl)but-3-enyl]-4-[2-(4-fluorophenyl)-2-(4-propan-2-ylpiperazin-1-
yl)ethyl]piperazine tetrahydrochloride
1-[2-(4-fluorophenyl)-2-(4-methylpiperazin-1-yl)ethyl]-4-(4-phenylbutyl)piperazine
1-benzyl-4-(1-phenylethyl)piperazine
1-[bis(4-fluorophenyl)methyl]-4-(3-chloropropyl)piperazine
(2R,5S)-1-[bis(4-fluorophenyl)methyl]-2,5-dimethylpiperazine
1-[bis(4-fluorophenyl)methyl]-2,5-dimethylpiperazine
1-[(1R,2S)-1,2-diphenyl-2-piperidin-1-ylethyl]piperidine
4-benzhydrylidene-1-benzylpiperidine
1-cyclohexyl-4-[2-(4-methylphenyl)-1-phenylethyl]piperazine
1-cyclohexyl-4-[2-(3-methylphenyl)-1-phenylethyl]piperazine
1-(1,2-diphenylethyl)-4-(2-methylpropyl)piperazine
1-cyclohexyl-4-(1,2-diphenylpropyl)piperazine
1-[2-(4-fluorophenyl)-2-(4-methylpiperazin-1-yl)ethyl]-4-(3-phenylpropyl)piperazine
(2R,5R)-2-benzhydryl-6-[(4-fluorophenyl)methyl]-1,6-diazabicyclo[3.3.1]nonane
1[6,6-bis(4-fluorophenyl)hexyl]-4-[[3,5-bis(trifluoromethyl)phenyl]methyl]piperazine
(2S)-N,N-dibenzyl-1-fluoro-3-phenylpropan-2-amine
1-benzyl-4-methyl-2-phenylpiperazine
1,4-dibenzyl-2-ethenylpiperazine
(3S,6S)-6-benzhydryl-N-[(4-fluorophenyl)methyl]piperidin-3-amine
(3R,6R)-6-benzhydryl-N-[(4-fluorophenyl)methyl]piperidin-3-amine
1-benzyl-4-[(3,4-difluorophenyl)methyl]piperidine
1-benzyl-4-[(2,6-difluorophenyl)methyl]piperidine
N-[[1-[(4-fluorophenyl)methyl]piperidin-4-yl]methyl]-N-methyl-2,2-diphenylethanamine
1-cyclohexyl-4-[[2-(2-phenylethyl)phenyl]methyl]piperazine
N-benzhydryl-2-[1-[(4-fluorophenyl)methyl]piperidin-4-yl]ethanamine
1-benzhydryl-4-phenylpiperidine
(3R)-6-benzhydryl-N-[(4-fluorophenyl)methyl]piperidin-3-amine
1-(1-benzhydrylazetidin-3-yl)-4-benzylpiperidine
1-benzyl-4-[(E)-3-(4-phenylphenyl)prop-2-enyl]piperazine
1-[4-(4-fluorophenyl)cyclohexen-1-yl]-4-[(4-fluorophenyl)methyl]piperazine
1-benzyl-4-[(1R)-1-phenylethyl]piperazine
1-benzyl-4-[(1S)-1-phenylethyl]piperazine
1-[(S)-(4-fluorophenyl)-phenylmethyl]piperazine
1-[(S)-(4-fluorophenyl)-phenylmethyl]piperazine-1,4-diium
1-[(R)-(4-fluorophenyl)-phenylmethyl]piperazine
1-[(R)-(4-fluorophenyl)-phenylmethyl]piperazine-1,4-diium
1-benzhydryl-4-benzylpiperazine-1,4-diium
1-benzhydryl-4-methylpiperazine hydrochloride
1-[(4-phenylphenyl)methyl]-4-[(E)-3-phenylprop-2-enyl]piperazine
1-benzhydryl-4-[(E)-3-phenylprop-2-enyl]piperazine-1,4-diium
1-[(3-fluorophenyl)-phenylmethyl]piperazine
1-[(4-ethylphenyl)methyl]-4-[(3-fluorophenyl)methyl]piperazine
1-[(3-fluorophenyl)-(4-phenylphenyl)methyl]piperazine
1-[bis(2,4-difluorophenyl)methyl]piperazine
1-[(4-fluorophenyl)-(4-phenylphenyl)methyl]piperazine
1-[(2-fluorophenyl)-(4-phenylphenyl)methyl]piperazine
1-[(4-phenylphenyl)-[4-(trifluoromethyl)phenyl]methyl]piperazine
1-[(2-fluorophenyl)-phenylmethyl]piperazine
N,N-dibenzyl-4,4,5,5,6,6,7,7,7-nonafluoro-1-phenylheptan-3-amine
1-[(1S)-1-phenylethyl]-4-[(1R)-1-henylethyl]piperazine
1-benzyl-2-phenyl-4-propan-2-ylpiperazine4-benzhydryl-1-methyl-1-propan-2-ylpiperazin-1-ium iodide
4-benzhydryl-1-ethyl-1-propylpiperazin-1-ium iodide
4-benzhydryl-1-ethyl-1-methylpiperazin-1-ium iodide
4-benzhydryl-1-ethyl-1-propan-2-ylpiperazin-1-ium iodide
4-benzhydryl-1,1-diethyl-2,5-dimethylpiperazin-1-ium
4-benzhydryl-1,1-diethylpiperazin-1-ium iodide
4-benzhydryl-1-butyl-1-methylpiperazin-1-ium iodide
1-[(4-ethylphenyl)methyl]-4-[(2-fluorophenyl)methyl]piperazine
N-benzyl-N,1-bis(2-phenylethyl)piperidin-4-amine
1-benzyl-4-(4-phenylbutan-2-yl)piperazine
1-[(4-phenylphenyl)methyl]-4-(3-phenylprop-2-enyl)piperazine
1-benzhydryl-4-(3-phenylprop-2-ynyl)piperazine dihydrochloride
1-[(4-fluorophenyl)-phenylmethyl]piperazine
1-benzyl-4-[(2R)-4-phenylbutan-2-yl]piperazine
1-benzyl-4-[(2S)-4-phenylbutan-2-yl]piperazine
4-benzyl-1-[(3,4-difluorophenyl)methyl]piperidine
1-benzyl-4-[(4-phenylphenyl)methyl]piperazine
(4-benzhydrylpiperazin-1-yl)-phenylmethanethione
1-cyclohexyl-4-[(1R,2R)-1,2-diphenylpropyl]piperazine
1-cyclohexyl-4-[(1R,2S)-1,2-diphenylpropyl]piperazine
1-cyclohexyl-4-[(1S,2R)-1,2-diphenylpropyl]piperazine
1-cyclohexyl-4-[(1S,2S)-1,2-diphenylpropyl]piperazine
1-[(4-ethylphenyl)methyl]-4-[(4-fluorophenyl)methyl]piperazine
1-(1,2-diphenyl-2-piperidin-1-ylethyl)piperidine
1,4-bis(1-phenylethyl)piperazine
1-benzyl-4-[(2-benzylphenyl)methyl]piperazine
1-benzyl-4-(1-phenylpropan-2-yl)piperazine
1-benzhydryl-4-prop-2-enylpiperazine dihydrochloride

Additional compounds with Ca²⁺-channel blocking activity that are useful in combination with TAAs (and cells which interact with TAAs) include amlodipine, aranidipine, azelnidipine, barnidipine, benidipine, cilnidipine, clevidipine, darodipine, efonidipine, felodipine, isradipine, lacidipine, manidipine, lercanidipine, mepirodipine, nicardipine, nifedipine, niludipin, nilvadipine, nimodipine, nisoldipine, nitrendipine, oxodipine, pranidipine, ryodipine, anipamil, devapamil, emopamil, falipamil, gallopamil, norverapamil, verapamil, clentiazem, diltiazem, bepridil, fendiline, lidoflazine, perhexiline, amrinone, anandamide, azimilide, bencyclane, berbamine, bevantolol, canadine, carboxyamidotriazole, caroverine, cinnarizine, conotoxins, dauricine, dimeditiapramine, dotarizine, enpiperate, eperisone, fantofarone, fasudil, fenamic acid, fostedil, gabapentin, lamotrigine, magnesium sulfate, manoalide, mibefradil, monatepil, naftopidil, niguldipine, ochratoxin a, octylonium, osthol, pinaverium, piperidine, pregabalin, prenylamine, risedronic acid, sesamodil, stepholidine, terodiline, tetrahydropalmatine, tetrandrine, tolfenamic acid, tranilast, trox-1, and ziconotide.

Flunarizine and several flunarizine analogs are commercially available. Standard doses of flunarizine and several flunarizine analogs are known in the art (e.g., 0.5 mg, 1.0 mg, 2.0 mg, 3.0 mg, 4.0 mg, 5.0 mg, 7.5 mg, 10 mg, 15 mg, 20 mg, 25 mg, 50 mg, 75 mg, 100 mg, 125 mg, 150 mg, 175 mg, 200 mg, 225 mg, 250 mg, 275 mg, 300 mg, 350 mg, 400 mg, 450 mg, 500 mg, 550 mg, 600 mg, 650 mg, 700 mg, 750 mg, 800 mg, 850 mg, 900 mg, or any range between any pair of recited doses) and can range, e.g., from 0.1 mg to 300 mg (e.g., 0.1 mg to 200 mg, 0.1 mg to 150 mg, 0.1 mg to 100 mg, and 1.0 mg to 50 mg).

### Aphidicolin

The methods, compositions, and kits of the invention may employ aphidicolin or an aphidicolin analog. A variety of aphidicolin analogs are known in the art. Nonlimiting examples of aphidicolin analogs are described in U.S. Pat. No. 3,761,512 (e.g., the 9α-monoacetate and 9α-hemisuccinate forms of formula (I)); and U.S. Pat. No. 5,039,710 (e.g., compounds of general formula (II)), each of which is incorporated by reference.

Additional aphidicolin analogs are set forth in Table 4 below.

TABLE 4
Aphidicolin analogs

6beta-hydroxy-3-ketoaphidicolin
Aphidicolin glycinate
Aphidicolin-17-acetate
3-Ketoaphidicolin
18-Nor-3-ketoaphidicolin
18-Carboxy aphidicolin
18-Acetoxyaphidicolin
6-beta-Hydroxyaphidicolin
aphidicolin
6-beta-Hydroxyaphidicolin
2alpha,3beta,13-Trihydroxystemodane
(3S,5S,6S,8R,9S,10R,13S,14S,17S)-17-[(2R,3R)-2,3-dihydroxy-6-methylheptan-2-yl]-10,13-dimethyl-
2,3,4,5,6,7,8,9,11,12,14,15,16,17-tetradecahydro-1H-cyclopenta[a]phenanthrene-3,6-diol
(8R,9S,10S,13R,14S,17R)-10,13-dimethyl-17-[(2R)-5,6,7-trihydroxy-6-methylheptan-2-yl]-
2,3,4,5,6,7,8,9,11,12,14,15,16,17-tetradecahydro-1H-cyclopenta[a]phenanthrene-3,7,12-triol
(24R,S)-24-Methylcycloartane-3 beta,24,24-triol
(3R,5S,7R,8R,9S,10S,12S,13R,14S,17R)-10,13-dimethyl-17-[(2R)-5,6,7-trihydroxy-6-methylheptan-2-
yl]-2,3,4,5,6,7,8,9,11,12,14,15,16,17-tetradecahydro-1H-cyclopenta[a]phenanthrene-3,7,12-triol
(3R,5S,7R,8R,9S,10S,12S,13R,14S,17R)-17-[(2R)-6,7-dihydroxy-6-(hydroxymethyl)heptan-2-yl]-10,13-
dimethyl-2,3,4,5,6,7,8,9,11,12,14,15,16,17-tetradecahydro-1H-cyclopenta[a]phenanthrene-3,7,12-triol
(24S)-Cycloartanetriol
(24R)-Cycloartanetriol
(3R,5S,7R,8R,9S,10S,12S,13R,14S,17R)-10,13-dimethyl-17-[(2R,5S)-5,6,7-trihydroxy-6-methylheptan-
2-yl]-2,3,4,5,6,7,8,9,11,12,14,15,16,17-tetradecahydro-1H-cyclopenta[a]phenanthrene-3,7,12-triol
Stemodin
3-Deoxyaphidicolin
(3R,5S,7R,12S)-17-(6,7-dihydroxy-6-methylheptan-2-yl)-10,13-dimethyl-
2,3,4,5,6,7,8,9,11,12,14,15,16,17-tetradecahydro-1H-cyclopenta[a]phenanthrene-3,7,12-triol
(3R,5R,7R,12S)-17-(6,7-dihydroxy-6-methylheptan-2-yl)-10,13-dimethyl-
2,3,4,5,6,7,8,9,11,12,14,15,16,17-tetradecahydro-1H-cyclopenta[a]phenanthrene-3,7,12-triol
(3S,5S,8R,9S,10S,13S,14S,17R)-17-[(1S)-1,2-dihydroxyethyl]-10,13-dimethyl-
1,2,3,4,5,6,7,8,9,11,12,14,15,16-tetradecahydrocyclopenta[a]phenanthrene-3,17-diol
(3R,7R,8R,9S,10S,12S,13R,14S,17R)-17-[(2R)-5,6-dihydroxy-6-methylheptan-2-yl]-10,13-dimethyl-
2,3,4,5,6,7,8,9,11,12,14,15,16,17-tetradecahydro-1H-cyclopenta[a]phenanthrene-3,7,12-triol
(8R,9S,10S,13R,14S,17R)-17-[(2R)-5,6-dihydroxy-6-methylheptan-2-yl]-10,13-dimethyl-
2,3,4,5,6,7,8,9,11,12,14,15,16,17-tetradecahydro-1H-cyclopenta[a]phenanthrene-3,7-diol
MW: 436.667620 g/mol|MF: C27H48O4
(8R,9S,10S,13R,14S,17R)-17-[(2R)-6,7-dihydroxy-6-methylheptan-2-yl]-10,13-dimethyl-
2,3,4,5,6,7,8,9,11,12,14,15,16,17-tetradecahydro-1H-cyclopenta[a]phenanthrene-3,7,12-triol
(8R,9S,10S,13R,14S,17R)-10,13-dimethyl-17-[(2R)-5,6,7-trihydroxyheptan-2-yl]-
2,3,4,5,6,7,8,9,11,12,14,15,16,17-tetradecahydro-1H-cyclopenta[a]phenanthrene-3,7,12-triol
(3R,5R,8R,9S,10S,13S,14S,17R)-17-(1,2-dihydroxyethyl)-10,13-dimethyl-
1,2,3,4,5,6,7,8,9,11,12,14,15,16-tetradecahydrocyclopenta[a]phenanthrene-3,17-diol
(8R,9S,10S,13R,14S,17R)-17-[(2R)-6,7-dihydroxy-6-methylheptan-2-yl]-10,13-dimethyl-
2,3,4,5,6,7,8,9,11,12,14,15,16,17-tetradecahydro-1H-cyclopenta[a]phenanthrene-3,7-diol
(8R,9S,10S,13R,14S,17R)-17-[(2R)-5,6-dihydroxy-6-methylheptan-2-yl]-10,13-dimethyl-
2,3,4,5,6,7,8,9,11,12,14,15,16,17-tetradecahydro-1H-cyclopenta[a]phenanthrene-3,7,12-triol
(8R,9S,10S,13S,14S,17R)-17-(1,2-dihydroxyethyl)-10,13-dimethyl-1,2,3,4,5,6,7,8,9,11,12,14,15,16-
tetradecahydrocyclopenta[a]phenanthrene-3,17-diol
(8R,9S,10S,13S,14S)-17-(1,2-dihydroxyethyl)-10,13-dimethyl-1,2,3,4,5,6,7,8,9,11,12,14,15,16-
tetradecahydrocyclopenta[a]phenanthrene-3,17-diol
(3S,5S,8R,9S,10S,13S,14S,17R)-17-(1,2-dihydroxyethyl)-10,13-dimethyl-
1,2,3,4,5,6,7,8,9,11,12,14,15,16-tetradecahydrocyclopenta[a]phenanthrene-3,17-diol
17-(5,6-dihydroxy-6-methylheptan-2-yl)-10,13-dimethyl-2,3,4,5,6,7,8,9,11,12,14,15,16,17-
tetradecahydro-1H-cyclopenta[a]phenanthrene-3,7-diol
(3R,5S,7R,8R,9S,10S,12S,13R,14S,17R)-17-[(2R,5R)-5,6-dihydroxy-6-methylheptan-2-yl]-10,13-
dimethyl-2,3,4,5,6,7,8,9,11,12,14,15,16,17-tetradecahydro-1H-cyclopenta[a]phenanthrene-3,7,12-triol
17-(5,6-dihydroxy-6-methylheptan-2-yl)-10,13-dimethyl-2,3,4,5,6,7,8,9,11,12,14,15,16,17-
tetradecahydro-1H-cyclopenta[a]phenanthrene-3,7,12-triol
17-(6,7-dihydroxy-6-methylheptan-2-yl)-10,13-dimethyl-2,3,4,5,6,7,8,9,11,12,14,15,16,17-
tetradecahydro-1H-cyclopenta[a]phenanthrene-3,7,12-triol
(3S,5R,8S,9S,10R,13S,14S,17R)-17-[(3S,4S,5S)-5-ethyl-3,4-dihydroxy-6-methylheptan-2-yl]-10,13-
dimethyl-1,2,3,4,6,7,8,9,11,12,14,15,16,17-tetradecahydrocyclopenta[a]phenanthrene-3,5-diol
(3R,5S,7R,8R,9S,10S,12S,13R,14S,17R)-17-[(2R,5S)-5,6-dihydroxy-6-methylheptan-2-yl]-10,13-
dimethyl-2,3,4,5,6,7,8,9,11,12,14,15,16,17-tetradecahydro-1H-cyclopenta[a]phenanthrene-3,7,12-triol
17-(5,6-dihydroxy-5,6-dimethylheptan-2-yl)-10,13-dimethyl-2,3,4,5,6,7,8,9,11,12,14,15,16,17-
tetradecahydro-1H-cyclopenta[a]phenanthrene-3,7,12-triol
(5S,10S,13R,17R)-17-(6,7-dihydroxy-6-methylheptan-2-yl)-10,13-dimethyl-
2,3,4,5,6,7,8,9,11,12,14,15,16,17-tetradecahydro-1H-cyclopenta[a]phenanthrene-3,7,12-triol
(3R,5S,7R,8R,9S,10S,13R,14S,17R)-17-[(2R)-5,6-dihydroxy-6-methylheptan-2-yl]-10,13-dimethyl-
2,3,4,5,6,7,8,9,11,12,14,15,16,17-tetradecahydro-1H-cyclopenta[a]phenanthrene-3,7-diol
(3R,5S,7R,8R,9S,10S,12S,13R,14S,17R)-17-[(2R)-5,6-dihydroxy-6-methylheptan-2-yl]-10,13-dimethyl-
2,3,4,5,6,7,8,9,11,12,14,15,16,17-tetradecahydro-1H-cyclopenta[a]phenanthrene-3,7,12-triol
(3R,5S,7R,8R,9S,10S,13R,14S,17R)-17-[(2R)-6,7-dihydroxy-6-methylheptan-2-yl]-10,13-dimethyl-
2,3,4,5,6,7,8,9,11,12,14,15,16,17-tetradecahydro-1H-cyclopenta[a]phenanthrene-3,7-diol
(3R,5S,7R,8R,9S,10S,12S,13R,14S,17R)-17-[(2R)-6,7-dihydroxy-6-methylheptan-2-yl]-10,13-dimethyl-
2,3,4,5,6,7,8,9,11,12,14,15,16,17-tetradecahydro-1H-cyclopenta[a]phenanthrene-3,7,12-triol
(3R,5R,7R,8R,9S,10S,12S,13R,14S,17R)-17-[(2R)-6,7-dihydroxy-6-methylheptan-2-yl]-10,13-dimethyl-
2,3,4,5,6,7,8,9,11,12,14,15,16,17-tetradecahydro-1H-cyclopenta[a]phenanthrene-3,7,12-triol
(3S,5R,7R,8R,9S,10S,12S,13R,14S,17R)-17-[(2R)-6,7-dihydroxy-6-methylheptan-2-yl]-10,13-dimethyl-
2,3,4,5,6,7,8,9,11,12,14,15,16,17-tetradecahydro-1H-cyclopenta[a]phenanthrene-3,7,12-triol
17-(1,2-dihydroxyethyl)-10,13-dimethyl-1,2,3,4,5,6,7,8,9,11,12,14,15,16-
tetradecahydrocyclopenta[a]phenanthrene-3,6,17-triol
17-(1,2-dihydroxyethyl)-10,13-dimethyl-1,2,3,4,5,6,7,8,9,11,12,14,15,16-
tetradecahydrocyclopenta[a]phenanthrene-3,17-diol
(3R,5S,7R,8R,9S,10S,12S,13R,14S,17R)-10,13-dimethyl-17-[(2R)-5,6,7-trihydroxyheptan-2-yl]-
2,3,4,5,6,7,8,9,11,12,14,15,16,17-tetradecahydro-1H-cyclopenta[a]phenanthrene-3,7,12-triol
(3R,5R,8R,9S,10S,13S,14S,17R)-17-[(1S)-1,2-dihydroxyethyl]-10,13-dimethyl-
1,2,3,4,5,6,7,8,9,11,12,14,15,16-tetradecahydrocyclopenta[a]phenanthrene-3,17-diol
(3R,5S,7R,8R,9S,10S,12S,13R)-10,13-dimethyl-17-[(2R)-5,6,7-trihydroxyheptan-2-yl]-
2,3,4,5,6,7,8,9,11,12,14,15,16,17-tetradecahydro-1H-cyclopenta[a]phenanthrene-3,7,12-triol
(6S)-10,13-dimethyl-17-[(2R)-6-methylheptan-2-yl]-1,2,3,4,6,7,8,9,11,12,14,15,16,17-
tetradecahydrocyclopenta[a]phenanthrene-5,6-diol
(4R)-10,13-dimethyl-17-[(2R)-6-methylheptan-2-yl]-1,2,3,4,6,7,8,9,11,12,14,15,16,17-
tetradecahydrocyclopenta[a]phenanthrene-4,5-diol
(5R)-10,13-dimethyl-17-[(2R)-6-methylheptan-2-yl]-1,2,3,4,5,6,7,9,11,12,14,15,16,17-
tetradecahydrocyclopenta[a]phenanthrene-7,8-diol
(5R)-10,13-dimethyl-17-[(2R)-6-methylheptan-2-yl]-1,2,3,4,6,7,8,9,11,12,14,15,16,17-
tetradecahydrocyclopenta[a]phenanthrene-5,6-diol
(5R,6S)-10,13-dimethyl-17-[(2R)-6-methylheptan-2-yl]-1,2,3,4,6,7,8,9,11,12,14,15,16,17-
tetradecahydrocyclopenta[a]phenanthrene-5,6-diol
(5S)-10,13-dimethyl-17-[(2R)-6-methylheptan-2-yl]-1,2,3,4,6,7,8,9,11,12,14,15,16,17-
tetradecahydrocyclopenta[a]phenanthrene-4,5-diol
(4R,5S)-10,13-dimethyl-17-[(2R)-6-methylheptan-2-yl]-1,2,3,4,6,7,8,9,11,12,14,15,16,17-
tetradecahydrocyclopenta[a]phenanthrene-4,5-diol
(5R,10R,13R)-10,13-dimethyl-17-(6-methylheptan-2-yl)-1,2,3,4,6,7,8,9,11,12,14,15,16,17-
tetradecahydrocyclopenta[a]phenanthrene-5,6-diol
(3S,4R,5R,10R,13R)-17-[(2R,5S)-5,6-dimethylheptan-2-yl]-4,10,13-trimethyl-
1,2,3,5,6,7,8,9,11,12,14,15,16,17-tetradecahydrocyclopenta[a]phenanthrene-3,4-diol
(3S,4R,5R,10R,13R)-17-[(2R,5S)-5-ethyl-6-methylheptan-2-yl]-4,10,13-trimethyl-
1,2,3,5,6,7,8,9,11,12,14,15,16,17-tetradecahydrocyclopenta[a]phenanthrene-3,4-diol
(4S,5S,8S,9S,10R,13R,14S,17R)-10,13-dimethyl-17-[(2R)-6-methylheptan-2-yl]-
1,2,3,4,6,7,8,9,11,12,14,15,16,17-tetradecahydrocyclopenta[a]phenanthrene-4,5-diol
(6R)-6-[(3R,5R,8R,9S,10S,13R,14S,17R)-3-hydroxy-10,13-dimethyl-2,3,4,5,6,7,8,9,11,12,14,15,16,17-
tetradecahydro-1H-cyclopenta[a]phenanthren-17-yl]-2-methylheptane-1,2-diol
(2R,3S)-2-[(3S,5S,8R,9S,10S,13S,14S,17S)-3-hydroxy-10,13-dimethyl-
2,3,4,5,6,7,8,9,11,12,14,15,16,17-tetradecahydro-1H-cyclopenta[a]phenanthren-17-yl]-6-methylheptane-
2,3-diol
(2R,3R)-2-[(3S,5S,8R,9S,10S,13S,14S,17S)-3-hydroxy-10,13-dimethyl-
2,3,4,5,6,7,8,9,11,12,14,15,16,17-tetradecahydro-1H-cyclopenta[a]phenanthren-17-yl]-6-methylheptane-
2,3-diol
(4R,5S,8S,9S,10R,13R,14S,17R)-10,13-dimethyl-17-[(2R)-6-methylheptan-2-yl]-
1,2,3,4,6,7,8,9,11,12,14,15,16,17-tetradecahydrocyclopenta[a]phenanthrene-4,5-diol
6-(3-hydroxy-10,13-dimethyl-2,3,4,5,6,7,8,9,11,12,14,15,16,17-tetradecahydro-1H-
cyclopenta[a]phenanthren-17-yl)-2-methylheptane-2,3-diol
(5R,6S,8S,9S,10R,13R,14S,17R)-10,13-dimethyl-17-[(2R)-6-methylheptan-2-yl]-
1,2,3,4,6,7,8,9,11,12,14,15,16,17-tetradecahydrocyclopenta[a]phenanthrene-5,6-diol
(5R,6R,8S,9S,10R,13R,14S,17R)-10,13-dimethyl-17-[(2R)-6-methylheptan-2-yl]-
1,2,3,4,6,7,8,9,11,12,14,15,16,17-tetradecahydrocyclopenta[a]phenanthrene-5,6-diol
(5R,6S,8R,9R,10R,13R,14S,17R)-10,13-dimethyl-17-[(2S)-6-methylheptan-2-yl]-
1,2,3,4,6,7,8,9,11,12,14,15,16,17-tetradecahydrocyclopenta[a]phenanthrene-5,6-diol
(5R,6S,8R,9R,10R,13R,14S,17R)-10,13-dimethyl-17-[(2R)-6-methylheptan-2-yl]-
1,2,3,4,6,7,8,9,11,12,14,15,16,17-tetradecahydrocyclopenta[a]phenanthrene-5,6-diol
(5R,6S,8R,9R,10R,13R,14S,17S)-10,13-dimethyl-17-[(2S)-6-methylheptan-2-yl]-
1,2,3,4,6,7,8,9,11,12,14,15,16,17-tetradecahydrocyclopenta[a]phenanthrene-5,6-diol
(5R,6S,8R,9R,10R,13R,14S,17S)-10,13-dimethyl-17-[(2R)-6-methylheptan-2-yl]-
1,2,3,4,6,7,8,9,11,12,14,15,16,17-tetradecahydrocyclopenta[a]phenanthrene-5,6-diol
(3R,6R)-6-[(3R,5R,8R,9S,10S,12S,13R,14S,17R)-3,12-dihydroxy-10,13-dimethyl-
2,3,4,5,6,7,8,9,11,12,14,15,16,17-tetradecahydro-1H-cyclopenta[a]phenanthren-17-yl]-2-
(hydroxymethyl)heptane-1,3-diol
(6R)-6-[(3R,5R,8R,9S,10S,13R,14S,17R)-3-hydroxy-10,13-dimethyl-2,3,4,5,6,7,8,9,11,12,14,15,16,17-
tetradecahydro-1H-cyclopenta[a]phenanthren-17-yl]-2-methylheptane-2,3-diol
10,13-dimethyl-17-(6-methylheptan-2-yl)-1,2,3,4,5,6,7,9,11,12,14,15,16,17-
tetradecahydrocyclopenta[a]phenanthrene-7,8-diol
10,13-dimethyl-17-(6-methylheptan-2-yl)-1,2,3,4,6,7,8,9,11,12,14,15,16,17-
tetradecahydrocyclopenta[a]phenanthrene-5,6-diol
10,13-dimethyl-17-(6-methylheptan-2-yl)-1,2,3,4,6,7,8,9,11,12,14,15,16,17-
tetradecahydrocyclopenta[a]phenanthrene-4,5-diol
(3R,5S,7R,9R,10S,12S,13R,14S,17S)-17-[(2R)-5,6-dihydroxyheptan-2-yl]-10,13-dimethyl-
2,3,4,5,6,7,8,9,11,12,14,15,16,17-tetradecahydro-1H-cyclopenta[a]phenanthrene-3,7,12-triol
(5S,7S)-10,13-dimethyl-17-[(2R)-6-methylheptan-2-yl]-2,3,4,5,6,7,8,9,11,12,14,15,16,17-tetradecahydro-
1H-cyclopenta[a]phenanthrene-6,7-diol
(5S,6R)-10,13-dimethyl-17-[(2R)-6-methylheptan-2-yl]-2,3,4,5,6,7,8,9,11,12,14,15,16,17-
tetradecahydro-1H-cyclopenta[a]phenanthrene-6,7-diol
(5R)-10,13-dimethyl-17-[(2R)-6-methylheptan-2-yl]-2,3,4,5,6,7,8,9,11,12,14,15,16,17-tetradecahydro-
1H-cyclopenta[a]phenanthrene-3,4-diol
(4S,5R)-10,13-dimethyl-17-[(2R)-6-methylheptan-2-yl]-2,3,4,5,6,7,8,9,11,12,14,15,16,17-
tetradecahydro-1H-cyclopenta[a]phenanthrene-3,4-diol
(5S)-10,13-dimethyl-17-[(2R)-6-methylheptan-2-yl]-2,3,4,5,6,7,8,9,11,12,14,15,16,17-tetradecahydro-
1H-cyclopenta[a]phenanthrene-1,2-diol
(2S,5S)-10,13-dimethyl-17-[(2R)-6-methylheptan-2-yl]-2,3,4,5,6,7,8,9,11,12,14,15,16,17-tetradecahydro-
1H-cyclopenta[a]phenanthrene-1,2-diol
(1R,5S)-10,13-dimethyl-17-[(2R)-6-methylheptan-2-yl]-2,3,4,5,6,7,8,9,11,12,14,15,16,17-
tetradecahydro-1H-cyclopenta[a]phenanthrene-1,2-diol
(3R,6S,17R)-17-(1-hydroxyethyl)-10,13-dimethyl-1,2,3,4,5,6,7,8,9,11,12,14,15,16-
tetradecahydrocyclopenta[a]phenanthrene-3,6,17-triol
(2R)-2-[(1R,3aR,5aR,5bR,7aR,9R,11aR,11bR,13aR,13bS)-9-hydroxy-3a,5a,5b,8,8,11a-hexamethyl-
1,2,3,4,5,6,7,7a,9,10,11,11b,12,13,13a,13b-hexadecahydrocyclopenta[a]chrysen-1-yl]propane-1,2-diol
(2S)-2-[(1R,3aR,5aR,5bR,7aR,9R,11aR,11bR,13aR,13bS)-9-hydroxy-3a,5a,5b,8,8,11a-hexamethyl-
1,2,3,4,5,6,7,7a,9,10,11,11b,12,13,13a,13b-hexadecahydrocyclopenta[a]chrysen-1-yl]propane-1,2-diol
(2R,3S,10S,13S)-17-[(2S,3R,4R)-3,4-dihydroxy-5,6-dimethylheptan-2-yl]-10,13-dimethyl-
2,3,4,5,6,7,8,9,11,12,14,15,16,17-tetradecahydro-1H-cyclopenta[a]phenanthrene-2,3-diol
(3S,6S,8R,9S,10R,12R,13R,14R,17S)-17-[(2S)-2-hydroxy-6-methylheptan-2-yl]-4,4,10,14-tetramethyl-
1,2,3,5,6,7,8,9,11,12,13,15,16,17-tetradecahydrocyclopenta[a]phenanthrene-3,6,12-triol
(2R)-2-[(3aS,5aR,5bR,6S,8R,11aS,11bR)-6-hydroxy-5a,5b,8,11a-tetramethyl-
1,2,3,3a,4,5,6,7,7a,8,9,10,11,11b,12,13,13a,13b-octadecahydrocyclopenta[a]chrysen-1-yl]propane-1,2-
diol
(2S,3R,4R,5S)-2-[(3R,5S,10S,13S)-3-hydroxy-10,13-dimethyl-2,3,4,5,6,7,8,9,11,12,14,15,16,17-
tetradecahydro-1H-cyclopenta[a]phenanthren-17-yl]-5,6-dimethylheptane-3,4-diol
(2R,3S,5S,10S,13S)-17-[(2S,3R,4R,5S)-3,4-dihydroxy-5,6-dimethylheptan-2-yl]-10,13-dimethyl-
2,3,4,5,6,7,8,9,11,12,14,15,16,17-tetradecahydro-1H-cyclopenta[a]phenanthrene-2,3-diol
(2S,3R,4R,5S)-2-[(3S,5S,10S,13S)-3-hydroxy-10,13-dimethyl-2,3,4,5,6,7,8,9,11,12,14,15,16,17-
tetradecahydro-1H-cyclopenta[a]phenanthren-17-yl]-5,6-dimethylheptane-3,4-diol
(3S,6S,10R,13S)-17-[(2S)-2,7-dihydroxy-6,6-dimethylheptan-2-yl]-10,13-dimethyl-
2,3,4,5,6,7,8,9,11,12,14,15,16,17-tetradecahydro-1H-cyclopenta[a]phenanthrene-3,6-diol
(2R,3S,5S,8R,9S,10S,13S,17R)-17-[(2S,3R,4R,5S)-3,4-dihydroxy-5,6-dimethylheptan-2-yl]-10,13-
dimethyl-2,3,4,5,6,7,8,9,11,12,14,15,16,17-tetradecahydro-1H-cyclopenta[a]phenanthrene-2,3-diol
(2S,3R,4R,5S)-2-[(3R,5S,8R,9S,10S,13S,17R)-3-hydroxy-10,13-dimethyl-
2,3,4,5,6,7,8,9,11,12,14,15,16,17-tetradecahydro-1H-cyclopenta[a]phenanthren-17-yl]-5,6-
dimethylheptane-3,4-diol
(2S,3R,4R,5S)-2-[(3S,5S,8R,9S,10S,13S,17R)-3-hydroxy-10,13-dimethyl-
2,3,4,5,6,7,8,9,11,12,14,15,16,17-tetradecahydro-1H-cyclopenta[a]phenanthren-17-yl]-5,6-
dimethylheptane-3,4-diol
(3S,4S,10R,13S)-17-[(2S,5S)-5-ethyl-2-hydroxy-6-methylheptan-2-yl]-4,10,13-trimethyl-
2,3,4,5,6,7,8,9,11,12,14,15,16,17-tetradecahydro-1H-cyclopenta[a]phenanthren-3-ol
(5S,7S,8R,9R,10S,13R,14R,17S)-17-[(2S)-2-hydroxy-6-methylheptan-2-yl]-4,4,8,10,14-pentamethyl-
2,3,5,6,7,9,11,12,13,15,16,17-dodecahydro-1H-cyclopenta[a]phenanthren-7-ol
(5S,6S,7S,8S,9S,10R,13R,14S,17R)-10,13-dimethyl-17-[(2R)-6-methylheptan-2-yl]-
2,3,4,5,6,7,8,9,11,12,14,15,16,17-tetradecahydro-1H-cyclopenta[a]phenanthrene-6,7-diol
(5S,6R,7S,8S,9S,10R,13R,14S,17R)-10,13-dimethyl-17-[(2R)-6-methylheptan-2-yl]-
2,3,4,5,6,7,8,9,11,12,14,15,16,17-tetradecahydro-1H-cyclopenta[a]phenanthrene-6,7-diol
(3S,4R,5R,8S,9S,10R,13R,14S,17R)-10,13-dimethyl-17-[(2R)-6-methylheptan-2-yl]-
2,3,4,5,6,7,8,9,11,12,14,15,16,17-tetradecahydro-1H-cyclopenta[a]phenanthrene-3,4-diol
(3R,4R,5R,8S,9S,10R,13R,14S,17R)-10,13-dimethyl-17-[(2R)-6-methylheptan-2-yl]-
2,3,4,5,6,7,8,9,11,12,14,15,16,17-tetradecahydro-1H-cyclopenta[a]phenanthrene-3,4-diol
(1R,2R,5S,8S,9S,10S,13R,14S,17R)-10,13-dimethyl-17-[(2R)-6-methylheptan-2-yl]-
2,3,4,5,6,7,8,9,11,12,14,15,16,17-tetradecahydro-1H-cyclopenta[a]phenanthrene-1,2-diol
(3S,5R,6S,8R,9R,10R,12R,13R,14R,17S)-17-[(2R)-2-hydroxy-6-methylheptan-2-yl]-4,4,8,10,14-
pentamethyl-2,3,5,6,7,9,11,12,13,15,16,17-dodecahydro-1H-cyclopenta[a]phenanthrene-3,6,12-triol
(3S,5R,6S,8R,9R,10R,12R,13R,14R,17S)-17-[(2S)-2-hydroxy-6-methylheptan-2-yl]-4,4,8,10,14-
pentamethyl-2,3,5,6,7,9,11,12,13,15,16,17-dodecahydro-1H-cyclopenta[a]phenanthrene-3,6,12-triol
6-[(3R,5R,8R,9R,10R,13R,14R,17S)-3-hydroxy-4,4,8,10,14-pentamethyl-2,3,5,6,7,9,11,12,13,15,16,17-
dodecahydro-1H-cyclopenta[a]phenanthren-17-yl]-2-methylheptane-2,3,6-triol
10,13-dimethyl-17-(6-methylheptan-2-yl)-1,2,3,4,5,6,7,9,11,12,14,15,16,17-
tetradecahydrocyclopenta[a]phenanthrene-3,8-diol
17-(5-hydroxy-5-methylhexan-2-yl)-10,13-dimethyl-2,3,4,5,6,7,8,9,11,12,14,15,16,17-tetradecahydro-
1H-cyclopenta[a]phenanthrene-3,12-diol
(3R,5S,7R,8R,9S,10S,12S,13R,14S,17R)-17-[(2R)-5,6-dihydroxyheptan-2-yl]-10,13-dimethyl-
2,3,4,5,6,7,8,9,11,12,14,15,16,17-tetradecahydro-1H-cyclopenta[a]phenanthrene-3,7,12-triol
(3R,5R,7R,8R,9S,10S,12S,13R,14S,17R)-17-[(2S)-4,6-dihydroxy-6-methylheptan-2-yl]-10,13-dimethyl-
2,3,4,5,6,7,8,9,11,12,14,15,16,17-tetradecahydro-1H-cyclopenta[a]phenanthrene-3,7,12-triol
(3R,5S,7R,8R,9S,10S,12S,13R,14S,17R)-17-[(2R,4R)-4,6-dihydroxy-6-methylheptan-2-yl]-10,13-
dimethyl-2,3,4,5,6,7,8,9,11,12,14,15,16,17-tetradecahydro-1H-cyclopenta[a]phenanthrene-3,7,12-triol
(3S,5S,8S,9S,10S,11S,13S,14S,17S)-17-(1,2-dihydroxyethyl)-10,13-dimethyl-
2,3,4,5,6,7,8,9,11,12,14,15,16,17-tetradecahydro-1H-cyclopenta[a]phenanthrene-3,11-diol
(3R,5R,6S,8R,9S,10R,13S,14S,17R)-17-[(1R)-1-hydroxyethyl]-10,13-dimethyl-
1,2,3,4,5,6,7,8,9,11,12,14,15,16-tetradecahydrocyclopenta[a]phenanthrene-3,6,17-triol
(8R,9S,10S,13R,14S,17R)-17-[(2R)-5,6-dihydroxyheptan-2-yl]-10,13-dimethyl-
2,3,4,5,6,7,8,9,11,12,14,15,16,17-tetradecahydro-1H-cyclopenta[a]phenanthrene-3,7,12-triol
(3R,5R,7R,8R,9S,10S,12S,13R,14S,17R)-17-[(2R,4S)-4,6-dihydroxy-6-methylheptan-2-yl]-10,13-
dimethyl-2,3,4,5,6,7,8,9,11,12,14,15,16,17-tetradecahydro-1H-cyclopenta[a]phenanthrene-3,7,12-triol
(3R,5S,7R,8R,9S,10S,12S,13R,14S,17R)-17-[(2R,4S)-4,6-dihydroxy-6-methylheptan-2-yl]-10,13-
dimethyl-2,3,4,5,6,7,8,9,11,12,14,15,16,17-tetradecahydro-1H-cyclopenta[a]phenanthrene-3,7,12-triol
(4S,8R,9S,10R,13S,14S,17S)-17-[(2S)-5-ethyl-2-hydroxy-6-methylheptan-2-yl]-4,10,13-trimethyl-
2,3,4,5,6,7,8,9,11,12,14,15,16,17-tetradecahydro-1H-cyclopenta[a]phenanthren-3-ol
6,7,10,13,17-pentamethyl-2,3,4,5,6,7,8,9,11,12,14,15,16,17-tetradecahydro-1H-
cyclopenta[a]phenanthrene-3,4-diol
(3R,4S,5R,8S,9S,10R,13R,14S,17R)-10,13-dimethyl-17-[(2R)-6-methylheptan-2-yl]-
2,3,4,5,6,7,8,9,11,12,14,15,16,17-tetradecahydro-1H-cyclopenta[a]phenanthrene-3,4-diol
(3S,4S,5R,8S,9S,10R,13R,14S,17R)-10,13-dimethyl-17-[(2R)-6-methylheptan-2-yl]-
2,3,4,5,6,7,8,9,11,12,14,15,16,17-tetradecahydro-1H-cyclopenta[a]phenanthrene-3,4-diol
(2S,3R,4R,5S)-2-[(3R,5S,8R,9S,10S,13S,14S,17R)-3-hydroxy-10,13-dimethyl-
2,3,4,5,6,7,8,9,11,12,14,15,16,17-tetradecahydro-1H-cyclopenta[a]phenanthren-17-yl]-5,6-
dimethylheptane-3,4-diol
17-(4,6-dihydroxy-6-methylheptan-2-yl)-10,13-dimethyl-2,3,4,5,6,7,8,9,11,12,14,15,16,17-
tetradecahydro-1H-cyclopenta[a]phenanthrene-3,7,12-triol
17-(5,6-dihydroxyheptan-2-yl)-10,13-dimethyl-2,3,4,5,6,7,8,9,11,12,14,15,16,17-tetradecahydro-1H-
cyclopenta[a]phenanthrene-3,7,12-triol
(2R,3S,5S,8R,9S,10S,13S,14S,17R)-17-[(2S,3R,4R,5S)-3,4-dihydroxy-5,6-dimethylheptan-2-yl]-10,13-
dimethyl-2,3,4,5,6,7,8,9,11,12,14,15,16,17-tetradecahydro-1H-cyclopenta[a]phenanthrene-2,3-diol
(3R,5R,8S,9R,10R,13R,14S,16R)-16-[(1R)-1-hydroxyethyl]-5,10,13-trimethyl-
2,3,4,6,7,8,9,11,12,14,15,17-dodecahydro-1H-cyclopenta[a]phenanthrene-3,16-diol
(3R,5R,8S,9R,10R,13R,14S,16R)-16-[(1S)-1-hydroxyethyl]-5,10,13-trimethyl-
2,3,4,6,7,8,9,11,12,14,15,17-dodecahydro-1H-cyclopenta[a]phenanthrene-3,16-diol
(3R,5R,8S,9R,10R,13R,14R,16R)-16-[(1R)-1-hydroxyethyl]-5,10,13-trimethyl-
2,3,4,6,7,8,9,11,12,14,15,17-dodecahydro-1H-cyclopenta[a]phenanthrene-3,16-diol
(3R,5R,8S,9R,10R,13R,14R,16R)-16-[(1S)-1-hydroxyethyl]-5,10,13-trimethyl-
2,3,4,6,7,8,9,11,12,14,15,17-dodecahydro-1H-cyclopenta[a]phenanthrene-3,16-diol
(3S,6S,8R,9R,10R,12R,14R,17S)-4,4,8,10,14-pentamethyl-17-[(2S)-2,5,6-trihydroxy-6-methylheptan-2-
yl]-2,3,5,6,7,9,11,12,13,15,16,17-dodecahydro-1H-cyclopenta[a]phenanthrene-3,6,12-triol
(2R,3S,5S,8R,9S,10S,13S,14S,17R)-17-[(2S,3R,4R,5S)-5-ethyl-3,4-dihydroxy-6-methylheptan-2-yl]-
10,13-dimethyl-2,3,4,5,6,7,8,9,11,12,14,15,16,17-tetradecahydro-1H-cyclopenta[a]phenanthrene-2,3-diol
(2S,3R,4R,5S)-2-[(3S,5S,8R,9S,10S,13S,14S,17R)-3-hydroxy-10,13-dimethyl-
2,3,4,5,6,7,8,9,11,12,14,15,16,17-tetradecahydro-1H-cyclopenta[a]phenanthren-17-yl]-5,6-
dimethylheptane-3,4-diol
(3R,4R,8S,9S,10R,13S,14S,17R)-17-[(2R)-1-hydroxy-6-methylheptan-2-yl]-10,13-dimethyl-
2,3,4,5,6,7,8,9,11,12,14,15,16,17-tetradecahydro-1H-cyclopenta[a]phenanthrene-3,4-diol
(2R,3S,5S,8R,9S,10S,13S,14S,17R)-17-[(2S,3R,4R,5S)-3,4-dihydroxy-5-methylheptan-2-yl]-10,13-
dimethyl-2,3,4,5,6,7,8,9,11,12,14,15,16,17-tetradecahydro-1H-cyclopenta[a]phenanthrene-2,3-diol
(3S,6S,8R,9R,10R,13R,14R,17S)-17-[(2S)-2-hydroxy-6-methylheptan-2-yl]-4,4,8,10,14-pentamethyl-
2,3,5,6,7,9,11,12,13,15,16,17-dodecahydro-1H-cyclopenta[a]phenanthrene-3,6,12-triol
(2R)-2-[(1R,3aS,5aR,5bR,6S,8R,11aS,11bR)-6-hydroxy-5a,5b,8,11a-tetramethyl-
1,2,3,3a,4,5,6,7,7a,8,9,10,11,11b,12,13,13a,13b-octadecahydrocyclopenta[a]chrysen-1-yl]propane-1,2-
diol
(3S,5S,8S,9R,10S,13R,14R,17R)-17-[(2R)-5,6-dimethylheptan-2-yl]-10,13-dimethyl-
1,2,3,4,5,6,7,9,11,12,14,15,16,17-tetradecahydrocyclopenta[a]phenanthrene-3,8-diol
(3R,5R,8R,95,10S,125,13S,14S,17R)-17-[(2S)-3,4-dihydroxybutan-2-yl]-10,13-dimethyl-
2,3,4,5,6,7,8,9,11,12,14,15,16,17-tetradecahydro-1H-cyclopenta[a]phenanthrene-3,12-diol
(3R,5S,7S,8R,9S,10S,13R,14S,17R)-17-[(2S)-3,4-dihydroxybutan-2-yl]-10,13-dimethyl-
2,3,4,5,6,7,8,9,11,12,14,15,16,17-tetradecahydro-1H-cyclopenta[a]phenanthrene-3,7-diol
(3R,5S,7R,8R,9S,10S,13R,145,17R)-17-[(2S)-3,4-dihydroxybutan-2-yl]-10,13-dimethyl-
2,3,4,5,6,7,8,9,11,12,14,15,16,17-tetradecahydro-1H-cyclopenta[a]phenanthrene-3,7-diol
(3R,5S,7R,8R,9S,10S,12S,13R,14S,17R)-17-[(2S)-3,4-dihydroxybutan-2-yl]-10,13-dimethyl-
2,3,4,5,6,7,8,9,11,12,14,15,16,17-tetradecahydro-1H-cyclopenta[a]phenanthrene-3,7,12-triol
(3R,5R,7R,8R,9S,10S,12S,13R,14S,17R)-17-[(2R)-4,6-dihydroxy-6-methylheptan-2-yl]-10,13-dimethyl-
2,3,4,5,6,7,8,9,11,12,14,15,16,17-tetradecahydro-1H-cyclopenta[a]phenanthrene-3,7,12-triol
(3R,5S,7R,8R,9S,10S,12S,13R,14S,17R)-17-[(2R)-4,6-dihydroxy-6-methylheptan-2-yl]-10,13-dimethyl-
2,3,4,5,6,7,8,9,11,12,14,15,16,17-tetradecahydro-1H-cyclopenta[a]phenanthrene-3,7,12-triol
(2S,3S,5R,7R,8R,9S,10S,12S,13R,14S,17R)-17-[(2R)-7-hydroxy-6-(hydroxymethyl)heptan-2-yl]-10,13-
dimethyl-2,3,4,5,6,7,8,9,11,12,14,15,16,17-tetradecahydro-1H-cyclopenta[a]phenanthrene-2,3,7,12-tetrol
(3R,5S,7R,8R,9S,10S,13R,14S,17R)-17-[(2R)-4,5-dihydroxypentan-2-yl]-10,13-dimethyl-
2,3,4,5,6,7,8,9,11,12,14,15,16,17-tetradecahydro-1H-cyclopenta[a]phenanthrene-3,7-diol
10,13-dimethyl-17-(6-methylheptan-2-yl)-2,3,4,5,6,7,8,9,11,12,14,15,16,17-tetradecahydro-1H-
cyclopenta[a]phenanthrene-1,2-diol
17-(1-hydroxyethyl)-10,13-dimethyl-1,2,3,4,5,6,7,8,9,11,12,14,15,16-
tetradecahydrocyclopenta[a]phenanthrene-3,6,17-triol
1-(3-hydroxy-10,13-dimethyl-2,3,4,5,6,7,8,9,11,12,14,15,16,17-tetradecahydro-1H-
cyclopenta[a]phenanthren-17-yl)ethane-1,2-diol
16-(1-hydroxyethyl)-5,10,13-trimethyl-2,3,4,6,7,8,9,11,12,14,15,17-dodecahydro-1H-
cyclopenta[a]phenanthrene-3,16-diol
10,13-dimethyl-17-(6-methylheptan-2-yl)-2,3,4,5,6,7,8,9,11,12,14,15,16,17-tetradecahydro-1H-
cyclopenta[a]phenanthrene-3,4-diol
10,13-dimethyl-17-(6-methylheptan-2-yl)-2,3,4,5,6,7,8,9,11,12,14,15,16,17-tetradecahydro-1H-
cyclopenta[a]phenanthrene-6,7-diol
(3R,16R)-16-(1-hydroxyethyl)-5,10,13-trimethyl-2,3,4,6,7,8,9,11,12,14,15,17-dodecahydro-1H-
cyclopenta[a]phenanthrene-3,16-diol
(3S,5S,8S,9S,10S,11S,13S,14S,17S)-17-[(1S)-1,2-dihydroxyethyl]-10,13-dimethyl-
2,3,4,5,6,7,8,9,11,12,14,15,16,17-tetradecahydro-1H-cyclopenta[a]phenanthrene-3,11-diol
(8R,9S,10R,13S,14S,17R)-17-(1-hydroxyethyl)-10,13-dimethyl-1,2,3,4,5,6,7,8,9,11,12,14,15,16-
tetradecahydrocyclopenta[a]phenanthrene-3,6,17-triol
(3R,5S,7R,8R,9S,10S,12S,13R,14S,17S)-17-[(2R)-5,6-dihydroxyheptan-2-yl]-10,13-dimethyl-
2,3,4,5,6,7,8,9,11,12,14,15,16,17-tetradecahydro-1H-cyclopenta[a]phenanthrene-3,7,12-triol
(3R,5R,65,7S,9S,10R,13R,14S,17R)-17-[(2R)-6,7-dihydroxy-6-methylheptan-2-yl]-10,13-dimethyl-
2,3,4,5,6,7,8,9,11,12,14,15,16,17-tetradecahydro-1H-cyclopenta[a]phenanthrene-3,6,7-triol
(3R,5R,8S,10S,11S,13S,14S,17R)-17-[(1S)-1,2-dihydroxyethyl]-10,13-dimethyl-
1,2,3,4,5,6,7,8,9,11,12,14,15,16-tetradecahydrocyclopenta[a]phenanthrene-3,11,17-triol
(3S,5R,6R,7R,9S,10R,13R,14S)-17-[(2R,5S)-5,6-dimethylheptan-2-yl]-10,13-dimethyl-
1,2,3,4,6,7,8,9,11,12,14,15,16,17-tetradecahydrocyclopenta[a]phenanthrene-3,5,6,7-tetrol
17-(1,6-dihydroxy-2,6-dimethylheptan-2-yl)-4,4,8,10,14-pentamethyl-2,3,5,6,7,9,11,12,13,15,16,17-
dodecahydro-1H-cyclopenta[a]phenanthrene-3,12-diol
(3S,5R,6R,8S,9S,10R,11R,13R,14S,17R)-10,13-dimethyl-17-[(2R)-6-methylheptan-2-yl]-
1,2,3,4,6,7,8,9,11,12,14,15,16,17-tetradecahydrocyclopenta[a]phenanthrene-3,5,6,11-tetrol
MW: 436.667620 g/mol|MF: C27H48O4
(3S,5R,6R,8R,9R,10R,12R,13R,14R,17R)-17-[(2R)-6-methoxy-6-methylheptan-2-yl]-4,4,8,10,14-
pentamethyl-2,3,5,6,7,9,11,12,13,15,16,17-dodecahydro-1H-cyclopenta[a]phenanthrene-3,6,12-triol
(3S,5S,6R,8S,9S,10R,13S,14S,17S)-17-[(2S)-2-hydroxyoctan-2-yl]-6,10,13-trimethyl-
1,2,3,4,5,7,8,9,11,12,14,15,16,17-tetradecahydrocyclopenta[a]phenanthrene-3,6-diol
(3S,5R,7S,8R,9S,10S,12R,13R,14S,17R)-17-[(2R)-6-hydroxy-6-methylheptan-2-yl]-10,13-dimethyl-
2,3,4,5,6,7,8,9,11,12,14,15,16,17-tetradecahydro-1H-cyclopenta[a]phenanthrene-3,7,12-triol
17-[(2R)-4-hydroxy-4-methylpentan-2-yl]-10,13-dimethyl-2,3,4,5,6,7,8,9,11,12,14,15,16,17-
tetradecahydro-1H-cyclopenta[a]phenanthren-3-ol
(5R,7R,8R)-17-[(2R,5S)-5,6-dimethylheptan-2-yl]-10,13-dimethyl-1,2,3,4,5,6,7,9,11,12,14,15,16,17-
tetradecahydrocyclopenta[a]phenanthrene-3,7,8-triol
(5S)-17-[(2R,5S)-5,6-dimethylheptan-2-yl]-10,13-dimethyl-2,3,4,5,6,7,8,9,11,12,14,15,16,17-
tetradecahydro-1H-cyclopenta[a]phenanthrene-3,11-diol
(5S,8R,11R)-17-[(2R,5S)-5,6-dimethylheptan-2-yl]-10,13-dimethyl-2,3,4,5,6,7,8,9,11,12,14,15,16,17-
tetradecahydro-1H-cyclopenta[a]phenanthrene-3,11-diol
(5S,11R)-17-[(2R,5S)-5,6-dimethylheptan-2-yl]-10,13-dimethyl-2,3,4,5,6,7,8,9,11,12,14,15,16,17-
tetradecahydro-1H-cyclopenta[a]phenanthrene-3,11-diol
(5S)-10,13-dimethyl-17-[(2R)-6-methylheptan-2-yl]-2,3,4,5,6,7,8,9,11,12,14,15,16,17-tetradecahydro-
1H-cyclopenta[a]phenanthrene-2,3-diol
(10R)-17-[(2R,5S)-5,6-dimethylheptan-2-yl]-10,13-dimethyl-2,3,4,5,6,7,8,9,11,12,14,15,16,17-
tetradecahydro-1H-cyclopenta[a]phenanthrene-2,3-diol
(4S,5R)-10,13-dimethyl-2,3,4,5,6,7,8,9,11,12,14,15,16,17-tetradecahydro-1H-
cyclopenta[a]phenanthrene-3,4-diol
(3S,5R,6R,10R,13R)-17-(7-hydroxy-6-methylheptan-2-yl)-10,13-dimethyl-
1,2,3,4,6,7,8,9,11,12,14,15,16,17-tetradecahydrocyclopenta[a]phenanthrene-3,5,6-triol
(3S,5R,6R,8S,9S,10R,13S,14S,17R)-17-[(2S,3R,4R)-5-ethyl-3,4-dihydroxy-6-methylheptan-2-yl]-10,13-
dimethyl-1,2,3,4,6,7,8,9,11,12,14,15,16,17-tetradecahydrocyclopenta[a]phenanthrene-3,5,6-triol
(1S,3S,5R,6R,10S,13S,17R)-17-[(2S,3R,5R)-5-ethyl-3-hydroxy-6-methylheptan-2-yl]-10,13-dimethyl-
1,2,3,4,6,7,8,9,11,12,14,15,16,17-tetradecahydrocyclopenta[a]phenanthrene-1,3,5,6-tetrol
(3R,5S,10S,12S,13R)-17-[(2R,5R,6R)-5,7-dihydroxy-6-methylheptan-2-yl]-10,13-dimethyl-
2,3,4,5,6,7,8,9,11,12,14,15,16,17-tetradecahydro-1H-cyclopenta[a]phenanthrene-3,7,12-triol
(3S,5S,8R,9R,10R,13S,14S,17R)-17-[(1S)-1-hydroxyethyl]-10,13-dimethyl-
1,2,3,4,5,6,7,8,9,11,12,14,15,16-tetradecahydrocyclopenta[a]phenanthrene-3,17-diol
(3S,5S,8R,9R,10R,13S,14S,17R)-17-[(1R)-1-hydroxyethyl]-10,13-dimethyl-
1,2,3,4,5,6,7,8,9,11,12,14,15,16-tetradecahydrocyclopenta[a]phenanthrene-3,17-diol
(3S,5S,8R,9R,10R,13S,14R,17R)-17-[(1S)-1-hydroxyethyl]-10,13-dimethyl-
1,2,3,4,5,6,7,8,9,11,12,14,15,16-tetradecahydrocyclopenta[a]phenanthrene-3,17-diol
(3S,5S,8R,9R,10R,13S,14R,17R)-17-[(1R)-1-hydroxyethyl]-10,13-dimethyl-
1,2,3,4,5,6,7,8,9,11,12,14,15,16-tetradecahydrocyclopenta[a]phenanthrene-3,17-diol
(5S,7S,8R,10S,13R)-17-[5,7-dihydroxy-6-(hydroxymethyl)heptan-2-yl]-10,13-dimethyl-
2,3,4,5,6,7,8,9,11,12,14,15,16,17-tetradecahydro-1H-cyclopenta[a]phenanthrene-3,7,12-triol
(2R,3R)-2-[(5R,8R,9S,10S,13S,14S,17S)-10,13-dimethyl-2,3,4,5,6,7,8,9,11,12,14,15,16,17-
tetradecahydro-1H-cyclopenta[a]phenanthren-17-yl]-6-methylheptane-2,3-diol
(2S,3S,10S,13R)-17-(7-hydroxy-6-methylheptan-2-yl)-10,13-dimethyl-2,3,4,5,6,7,8,9,11,12,14,15,16,17-
tetradecahydro-1H-cyclopenta[a]phenanthrene-2,3-diol
(3S,6R,10R,13S)-17-[(2S)-2-hydroxy-6-methylheptan-2-yl]-10,13-dimethyl-
2,3,4,5,6,7,8,9,11,12,14,15,16,17-tetradecahydro-1H-cyclopenta[a]phenanthrene-3,6-diol
(3S,6S,10R,13S)-17-[(2S)-2-hydroxy-6-methylheptan-2-yl]-10,13-dimethyl-
2,3,4,5,6,7,8,9,11,12,14,15,16,17-tetradecahydro-1H-cyclopenta[a]phenanthrene-3,6-diol
(3R,5S,7R,12S)-17-[7-hydroxy-6-(hydroxymethyl)heptan-2-yl]-10,13-dimethyl-
2,3,4,5,6,7,8,9,11,12,14,15,16,17-tetradecahydro-1H-cyclopenta[a]phenanthrene-3,7,12-triol
(3R,5R,7R,12S)-17-[7-hydroxy-6-(hydroxymethyl)heptan-2-yl]-10,13-dimethyl-
2,3,4,5,6,7,8,9,11,12,14,15,16,17-tetradecahydro-1H-cyclopenta[a]phenanthrene-3,7,12-triol
(3R,5R,10S,13S,17R)-17-(1,2-dihydroxyethyl)-10,13-dimethyl-1,2,3,4,5,6,7,8,9,11,12,14,15,16-
tetradecahydrocyclopenta[a]phenanthrene-3,11,17-triol
(3R,7R,8R,9S,10S,12S,13R,14S,17R)-17-[(2R)-7-hydroxy-6-(hydroxymethyl)heptan-2-yl]-10,13-
dimethyl-2,3,4,5,6,7,8,9,11,12,14,15,16,17-tetradecahydro-1H-cyclopenta[a]phenanthrene-3,7,12-triol
(5S,17R)-17-(1-hydroxyethyl)-10,13-dimethyl-1,2,3,4,5,6,7,8,9,11,12,14,15,16-
tetradecahydrocyclopenta[a]phenanthrene-3,17-diol
(3S,5S,6S,10R,13R)-17-[(2R,5S)-6-hydroxy-5,6-dimethylheptan-2-yl]-10,13-dimethyl-
1,2,3,4,6,7,8,9,11,12,14,15,16,17-tetradecahydrocyclopenta[a]phenanthrene-3,5,6-triol
(5R)-17-[(2R)-5,6-dimethylheptan-2-yl]-10,13-dimethyl-1,2,3,4,6,7,8,9,11,12,14,15,16,17-
tetradecahydrocyclopenta[a]phenanthrene-1,3,5,6-tetrol
(3S,5R,6R,10R,13R,17R)-17-[(2R)-5,6-dimethylheptan-2-yl]-10-(hydroxymethyl)-13-methyl-
1,2,3,4,6,7,8,9,11,12,14,15,16,17-tetradecahydrocyclopenta[a]phenanthrene-3,5,6-triol
(5R)-17-[(2R,5S)-6-hydroxy-5,6-dimethylheptan-2-yl]-10,13-dimethyl-1,2,3,4,6,7,8,9,11,12,14,15,16,17-
tetradecahydrocyclopenta[a]phenanthrene-3,5,6-triol
17-[(2R,5S)-5,6-dimethylheptan-2-yl]-10,13-dimethyl-1,2,3,4,6,7,8,9,11,12,14,15,16,17-
tetradecahydrocyclopenta[a]phenanthrene-1,3,5,6-tetrol
(5R)-10,13-dimethyl-17-[(2R)-6-methylheptan-2-yl]-1,2,3,4,6,7,8,9,11,12,14,15,16,17-
tetradecahydrocyclopenta[a]phenanthrene-1,3,5,6-tetrol
(3S,5R,6R,7R,10R,13R)-17-[(2R,5S)-5,6-dimethylheptan-2-yl]-10,13-dimethyl-
1,2,3,4,6,7,8,9,11,12,14,15,16,17-tetradecahydrocyclopenta[a]phenanthrene-3,5,6,7-tetrol
17-(2-hydroxypropan-2-yl)-10,13-dimethyl-2,3,4,5,6,7,8,9,11,12,14,15,16,17-tetradecahydro-1H-
cyclopenta[a]phenanthren-3-ol
(3S,5S,8R,9S,10S,13R,14S,17R)-17-[(2R)-6-hydroxy-6-methylheptan-2-yl]-10,13-dimethyl-
2,3,4,5,6,7,8,9,11,12,14,15,16,17-tetradecahydro-1H-cyclopenta[a]phenanthren-3-ol
(8R,9S,10S,13R,17R)-17-[(2R)-6-hydroxy-6-methylheptan-2-yl]-10,13-dimethyl-
2,3,4,5,6,7,8,9,11,12,14,15,16,17-tetradecahydro-1H-cyclopenta[a]phenanthrene-3,7,12-triol
(3S,5R,6R,9S,10R,11S,13R)-10,13-dimethyl-17-(6-methylheptan-2-yl)-1,2,3,4,6,7,8,9,11,12,14,15,16,17-
tetradecahydrocyclopenta[a]phenanthrene-3,5,6,11-tetrol
(5R)-17-[(2R,5S)-6,7-dihydroxy-5,6-dimethylheptan-2-yl]-10,13-dimethyl-
1,2,3,4,6,7,8,9,11,12,14,15,16,17-tetradecahydrocyclopenta[a]phenanthrene-3,5,6-triol
(5R)-17-(5,6-dimethylheptan-2-yl)-10,13-dimethyl-1,2,3,4,6,7,8,9,11,12,14,15,16,17-
tetradecahydrocyclopenta[a]phenanthrene-1,3,5,6-tetrol
(5R)-17-(6-hydroxy-5,6-dimethylheptan-2-yl)-10,13-dimethyl-1,2,3,4,6,7,8,9,11,12,14,15,16,17-
tetradecahydrocyclopenta[a]phenanthrene-3,5,6-triol
(3S,6R)-6-[(8R,9S,10S,13R,14S,17R)-10,13-dimethyl-2,3,4,5,6,7,8,9,11,12,14,15,16,17-tetradecahydro-
1H-cyclopenta[a]phenanthren-17-yl]-2-methylheptane-2,3-diol
(3R,5R,8R,9S,10S,12S,13R,14S,17R)-10,13-dimethyl-17-[(2R)-pentan-2-yl]-
2,3,4,5,6,7,8,9,11,12,14,15,16,17-tetradecahydro-1H-cyclopenta[a]phenanthrene-3,12-diol
(3S,5S,8R,9S,10S,11S,13R,14R,17R)-10,13-dimethyl-17-[(2R)-6-methylheptan-2-yl]-
2,3,4,5,6,7,8,9,11,12,14,15,16,17-tetradecahydro-1H-cyclopenta[a]phenanthrene-3,11-diol
(3S,5S,8S,9S,10S,11R,13R,14R,17R)-10,13-dimethyl-17-[(2R)-6-methylheptan-2-yl]-
2,3,4,5,6,7,8,9,11,12,14,15,16,17-tetradecahydro-1H-cyclopenta[a]phenanthrene-3,11-diol
(8S,9S,10S,13R,14S,17R)-10,13-dimethyl-17-[(2R)-6-methylheptan-2-yl]-
2,3,4,5,6,7,8,9,11,12,14,15,16,17-tetradecahydro-1H-cyclopenta[a]phenanthrene-3,11-diol
(3S,5S,6S,8R,9S,10R,13S,14S,17S)-17-[(2R)-2-hydroxy-6-methylheptan-2-yl]-10,13-dimethyl-
2,3,4,5,6,7,8,9,11,12,14,15,16,17-tetradecahydro-1H-cyclopenta[a]phenanthrene-3,6-diol
(8R,9S,10R,13S,14S,17S)-17-[(2S)-2-hydroxy-6-methylheptan-2-yl]-10,13-dimethyl-
2,3,4,5,6,7,8,9,11,12,14,15,16,17-tetradecahydro-1H-cyclopenta[a]phenanthrene-3,6-diol
(8S,9S,10R,13R,14S,17R)-17-[(2R,5S)-6-hydroxy-5,6-dimethylheptan-2-yl]-10,13-dimethyl-
1,2,3,4,6,7,8,9,11,12,14,15,16,17-tetradecahydrocyclopenta[a]phenanthrene-3,5,6-triol
(3S,5R,6R,8S,9S,10R,12S,13R,14S,17R)-17-[(2R,5S)-5,6-dimethylheptan-2-yl]-10,13-dimethyl-
1,2,3,4,6,7,8,9,11,12,14,15,16,17-tetradecahydrocyclopenta[a]phenanthrene-3,5,6,12-tetrol
(8S,9S,10R,13R,14S,17R)-17-[(2R,5S)-5,6-dimethylheptan-2-yl]-10,13-dimethyl-
1,2,3,4,6,7,8,9,11,12,14,15,16,17-tetradecahydrocyclopenta[a]phenanthrene-3,5,6,12-tetrol
(3R,5R,8R,9S,10S,12S,13R,14S,17R)-17-[(2R,5S)-5,6-dimethylheptan-2-yl]-10,13-dimethyl-
2,3,4,5,6,7,8,9,11,12,14,15,16,17-tetradecahydro-1H-cyclopenta[a]phenanthrene-3,12-diol
(8R,9S,10S,13R,14S,17R)-17-[(2R,5S)-5,6-dimethylheptan-2-yl]-10,13-dimethyl-
2,3,4,5,6,7,8,9,11,12,14,15,16,17-tetradecahydro-1H-cyclopenta[a]phenanthrene-3,12-diol
(8S,9S,10S,13S,14S,17R)-17-(1,2-dihydroxyethyl)-10,13-dimethyl-1,2,3,4,5,6,7,8,9,11,12,14,15,16-
tetradecahydrocyclopenta[a]phenanthrene-3,11,17-triol
17-ethyl-10,13-dimethyl-2,3,4,5,6,7,8,9,11,12,14,15,16,17-tetradecahydrocyclopenta[a]phenanthrene-
1,1,2-triol
(3S,5R,6R,10R,11S,13R,17R)-10,13-dimethyl-17-[(2R)-6-methylheptan-2-yl]-
1,2,3,4,6,7,8,9,11,12,14,15,16,17-tetradecahydrocyclopenta[a]phenanthrene-3,5,6,11-tetrol
(3S,4S,6S,10R,13R)-17-[(2R,5S)-6-hydroxy-5,6-dimethylheptan-2-yl]-4,10,13-trimethyl-
2,3,4,5,6,7,8,9,11,12,14,15,16,17-tetradecahydro-1H-cyclopenta[a]phenanthrene-3,6-diol
(1R,3S,5R,6R,10S,11R,13R,17R)-10,13-dimethyl-17-[(2R)-6-methylheptan-2-yl]-
1,2,3,4,6,7,8,9,11,12,14,15,16,17-tetradecahydrocyclopenta[a]phenanthrene-1,3,5,6,11-pentol
(3S,5R,6R,10R,13S)-17-[(2R,5S)-5,6-dimethylheptan-2-yl]-13-(hydroxymethyl)-10-methyl-
1,2,3,4,6,7,8,9,11,12,14,15,16,17-tetradecahydrocyclopenta[a]phenanthrene-3,5,6-triol
(3S,6R,10R,13R,17R)-17-(5,6-dimethylheptan-2-yl)-10,13-dimethyl-1,2,3,4,6,7,8,9,11,12,14,15,16,17-
tetradecahydrocyclopenta[a]phenanthrene-3,5,6,11-tetrol
(3S,6R,10R,13R,17R)-17-(7-hydroxy-6-methylheptan-2-yl)-10,13-dimethyl-
1,2,3,4,6,7,8,9,11,12,14,15,16,17-tetradecahydrocyclopenta[a]phenanthrene-3,5,6-triol
(3S,10S,13R,14S)-17-[(2R,5S)-6-hydroxy-5,6-dimethylheptan-2-yl]-10,13,14-trimethyl-
1,2,3,4,5,6,7,8,9,11,12,15,16,17-tetradecahydrocyclopenta[a]phenanthren-3-ol
(3R,4S,5S,8S,9S,10S,11R,12S,13S,14S,17R)-17-[(2R)-1-hydroxy-6-methylheptan-2-yl]-10,13-dimethyl-
2,3,4,5,6,7,8,9,11,12,14,15,16,17-tetradecahydro-1H-cyclopenta[a]phenanthrene-3,4,11,12-tetrol
(2S,5S,8R,9R,10S,13R,14R,17S)-17-[(2S)-2-hydroxy-6-methylheptan-2-yl]-4,4,8,10,14-pentamethyl-
2,3,5,6,7,9,11,12,13,15,16,17-dodecahydro-1H-cyclopenta[a]phenanthren-2-ol
(5S,8R,9R,10S,12R,13R,14R,17S)-17-[(2S)-2-hydroxy-6-methylheptan-2-yl]-4,4,8,10,14-pentamethyl-
2,3,5,6,7,9,11,12,13,15,16,17-dodecahydro-1H-cyclopenta[a]phenanthren-12-ol
(1R,3S,5R,6R,8S,9S,10S,13R,14S,17R)-17-[(2R)-5,6-dimethylheptan-2-yl]-10,13-dimethyl-
1,2,3,4,6,7,8,9,11,12,14,15,16,17-tetradecahydrocyclopenta[a]phenanthrene-1,3,5,6-tetrol
(3S,5R,6R,8S,9S,10R,13R,14S,17R)-17-[(2R)-6-hydroxy-5,6-dimethylheptan-2-yl]-10,13-dimethyl-
1,2,3,4,6,7,8,9,11,12,14,15,16,17-tetradecahydrocyclopenta[a]phenanthrene-3,5,6-triol
(3R,4S,5S,8S,9S,10S,11S,13S,14S,17R)-17-[(2R)-1-hydroxy-6-methylheptan-2-yl]-10,13-dimethyl-
2,3,4,5,6,7,8,9,11,12,14,15,16,17-tetradecahydro-1H-cyclopenta[a]phenanthrene-3,4,11-triol
(3S,3aS,5aR,5bR,7aS,11S,11aS,11bS,12R,13aR,13bS)-11a-(hydroxymethyl)-3-(2-hydroxypropan-2-yl)-
5a,5b,8,8,13b-pentamethyl-1,2,3,3a,4,5,6,7,7a,9,10,11,11b,12,13,13a-
hexadecahydrocyclopenta[a]chrysene-11,12-diol
10,13-dimethyl-17-(6-methylheptan-2-yl)-2,3,4,5,6,7,8,9,11,12,14,15,16,17-tetradecahydro-1H-
cyclopenta[a]phenanthrene-3,12-diol
10,13-dimethyl-17-(6-methylheptan-2-yl)-2,3,4,5,6,7,8,9,11,12,14,15,16,17-tetradecahydro-1H-
cyclopenta[a]phenanthrene-6,12-diol
2-(3-hydroxy-4,4,8,10,14-pentamethyl-2,3,5,6,7,9,11,12,13,15,16,17-dodecahydro-1H-
cyclopenta[a]phenanthren-17-yl)-6-methylheptane-2,5-diol
(3R,8S,9S,10S,11S,13S,14S,17R)-17-(1,2-dihydroxyethyl)-10,13-dimethyl-
1,2,3,4,5,6,7,8,9,11,12,14,15,16-tetradecahydrocyclopenta[a]phenanthrene-3,11,17-triol
(8S,9S,10R,13R,14S,17R)-17-[(2R)-6,7-dihydroxy-6-methylheptan-2-yl]-10,13-dimethyl-
2,3,4,5,6,7,8,9,11,12,14,15,16,17-tetradecahydro-1H-cyclopenta[a]phenanthrene-3,6,7-triol
(8R,9S,10S,13R,14S,17R)-17-[(2R)-7-hydroxy-6-(hydroxymethyl)heptan-2-yl]-10,13-dimethyl-
2,3,4,5,6,7,8,9,11,12,14,15,16,17-tetradecahydro-1H-cyclopenta[a]phenanthrene-3,7,12-triol
(5R,8S,9S,10S,13R,14S,17R)-10,13-dimethyl-17-[(2R)-6-methylheptan-2-yl]-
2,3,4,5,6,7,8,9,11,12,14,15,16,17-tetradecahydrocyclopenta[a]phenanthrene-1,1,2-triol
(8S,9S,10S,13R,14S,17R)-10,13-dimethyl-17-[(2R)-6-methylheptan-2-yl]-
2,3,4,5,6,7,8,9,11,12,14,15,16,17-tetradecahydrocyclopenta[a]phenanthrene-1,1,2-triol
(8R,9S,10S,13R,14S,17R)-17-[(2R)-5,7-dihydroxy-6-methylheptan-2-yl]-10,13-dimethyl-
2,3,4,5,6,7,8,9,11,12,14,15,16,17-tetradecahydro-1H-cyclopenta[a]phenanthrene-3,7-diol
(8R,9S,10S,13R,14S,17R)-17-[(2R)-7-hydroxy-6-(hydroxymethyl)heptan-2-yl]-10,13-dimethyl-
2,3,4,5,6,7,8,9,11,12,14,15,16,17-tetradecahydro-1H-cyclopenta[a]phenanthrene-3,7-diol
(3R,5S,7R,8R,9S,10S,12S,13R,14S,17R)-17-[(2R)-5-ethyl-5-hydroxy-6-methylheptan-2-yl]-10,13-
dimethyl-2,3,4,5,6,7,8,9,11,12,14,15,16,17-tetradecahydro-1H-cyclopenta[a]phenanthrene-3,7,12-triol
(8R,9S,10S,13R,14S,17R)-17-[(2R)-6-hydroxy-5,6-dimethylheptan-2-yl]-10,13-dimethyl-
2,3,4,5,6,7,8,9,11,12,14,15,16,17-tetradecahydro-1H-cyclopenta[a]phenanthrene-3,7,12-triol
(8R,9S,10S,13R,14S,17R)-17-[(2R)-5-hydroxy-5,6-dimethylheptan-2-yl]-10,13-dimethyl-
2,3,4,5,6,7,8,9,11,12,14,15,16,17-tetradecahydro-1H-cyclopenta[a]phenanthren-3-ol
(3S,5R,7R,8R,9S,10S,13S,14S,17R)-17-(1-hydroxyethyl)-10,13-dimethyl-
1,2,3,4,5,6,7,8,9,11,12,14,15,16-tetradecahydrocyclopenta[a]phenanthrene-3,7,17-triol
(8R,9S,10S,13S,14S,17R)-17-(1-hydroxyethyl)-10,13-dimethyl-1,2,3,4,5,6,7,8,9,11,12,14,15,16-
tetradecahydrocyclopenta[a]phenanthrene-3,7,17-triol
(8S,9S,10S,13R,14S,17S)-17-ethyl-10,13-dimethyl-2,3,4,5,6,7,8,9,11,12,14,15,16,17-
tetradecahydrocyclopenta[a]phenanthrene-1,1,2-triol
(5S,8S,9S,10S,13R,14S,17R)-10,13-dimethyl-17-[(2R)-6-methylheptan-2-yl]-
2,3,4,5,6,7,8,9,11,12,14,15,16,17-tetradecahydrocyclopenta[a]phenanthrene-1,1,2-triol
(3R,5S,8R,9S,10S,13S,14S,17R)-17-[(1R)-1-hydroxyethyl]-10,13-dimethyl-
1,2,3,4,5,6,7,8,9,11,12,14,15,16-tetradecahydrocyclopenta[a]phenanthrene-3,17-diol
(8S,9S,10S,13R,14S,17R)-17-[(2R)-6-hydroxy-5,6-dimethylheptan-2-yl]-10,13-dimethyl-
1,2,3,4,6,7,8,9,11,12,14,15,16,17-tetradecahydrocyclopenta[a]phenanthrene-1,3,5,6-tetrol
(3R,5R,8S,9S,10S,11S,13S,14S,17R)-17-(1,2-dihydroxyethyl)-10,13-dimethyl-
1,2,3,4,5,6,7,8,9,11,12,14,15,16-tetradecahydrocyclopenta[a]phenanthrene-3,11,17-triol
17-(4-hydroxybutan-2-yl)-10,13-dimethyl-2,3,4,5,6,7,8,9,11,12,14,15,16,17-tetradecahydro-1H-
cyclopenta[a]phenanthrene-3,12-diol
17-(5-hydroxypentan-2-yl)-10,17-dimethyl-1,2,3,4,5,6,7,8,11,12,13,14,15,16-
tetradecahydrocyclopenta[a]phenanthrene-3,7,9-triol
10,13,16-trimethyl-2,3,4,5,6,7,8,9,11,12,14,15,16,17-tetradecahydro-1H-cyclopenta[a]phenanthrene-
3,4,6,7-tetrol
10,13-dimethyl-2,3,4,5,6,7,8,9,11,12,14,15,16,17-tetradecahydro-1H-cyclopenta[a]phenanthrene-6,7-diol
6-(10,13-dimethyl-2,3,4,5,6,7,8,9,11,12,14,15,16,17-tetradecahydro-1H-cyclopenta[a]phenanthren-17-
yl)-2-methylheptane-2,3-diol
(8R,9S,10S,13S,14S)-17-(1-hydroxyethyl)-10,13-dimethyl-1,2,3,4,5,6,7,8,9,11,12,14,15,16-
tetradecahydrocyclopenta[a]phenanthrene-3,17-diol
(8S,9S,10S,13S,14S)-17-(1,2-dihydroxyethyl)-10,13-dimethyl-1,2,3,4,5,6,7,8,9,11,12,14,15,16-
tetradecahydrocyclopenta[a]phenanthrene-3,11,17-triol
(3S,5S,8S,9S,10S,11S,13S,14S,17R)-17-(1,2-dihydroxyethyl)-10,13-dimethyl-
1,2,3,4,5,6,7,8,9,11,12,14,15,16-tetradecahydrocyclopenta[a]phenanthrene-3,11,17-triol
17-(hydroxymethyl)-10,13-dimethyl-1,2,3,4,5,6,7,8,9,11,12,14,15,16-
tetradecahydrocyclopenta[a]phenanthrene-3,17-diol
(3R,5R,8R,9S,10S,13S,14S,17R)-17-(hydroxymethyl)-10,13-dimethyl-1,2,3,4,5,6,7,8,9,11,12,14,15,16-
tetradecahydrocyclopenta[a]phenanthrene-3,17-diol
(3R,5S,8R,9S,10S,13S,14S,17R)-17-(hydroxymethyl)-10,13-dimethyl-1,2,3,4,5,6,7,8,9,11,12,14,15,16-
tetradecahydrocyclopenta[a]phenanthrene-3,17-diol
10,13-dimethyl-17-(6-methylheptan-2-yl)-1,2,3,4,5,6,7,9,11,12,14,15,16,17-
tetradecahydrocyclopentaralphenanthrene-3,7,8-triol
(3S,4R,5R,6S)-6-[(3R,5R,6S,8S,9S,10R,13S,14S,17R)-3,6-dihydroxy-10,13-dimethyl-
2,3,4,5,6,7,8,9,11,12,14,15,16,17-tetradecahydro-1H-cyclopenta[a]phenanthren-17-yl]-3-propan-2-
ylheptane-1,4,5-triol
17-(6-hydroxy-6-methylheptan-2-yl)-10,13-dimethyl-2,3,4,5,6,7,8,9,11,12,14,15,16,17-tetradecahydro-
1H-cyclopenta[a]phenanthren-3-ol
17-(5,7-dihydroxy-6-(hydroxymethyl)heptan-2-yl]-10,13-dimethyl-2,3,4,5,6,7,8,9,11,12,14,15,16,17-
tetradecahydro-1H-cyclopenta[a]phenanthrene-3,7,12-triol
(3S,4S,5 S,8S,9S,10R,13R,14S,17R)-10,13-dimethyl-17-[(2R)-6-methylheptan-2-yl]-
1,2,3,4,6,7,8,9,11,12,14,15,16,17-tetradecahydrocyclopenta[a]phenanthrene-3,4,5-triol
(3R,4R,5R,8S,9S,10R,13R,14S,17R)-10,13-dimethyl-17-[(2R)-6-methylheptan-2-yl]-
1,2,3,4,6,7,8,9,11,12,14,15,16,17-tetradecahydrocyclopenta[a]-phenanthrene-3,4,5-triol
(3S,5R,6R,8S,9S,10R,13R,14S,17R)-17-[(2R,5S)-6-hydroxy-5,6-dimethylheptan-2-yl]-10,13-dimethyl-
1,2,3,4,6,7,8,9,11,12,14,15,16,17-tetradecahydrocyclopenta[a]phenanthrene-3,5,6-triol
17-(5-hydroxypentan-2-yl)-10,13-dimethyl-2,3,4,5,6,7,8,9,11,12,14,15,16,17-tetradecahydro-1H-
cyclopenta[a]phenanthrene-3,12-diol
(3R,5S,8R,9S,10S,135,14S,17R)-17-[(1S)-1-hydroxyethyl]-10,13-dimethyl-
1,2,3,4,5,6,7,8,9,11,12,14,15,16-tetradecahydrocyclopenta[a]phenanthrene-3,17-diol
(3S,5S,8R,9S,10S,13S,14S,17R)-17-[(1R)-1-hydroxyethyl]-10,13-dimethyl-
1,2,3,4,5,6,7,8,9,11,12,14,15,16-tetradecahydrocyclopenta[a]phenanthrene-3,17-diol
(3S,5S,8R,9S,10S,13S,14S,17R)-17-[(1S)-1-hydroxyethyl]-10,13-dimethyl-
1,2,3,4,5,6,7,8,9,11,12,14,15,16-tetradecahydrocyclopenta[a]phenanthrene-3,17-diol
(3S,5S,8S,9R,10S,13R,14R,17S)-17-[(1S)-1-hydroxyethyl]-10,13-dimethyl-
1,2,3,4,5,6,7,8,9,11,12,14,15,16-tetradecahydrocyclopenta[a]phenanthrene-3,17-diol
(3S,5S,8S,9R,10S,13R,14S,17S)-17-[(1S)-1-hydroxyethyl]-10,13-dimethyl-
1,2,3,4,5,6,7,8,9,11,12,14,15,16-tetradecahydrocyclopenta[a]phenanthrene-3,17-diol
(3S,5S,8S,9R,10S,13S,14R,17S)-17-[(1S)-1-hydroxyethyl]-10,13-dimethyl-
1,2,3,4,5,6,7,8,9,11,12,14,15,16-tetradecahydrocyclopenta[a]phenanthrene-3,17-diol
(3S,5S,8S,9R,10S,13S,14S,17S)-17-[(1S)-1-hydroxyethyl]-10,13-dimethyl-
1,2,3,4,5,6,7,8,9,11,12,14,15,16-tetradecahydrocyclopenta[a]phenanthrene-3,17-diol
(3S,5S,8S,9R,10R,13S,14S,17R)-17-[(1R)-1-hydroxyethyl]-5,10,13-trimethyl-
2,3,4,6,7,8,9,11,12,14,15,16-dodecahydro-1H-cyclopenta[a]phenanthrene-3,17-diol
(3S,5S,8S,9R,10R,13S,14S,17R)-17-[(1S)-1-hydroxyethyl]-5,10,13-trimethyl-
2,3,4,6,7,8,9,11,12,14,15,16-dodecahydro-1H-cyclopenta[a]phenanthrene-3,17-diol
(3S,5S,8S,9R,10R,13S,14S,17S)-17-[(1R)-1-hydroxyethyl]-5,10,13-trimethyl-
2,3,4,6,7,8,9,11,12,14,15,16-dodecahydro-1H-cyclopenta[a]phenanthrene-3,17-diol
(3S,5S,8S,9R,10R,13S,14S,17S)-17-[(1S)-1-hydroxyethyl]-5,10,13-trimethyl-
2,3,4,6,7,8,9,11,12,14,15,16-dodecahydro-1H-cyclopenta[a]phenanthrene-3,17-diol
(3R,5S,8S,9R,10R,13S,14R,17R)-17-[(1R)-1-hydroxyethyl]-5,10,13-trimethyl-
2,3,4,6,7,8,9,11,12,14,15,16-dodecahydro-1H-cyclopenta[a]phenanthrene-3,17-diol
(3R,5S,8S,9R,10R,13S,14R,17R)-17-[(1S)-1-hydroxyethyl]-5,10,13-trimethyl-
2,3,4,6,7,8,9,11,12,14,15,16-dodecahydro-1H-cyclopenta[a]phenanthrene-3,17-diol
(3R,5S,8S,9R,I0R,13S,14S,17R)-17-[(1R)-1-hydroxyethyl]-5,10,13-trimethyl-
2,3,4,6,7,8,9,11,12,14,15,16-dodecahydro-1H-cyclopenta[a]phenanthrene-3,17-diol
(3R,5S,8S,9R,10R,13S,14S,17R)-17-[(1S)-1-hydroxyethyl]-5,10,13-trimethyl-
2,3,4,6,7,8,9,11,12,14,15,16-dodecahydro-1H-cyclopenta[a]phenanthrene-3,17-diol
(2S,3S,5S,8R,9S,10S,13R,14S,17R)-10,13-dimethyl-17-[(2R)-6-methylheptan-2-yl]-
2,3,4,5,6,7,8,9,11,12,14,15,16,17-tetradecahydro-1H-cyclopenta[a]phenanthrene-2,3-diol
(2R,3R,5S,8R,9S,10S,13R,14S,17R)-10,13-dimethyl-17-[(2R)-6-methylheptan-2-yl]-
2,3,4,5,6,7,8,9,11,12,14,15,16,17-tetradecahydro-1H-cyclopenta[a]phenanthrene-2,3-diol
(3R,5R,10S,13S,17R)-17-(hydroxymethyl)-10,13-dimethyl-1,2,3,4,5,6,7,8,9,11,12,14,15,16-
tetradecahydrocyclopenta[a]phenanthrene-3,17-diol
(3R,5R,10S,13R,17R)-17-[(2R)-6-hydroxy-6-methylheptan-2-yl]-10,13-dimethyl-
2,3,4,5,6,7,8,9,11,12,14,15,16,17-tetradecahydro-1H-cyclopenta[a]phenanthren-3-ol
(24R,S)-24-Methoxy-24-methylcycloartane-3 beta,24-diol
(3S,5R,8R,9R,10R,12R,13R,14R,17R)-4,4,8,10,14-pentamethyl-17-[(2R)-6-methylheptan-2-yl]-
2,3,5,6,7,9,11,12,13,15,16,17-dodecahydro-1H-cyclopenta[a]phenanthrene-3,12-diol
(2S,3R,5S,8R,9S,10S,13R,14S,17R)-10,13-dimethyl-17-[(2R)-6-methylheptan-2-yl]-
2,3,4,5,6,7,8,9,11,12,14,15,16,17-tetradecahydro-1H-cyclopenta[a]phenanthrene-2,3-diol
(3R,5R,9S,10S,13S,14S,17R)-17-[(1S)-1-hydroxyethyl]-10,13-dimethyl-1,2,3,4,5,6,7,8,9,11,12,14,15,16-
tetradecahydrocyclopenta[a]phenanthrene-3,17-diol
(3S,5S,8R,9S,10S,12S,13R,14S,17R)-17-[(2R)-5-ethyl-6-methylheptan-2-yl]-10,13-dimethyl-
2,3,4,5,6,7,8,9,11,12,14,15,16,17-tetradecahydro-1H-cyclopenta[a]phenanthrene-3,12-diol
(3R,5S,7R,8R,9S,10S,12S,13R,14S,17R)-17-[(2R,5R)-5,7-dihydroxyheptan-2-yl]-10,13-dimethyl-
2,3,4,5,6,7,8,9,11,12,14,15,16,17-tetradecahydro-1H-cyclopenta[a]phenanthrene-3,7,12-triol
(1R,3S,5R,6R,8S,9S,10S,13R,14S,17R)-10,13-dimethyl-17-[(2R)-6-methylheptan-2-yl]-
1,2,3,4,6,7,8,9,11,12,14,15,16,17-tetradecahydrocyclopenta[a]phenanthrene-1,3,5,6-tetrol
(3S,5R,6R,8S,9S,10R,13R,14S,17R)-17-[(2R)-7-hydroxy-6-methylheptan-2-yl]-10,13-dimethyl-
1,2,3,4,6,7,8,9,11,12,14,15,16,17-tetradecahydrocyclopenta[a]phenanthrene-3,5,6-triol
(3S,5R,6R,8S,9S,10R,11R,13R,14S,17R)-17-[(2R,5S)-5,6-dimethylheptan-2-yl]-10,13-dimethyl-
1,2,3,4,6,7,8,9,11,12,14,15,16,17-tetradecahydrocyclopenta[a]phenanthrene-3,5,6,11-tetrol
(3S,5R,6R,8S,9S,10R,13S,14S,17R)-17-[(2R,5S)-5,6-dimethylheptan-2-yl]-13-(hydroxymethyl)-10-
methyl-1,2,3,4,6,7,8,9,11,12,14,15,16,17-tetradecahydrocyclopenta[a]phenanthrene-3,5,6-triol
(3R,5R,8R,9S,10S,12S,13R,17R)-17-[(2R)-5-hydroxypentan-2-yl]-10,13-dimethyl-
2,3,4,5,6,7,8,9,11,12,14,15,16,17-tetradecahydro-1H-cyclopenta[a]phenanthrene-3,12-diol
(3S,4R,5R,6R,8S,9S,10R,13R,14S)-10,13-dimethyl-17-[(2R)-6-methylheptan-2-yl]-
1,2,3,4,6,7,8,9,11,12,14,15,16,17-tetradecahydrocyclopenta[a]phenanthrene-3,4,5,6-tetrol
(3R,5S,10S,13S,17S)-17-(hydroxymethyl)-10,13-dimethyl-1,2,3,4,5,6,7,8,9,11,12,14,15,16-
tetradecahydrocyclopenta[a]phenanthrene-3,17-diol
(3R,5R,10S,13S,17S)-17-(hydroxymethyl)-10,13-dimethyl-1,2,3,4,5,6,7,8,9,11,12,14,15,16-
tetradecahydrocyclopenta[a]phenanthrene-3,17-diol
(3S,5S,10S,13S,17R)-17-(hydroxymethyl)-10,13-dimethyl-1,2,3,4,5,6,7,8,9,11,12,14,15,16-
tetradecahydrocyclopenta[a]phenanthrene-3,17-diol
(3R,5R,8R,9S,10S,12S,13R,14S,17R)-17-[(2R)-butan-2-yl]-10,13-dimethyl-
2,3,4,5,6,7,8,9,11,12,14,15,16,17-tetradecahydro-1H-cyclopenta[a]phenanthrene-3,12-diol
(3R,5R,7R,8R,9S,10S,12S,13R,14S)-17-[(2R,5S)-5,7-dihydroxy-6-(hydroxymethyl)heptan-2-yl]-10,13-
dimethyl-2,3,4,5,6,7,8,9,11,12,14,15,16,17-tetradecahydro-1H-cyclopenta[a]phenanthrene-3,7,12-triol
(10S,13R,17R)-3-(1-hydroxypropan-2-yl)-10,13-dimethyl-17-[(2R)-6-methylheptan-2-yl]-
1,2,4,5,6,7,8,9,11,12,14,15,16,17-tetradecahydrocyclopenta[a]phenanthren-3-ol
(3R,5S,7R,8R,9S,10S,12S,13R,14S,17R)-17-[(2R,5R,6S)-5,7-dihydroxy-6-methylheptan-2-yl]-10,13-
dimethyl-2,3,4,5,6,7,8,9,11,12,14,15,16,17-tetradecahydro-1H-cyclopenta[a]phenanthrene-3,7,12-triol
(3R,5S,7R,10S,12S,13R)-17-[(2R,5R,6R)-5,7-dihydroxy-6-methylheptan-2-yl]-10,13-dimethyl-
2,3,4,5,6,7,8,9,11,12,14,15,16,17-tetradecahydro-1H-cyclopenta[a]phenanthrene-3,7,12-triol
(3R,5S,10S,13S,17R)-17-(hydroxymethyl)-10,13-dimethyl-1,2,3,4,5,6,7,8,9,11,12,14,15,16-
tetradecahydrocyclopenta[a]phenanthrene-3,17-diol
(3R,5S,7R,10S,12S,13R)-17-[(2R,5R)-5,7-dihydroxy-6-(hydroxymethyl)heptan-2-yl]-10,13-dimethyl-
2,3,4,5,6,7,8,9,11,12,14,15,16,17-tetradecahydro-1H-cyclopenta[a]phenanthrene-3,7,12-triol
(3S,5S,8R,9S,10S,13S,14S,17R)-17-(1-hydroxyethyl)-10,13-dimethyl-1,2,3,4,5,6,7,8,9,11,12,14,15,16-
tetradecahydrocyclopenta[a]phenanthrene-3,17-diol
(3S,5S,10S,13S,17R)-17-[(1R)-1-hydroxyethyl]-10,13-dimethyl-1,2,3,4,5,6,7,8,9,11,12,14,15,16-
tetradecahydrocyclopenta[a]phenanthrene-3,17-diol
(3S,5S,10S,13S,17R)-17-[(1S)-1-hydroxyethyl]-10,13-dimethyl-1,2,3,4,5,6,7,8,9,11,12,14,15,16-
tetradecahydrocyclopenta[a]phenanthrene-3,17-diol
(3R,5R,10S,11S,13S,17R)-17-(1,2-dihydroxyethyl)-10,13-dimethyl-1,2,3,4,5,6,7,8,9,11,12,14,15,16-
tetradecahydrocyclopenta[a]phenanthrene-3,11,17-triol
18-Acetoxyaphidicolin
(3R,5S,7R,8R,10S,12S,13R,17R)-17-[(2R,5R)-5,7-dihydroxyheptan-2-yl]-10,13-dimethyl-
2,3,4,5,6,7,8,9,11,12,14,15,16,17-tetradecahydro-1H-cyclopenta[a]phenanthrene-3,7,12-triol
(5S,10S)-17-[7-hydroxy-6-(hydroxymethyl)heptan-2-yl]-10,13-dimethyl-
2,3,4,5,6,7,8,9,11,12,14,15,16,17-tetradecahydro-1H-cyclopenta[a]phenanthrene-3,7,12-triol
(10S)-17-[7-hydroxy-6-(hydroxymethyl)heptan-2-yl]-10,13-dimethyl-2,3,4,5,6,7,8,9,11,12,14,15,16,17-
tetradecahydro-1H-cyclopenta[a]phenanthrene-3,7,12-triol
(3R,5R,8R,9S,10S,12S,13R,14S,17R)-17-[(2R)-4-hydroxybutan-2-yl]-10,13-dimethyl-
2,3,4,5,6,7,8,9,11,12,14,15,16,17-tetradecahydro-1H-cyclopenta[a]phenanthrene-3,12-diol
(3R,5R,8R,9S,10S,12S,13R,14S,17R)-10,13-dimethyl-17-[(2R)-6-methylheptan-2-yl]-
2,3,4,5,6,7,8,9,11,12,14,15,16,17-tetradecahydro-1H-cyclopenta[a]phenanthrene-3,12-diol
(3S,5S,7R,8R,9S,10S,12S,13R,14S,17R)-17-[(2R)-6-hydroxy-6-methylheptan-2-yl]-10,13-dimethyl-
2,3,4,5,6,7,8,9,11,12,14,15,16,17-tetradecahydro-1H-cyclopenta[a]phenanthrene-3,7,12-triol
(3R,5S,7S,8R,9S,10S,12S,13R,14S,17R)-17-[(2R)-6-hydroxy-6-methylheptan-2-yl]-10,13-dimethyl-
2,3,4,5,6,7,8,9,11,12,14,15,16,17-tetradecahydro-1H-cyclopenta[a]phenanthrene-3,7,12-triol
(3R,5R,7R,8R,9S,10S,13R,14S,17R)-17-[(2R)-7-hydroxy-6-(hydroxymethyl)heptan-2-yl]-10,13-
dimethyl-2,3,4,5,6,7,8,9,11,12,14,15,16,17-tetradecahydro-1H-cyclopenta[a]phenanthrene-3,7-diol
(3R,5S,7R,8R,9S,10S,12S,13R,14S,17R)-17-[(2R)-7-hydroxy-6-(hydroxymethyl)heptan-2-yl]-10,13-
dimethyl-2,3,4,5,6,7,8,9,11,12,14,15,16,17-tetradecahydro-1H-cyclopenta[a]phenanthrene-3,7,12-triol
(3R,5S,7R,8R,9S,10S,12S,13R,14S,17R)-17-[(2R)-5,7-dihydroxy-6-(hydroxymethyl)heptan-2-yl]-10,13-
dimethyl-2,3,4,5,6,7,8,9,11,12,14,15,16,17-tetradecahydro-1H-cyclopenta[a]phenanthrene-3,7,12-triol
(3R,5S,7R,8R,9S,10S,12S,13R,14S,17R)-17-[(2R)-6-hydroxy-6-methylheptan-2-yl]-10,13-dimethyl-
2,3,4,5,6,7,8,9,11,12,14,15,16,17-tetradecahydro-1H-cyclopenta[a]phenanthrene-3,7,12-triol
(3R,5R,7R,8R,9S,10S,12S,13R,14S,17R)-17-[(2R)-6-hydroxy-6-methylheptan-2-yl]-10,13-dimethyl-
2,3,4,5,6,7,8,9,11,12,14,15,16,17-tetradecahydro-1H-cyclopenta[a]phenanthrene-3,7,12-triol
(3R,5R,6R,7R,8S,9S,10R,13R,14S,17R)-17-[(2R)-6,7-dihydroxy-6-methylheptan-2-yl]-10,13-dimethyl-
2,3,4,5,6,7,8,9,11,12,14,15,16,17-tetradecahydro-1H-cyclopenta[a]phenanthrene-3,6,7-triol
(3R,5R,6S,7R,8S,9S,10R,13R,14S,17R)-17-[(2R)-6,7-dihydroxy-6-methylheptan-2-yl]-10,13-dimethyl-
2,3,4,5,6,7,8,9,11,12,14,15,16,17-tetradecahydro-1H-cyclopenta[a]phenanthrene-3,6,7-triol
(3R,5S,7R,8R,9S,10S,13R,14S,17R)-17-[(2R)-5,7-dihydroxy-6-methylheptan-2-yl]-10,13-dimethyl-
2,3,4,5,6,7,8,9,11,12,14,15,16,17-tetradecahydro-1H-cyclopenta[a]phenanthrene-3,7-diol
(3R,5S,7R,8R,9S,10S,12S,13R,14S,17R)-17-[(2R)-5,7-dihydroxy-6-methylheptan-2-yl]-10,13-dimethyl-
2,3,4,5,6,7,8,9,11,12,14,15,16,17-tetradecahydro-1H-cyclopenta[a]phenanthrene-3,7,12-triol
(3R,5R,7R,8R,9S,10S,12S,13R,14S,17R)-17-[(2R)-5,7-dihydroxy-6-methylheptan-2-yl]-10,13-dimethyl-
2,3,4,5,6,7,8,9,11,12,14,15,16,17-tetradecahydro-1H-cyclopenta[a]phenanthrene-3,7,12-triol
(3S,5R,7R,8R,9S,10S,12S,13R,14S,17R)-17-[(2R)-7-hydroxy-6-(hydroxymethyl)heptan-2-yl]-10,13-
dimethyl-2,3,4,5,6,7,8,9,11,12,14,15,16,17-tetradecahydro-1H-cyclopentatalphenanthrene-3,7,12-triol
(2S,3S,5R,7R,8R,9S,10S,12S,13R,14S,17R)-17-[(2R)-7-hydroxy-6-methylheptan-2-yl]-10,13-dimethyl-
2,3,4,5,6,7,8,9,11,12,14,15,16,17-tetradecahydro-1H-cyclopenta[a]phenanthrene-2,3,7,12-tetrol
(3R,5R,8R,9S,10S,12S,13R,14S,17R)-17-[(2R)-5-hydroxypentan-2-yl]-10,13-dimethyl-
2,3,4,5,6,7,8,9,11,12,14,15,16,17-tetradecahydro-1H-cyclopenta[a]phenanthrene-3,12-diol
(3R,5S,7R,8S,9S,10R,13R,14S,17R)-17-[(2R)-7-hydroxy-6-(hydroxymethyl)heptan-2-yl]-5,10,13-
trimethyl-1,2,3,4,6,7,8,9,11,12,14,15,16,17-tetradecahydrocyclopenta[a]phenanthrene-3,7-diol
17-(5,6-dimethylheptan-2-yl)-10,13-dimethyl-2,3,4,5,6,7,8,9,11,12,14,15,16,17-tetradecahydro-1H-
cyclopenta[a]phenanthrene-2,3-diol
17-(5,6-dimethylheptan-2-yl)-10,13-dimethyl-1,2,3,4,5,6,7,9,11,12,14,15,16,17-
tetradecahydrocyclopenta[a]phenanthrene-3,7,8-triol
10,13-dimethyl-2,3,4,5,6,7,8,9,11,12,14,15,16,17-tetradecahydro-1H-cyclopenta[a]phenanthrene-3,4-diol
17-(1-hydroxyethyl)-5,10,13-trimethyl-2,3,4,6,7,8,9,11,12,14,15,16-dodecahydro-1H-
cyclopenta[a]phenanthrene-3,17-diol
2-(hydroxymethyl)-10,13,17-trimethyl-1,2,3,4,5,6,7,8,9,11,12,14,15,16-
tetradecahydrocyclopenta[a]phenanthrene-3,17-diol
17-(6-hydroxy-6-methylheptan-2-yl)-10,13-dimethyl-2,3,4,5,6,7,8,9,11,12,14,15,16,17-tetradecahydro-
1H-cyclopenta[a]phenanthrene-3,7,12-triol
17-(4-hydroxy-4-methylpentan-2-yl)-10,13-dimethyl-2,3,4,5,6,7,8,9,11,12,14,15,16,17-tetradecahydro-
1H-cyclopenta[a]phenanthren-3-ol
17-(5-hydroxy-5-methylhexan-2-yl)-10,13-dimethyl-2,3,4,5,6,7,8,9,11,12,14,15,16,17-tetradecahydro-
1H-cyclopenta[a]phenanthrene-3,7,12-triol
17-(5,6-dimethylheptan-2-yl)-10,13-dimethyl-2,3,4,5,6,7,8,9,11,12,14,15,16,17-tetradecahydro-1H-
cyclopenta[a]phenanthrene-3,11-diol
(8R,9S,10S,13S,14S,17R)-17-(1-hydroxyethyl)-10,13-dimethyl-1,2,3,4,5,6,7,8,9,11,12,14,15,16-
tetradecahydrocyclopenta[a]phenanthrene-3,17-diol
(3R,5R,8R,9S,10S,13S,14S,17R)-17-(1-hydroxyethyl)-10,13-dimethyl-1,2,3,4,5,6,7,8,9,11,12,14,15,16-
tetradecahydrocyclopenta[a]phenanthrene-3,17-diol
(17S)-17-(1-hydroxyethyl)-10,13-dimethyl-1,2,3,4,5,6,7,8,9,11,12,14,15,16-
tetradecahydrocyclopenta[a]phenanthrene-3,17-diol
(5S)-17-(1-hydroxyethyl)-5,10,13-trimethyl-2,3,4,6,7,8,9,11,12,14,15,16-dodecahydro-1H-
cyclopenta[a]phenanthrene-3,17-diol
(3R,17R)-17-(1-hydroxyethyl)-5,10,13-trimethyl-2,3,4,6,7,8,9,11,12,14,15,16-dodecahydro-1H-
cyclopenta[a]phenanthrene-3,17-diol
10,13-dimethyl-17-pentan-2-yl-2,3,4,5,6,7,8,9,11,12,14,15,16,17-tetradecahydro-1H-
cyclopenta[a]phenanthrene-3,12-diol
10,13-dimethyl-17-(6-methylheptan-2-yl)-2,3,4,5,6,7,8,9,11,12,14,15,16,17-tetradecahydro-1H-
cyclopenta[a]phenanthrene-3,11-diol
17-(2-hydroxy-6-methylheptan-2-yl)-10,13-dimethyl-2,3,4,5,6,7,8,9,11,12,14,15,16,17-tetradecahydro-
1H-cyclopenta[a]phenanthrene-3,6-diol
17-(6-hydroxy-5,6-dimethylheptan-2-yl)-10,13-dimethyl-1,2,3,4,6,7,8,9,11,12,14,15,16,17-
tetradecahydrocyclopenta[a]phenanthrene-3,5,6-triol
17-(5,6-dimethylheptan-2-yl)-10,13-dimethyl-1,2,3,4,6,7,8,9,11,12,14,15,16,17-
tetradecahydrocyclopenta[a]phenanthrene-3,5,6,12-tetrol
17-(5,6-dimethylheptan-2-yl)-10,13-dimethyl-2,3,4,5,6,7,8,9,11,12,14,15,16,17-tetradecahydro-1H-
cyclopenta[a]phenanthrene-3,12-diol
17-(1-hydroxyethyl)-10,13-dimethyl-1,2,3,4,5,6,7,8,9,11,12,14,15,16-
tetradecahydrocyclopenta[a]phenanthrene-3,17-diol
17-(1,2-dihydroxyethyl)-10,13-dimethyl-1,2,3,4,5,6,7,8,9,11,12,14,15,16-
tetradecahydrocyclopenta[a]phenanthrene-3,11,17-triol
(3R,17R)-17-(1,2-dihydroxyethyl)-10,13-dimethyl-1,2,3,4,5,6,7,8,9,11,12,14,15,16-
tetradecahydrocyclopenta[a]phenanthrene-3,11,17-triol
18-Acetoxyaphidicolin
10,13-dimethyl-17-(6-methylheptan-2-yl)-2,3,4,5,6,7,8,9,11,12,14,15,16,17-tetradecahydro-1H-
cyclopenta[a]phenanthrene-2,3-diol
(3R,5R,8S,9S,10S,11S,13S,14S,17R)-17-[(1S)-1,2-dihydroxyethyl]-10,13-dimethyl-
1,2,3,4,5,6,7,8,9,11,12,14,15,16-tetradecahydrocyclopenta[a]phenanthrene-3,11,17-triol
(3R,5R,8S,9S,10S,11S,13S,14S,17R)-17-[(1R)-1,2-dihydroxyethyl]-10,13-dimethyl-
1,2,3,4,5,6,7,8,9,11,12,14,15,16-tetradecahydrocyclopenta[a]phenanthrene-3,11,17-triol
(2S,3R,5S,8R,9S,10S,13S,14S,17S)-2-(hydroxymethyl)-10,13,17-trimethyl-
1,2,3,4,5,6,7,8,9,11,12,14,15,16-tetradecahydrocyclopenta[a]phenanthrene-3,17-diol
(3R,5R,6S,7S,8S,9S,10R,13R,14S,17R)-17-[(2R)-6,7-dihydroxy-6-methylheptan-2-yl]-10,13-dimethyl-
2,3,4,5,6,7,8,9,11,12,14,15,16,17-tetradecahydro-1H-cyclopenta[a]phenanthrene-3,6,7-triol
(3R,5S,7R,8R,9S,10S,12S,13R,14S)-17-[(2R)-6-hydroxy-6-methylheptan-2-yl]-10,13-dimethyl-
2,3,4,5,6,7,8,9,11,12,14,15,16,17-tetradecahydro-1H-cyclopenta[a]phenanthrene-3,7,12-triol
(3R,5S,7R,8R,9S,10S,12S,13R,14S,17R)-17-[(2R,5R)-5,7-dihydroxy-6-(hydroxymethyl)heptan-2-yl]-
10,13-dimethyl-2,3,4,5,6,7,8,9,11,12,14,15,16,17-tetradecahydro-1H-cyclopenta[a]phenanthrene-3,7,12-
triol
(3R,5R,7R,8R,9S,10S,12S,13R,14S,17R)-17-[(2R)-7-hydroxy-6-(hydroxymethyl)heptan-2-yl]-10,13-
dimethyl-2,3,4,5,6,7,8,9,11,12,14,15,16,17-tetradecahydro-1H-cyclopenta[a]phenanthrene-3,7,12-triol
(3R,5S,7R,8R,9S,10S,13R,14S,17S)-17-[(2R)-5,7-dihydroxy-6-methylheptan-2-yl]-10,13-dimethyl-
2,3,4,5,6,7,8,9,11,12,14,15,16,17-tetradecahydro-1H-cyclopenta[a]phenanthrene-3,7-diol
(3R,5S,7R,8R,9S,10S,13R,14S,17R)-17-[(2R)-7-hydroxy-6-(hydroxymethyl)heptan-2-yl]-10,13-
dimethyl-2,3,4,5,6,7,8,9,11,12,14,15,16,17-tetradecahydro-1H-cyclopenta[a]phenanthrene-3,7-diol
(8R,9S,10S,13R,14S,17R)-17-[(2R)-5-ethyl-5-hydroxy-6-methylheptan-2-yl]-10,13-dimethyl-
2,3,4,5,6,7,8,9,11,12,14,15,16,17-tetradecahydro-1H-cyclopenta[a]phenanthrene-3,7,12-triol
(3R,5S,7R,8R,9S,10S,12S,13R,14S,17R)-17-[(2R,5R)-6-hydroxy-5,6-dimethylheptan-2-yl]-10,13-
dimethyl-2,3,4,5,6,7,8,9,11,12,14,15,16,17-tetradecahydro-1H-cyclopenta[a]phenanthrene-3,7,12-triol
(3S,5R,6R,7R,8S,9S,10R,13R,14S,17R)-17-[(2R,5S)-5,6-dimethylheptan-2-yl]-10,13-dimethyl-
1,2,3,4,6,7,8,9,11,12,14,15,16,17-tetradecahydrocyclopenta[a]phenanthrene-3,5,6,7-tetrol
(8R,9S,10S,13R,14S,17R)-17-[(2R,5R)-5-hydroxy-5,6-dimethylheptan-2-yl]-10,13-dimethyl-
2,3,4,5,6,7,8,9,11,12,14,15,16,17-tetradecahydro-1H-cyclopenta[a]phenanthren-3-ol
(3S,5R,7R,8R,9S,10S,13S,14S,17R)-17-[(1R)-1-hydroxyethyl]-10,13-dimethyl-
1,2,3,4,5,6,7,8,9,11,12,14,15,16-tetradecahydrocyclopenta[a]phenanthrene-3,7,17-triol
(3R,5R,8R,9S,10S,13S,14S,17R)-17-[(1R)-1-hydroxyethyl]-10,13-dimethyl-
1,2,3,4,5,6,7,8,9,11,12,14,15,16-tetradecahydrocyclopenta[a]phenanthrene-3,17-diol
(3R,5R,8R,9S,10S,13S,14S,17R)-17-[(1S)-1-hydroxyethyl]-10,13-dimethyl-
1,2,3,4,5,6,7,8,9,11,12,14,15,16-tetradecahydrocyclopenta[a]phenanthrene-3,17-diol

Aphidicolin and several aphidicolin analogs are commercially available. Standard doses of aphidicolin and several aphidicolin analogs are known in the art (e.g., 0.5 mg, 1.0 mg, 2.0 mg, 3.0 mg, 4.0 mg, 5.0 mg, 7.5 mg, 10 mg, 15 mg, 20 mg, 25 mg, 50 mg, 75 mg, 100 mg, 125 mg, 150 mg, 175 mg, 200 mg, 225 mg, 250 mg, 275 mg, 300 mg, 350 mg, 400 mg, 450 mg, 500 mg, 550 mg, 600 mg, 650 mg, 700 mg, 750 mg, 800 mg, 850 mg, 900 mg, or any range between any pair of recited doses) and can range, e.g., from 0.1 mg to 300 mg (e.g., 0.1 mg to 200 mg, 0.1 mg to 150 mg, 0.1 mg to 100 mg, and 1.0 mg to 50 mg).

### Damnacanthal

The methods, compositions, and kits of the invention may employ damnacanthal or a damnacanthal analog. A variety of damnacanthal analogs are known in the art. Nonlimiting examples of damnacanthal analogs are listed in Table 5.

TABLE 5
Damnacanthal analogs

3-hydroxy-1-methoxy-9,10-dioxoanthracene-2-carbaldehyde
3,5-dihydroxy-1-methoxy-9,10-dioxoanthracene-2-carbaldehyde
4-hydroxy-5,7-dimethoxy-9,10-dioxoanthracene-2-carbaldehyde
1-acetyl-5-hydroxy-7-methoxy-2-methylanthracene-9,10-dione
3-hydroxy-1-methoxy-2-(methoxymethyl)anthracene-9,10-dione
1-hydroxy-6,8-dimethoxy-2-methylanthracene-9,10-dione
1-hydroxy-3-methoxy-2-(1-methoxyethyl)anthracene-9,10-dione
1-hydroxy-2-(1-hydroxyethyl)-3-methoxyanthracene-9,10-dione
2-ethyl-1-hydroxy-3-methoxyanthracene-9,10-dione
3,5-dihydroxy-2-(hydroxymethyl)-1-methoxyanthracene-9,10-dione
6,8-dihydroxy-10-methoxy-3,4-dihydro-2H-benzo[a]anthracene-1,7,12-trione
10,12-dihydroxy-8-methoxy-3,4-dihydro-2H-tetracene-1,6,11-trione
methyl 3-hydroxy-1-methoxy-9,10-dioxoanthracene-2-carboxylate
3,6-dihydroxy-1-methoxy-2-methylanthracene-9,10-dione
3,6-dihydroxy-2-(hydroxymethyl)-1-methoxyanthracene-9,10-dione
1-hydroxy-3-methoxy-2-methylanthracene-9,10-dione
1-hydroxy-2-(hydroxymethyl)-3-methoxyanthracene-9,10-dione
1-hydroxy-3-methoxy-2-prop-2-enylanthracene-9,10-dione
1,3-dimethoxy-9,10-dioxoanthracene-2-carbaldehyde
2-(ethoxymethyl)-3-hydroxy-1-methoxyanthracene-9,10-dione
1-hydroxy-3-methoxy-6-methylanthracene-9,10-dione
3-hydroxy-2-(hydroxymethyl)-1-methoxyanthracene-9,10-dione
2-(ethoxymethyl)-3,5-dihydroxy-1-methoxyanthracene-9,10-dione
2-(ethoxymethyl)-3,8-dihydroxy-1-methoxyanthracene-9,10-dione
2-hexanoyl-1,3,8-trihydroxy-6-methoxyanthracene-9,10-dione
3-hydroxy-1-methoxy-2-methylanthracene-9,10-dione
1,8-dihydroxy-3-methyl-6-(trideuteriomethoxy)anthracene-9,10-dione
6-hydroxy-8-methoxy-3-methyl-9,10-dioxoanthracen-1-olate
3-hydroxy-1,8-dimethoxy-6-methylanthracene-9,10-dione
1,8-dihydroxy-3-methoxy-2,6-dimethylanthracene-9,10-dione
5,7-dimethoxy-9,10-dioxoanthracene-2-carboxylic acid
1-hydroxy-3,6,8-trimethoxy-2-(3-oxobutanoyl)anthracene-9,10-dione
1,5-dihydroxy-3-methoxy-7-methylanthracene-9,10-dione
1,3-dihydroxy-6,8-dimethoxy-2-[(E)-5-oxohex-1-enyl]anthracene-9,10-dione
1,8-dihydroxy-3-methoxy-6-methyl-2-(3-methylbut-2-enyl)anthracene-9,10-dione
2-(ethoxymethyl)-1,3-dihydroxy-6-methoxyanthracene-9,10-dione
1,3-dihydroxy-6-(hydroxymethyl)-7-methoxyanthracene-9,10-dione
1-hydroxy-2-(hydroxymethyl)-6,8-dimethoxy-3-methylanthracene-9,10-dione
4,5,7-trimethoxy-9,10-dioxoanthracene-2-carbaldehyde
1,8-dihydroxy-6-methoxy-3-methyl-9,10-dioxoanthracene-2-carboxylic acid
2-ethyl-1,3,8-trihydroxy-6-methoxyanthracene-9,10-dione
1,3-dihydroxy-8-methoxy-6-methylanthracene-9,10-dione
8-hydroxy-1,6-dimethoxy-9,10-dioxo-3-[(E)-prop-1-enyl]anthracene-2-carboxylic acid
1-hydroxy-3,8-dimethoxyanthracene-9,10-dione
1,3-dihydroxy-6-(hydroxymethyl)-8-methoxyanthracene-9,10-dione
1,8-dihydroxy-3-methoxy-6-methyl-2-[(E)-3-methylbut-1-enyl]anthracene-9,10-dione
1,8-dihydroxy-6-methoxy-3-methyl-2-[(E)-3-methylbut-1-enyl]anthracene-9,10-dione
1,8-dihydroxy-3-(hydroxymethyl)-6-methoxyanthracene-9,10-dione
1-hydroxy-3,8-dimethoxy-6-methylanthracene-9,10-dione
1-hydroxy-6,8-dimethoxy-3-methylanthracene-9,10-dione
8-hydroxy-1,3-dimethoxyanthracene-9,10-dione
1-butanoyl-2,4,5-trihydroxy-7-methoxyanthracene-9,10-dione
1,6-dihydroxy-8-methoxy-3-methylanthracene-9,10-dione
1,7-dihydroxy-3-methoxy-6-methylanthracene-9,10-dione
2-(hydroxymethyl)-1,3-dimethoxyanthracene-9,10-dione
1,6-dihydroxy-3-(hydroxymethyl)-8-methoxyanthracene-9,10-dione
1,8-dihydroxy-3-methoxy-6-methylanthracene-9,10-dione
1,3,8-trihydroxy-2-(1-hydroxyhexyl)-6-methoxyanthracene-9,10-dione
10-hydroxy-5,8-dimethoxy-2-methylnaphtho[2,3-h]chromene-4,7,12-trione
5,8,10-trihydroxy-2-methylnaphtho[2,3-h]chromene-4,7,12-trione
5,7-dihydroxy-4-methoxy-9,10-dioxoanthracene-2-carboxylate
5,7-dihydroxy-4-methoxy-9,10-dioxoanthracene-2-carboxylic acid
(1-butanoyl-4,5-dihydroxy-7-methoxy-9,10-dioxoanthracen-2-yl) sulfate
2-benzyl-1-hydroxy-3-methoxyanthracene-9,10-dione
5-hydroxy-1,3-dimethoxy-10H-anthracen-9-one
1,8-dihydroxy-3-[(2E,5E)-7-hydroxy-3,7-dimethylocta-2,5-dienoxy]-6-methylanthracene-9,10-dione
1-hydroxy-2-(1-hydroxyethyl)-3,6,8-trimethoxyanthracene-9,10-dione
1,8-dihydroxy-3-methyl-6-propoxyanthracene-9,10-dione
1,8-dihydroxy-3-methoxy-6-methyl-2-(3-methyl-2-oxobutyl)anthracene-9,10-dione
1-hydroxy-3,6,8-trimethoxyanthracene-9,10-dione
1,8-dihydroxy-3-methoxyanthracene-9,10-dione
1-hydroxy-3-methoxyanthracene-9,10-dione
3-ethyl-1-hydroxy-6,8-dimethoxy-9,10-dioxoanthracene-2-carboxylic acid
ethyl 4,5-dihydroxy-7-methoxy-9,10-dioxoanthracene-2-carboxylate
4,5-dihydroxy-7-methoxy-9,10-dioxoanthracene-2-carboxylic acid
3-(4,5-dihydroxy-7-methoxy-9,10-dioxoanthracen-2-yl)propanoic acid
5,7-dihydroxy-9-methoxy-2,3-dihydrocyclopenta[a]anthracene-1,6,11-trione
1,6,8-trimethoxy-3-methyl-9,10-dioxoanthracene-2-carboxylic acid
4,5-dihydroxy-1-(2-hydroxy-4,6-dimethoxyphenyl)-2-methylanthracene-9,10-dione
1,6,8-trihydroxy-3-methoxy-2-propan-2-ylanthracene-9,10-dione
methyl 3-ethyl-1-hydroxy-6,8-dimethoxy-9,10-dioxoanthracene-2-carboxylate
methyl 3-acetyl-1-hydroxy-6,8-dimethoxy-9,10-dioxoanthracene-2-carboxylate
3,6-dihydroxy-2-[(1S)-1-hydroxyhexyl]-1,8-dimethoxyanthracene-9,10-dione
1-ethoxy-3,8-dihydroxy-6-methoxy-2-methylanthracene-9,10-dione
ethyl 4-hydroxy-5,7-dimethoxy-9,10-dioxoanthracene-2-carboxylate
methyl 3-ethyl-1,8-dihydroxy-6-methoxy-9,10-dioxoanthracene-2-carboxylate
7-hydroxy-6,8-dimethoxy-9,10-dioxoanthracene-2-carbaldehyde
methyl 3-ethyl-8-hydroxy-1,6-dimethoxy-9,10-dioxoanthracene-2-carboxylate
5,9,11-trihydroxy-2-methylnaphtho[3,2-h]chromene-4,7,12-trione
4,5,7-trimethoxy-9,10-dioxoanthracene-2-carboxylic acid
1,3-dihydroxy-9,10-dioxoanthracene-2-carbaldehyde; 3-hydroxy-1-methoxy-9,10-dioxoanthracene-2-
carbaldehyde
5,7,9-trihydroxy-2-methylnaphtho[2,3-g]chromene-4,6,11-trione
3-[(2E)-3,7-dimethylocta-2,6-dienoxy]-1,8-dihydroxy-6-methylanthracene-9,10-dione
1-hydroxy-2-methyl-3-propan-2-yloxyanthracene-9,10-dione
3-hydroxy-1-methoxyanthracene-9,10-dione
1,4,5-trihydroxy-7-methoxy-2,3-dimethylanthracene-9,10-dione
1,8-dihydroxy-3-methyl-6-(3-methylbut-2-enoxy)anthracene-9,10-dione
3-(3,7-dimethylocta-2,6-dienoxy)-1,8-dihydroxy-6-methylanthracene-9,10-dione
1,8-dihydroxy-3,6-dimethoxyanthracene-9,10-dione
1,4,7-trihydroxy-5-methoxy-2-methylanthracene-9,10-dione
1,4,5-trihydroxy-7-methoxy-2-methylanthracene-9,10-dione
1,4-dihydroxy-5,7-dimethoxy-2,3-dimethylanthracene-9,10-dione
1,3,5-trihydroxy-2-(methoxymethyl)anthracene-9,10-dione
(1-butanoyl-4-hydroxy-5,7-dimethoxy-9,10-dioxoanthracen-2-yl) sulfate
1,8-dihydroxy-3-methoxy-2,6-dimethyl-10H-anthracen-9-one
1-hydroxy-2-(hydroxymethyl)-3-(methoxymethoxy)anthracene-9,10-dione
7-hydroxy-5,6-dimethyl-3-(4-methylphenyl)chromen-4-one
3,4-dihydroxy-1-methoxy-9,10-dioxoanthracene-2-carbaldehyde
1-(1-hydroxy-3-methoxy-9,10-dioxoanthracen-2-yl)ethyl acetate
1-(3-acetyl-2,4-dihydroxy-6-methoxyphenyl)-4,5-dihydroxy-2-methylanthracene-9,10-dione
(5,7-dihydroxy-4-methoxy-9,10-dioxoanthracen-2-yl)methyl acetate
4-(4,5-dihydroxy-7-methoxy-9,10-dioxoanthracen-2-yl)butanoic acid
1,3-dimethoxy-2-(methoxymethyl)anthracene-9,10-dione
1-(3-acetyl-2-hydroxy-4,6-dimethoxyphenyl)-4,5-dihydroxy-2-methylanthracene-9,10-dione
1,3,6-trihydroxy-2-(methoxymethyl)anthracene-9,10-dione
6-hydroxy-8-methoxy-3,4-dihydro-2H-benzo[a]anthracene-1,7,12-trione
1-acetyl-4-hydroxy-5-methoxy-2-(2-oxopropyl)anthracene-9,10-dione
1,8-dihydroxy-3-[(E)-4-hydroxy-3-methylbut-2-enoxy]-6-methylanthracene-9,10-dione
1,3-dimethoxy-2-methylanthracene-9,10-dione
1,8-dihydroxy-3-[4-hydroxy-3-(hydroxymethyl)but-2-enoxy]-6-methylanthracene-9,10-dione
methyl 8-hydroxy-3-[(2S)-2-hydroxypropyl]-1,6-dimethoxy-9,10-dioxoanthracene-2-carboxylate
2-hydroxy-6-(7-hydroxy-4-oxochromen-3-yl)benzaldehyde
1,3,8-trihydroxy-6-methoxyanthracene-9,10-dione
1-(3-acetyl-2,6-dihydroxy-4-methoxyphenyl)-4,5-dihydroxy-2-methylanthracene-9,10-dione
1,3-dihydroxy-2-(methoxymethyl)anthracene-9,10-dione
2-(ethoxymethyl)-1,3-dihydroxyanthracene-9,10-dione
1,8-dihydroxy-3-methoxy-6-methyl-10H-anthracen-9-one
2-acetyl-3-hydroxy-8-methoxy-1-methylanthracene-9,10-dlone
1,8,10-trihydroxy-3-methoxy-6-methyl-10-(3-methylbut-2-enyl)anthracen-9-one
1-(3-acetyl-4,5-dihydroxy-7-methoxynaphthalen-2-yl)propan-2-one
(10R)-1,8,10-trihydroxy-3-methoxy-6-methyl-10-(3-methylbut-2-enyl)anthracen-9-one
(10S)-1,8,10-trihydroxy-3-methoxy-6-methyl-10-(3-methylbut-2-enyl)anthracen-9-one
(3R)-3,6-dihydroxy-8-methoxy-3-methyl-2,4-dihydrobenzo[a]anthracene-1,7,12-trione
(3S)-3,6-dihydroxy-8-methoxy-3-methyl-2,4-dihydrobenzo[a]anthracene-1,7,12-trione
(3E)-2,5-dihydroxy-7-methoxy-3-(methoxymethylidene)-2-(2-oxopropyl)naphthalene-1,4-dione
(8-hydroxy-6-methoxy-3-methyl-9,10-dioxoanthracen-2-yl) hydrogen sulfate
sodium (8-hydroxy-6-methoxy-3-methyl-9,10-dioxoanthracen-2-yl) sulfate
methyl 5,7-dihydroxy-2,2,9-trimethyl-6,11-dioxonaphtho[3,2-g]chromene-8-carboxylate
3-[(2E)-3,7-dimethylocta-2,6-dienoxy]-1,8-dihydroxy-6-methyl-10H-anthracen-9-one
(1-butanoyl-4,5-dihydroxy-7-methoxy-9,10-dioxoanthracen-2-yl) hydrogen sulfate
5,7,12-trihydroxy-9-methoxy-3,4-dihydro-1H-tetracene-2,6,11-trione
2-hydroxy-4-methoxy-5-(3,5,5,8,8-pentamethyl-6,7-dihydronaphthalen-2-yl)benzaldehyde
(1-butanoyl-4-hydroxy-5,7-dimethoxy-9,10-dioxoanthracen-2-yl) hydrogen sulfate
sodium (1-butanoyl-4-hydroxy-5,7-dimethoxy-9,10-dioxoanthracen-2-yl) sulfate
2-[(E)-hex-1-enyl]-1,3-dimethoxyanthracene-9,10-dione
1,3-dimethoxy-2-prop-2-enylanthracene-9,10-dione
1,3,6,8-tetrahydroxy-2-(1-methoxyethyl)anthracene-9,10-dione
1,3,6,8-tetrahydroxy-2-(1-methoxyhexyl)anthracene-9,10-dione
1-[1-[(E)-but-2-en-2-yl]oxyethenyl]-4,5,7-trihydroxy-2-methylanthracene-9,10-dione
3-(4-hydroxyphenyl)-7-methoxy-5,6-dimethylchromen-4-one
5,7-dihydroxy-3-(3-hydroxy-4-methylphenyl)-6-methylchromen-4-one
3-(bromomethyl)-1,8-dihydroxy-6-methoxyanthracene-9,10-dione
6-(bromomethyl)-1-hydroxy-3,8-dimethoxyanthracene-9,10-dione
methyl 8-hydroxy-3-methoxy-1-methyl-9,10-dioxoanthracene-2-carboxylate
1-hydroxy-3-(methoxymethoxy)anthracene-9,10-dione
1-hydroxy-5,8-dimethoxy-3-methyl-9,10-dioxoanthracene-2-carbaldehyde
6-hydroxy-8,9-dimethoxy-3,4-dihydro-2H-anthracen-1-one
5,7-dihydroxy-2,9-dimethyl-2-(4-methylpent-3-enyl)naphtho[2,3-g]chromene-6,11-dione
(3S)-10,12-dihydroxy-8-methoxy-3-methyl-3,4-dihydronaphtho[3,2-g]isochromene-1,6,11-trione
1-hydroxy-8-methoxy-2-(3-oxobutanoyl)anthracene-9,10-dione
methyl 3-hydroxy-8-methoxy-1-methyl-9,10-dioxoanthracene-2-carboxylate
1,6,8-trihydroxy-3-methoxy-2-propan-2-yl-10H-anthracen-9-one
11-hydroxy-7,9-dimethoxy-1H-naphtho[2,3-f][2]benzofuran-3,5,10-trione
tert-butyl 2-(3-acetyl-4-hydroxy-5,7-dimethoxy-9,10-dioxoanthracen-2-yl)acetate
1-(2-hydroxy-4,6-dimethoxyphenyl)-2-methyl-4,5-di(propan-2-yloxy)anthracene-9,10-dione
2-[(2S,3S)-2,3-dimethyloxiran-2-yl]-5,11-dihydroxynaphtho[2,3-h]chromene-4,7,12-trione
8-hydroxy-6-methoxy-2-(4-methoxyphenyl)-3,4-dihydro-2H-naphthalen-1-one
1-hydroxy-2-(1-hydroxyethyl)-8-methoxyanthracene-9,10-dione
tert-butyl 2-[4-hydroxy-5,7-dimethoxy-9,10-dioxo-3-(3-oxobutanoyl)anthracen-2-yl]acetate
1,2,3-trihydroxy-8-methoxy-6-methylanthracene-9,10-dione
1-(3-acetyl-2-hydroxy-4,6-dimethoxyphenyl)-2-methylanthracene-9,10-dione
7,10-dihydroxy-5-methoxy-2-methylanthracene-1,4-dione
3-ethyl-6-hydroxy-8-methoxy-3,4-dihydro-2H-benzo[a]anthracene-1,7,12-trione
methyl 3-ethyl-1-hydroxy-6,8-dimethoxy-9-oxo-10H-anthracene-2-carboxylate
5,7-dihydroxy-2-(4-hydroxy-4-methylpentyl)-2,9-dimethylnaphtho[2,3-g]chromene-6,11-dione
3-ethyl-1-hydroxy-6,8-dimethoxy-9-oxo-10H-anthracene-2-carboxylic acid
7,9-dihydroxy-1,11-dimethoxy-3-methyl-5,6-dihydrobenzo[a]tetracene-8,13-dione
1,8-dihydroxy-3-(2-methoxy-3-methylbut-3-enoxy)-6-methylanthracene-9,10-dione
methyl 4-[3-(3,5-dioxohexyl)-1,5-dihydroxy-7-methoxy-9,10-dioxoanthracen-2-yl]-3-oxobutanoate
6-hydroxy-8-methoxy-3-methyl-3,4-dihydro-2H-benzo[a]anthracene-1,7,12-trione
9,14-dihydroxy-11-methoxy-5,6-dihydrobenzo[a]tetracene-8,13-dione
(3S)-3-ethyl-6-hydroxy-8-methoxy-3,4-dihydro-2H-benzo[a]anthracene-1,7,12-trione
methyl 2-(9,11-dihydroxy-2-methyl-4,7,12-trioxonaphtho[2,3-h]chromen-5-yl)acetate
2-[(2,4-dimethoxyphenyl)methyl]-1,8-dihydroxyanthracene-9,10-dione
3-[(2,4-dimethoxyphenyl)methyl]-1,8-dihydroxyanthracene-9,10-dione
methyl 2-(1-hydroxy-3,6,8-trimethoxy-9,10-dioxoanthracen-2-yl)-5-oxohexanoate
1,3,6,8-tetrahydroxy-2-[(1R)-1-methoxyethyl]anthracene-9,10-dione
(3S)-12-hydroxy-8,10-dimethoxy-3-methyl-3,4-dihydronaphtho[3,2-g]isochromene-1,6,11-trione
1,8-dihydroxy-3-(4-methoxybenzoyl)anthracene-9,10-dione
1,10,11-trihydroxy-3,8-dimethoxytetracene-5,12-dione
(4-hydroxy-5,7-dimethoxy-9,10-dioxoanthracen-2-yl)methyl acetate
ethyl 1,3,5-trihydroxy-9,10-dioxoanthracene-2-carboxylate
4-(3-acetyl-2,6-dihydroxy-4-methoxyphenyl)-1,8-dihydroxy-3-methyl-10H-anthracen-9-one
1-(3-acetyl-4,6-dihydroxy-2-methoxyphenyl)-4,5-dihydroxy-2-(hydroxymethyl)anthracene-9,10-dione
ethyl 1,3-dihydroxy-9,10-dioxoanthracene-2-carboxylate
1-hydroxy-2-(hydroxymethyl)-8-methoxyanthracene-9,10-dione
methyl 2-(8-hydroxy-1,3,6-trimethoxy-9,10-dioxoanthracen-2-yl)-5-oxohexanoate
methyl 3,6,8-trihydroxy-1-methyl-9,10-dioxoanthracene-2-carboxylate
3-docosoxy-1,8-dihydroxy-6-methylanthracene-9,10-dione
1,3,5-trihydroxy-9,10-dioxoanthracene-2-carbaldehyde
1,8-dihydroxy-3-(2-hydroxyethoxy)-6-methylanthracene-9,10-dione
1-(3-acetyl-2,6-dihydroxy-4-methoxyphenyl)-4,5-dihydroxy-2-(hydroxymethyl)anthracene-9,10-dione
10-hydroxy-5,7-dimethoxy-2,3-dimethylanthracene-1,4-dione
1,4,8-trihydroxy-6-methoxy-3-methyl-10H-anthracen-9-one
1,8,11-trihydroxy-3-methoxy-10-methyltetracene-5,12-dione
3,6-dihydroxy-8-methoxy-3-methyl-2,4-dihydrobenzo[a]anthracene-1,7,12-trione
1,2,8-trihydroxy-6-methoxy-3-methylanthracene-9,10-dione
1,3-dihydroxy-9,10-dioxoanthracene-2-carbaldehyde
methyl 1,3-dihydroxy-9,10-dioxoanthracene-2-carboxylate
(3S)-6-hydroxy-8-methoxy-3-methyl-3,4-dihydro-2H-benzo[a]anthracene-
1,8-dihydroxy-3-methoxy-6-methylanthracene-9,10-dione; 1,3,8-trihydroxy-6-methylanthracene-9,10-
dione
[7-hydroxy-5-methoxy-6-methyl-8-(3-methylbutyl)-9,10-dioxoanthracen-1-yl] acetate
1,3,6,11-tetrahydroxy-10-methoxytetracene-5,12-dione
3,8,9-trihydroxy-6-methoxy-3-methyl-10-(3-methylbut-2-enyl)-2,4-dihydroanthracen-1-one
6-hydroxy-1-methoxy-2-methylanthracene-9,10-dione
4,5,7-triacetyloxy-9,10-dioxoanthracene-1-carboxylic acid
2-hydroxy-5-(5-hydroxy-7-methoxy-4-oxochromen-3-yl)benzaldehyde
1,8-dihydroxy-3-methyl-6-phenacyloxyanthracene-9,10-dione
8-hydroxy-3-methoxy-1-[(E)-oct-1-enyl]-9,10-dioxoanthracene-2-carboxylic acid
1-[(E)-but-1-enyl]-8-hydroxy-3-methoxy-9,10-dioxoanthracene-2-carboxylic acid
(3R)-3,8,9-trihydroxy-6-methoxy-3-methyl-10-(3-methylbut-2-enyl)-2,4-dihydroanthracen-1-one
(3S)-3,8,9-trihydroxy-6-methoxy-3-methyl-10-(3-methylbut-2-enyl)-2,4-dihydroanthracen-1-one
5-hydroxy-2-(2-hydroxybenzoyl)-3-methoxy-4-methylbenzoic acid
5-hydroxy-2-(2-hydroxybenzoyl)-3-methoxy-4-methylbenzoate
1,3,8-trihydroxy-2-methoxy-7-methylanthracene-9,10-dione
2,8-dihydroxy-1,3-dimethoxy-7-methylanthracene-9,10-dione
1,2,8-trihydroxy-3-methoxy-7-methylanthracene-9,10-dione
[(2R)-2,3-dihydroxypropyl] 1,6-dihydroxy-8-methoxy-3-methyl-9,10-dioxoanthracene-2-carboxylate
(3R)-1,3,6-trihydroxy-8-methoxy-3-methyl-2,4-dihydro-1H-benzo[a]anthracene-7,12-dione
methyl 9-(acetyloxymethyl)-5,7-dihydroxy-2,2-dimethyl-
trimethyl 2,4-dihydroxy-7-(2-methoxyphenyl)-5-oxo-7,8-dihydro-6H-naphthalene-1,3,6-tricarboxylate
sodium [4-acetyl-2-(4,5-dihydroxy-2-methyl-9,10-dioxoanthracen-1-yl)-3-hydroxy-5-methoxyphenyl]
sulfate
sodium [4-acetyl-2-(4,5-dihydroxy-2-methyl-9,10-dioxoanthracen-1-yl)-5-hydroxy-3-methoxyphenyl]
sulfate
methyl 1,6,8-trihydroxy-3-methyl-7-(3-methylbut-2-enyl)-9,10-dioxoanthracene-2-carboxylate
[(2S)-2,3-dihydroxypropyl] 1,6-dihydroxy-8-methoxy-
2-[(E)-but-2-enoyl]-1-hydroxy-8-methoxy-3-methylanthracene-
9,10-dione
2-[(E)-1-hydroxybut-2-enyl]-1,8-dimethoxy-3-methylanthracene-9,10-dione
2-(1-hydroxyethyl)-1,8-dimethoxy-3-methylanthracene-9,10-dione
3,8,9-trihydroxy-6-methoxy-3,7-dimethyl-2,4-dihydroanthracen-1-one
1-hydroxy-2-[(2E,4E)-3-hydroxy-4-methylhexa-2,4-dienoyl]-8-methoxy-3-methylanthracene-9,10-dione
3,9-dihydroxy-6,8-dimethoxy-3-methyl-2,4-dihydroanthracen-1-one
ethyl 3,8-dihydroxy-1-(2-methoxyphenyl)-9,10-dioxoanthracene-2-carboxylate
ethyl 3-hydroxy-1-(2-methoxyphenyl)-9,10-dioxoanthracene-2-carboxylate
methyl 2-(11-hydroxy-9-methoxy-2-methyl-4,7,12-trioxonaphtho[2,3-h]chromen-5-yl)acetate
3-(4-hydroxyphenyl)-7-methoxy-5-methylchromen-4-one
3,8-dimethoxy-1-methyl-9,10-dioxoanthracene-2-carboxylic acid
11-hydroxy-5-methylnaphtho[2,3-h]chromene-4,7,12-trione
2-(3-acetyl-4-hydroxy-5-methoxy-9,10-dioxoanthracen-2-yl)acetic acid
1-hydroxy-8-methoxy-3-methyl-2-(4-methyl-3-oxohexanoyl)anthracene-9,10-dione
2-acetyl-1-hydroxy-8-methoxy-3-methylanthracene-9,10-dione
7-hydroxy-4-oxo-3-phenylchromene-8-carbaldehyde
7-hydroxy-3-(4-methoxyphenyl)-5-methylchromen-4-one
3-(dibromomethyl)-1,8-dihydroxy-6-methoxyanthracene-9,10-dione
6-(dibromomethyl)-1-hydroxy-3,8-dimethoxyanthracene-9,10-dione
3,8-dihydroxy-6,9-dimethoxy-3-methyl-2,4-dihydroanthracen-1-one
(3S)-3,6,9-trihydroxy-8-methoxy-3-methyl-2,4-dihydroanthracen-1-one
7-hydroxy-5-methyl-3-phenylchromen-4-one
methyl 2-[6-[3-(4-acetyl-3-hydroxy-2-propylphenoxy)propoxy]-1-oxo-3,4-dihydro-2H-naphthalen-2-
yl]acetate
2-[6-[3-(4-acetyl-3-hydroxy-2-propylphenoxy)propoxy]-1-oxo-3,4-dihydro-2H-naphthalen-2-yl]acetic
acid
(3S)-3,8,9-trihydroxy-6-methoxy-3-methyl-2,4-dihydroanthracen-1-one
1,3,7-trimethoxyanthracene-9,10-dione
5-hydroxy-3-methoxy-4-methylphthalaldehyde
[3-ethyl-4,6-dihydroxy-2-(2-methoxyethyl)phenyl]-(4-methoxyphenyl)methanone
1,3-dihydroxy-5,6-dimethoxy-2-methylanthracene-9,10-dione
1-hydroxy-2,3-dimethoxy-7-methylanthracene-9,10-dione
3-hydroxy-1,5,6-trimethoxy-2-methylanthracene-9,10-dione
(4-methoxyphenyl)methyl 3-hydroxy-8-methoxy-9,10-dioxo-1-propylanthracene-2-carboxylate
1-hydroxy-3,6-dimethoxynaphthalene-2-carbaldehyde
2-acetyl-1-hydroxy-8-methoxyanthracene-9,10-dione
[4-acetyl-2-(4,5-dihydroxy-2-methyl-9,10-dioxoanthracen-1-yl)-3-hydroxy-5-methoxyphenyl] hydrogen
sulfate
3-hydroxy-1,6-dimethoxynaphthalene-2-carbaldehyde
(6-formyl-5-hydroxy-7-methoxy-4-oxo-2,3-dihydro-1H-naphthalen-1-yl) 2,2-dimethylpropanoate
tert-butyl 2-(3-acetyl-4-hydroxy-5,7-dimethoxy-10-oxo-9H-anthracen-2-yl)acetate
[4-acetyl-2-(4,5-dihydroxy-2-methyl-9,10-dioxoanthracen-1-yl)-5-hydroxy-3-methoxyphenyl]hydrogen
sulfate
1,2,6-trihydroxy-7,8-dimethoxy-3-methylanthracene-9,10-dione
(2R,4S)-2-ethyl-2,4,5-trihydroxy-7-methoxy-3,4-dihydrotetracene-1,6,11-trione
11-hydroxy-7,9-dimethoxy-3-methyl-3H-naphtho[3,2-f][2]benzofuran-1,5,10-trione
2-ethyl-2,4,5-trihydroxy-7-methoxy-3,4-dihydrotetracene-1,6,11-trione
7-hydroxy-3-[2,4,5-tri(propan-2-yl)phenyl]chromen-4-one
methyl 3-ethyl-1,6,8-trimethoxy-9,10-dioxoanthracene-2-carboxylate
5,7-dihydroxy-2-(3-hydroxy-4-methylpent-4-enyl)-2,9-dimethylnaphtho[2,3-g]chromene-6,11-dione
1-hydroxy-7-methoxy-2-methylanthracene-9,10-dione
(3S)-11-hydroxy-7,9-dimethoxy-3-methyl-3H-naphtho[3,2-f][2]benzofuran-1,5,10-trione
3-[3,4-di(propan-2-yl)phenyl]-7-hydroxy-8-propan-2-ylchromen-4-one
3-[2,5-di(propan-2-yl)phenyl]-7-hydroxychromen-4-one
1,3,6,8-tetramethoxy-2-propan-2-ylanthracene-9,10-dione
methyl 3-hydroxy-8-methoxy-9,10-dioxo-1-propylanthracene-2-carboxylate
(5-hydroxy-3-methoxy-7-methyl-9,10-dioxoanthracen-1-yl) acetate
7-hydroxy-6,8-dimethoxy-9,10-dioxoanthracene-2-carboxylic acid
(3R)-3,9-dihydroxy-6,8-dimethoxy-3-methyl-2,4-dihydroanthracen-1-one
(3S)-3,8,9-trihydroxy-6-methoxy-3-methyl-2,4-dihydroanthracen-1-one
1,8-dihydroxy-3-(2-hydroxy-3-methylbut-3-enoxy)-6-methylanthracene-9,10-dione
2,5-dihydroxy-6-(hydroxymethyl)-1,3-dimethoxyanthracene-9,10-dione
(3R)-3,8,9-trihydroxy-6-methoxy-3-methyl-2,4-dihydroanthracen-1-one
methyl 4-[3-(3,5-dioxohexyl)-4-ethoxy-5-hydroxy-7-methoxy-9,10-dioxoanthracen-2-yl]-3-oxobutanoate
1,3,6-trimethoxyanthracene-9,10-dione
methyl 8-hydroxy-3-methoxy-9,10-dioxo-1-propylanthracene-2-carboxylate
3,8,9-trihydroxy-6-methoxy-3-methyl-2,4-dihydroanthracen-1-one
2-hydroxy-1,3-dimethoxy-7-methylanthracene-9,10-dione
2,5-dihydroxy-1,3-dimethoxy-6-methylanthracene-9,10-dione
2,6,8-trihydroxy-1-methoxy-3-methylanthracene-9,10-dione
methyl 2-(6,8-dihydroxy-3-methyl-1-oxoisochromene-7-carbonyl)-3-hydroxy-5-methoxybenzoate
3-(4,5-dihydroxy-2-methyl-9,10-dioxoanthracen-1-yl)-
1,3,5-trihydroxy-2-methoxy-6-methylanthracene-9,10-dione
methyl 4-hydroxy-2-(1-hydroxy-3,6,8-trimethoxy-9,10-dioxoanthracen-2-yl)butanoate
3,8,9-trihydroxy-6-methoxy-3-methyl-7-[(E)-3-methylbut-1-enyl]-2,4-dihydroanthracen-1-one
methyl 4-hydroxy-2-(8-hydroxy-1,3,6-trimethoxy-9,10-dioxoanthracen-2-yl)butanoate
1,2-dihydroxy-3-methoxy-6-methylanthracene-9,10-dione
7-hydroxy-3-(4-hydroxyphenyl)-5-methylchromen-4-one
1,3,6-trimethoxy-8-methylanthracene-9,10-dione
1,3,8-trimethoxy-6-methylanthracene-9,10-dione
8-hydroxy-3-methyl-5-(3-methylbut-1-enyl)-9-oxoxanthene-
methyl 3-[(Z)-2-ethylbut-2-enyl]-1,6,8-trihydroxy-9,10-dioxoanthracene-2-carboxylate
3,8,9-trihydroxy-6-methoxy-7-[(E)-3-methylbut-1-enyl]-3,4-dihydro-2H-anthracen-1-one
3-[(E)-5-(3,3-dimethyloxiran-2-yl)-3-methylpent-2-enoxy]-1,8-dihydroxy-6-methylanthracene-9,10-dione
(E)-3-(2,4-dimethoxyphenyl)-1-(1-hydroxynaphthalen-2-yl)prop-2-en-1-one
8-hydroxy-3-methyl-5-[(E)-3-methylbut-1-enyl]-9-oxoxanthene-1-carbaldehyde
3-[(Z)-5-(3,3-dimethyloxiran-2-yl)-3-methylpent-2-enoxy]-1,8-dihydroxy-6-methylanthracene-9,10-dione
5,7-dihydroxy-3-[(4-methoxyphenyl)methyl]-8-methyl-4-oxochromene-6-carbaldehyde
8-hydroxy-1,2,3-trimethoxy-6-methylanthracene-9,10-dione
3-(2,4-dimethoxyphenyl)-1-(1-hydroxynaphthalen-2-yl)prop-2-en-1-one
1,2,6,7-tetrahydroxy-8-methoxy-3-methylanthracene-9,10-dione
2-(hydroxymethyl)-1,8-dimethoxyanthracene-9,10-dione
5-methoxy-2H-naphtho[2,3-h]chromene-7,12-dione
1,3-dimethoxyanthracene-9,10-dione
MW: 268.264080 g/mol|MF: C16H12O4
3-[5-(3,3-dimethyloxiran-2-yl)-3-methylpent-2-enoxy]-1,8-dihydroxy-6-methylanthracene-9,10-dione
8-acetyl-2-(2-acetyl-3,5-dihydroxyphenyl)-5-hydroxychromen-4-one
3,6,9-trihydroxy-8-methoxy-3-methyl-2,4-dihydroanthracen-1-one
1,3,7-trihydroxy-2,8-dimethoxy-6-methylanthracene-9,10-dione
(1,3-dihydroxy-9,10-dioxoanthracen-2-yl)methyl acetate
3,8-dihydroxy-1,2-dimethoxy-7-methylanthracene-9,10-dione
2,4,9-trihydroxy-6-methoxy-7-methyl-2-(2-oxopropyl)phenalene-1,3-dione
1,2,8-trihydroxy-3-methoxy-6-methylanthracene-9,10-dione
7-hydroxy-2-(4-methoxyphenyl)-3-methyl-4-oxochromene-8-carbaldehyde
1,8-dihydroxy-3-[(E)-7-methoxy-3,7-dimethyloct-2-enoxy]-6-methyl-10H-anthracen-9-one
(8-acetyloxy-6-hydroxy-3-methyl-9,10-dioxoanthracen-1-yl) acetate
2,6-dihydroxy-1,7,8-trimethoxy-3-methylanthracene-9,10-dione
methyl 4-[2-(3-formyl-2,4-dihydroxy-6-methylphenyl)-2-oxoethyl]-2-hydroxy-3-methylbenzoate
2-hydroxy-5-(7-hydroxy-4-oxochromen-3-yl)benzaldehyde
(E)-3-(4-hydroxyphenyl)-1-(5-hydroxy-2,2,7-trimethylchromen-6-yl)prop-2-en-1-one
methyl 7-[3-(4-acetyl-3-hydroxy-2-propylphenoxy)propoxy]-6-propyl-1,2,3,4-tetrahydronaphthalene-2-
carboxylate
7-[3-(4-acetyl-3-hydroxy-2-propylphenoxy)propoxy]-6-propyl-1,2,3,4-tetrahydronaphthalene-2-
carboxylic acid
(4R)-4,6-dihydroxy-7-(hydroxymethyl)-8-methoxy-3,4-dihydro-2H-naphthalen-1-one
(4S)-4,6-dihydroxy-7-(hydroxymethyl)-8-methoxy-3,4-dihydro-2H-naphthalen-1-one
(4R)-4,8-dihydroxy-7-(hydroxymethyl)-6-methoxy-3,4-dihydro-2H-naphthalen-1-one
(4S)-4,8-dihydroxy-7-(hydroxymethyl)-6-methoxy-3,4-dihydro-2H-naphthalen-1-one
2,8-dihydroxy-1,3-dimethoxyanthracene-9,10-dione
(3R,4S)-4,6,9-trihydroxy-8-methoxy-3-methyl-3,4-dihydro-2H-anthracen-1-one
(8-hexanoyloxy-6-hydroxy-3-methyl-9,10-dioxoanthracen-1-yl) hexanoate
9-[tert-butyl(dimethyl)silyl]oxy-11-hydroxy-5-methoxy-2-methylnaphtho[3,2-h]chromene-4,7,12-trione
5,7-dihydroxy-3-(3-methoxyphenyl)-6-(3-methylbut-2-enyl)chromen-4-one
4,6-dihydroxy-7-(hydroxymethyl)-8-methoxy-3,4-dihydro-2H-naphthalen-1-one
3-acetyl-1,4-dihydroxy-5-methoxy-2-methylanthracene-9,10-dione
1-hydroxy-3-(hydroxymethyl)-8-methoxyanthracene-9,10-dione
6-hydroxy-8-methoxy-1,7,12-trioxo-3,4-dihydro-2H-benzo[a]anthracene-3-carboxylic acid
1,5-dihydroxy-2,7-dimethoxy-4-methylanthracene-9,10-dione
1,8-dihydroxy-2,6-dimethoxy-4-methylanthracene-9,10-dione
4-hydroxy-6,8-dimethoxy-3-(3-oxobutyl)naphtho[2,3-f][1]benzofuran-5,10-dione
1-[4,6-dihydroxy-2-(2-methoxyethyl)-3-(3-methoxyphenyl)phenyl]ethanone
7-hydroxy-3-naphthalen-2-ylchromen-4-one
(3,8-dihydroxy-6-methyl-9,10-dioxoanthracen-1-yl) acetate
1-(2,6-dihydroxy-4-methylphenyl)-3-(6-methoxy-1,2,3,4-tetrahydronaphthalen-2-yl)propan-1-one
7-ethoxy-2,4-dihydroxy-3-methylnaphthalene-1-carbaldehyde
1,3,8-trihydroxy-6-propanoylanthracene-9,10-dione
2-[(2S,3R)-2,3-dimethyl-4-oxo-2,3-dihydropyran-6-yl]-1-hydroxy-8-methoxy-3-methylanthracene-9,10-
dione
2-[(2S,3S)-2,3-dimethyl-4-oxo-2,3-dihydropyran-6-yl]-1-hydroxy-8-methoxy-3-methylanthracene-9,10-
dione
6-hydroxy-5,8-dimethoxy-2-methylbenzo[g]chromen-4-one
8-hydroxy-5,6-dimethoxy-2-methylbenzo[g]chromen-4-one
(2-hydroxy-4-methoxyphenyl)-naphthalen-2-ylmethanone
9-acetyl-6,7,9-trihydroxy-2-methoxy-8,10-dihydro-7H-tetracene-5,12-dione
1,3,6,8-tetrahydroxy-2-(6-methyloxan-2-yl)anthracene-9,10-dione
(4,5-dihydroxy-2-methoxy-7-methyl-10-oxo-9H-anthracen-9-yl) (Z)-octadec-9-enoate
2-hydroxy-4-[2-oxo-2-(5,5,8,8-tetramethyl-6,7-dihydronaphthalen-2-yl)ethoxy]benzaldehyde
4,5,7-trihydroxy-1-[1-[(E)-1-hydroxyprop-1-enoxy]ethenyl]-2-methylanthracene-9,10-dione
7-hydroxy-8-methyl-3-(4-methylphenyl)chromen-4-one
1,3,6,8-tetrahydroxy-2-[(2R,6R)-6-methyloxan-2-yl]anthracene-9,10-dione
(2,4-dimethylphenyl)-(4-ethoxy-6-hydroxy-2,3-dimethylphenyl)methanone
(2-hydroxy-4-methoxyphenyl)-(4-methyl-2-phenylphenyl)methanone
1,8-dihydroxy-2,3-dimethoxyanthracene-9,10-dione
5-hydroxy-9-methoxy-3,4-dihydro-1H-tetracene-2,6,11-trione
3-(3,5-dihydroxyphenyl)-8-hydroxy-6-methoxy-4,4-dimethyl-2,3-dihydronaphthalen-1-one
3-(hydroxymethyl)-1,8-dimethoxyanthracene-9,10-dione
4,5-dimethoxy-9,10-dioxoanthracene-2-carbaldehyde
(1,3-dihydroxy-6,8-dimethoxynaphthalen-2-yl)-[1-hydroxy-6,8-dimethoxy-3-
(methoxymethyl)naphthalen-2-yl]methanone
5,9,11-trimethoxy-2-methylnaphtho[3,2-h]chromene-4,7,12-trione
3-[(2E)-3,7-dimethyl-5-oxoocta-2,6-dienyl]-2-hydroxy-6-(hydroxymethyl)-4-methoxybenzaldehyde
4,5,7-trihydroxy-1-(6-hydroxy-4-oxopyran-2-yl)-2-methylanthracene-9,10-dione
6-ethyl-5-hydroxy-7-methoxynaphthalene-1,4-dione
5,10-dihydroxy-8-methoxy-2,7-dimethylbenzo[h]chromen-4-one
4-hydroxy-1-methoxy-9,10-dioxoanthracene-2-carbaldehyde
6-acetyl-7-[5-(4-acetyl-3-hydroxy-2-propylphenoxy)pentoxy]-1,2,3,4-tetrahydronaphthalene-2-carboxylic
acid
MW: 496.591940 g/mol|MF: C29H36O7
3-hydroxy-6,8,9-trimethoxy-3-methyl-2,4-dihydroanthracen-1-one
1,8-dihydroxy-2-(hydroxymethyl)-5-methoxyanthracene-9,10-dione
MW: 300.262880 g/mol|MF: C16H12O6
3,8,9-trihydroxy-3-methyl-6-(3-methylbut-2-enoxy)-2,4-dihydroanthracen-1-one
MW: 342.385680 g/mol|MF: C20H22O5
1,2,6,8-tetrahydroxy-7-methoxy-3-methylanthracene-9,10-dione
1,2-dihydroxy-6,7,8-trimethoxy-3-methylanthracene-9,10-dione
9-acetyl-6,7,9-trihydroxy-3-methoxy-8,10-dihydro-7H-tetracene-5,12-dione
5-hydroxy-8-methoxy-3,4-dihydro-1H-tetracene-2,6,11-trione
8-hydroxy-1-methoxy-3-methylanthracene-9,10-dione
1-hydroxy-3-methoxynaphthalene-2-carbaldehyde
1-hydroxy-8-methoxy-2-methylanthracene-9,10-dione
2-acetyl-1,6,8-trihydroxy-3-methylanthracene-9,10-dione
2-hydroxy-4-methoxynaphthalene-1-carbaldehyde
1-hydroxy-2-(1-hydroxy-3-oxobutyl)-5,8-dimethoxy-3-methylanthracene-9,10-dione
(3,6-dihydroxy-2,4-dimethylphenyl)-(2,4-dimethoxyphenyl)methanone
ethyl 3-hydroxy-1-(2-methoxyphenyl)-6-methyl-5,8-dioxonaphthalene-2-carboxylate
methyl 2-[2-ethyl-3,5-dihydroxy-6-(4-methoxybenzoyl)phenyl]acetate
methyl 2-[2-ethyl-3,5-dihydroxy-6-(3-methoxybenzoyl)phenyl]acetate
(4S,4aS,9aR)-5-hydroxy-4-[(1R)-1-(4-methoxyphenyl)ethoxy]-3,4,4a,9a-tetrahydro-1H-anthracene-
2,9,10-trione
(4S,4aS,9aR)-5-hydroxy-4-[(1S)-1-(4-methoxyphenyl)ethoxy]-3,4,4a,9a-tetrahydro-1H-anthracene-
2,9,10-trione
(4R,4aS,9aR)-5-hydroxy-4-[(1R)-1-(4-methoxyphenyl)ethoxy]-3,4,4a,9a-tetrahydro-1H-anthracene-
2,9,10-trione
(4R,4aS,9aR)-5-hydroxy-4-[(1S)-1-(4-methoxyphenyl)ethoxy]-3,4,4a,9a-tetrahydro-1H-anthracene-
2,9,10-trione
methyl 3-[(2S)-2-[tert-butyl(dimethyl)silyl]oxypropyl]-8-hydroxy-1,6-dimethoxy-9,10-dioxoanthracene-
2-carboxylate
methyl 9-hydroxy-1,11-dimethoxy-3-methyl-8,13-dioxo-5,6-dihydrobenzo[a]tetracene-2-carboxylate
[4,7-diacetyloxy-5-hydroxy-9,10-dioxo-6-[(E)-3-oxobut-1-enyl]anthracen-2-yl] acetate
1-(2-hydroxy-4,6-dimethoxyphenyl)-2-methylanthracene-9,10-dione
(12-hydroxy-8-methoxy-3-methyl-1,6,11-trioxo-3,4-dihydronaphtho[6,7-g]isochromen-10-yl) acetate
(10-hydroxy-8-methoxy-3-methyl-1,6,11-trioxo-3,4-dihydronaphtho[2,3-g]isochromen-12-yl) acetate
2-[(2,4-dimethoxyphenyl)methyl]-1,8-dihydroxy-10H-anthracen-9-one
1,6-dihydroxy-8-methoxy-3-methylbenzo[a]anthracene-7,12-dione
methyl 4-(4,5,7-trimethoxy-9,10-dioxoanthracen-2-yl)butanoate
methyl 3-(4,5,7-trimethoxy-9,10-dioxoanthracen-2-yl)propanoate
1-hydroxy-5,8-dimethoxy-3-methyl-2-(3-oxobutanoyl)anthracene-9,10-dione
3-(2,4-dihydroxy-3-methylphenyl)-5-hydroxy-7-methoxychromen-4-one
1-hydroxy-8-methoxy-3-methylanthracene-9,10-dione
(3-hydroxy-1,6,8-trimethoxynaphthalen-2-yl)-[1,6,8-trimethoxy-3-(methoxymethyl)naphthalen-2-
yl]methanone
methyl (E)-3-(4,5,7-trimethoxy-9,10-dioxoanthracen-2-yl)prop-2-enoate
(E)-1-(2-hydroxy-4,6-dimethoxyphenyl)-3-naphthalen-2-ylprop-2-en-1-one
4-hydroxy-6,8-dimethoxy-3-(4-methoxyphenyl)-3,4-dihydro-2H-naphthalen-1-one
1-[3-(3-acetyl-2-hydroxy-6-methoxy-4-methylphenyl)-2-hydroxy-4-methoxy-6-methylphenyl]ethanone
methyl 3-acetyl-1,6,8-trimethoxy-9,10-dioxoanthracene-2-carboxylate
1-[3-(3-acetyl-4-hydroxy-6-methoxy-2-methylphenyl)-2-hydroxy-4-methoxy-6-methylphenyl]ethanone
1-[3-(3-acetyl-4,6-dihydroxy-2-methylphenyl)-2-hydroxy-4-methoxy-6-methylphenyl]ethanone
4-hydroxy-5-methoxy-9,10-dioxoanthracene-2-carboxylic acid
2-acetyl-1,3,6,8-tetrahydroxyanthracene-9,10-dione
3-hydroxy-8-methoxy-1-methylanthracene-9,10-dione
7-hydroxy-8-propan-2-yl-3-[2,4,5-tri(propan-2-yl)phenyl]chromen-4-one
2-hydroxy-6-(2-oxononyl)-4-phenylmethoxybenzaldehyde
1-(2-hydroxy-4,6-dimethoxyphenyl)-2-(hydroxymethyl)anthracene-9,10-dione
1-hydroxy-8-methoxy-2-(4-methoxybutyl)anthracene-9,10-dione
2-hydroxy-4-methoxy-6-methyl-3-[(2E,4E)-3-methyl-5-[(1R,2R,6R)-1,2,6-trimethyl-3-
oxocyclohexyl]penta-2,4-dienyl]benzaldehyde
1-(2-hydroxy-4-methoxyphenyl)-4-naphthalen-2-ylbutane-1,3-dione
6-hydroxy-8-methoxy-1,2,3,4-tetrahydrobenzo[a]anthracene-7,12-dione
1,8-dimethoxy-9,10-dioxoanthracene-2-carbaldehyde
3-(3-bromopropoxy)-1-hydroxyanthracene-9,10-dione
dimethyl 1-[2,4-dimethoxy-6-(methoxymethyl)phenyl]-4-hydroxy-5-methoxynaphthalene-2,3-
dicarboxylate
1-hydroxy-8-methoxy-2-prop-2-enylanthracene-9,10-dione
dimethyl 1-[2,4-dimethoxy-6-(methoxymethyl)phenyl]-4-hydroxynaphthalene-2,3-dicarboxylate
3-(2-bromoethoxy)-1-hydroxyanthracene-9,10-dione
1-hydroxy-3-(oxiran-2-ylmethoxy)anthracene-9,10-dione
1-(1,6-dihydroxy-8-methoxy-3-methylnaphthalen-2-yl)ethanone
(7S,9S)-9-acetyl-6,7,9-trihydroxy-4-methoxy-8,10-dihydro-7H-tetracene-5,12-dione
1,3-dihydroxy-6,7-dimethoxyanthracene-9,10-dione
8,10,12-trihydroxy-3-methoxy-1-methyl-6,11-dioxotetracene-2-carboxylic acid
(3S)-3,8,9-trihydroxy-3-methyl-6-(3-methylbut-2-enoxy)-2,4-dihydroanthracen-1-one
4,8-dihydroxy-6-methoxy-7-methyl-3,4-dihydro-2H-naphthalen-1-one
3-[(2,4-dimethoxyphenyl)methyl]-1,8-dihydroxy-10H-anthracen-9-one
1-hydroxy-8-methoxy-2-[(E)-prop-1-enyl]anthracene-9,10-dione
3-(2,5-dihydroxy-4-methoxy-7-methylnaphthalen-1-yl)-5-methoxy-2-methylnaphthalene-1,4-dione
11-hydroxy-1,10-dimethoxy-8-methyltetracene-5,12-dione
4-hydroxy-5,7-dimethoxynaphthalene-2-carbaldehyde
3-hydroxy-2-(7-hydroxy-2,2-dimethylchromene-8-carbonyl)-6-methoxy-5-methylbenzaldehyde
4,8-dihydroxy-7-(hydroxymethyl)-6-methoxy-3,4-dihydro-2H-naphthalen-1-one
8-hydroxy-1,3,6-trimethoxy-2-(2-oxooxolan-3-yl)anthracene-9,10-dione
7-ethyl-4,8-dihydroxy-6-methoxy-3,4-dihydro-2H-naphthalen-1-one
5-hydroxy-8,10-dimethoxy-2-methylbenzo[h]chromen-4-one
5,7-dihydroxy-3-(2-methoxyphenyl)-6-methylchromen-4-one
5-(5,7-dihydroxy-4-oxochromen-3-yl)-2-hydroxybenzaldehyde
1-(1,8-dihydroxy-6-methoxy-3-methylnaphthalen-2-yl)ethanone
(4,5-dihydroxy-2-methoxy-7-methyl-10-oxo-9H-anthracen-9-yl) (E)-octadec-9-enoate
4-hydroxy-5-[(E)-3-(4-hydroxyphenyl)prop-2-enoyl]-2-methoxybenzaldehyde
2-hydroxy-5-[(E)-3-(4-hydroxyphenyl)prop-2-enoyl]-4-methoxybenzaldehyde
6-[(2E)-3,7-dimethylocta-2,6-dienoxy]-3,8,9-trihydroxy-3-methyl-2,4-dihydroanthracen-1-one
1,3,6,8-tetramethoxyanthracene-9,10-dione
3-methoxy-1-prop-2-enoxyanthracene-9,10-dione
5-hydroxy-4-[1-(4-methoxyphenyl)ethoxy]-3,4,4a,9a-tetrahydro-1H-anthracene-2,9,10-trione
5,6-dihydroxy-8-methoxy-2,9-dimethylbenzo[g]chromen-4-one
(4S,4aR,9aR)-5-hydroxy-4-[(1S)-1-(4-methoxyphenyl)ethoxy]-3,4,4a,9a-tetrahydro-1H-anthracene-
2,9,10-trione
1-(3-hydroxy-1-methoxynaphthalen-2-yl)butane-1,3-dione
2-hydroxy-3,6-dimethyl-4-phenylmethoxybenzaldehyde
2-(1,2-dihydroxypentyl)-1-hydroxy-8-methoxyanthracene-9,10-dione
1-hydroxy-2,5,7-trimethoxyanthracene-9,10-dione
2,5,7-trihydroxy-3,9-dimethoxy-2-methyl-3,4-dihydrotetracene-1,6,11-trione
9-hydroxy-6,8-dimethoxy-3,3-dimethyl-2,4-dihydroanthracen-1-one
1-(2-hydroxy-4,6-dimethoxyphenyl)naphthalene-2-carbaldehyde
2-[(1S)-1,2-dihydroxypentyl]-1-hydroxy-8-methoxyanthracene-9,10-dione
6-(3,7-dimethylocta-2,6-dienoxy)-3,8,9-trihydroxy-3-methyl-2,4-dihydroanthracen-1-one
1,3-diethoxyanthracene-9,10-dione
3,10,12-trihydroxy-8-methoxy-1-methyl-6,11-dioxotetracene-2-carboxylic acid
5-hydroxy-6,8-dimethoxy-2-methylbenzo[g]chromen-4-one
4,6,8-trihydroxy-3-(3-oxobutyl)-2,3-dihydronaphtho[2,3-f][1]benzofuran-5,10-dione
2-hydroxy-1,6,7,8-tetramethoxy-3-methylanthracene-9,10-dione
1,7-dihydroxy-2,3,8-trimethoxy-6-methylanthracene-9,10-dione
1,3,5-trihydroxy-6-methoxy-2-methylanthracene-9,10-dione
1,3,6,8-tetrahydroxy-2-[(2S,6S)-6-methyloxan-2-yl]anthracene-9,10-dione
5,6-dihydroxy-8-methoxy-2,7-dimethylbenzo[g]chromen-4-one

Damnacanthal and several damnacanthal analogs are commercially available. Standard doses of damnacanthal and several damnacanthal analogs are known in the art (e.g., 0.5 mg, 1.0 mg, 2.0 mg, 3.0 mg, 4.0 mg, 5.0 mg, 7.5 mg, 10 mg, 15 mg, 20 mg, 25 mg, 50 mg, 75 mg, 100 mg, 125 mg, 150 mg, 175 mg, 200 mg, 225 mg, 250 mg, 275 mg, 300 mg, 350 mg, 400 mg, 450 mg, 500 mg, 550 mg, 600 mg, 650 mg, 700 mg, 750 mg, 800 mg, 850 mg, 900 mg, or any range between any pair of recited doses) and can range, e.g., from 0.1 mg to 300 mg (e.g., 0.1 mg to 200 mg, 0.1 mg to 150 mg, 0.1 mg to 100 mg, and 1.0 mg to 50 mg).

### Dantrolene

The methods, compositions, and kits of the invention may employ dantrolene or a dantrolene analog. A variety of dantrolene analogs are known in the art. Nonlimiting examples of dantrolene analogs are azumolene and those described in U.S. Pat. No. 3,415,821 (e.g., compounds of the general formula of claim 1 and examples I through XX); U.S. Pat. No. 4,001,222 (e.g., compounds of the general formula of claim 1 and examples I through IX); and U.S. Pat. No. 4,049,650 (e.g., compounds of the general formula of claim 1 and examples I through IX) (each of which is incorporated by reference).

Dantrolene and several dantrolene analogs are commercially available. Standard doses of dantrolene and several dantrolene analogs are known in the art (e.g., 0.5 mg, 1.0 mg, 2.0 mg, 3.0 mg, 4.0 mg, 5.0 mg, 7.5 mg, 10 mg, 15 mg, 20 mg, 25 mg, 50 mg, 75 mg, 100 mg, 125 mg, 150 mg, 175 mg, 200 mg, 225 mg, 250 mg, 275 mg, 300 mg, 350 mg, 400 mg, 450 mg, 500 mg, 550 mg, 600 mg, 650 mg, 700 mg, 750 mg, 800 mg, 850 mg, 900 mg, or any range between any pair of recited doses) and can range, e.g., from 0.1 mg to 300 mg (e.g., 0.1 mg to 200 mg, 0.1 mg to 150 mg, 0.1 mg to 100 mg, and 1.0 mg to 50 mg).

### Tumor-Associated Antigens (TAAs)

The methods, compositions, and kits of the invention provide an HSP90 inhibitor, OBAA, flunarizine, aphidicolin, damnacanthal, dantrolene, or an analog thereof, in combination with a tumor-associated antigen (TAA).

TAAs are antigenic molecules whose expression facilitates interaction of immune cells or immune molecules (e.g., antibodies) with tumor cells. TAAs are molecules or portions of molecules that immune targeting molecules (i.e., receptors on immune cells and antibodies) bind. TAAs may be present in or on normal cells; tumor TAA expression may, but need not, deviate from normal (non-tumor) counterpart cells (e.g., a normal cell not expressing TAA, expressing less of the TAA than a tumor cell, or expressing the same or more TAA than tumor).

A TAA can be expressed during an earlier developmental or different differentiation stage of the cell; after progressing through the developmental stage, expression of the TAA is typically altered. For example, a melanoma differentiation associated (mda) gene displaying enhanced or suppressed expression during growth inhibition and differentiation, such as MAGE and Melan-A/MART-1. As disclosed herein, TAA expression can also be induced or increased in response to a stimulus (e.g., with an HSP90 inhibitor, OBAA, flunarizine, aphidicolin, damnacanthal, dantrolene, or an analog thereof). In addition, kinase inhibitors can up-regulate expression of TAAs Melan-A/MART-1, gp100, tyrosinase, TRP-1, and TRP-2 on melanomas, and TAA expression has been reported to be up-regulated by IFN-γ and IFN-β. Tumor cell expression of one or more TAAs that are atypical for the cell is presumably due to aberrant gene regulation of the TAA.

Specific non-limiting examples of TAAs whose expression can be increased or induced in accordance with the invention are, for melanoma, tumor-associated testis-specific antigen (e.g., MAGE, BAGE, and GAGE), melanocyte differentiation antigen (e.g., tyrosinase, Melan-N MART-I), a mutated or aberrantly expressed molecule (e.g., CDK4, MUM-I, β-catenin), gp100/pmel 17, TRP-1, TRP-2, an MITF, MITF-A and MITF-M (King, et al. (1999). Am J Pathol 155:731). Additional specific examples of TAAs expressed by tumors include melanoma GP75, Annexin I, Annexin II, adenosine deaminase-binding protein (ADAbp), PGP 9.5 (Rode, et al. (1985). Histopathology 9:147), colorectal associated antigen (CRC)-C017-1N GA733, Ab2 BR3E4, CI17-1A/GA733, Hsp70 (Chen, et al. (2002). Immunol Lett 84:81), Hsp90, Hsp96, Hsp105, Hsp110, HSPPC-96 (Caudill, M. M. and Z. Li (2001). Expert Opin Biol Ther 1:539), stress protein gp96 (a human colorectal cancer tumor rejection antigen, Heike et al. (2000). Int J Can 86:489), gp96-associated cellular peptides, G250, Dipeptidyl peptidase IV (DPPIV), Mammaglobin (Tanaka, et al. (2003). Surgery 133:74), thyroglobulin, STn (Morse, M. A. (2000). Curr Opin Mol Ther 2:453), Carcinoembryonic Antigen (CEA), CEA epitope CAP-I, CEA epitope CAP-2, etv6, amI1, Prostate Specific Antigen (PSA), PSA epitope PSA-1, PSA epitope PSA-2, PSA epitope PSA-3 (Correale, et al. (1998). J Immunol 161:3186) (Roehrbom, et al. (1996). Urology 47:59), Ad5-PSA, prostate-specific membrane antigen (PSMA), Prostatic Acid Phosphatase (PAP), Prostate epithelium-derived Ets transcription factor (PDEF), Parathyroid-hormone-related protein (PTHrP), EGFR (Plunkett, et al. (2001). J Mammary Gland Biol Neoplasia 6:467), PLU1 (Plunkett, et al. (2001). J Mammary Gland Biol Neoplasia 6:467), Oncofetal antigen-immature laminin receptor (OFA-iLR), MN/CA IX (CA9) (Shimizu et al., (2003). Oncol. Rep. September-October; 10:1307), HP59, Cytochrome oxidase 1, sp100, msa (Devine, et al. (1991). Cancer Res 51:5826), Ran GTPase activating protein, a Rab-GAP (Rab GTPase-activating) protein, PARIS-1 (Zhou, et al. (2002). Biochem Biophys Res Commun 290:830), T cell receptor/CD3-zeta chain, cTAGE-1, SCP-1, Glycolipid antigen-GM2, GD2, or GD3, GM3 (Bada, et al. (2002). Hum Exp Toxicol 21:263), Fucosyl GM1, Glycoprotein (mucin) antigens-Tn, Sialyl-Tn (Lundin, et al. (1999). Oncology 57:70), TF, and Mucin-1 (Mukherjee, et al. (2003). J Immunother 26:47), CA125 (MUC-16) (Reinartz, et al. (2003). Cancer Res 63:3234), a MAGE family antigen, GAGE-1,2, BAGE, RAGE, LAGE-1 (Eichmuller, et al. (2003). Int J Cancer 104:482) (Chen, et al. (1998). Proc Natl Acad Sci USA 95:6919), GnT-V (Murata, et al. (2001). Dis Colon Rectum 44:A2-A4), MUM-1 (Kawakami, et al. (1996). Keio J Med 45:100), EP-CAM/KSA (Ullenhag, et al. (2003). Clin Cancer Res 9:2447), CDK4, a MUC family antigen, HER2/neu, ErbB-2/neu, p21ras, RCAS1, α-fetoprotein, E-cadherin, α-catenin, β-catenin, and γ-catenin, NeuGcGM3 (Carr, et al. (2003). J Clin OncoI21:1015), Fos related antigen (Luo, et al. (2003). Proc Natl Acad Sci USA100:8850), Cyclophilin B (Tamura, et al. (2001). Jpn J Cancer Res 92:762), RCAS1, S2 (Koga, et al. (2003). Tissue Antigens 61:136), L10a (Koga, et al. (2003). supra), Telomerase rt peptide (Wang, et al. (2001). Oncogene 20:7699), cdc27, fodrin, p120ctn, PRAME, GA733/EoCam (Ross, et al. (1986). Biochem Biophys Res Commun 135:297), NY-BR-1, NY-BR-2, NY-BR-3, NY-BR-4, NYBR-5, NY-BR-6, NY-BR-7 (Jager, et al. (2001). Cancer Res 61:2055), NY-ESO-1, L19H1, MAZ (Daheron, et al. (1998). Leukemia 12:326), PINCH (Greiner, et al (2000). Exp Hematol 28:1413), PRAME (Ikeda, et al. (1997). Immunity 6:199), Prp1p/Zer1p, WT1 (Oka, et al. (2002). Curr Cancer Drug Targets 2:45), adenomatous polyposis coli protein (APC), PHF3, LAGE-1, SART3 (Miyagi, et al. (2001). Clin Cancer Res 7:3950), SCP-1 (Jager, et al. (2002). Cancer Immun 2:5), SSX-1, SSX-2, SSX-4, TAG-72 (Buchsbaum, et al. (1999). Clin Cancer Res 5(10 Suppl): 3048s-3055s), TRAG-3 (Chen, et al. (2002). Lung Cancer 38:101), MBTAA (Basu, et al. (2003). Int J Cancer 105:377), a Smad tumor antigen, lmp-L, HPV-16 E7, c-erbB-2, EBV-encoded nuclear antigen (EBNA)-1, Herpes simplex thymidine kinase (HSVtk), alternatively spliced isoform of XAGE-1 (L552S; Wang, (2001). Oncogene 20:7699), TGF beta RII frame shift mutation (Saeterdal, et al. (2001). Proc Natl Acad Sci USA 98: 13255), BAX frame shift mutation (Saeterdal, et al. (2001). Proc Natl Acad Sci USA 98:13255).

Table 6 lists selected tumor types and non-limiting exemplary TAAs present in or on each such tumor type. Any of the TAAs listed in Table 6 may also be present on other tumor types, and additional TAAs may be present on each listed tumor type. Also included are immunogenic fragments of any of the TAAs listed in Table 6.

TABLE 6
Selected Tumor Types and Exemplary TAAs

| Tumor Type | TAAs |
|---|---|
| Melanoma | MAGE-A1, -A2, -A3, -A4, -A6, -A10, -A12 (multiple |
| | epitopes of each MAGE antigen) |
| | BAGE |
| | DAM-6, -10 |
| | GAGE-1, -2, -3, -4, -5, -6, -7B, 8 |
| | NA88-A |
| | NY-ES0-1 and, -1a I (aka CAG-3) |
| | MC1R |
| | Gp100 (including splice variants) |
| | SP110 (including splice variants) |
| | PSA |
| | PSM |
| | Tyrosinase |
| | TRP-1 (or gp75) |
| | TRP-2 |
| | CAMEL |
| | CEA |
| | Her2/neu |
| | hTERT |
| | hTRT |
| | PRAME |
| | P15 |
| | RU1, RU2 |
| | SART-3 |
| | CDK-4/mGnT-V |
| | HSP70-2M |
| | MUM-1, -2, -3 |
| | Myosin/m |
| | RAGE |
| | SART-2 |
| | TRP-2/INT2 |
| | 707-AP |
| | Annexin II |
| | MAGE-1, -2, -3, -6 |
| | CDC27/m |
| | TPI/m |
| | LDLR/FUT |
| | OA1 |
| | CSPG4 (MCSP) |
| | P Polypeptide |
| | TRAG-3 |
| | SSX-2 |
| | Adipophilin |
| | AIM-2 |
| | EphA2, EphA3 |
| | FN(MutantMIFEKHGFRRTTPP) |
| | Neo-PAP |
| | PTPRK |
| | TPI |
| | GM2 Ganglioside |
| | GM3 Ganglioside |
| | GD2 ganglioside |
| | GD3 ganglioside |
| | N-glycolyl (NGc)GM3Ganglioside |
| | human melanoma proteoglycan (HMP) |
| | STEAP |
| | Livin (ML-IAP) |
| | SOX10 |
| | Beta-catenin |
| | MART-2 |
| | Survivin and -2B |
| | Myosin |
| | OS-9 |
| | N-RAS |
| | TRP-2-6b |
| | LAGE-1 |
| | ErbB2 |
| | EGFR |
| | BRAF mutants (V600E, V600K, V600R, V600M) |
| | NRAS |
| | Spliced peptides, IYMDGTADFSF and |
| | IYMDGAADFSF |
| Brain Tumor | IL-13Rα2 |
| | Gp100 |
| | GnT-V |
| | RU2 |
| | SART-2, -3 |
| | TRP-2/INT2 |
| | TRP-2-6b |
| | ErbB2 |
| | EGFR |
| | CSPG4 (NG2) |
| | PRAME |
| | BST2 (bone marrow stromal cell antigen 2; also |
| | known as PDCA-1, CD137 and HM1.24 |
| | GM2 Ganglioside |
| | GM3 Ganglioside |
| | GD2 ganglioside |
| | GD3 ganglioside |
| | EphA2 |
| | YKL40 |
| | hTERT |
| Lung Cancer | BAGE |
| | DAM-6, -10 |
| | GAGE-1, -2, -8 |
| | GAGE-3, -4, -5, -6, -7B |
| | MAGE-A1, -A2, -A3, -A4, -A6, -A10, -A12 (multiple |
| | epitopes of each MAGE antigen) |
| | STEAP |
| | EZH2 |
| | TRAG-3 |
| | Ep-CAM |
| | BCP-20 (FBXO39) |
| | MUC1 |
| | ART-4 |
| | CLCA2 |
| | Cyp-B |
| | Malic enzyme |
| | WT1 |
| | NeuGc-GM3 |
| | TRG |
| | ALK |
| | EphA2 |
| | RU2 |
| | PRAME |
| | hTERT |
| | SART-1 |
| | SART-2, -3 |
| | Survivin, -2B |
| | α-actinin-4 |
| | ELF2 |
| | EphA3 |
| | ErbB2 and ErbB1 |
| | EGFR |
| | EGF |
| | SP70 of NSCLC |
| | PRAME |
| | Cell surface chondroitin sulfate proteoglycan 4 |
| | (CSPG4) oncofoetal antigen 5T4 |
| | Lengsin |
| | Dickkopf-1 (DKK1) |
| | BST2 (bone marrow stromal cell antigen 2; also |
| | known as PDCA-1, CD137 and HM1.24 |
| | TTK |
| | LY6K |
| | IGF (insulin-like growth factor) |
| | IMP-3 |
| | GD3 |
| | RHAMM |
| | G250/CAIX |
| | ULBPs |
| | NKGD2 |
| | CDC45L |
| | carcinoembryonic antigen (CEA) |
| Breast Cancer | BAGE, |
| | CAMEL |
| | NY-ESO-1 |
| | NeuGc-GM3 |
| | LAGE-1 |
| | Adipophilin |
| | Mammaglobin-A |
| | DAM-6, -10 |
| | GAGE-1, -2, -8 |
| | GAGE-3, -4, -5, -6, -7B |
| | MAGE-A1, -A2, -A3, -A4, -A6, -A10, -A12 (multiple |
| | epitopes of each MAGE antigen) |
| | TRAG-3 |
| | BCP-20 (FBXO39) |
| | Ep-CAM |
| | AIM-2a |
| | ART-4 |
| | carcinoembryonic antigen (CEA) |
| | EphA2 |
| | FGF-5 |
| | HER2/neu |
| | WT1 |
| | CSPG4 |
| | CHST11 |
| | Livin (ML-IAP) |
| | MUC1 |
| | MUC2 |
| | RAGE |
| | RU2 |
| | Survivin, -2B |
| | TRG |
| | MUC1 |
| | ErbB2 and ErbB1 |
| | EGFR |
| | topoisomerase II alpha (TOP2A), |
| | cyclin a2 (CCNA2), |
| | v-fos fbj murine osteosarcoma viral oncogene homolog |
| | (FOS), |
| | wingless-type mmtv integration site family, member 5a |
| | (WNT5A), |
| | growth factor receptor-bound protein 7 (GRB7), |
| | cell division cycle 2 (CDC2), |
| | baculoviral iap repeat-containing protein 5 (BIRC5). |
| | v-myc |
| | avian myelocytomatosis viral oncogene homolog |
| | (MYC), tumor protein p53 (TP53), |
| | estrogen receptor α (ESR1) |
| | PGRMC1 |
| | Androgen receptors |
| | Estrogen receptors |
| | hTERT |
| Esophageal, | GAGE-1, -2, -8 |
| Gastric, or | GAGE-3, -4, -5, -6, -7B |
| Gastroesophageal | M MAGE-A1, -A2, -A3, -A4, -A6, -A10, -A12 |
| Cancer | (multiple epitopes of each MAGE antigen) |
| | Ep-CAM |
| | ART-4 |
| | CLCA2 |
| | BCP-20 (FBXO39) |
| | Cyp-B |
| | HER2/neu |
| | EphA2 |
| | HST-2 (FGF-6) |
| | P53 |
| | RU2 |
| | WT1 |
| | carcinoembryonic antigen (CEA) |
| | NY-ESO-1 |
| | SART-1 |
| | SART-2, -3 |
| | Survivin, -2B |
| | ErbB2 |
| | EGFR |
| | hTERT |
| Non-Hodgkin | SSX-2 |
| Lymphoma | Cyp-B |
| | hTERT |
| | Survivin, -2B |
| | Livin (ML-IAP) |
| | MUC1 |
| | NPM/ALKb |
| | EBV protein LMP1 |
| | CD19 |
| | CD20 |
| | ROR1 |
| | WT1 |
| | RHAMM |
| | PR1 (Proteinase 1) |
| | Her2 |
| | ALK |
| | CD30 |
| | hTERT |
| Prostate Cancer | CAMEL |
| | NY-ESO-1 |
| | LAGE-1 |
| | ISG15 |
| | GAGE-1, -2, -8 |
| | GAGE-3, -4, -5, -6, -7B |
| | MAGE-A1, -A2, -A3, -A4, -A6, -A10, -A12 (multiple |
| | epitopes of each MAGE antigen) |
| | TRAG-3 |
| | AFP |
| | EphA2 |
| | FGF-5 |
| | hTERT |
| | Livin (ML-IAP) |
| | PSMA |
| | RU2 |
| | Survivin, -2B |
| | TRG |
| | PAP (Prostatic Acid Phosphatase) |
| | PSA |
| | PSCA (Prostate Stem Cell Ag) |
| | ErbB2 |
| | 5T4 |
| | Her2/neu |
| | Androgen Receptors |
| | Estrogen Receptors |
| | carcinoembryonic antigen (CEA |
| | hTERT |
| | PSCA |
| | GRP/MUC family, erbB2/HER-2/neu GnRH/ |
| | testosterone |
| | BCP-20 (FBXO39) |
| | PAGE5 |
| | STEAP |
| | EZH2 |
| | synovial sarcoma X chromosome breakpoint (SSX) |
| Renal Cell Cancer | Adipophilin |
| | FGF-5 (including splice variants) |
| | G250 |
| | iCE |
| | BCP-20 (FBXO39) |
| | M-CSF |
| | MAG-A6 |
| | EphA2 |
| | PRAME |
| | RAGE |
| | RU1 |
| | RU2 |
| | SART-2, -3 |
| | HLA-A*0201-R170I |
| | HSP70-2M |
| | EphA3 |
| | MMP |
| | TIMP |
| | E-cadherin |
| | hTERT |
| | 5T4 |
| | mucin 1(MUC1), |
| | carcinoembryonic (CEA), (Her-2/neu), |
| | telomerase, |
| | survivin, |
| | MAGE-A1 |
| | Carbonic anhydrase 9 (CA9) |
| | Mage9 |
| | GOLGA4 |
| | OFA |
| | von Hippel-Lindau (VHL) mutant |
| | surviving |
| | STEAP |
| Bladder Cancer | BAGE, |
| | CAMEL |
| | NY-ESO-1 |
| | LAGE-1 |
| | EpCAM |
| | MAGE-A1, -A2, -A3, -A4, -A6, -A10, -A12 (multiple |
| | epitopes of each MAGE antigen) |
| | AFP |
| | Livin (ML-IAP) |
| | KIAA0205 |
| | RAGE |
| | RU1 |
| | RU2 |
| | Survivin, -2B |
| | ErbB2 |
| | Her2/neu |
| | hTERT |
| | mannose receptor |
| | SART3 |
| | EZH2 |
| | MRP3 |
| | STEAP |
| Pancreatic Cancer | Ep-CAM |
| | MUC1 |
| | CLCA2 |
| | AFP |
| | HER2/neu |
| | MUC1 |
| | MUC2 |
| | hTERT |
| | P53 |
| | carcinoembryonic antigen (CEA |
| | Survivin, -2B |
| | K-RAS |
| | ErbB2 |
| | PSCA |
| | BCP-20 (FBXO39) |
| Ovarian Cancer | DAM-6, -10 |
| | ART-4 |
| | Mesothelin |
| | Adipophilin |
| | AIM-2a |
| | Cyp-B |
| | G250 |
| | HER2/neu |
| | hTERT |
| | MUC1 |
| | MUC2 |
| | Survivin, -2B |
| | folate receptor-α (FRα) |
| | WT1 |
| | MUC1 |
| | ErbB2 |
| | CSPG4 |
| | BCP-20 (FBXO39) |
| | carcinoembryonic antigen (CEA) |
| | STEAP |
| Colorectal Cancer | MAGE-A1, -A2, -A3, -A4, -A6, -A10, -A12 (multiple |
| | epitopes of each MAGE antigen) |
| | NY-ESO-1 |
| | SSX-2 |
| | carcinoembryonic antigen (CEA) |
| | EP-CAM |
| | AIM-2a |
| | hTERT |
| | P53 |
| | EphA2 |
| | Livin (ML-IAP) |
| | MUC1 |
| | MUC2 |
| | TGFβ RII |
| | RAGE |
| | RU2 |
| | TRG |
| | 5T4 |
| | WT1 |
| | K-RAS |
| | OGT |
| | TGFβ RII |
| | ErbB2 |
| | folate receptor-α (FRα) |
| | BCP-20 (FBXO39) |
| | STEAP |
| Leukemia | BAGE(Myeloma), |
| | CAMEL(Myeloma) |
| | NY-ESO-1 |
| | LAGE-1 |
| | XBP1 (myeloma) |
| | GAGE-3, -4, -5, -6, -7B (myeloma) |
| | GAGE-1, -2, -8 |
| | MAGE-A1, -A2, -A3, -A4, -A6, -A10, -A12 (multiple |
| | epitopes of each MAGE antigen) |
| | Adipophilin |
| | Cyp-B |
| | hTERT |
| | Livin (ML-IAP) |
| | PRAME |
| | WT1 |
| | RU2 |
| | MUC1 |
| | hTERT |
| | abl-bcr alb-b3(b2a2) |
| | abl-bcr alb-b4(b3a2) |
| | bcr-abla |
| | bcr-abl p210(b3a2) |
| | ETV6/AML |
| | Dek-can |
| | Pml/RARα |
| | TEL/AML1 |
| | BST2 (bone marrow stromal cell antigen 2; also |
| | known as PDCA-1, CD137 and HM1.24 |
| | EBV protein LMP1 and LMP2 |
| | EBV EBNA-1 |
| | TEX 101 |
| | ODF4 |
| | RHAMM |
| | CD19 |
| | CD20 |
| | ROR1 |
| | PR1 (Proteinase 1) |
| | Her2 |
| | ErbB2 |
| | (CSPG4) |
| | CD25 (T-Cell Leukemia) |
| | HTLV1 antigens |
| | HTLV2 antigens |
| Uterine Cancer | ART-4 |
| | Cyp-B |
| | G250 |
| | Livin (ML-IAP) |
| | SART-1 |
| | SART-2, −3 |
| | Survivin, −2B |
| | WT1 |
| | HPV E6 and E7 |
| | HPV L1 |
| | ErbB2 |
| | Ha-RAS |
| | c-MYC |
| | RAS p21 |
| | Her2/neu |
| | BST2 (bone marrow stromal cell antigen 2; also |
| | known as PDCA-1, CD137 and HM1.24 |
| | hTERT |
| Thyroid Cancer | AFP |
| | RU2 |
| | WT1 |
| | BCP-20 (FBXO39) |
| | Calcitonin |
| | BAGE |
| | GAGE 1-6 |
| | MAGE-1 |
| | MAGE-2 |
| | MAGE-3 |
| | SSX 1-5 |
| | NY-ESO-1 |
| | hTERT |

The invention features the administration of each of the above listed TAAs (or cells that interact with each of the above-listed TAAs, or antigen-binding scaffolds, e.g., an antibody, soluble T cell receptor, or chimeric receptor, specific for each of the above-listed TAAs), including the TAAs of Table 6, in combination with any of the above listed HSP90 inhibitors, OBAA, flunarizine, aphidicolin, damnacanthal, dantrolene, or an analog thereof, as if each individual pair of TAAs and HSP90 inhibitor, OBAA, flunarizine, aphidicolin, damnacanthal, dantrolene, or an analog thereof were specifically recited. Based on the discoveries disclosed herein, each of the recited pairs would be expected to have a greater therapeutic efficacy than the administration of either compound alone (e.g., act in synergy). This is based on the observation, disclosed herein, that an HSP90 inhibitor, OBAA, flunarizine, aphidicolin, damnacanthal, and dantrolene upregulate TAA expression on cancer cells, making them more sensitive to an immune response (e.g., an immune response triggered by administration of a TAA or a cell that interacts with a TAA).

In order to stimulate an immune response against tumor cells, TAAs (e.g., those disclosed herein) can be delivered by a variety of methods. For example, when administering one or more TAAs with an HSP90 inhibitor, OBAA, flunarizine, aphidicolin, damnacanthal, dantrolene, or an analog thereof, the TAA can be formulated to be presented to the immune system to stimulate an immune response towards the TAA. Thus, a TAA or antigenic fragment, or tumor or other cell having TAA can be administered in vivo. Tumor cells expressing TAA can optionally be treated ex vivo (e.g., with an HSP90 inhibitor, OBAA, flunarizine, aphidicolin, damnacanthal, dantrolene, or an analog thereof) and transfused into a patient during therapy. Any agent that enhances antigen expression or antigenicity of the tumor can be used to treat the tumor in vivo or ex vivo. Tumor cell lysates or extracts, or irradiated or heat killed cells that renders them incapable of growth, but still able to induce an immune response, can also be administered.

TAAs can be delivered as peptides (Jaeger et al. (1996) Int J Cancer 66:162; Jager et al. (2000) Proc Natl Acad Sci USA 97:12198; Marchand et al. (1999) Int J Cancer. 80:219), or as peptides in combination with adjuvants (Jager et al. (1996). Int J Cancer 67:54; Rosenberg et al. (1998). Nat Med 4:321; Cormier et al. (1997). Cancer J Sci Am. 3:37; Wang et al. (1999). Clin Cancer Res. 5:2756).

TAAs can also be delivered with other cells. For example, TAA peptides can be loaded into dendritic cells (Chen et al. (2001) Gene Ther 8:316; Fong et al. (2001). 1 Immunol 167:7150; Themer et al. (1999). 1 Exp Med 190:1669; Tso et al. (2001). Cancer Res 61:7925), or loaded into other antigen presenting cells (Pardoll (2002). Nature Rev Immunol 2:227).

Immunogenic fragments (subsequences, including antigenic peptides that can be targeted) of TAAs are also included. In addition, variants and modified forms of TAA capable of eliciting, increasing, or stimulating an immune response are also included.

Three types of DNA-based recombinant cancer vaccines have been used to deliver TAAs: DNA encoding TAAs can be used 1) to modify dendritic cells, 2) as ‘naked’ DNA-vaccine, or 3) to construct recombinant viral vaccines. Recombinant vaccines and vaccine strategies have been developed to induce and potentiate T cell responses of a host to TAAs. A particular example of such a strategy is recombinant poxvirus vectors in which the tumor-associated antigen (TAA) is inserted as a transgene. Recombinant vaccinia vaccines and recombinant avipox (replication-defective) vaccines have been employed to stimulate immune response towards the TAA; the use of diversified prime and boost strategies using different vaccines; and the insertion of multiple T cell co-stimulatory molecules into recombinant poxvirus vectors, along with the TAA gene, to enhance T cell immune response to the TAA, and enhance or induce anti-tumor immunity.

Additional TAAs are described, e.g., in Renkvist and Robbins, Cancer Immunol. Immunother. 50:3-15, 2001, and in Novellino et al., Cancer Immunol. Immunother. 54:187-207, 2005.

### Cells

The methods, compositions, and kits of the invention also provide an HSP90 inhibitor, OBAA, flunarizine, aphidicolin, damnacanthal, dantrolene, or an analog thereof, in combination with cells (e.g., white blood cells) that interact with a tumor cell (e.g., by interacting with a TAA selected from, e.g., Melan-A/MART-1, tyrosinase, gp100/pmel 17, TRP-1, TRP-2, an MITF, MITF-A, MITF-M, melanoma GP75, Annexin I, Annexin II, ADAbp, PGP 9.5, CRC-C017-1A/GA733, Ab2 BR3E4, CI17-1A/GA733, Hsp70, Hsp90, Hsp96, Hsp105, Hsp110, HSPPC-96, stress protein gp96, gp96-associated cellular peptide, G250, DPPIV, Mammaglobin, thyroglobulin, STn, CEA, CEA epitope CAP-I, CEA epitope CAP-2, etv6, amI1, PSA, PSA epitope PSA-1, PSA epitope PSA-2, PSA epitope PSA-3, Ad5-PSA, PSMA, PAP, PDEF, PTH-rP, EGFR, PLU1, OFA-iLR, MN/CA IX (CA9), HP59, Cytochrome oxidase 1, sp100, msa, Ran GTPase activating protein, a Rab-GAP protein, PARIS-I, T cell receptor/CD3-zeta chain, cTAGE-1, SCP-1, Glycolipid antigen-GM2, GD2 or GD3, GM3, FucosylGM1, Glycoprotein (mucin) antigens-Tn, Sialyl-Tn, TF, and Mucin-I, CA125 (MUC-16), a MAGE family antigen, GAGE-1,2, BAGE, RAGE, LAGE-1, GnT-V, EP-CAM/KSA, CDK4, a MUC family antigen, HER2/neu, ErbB-2/neu, p21ras, RCAS1, α-fetoprotein, E-cadherin, α-catenin, β-catenin, NeuGcGM3, Fos related antigen, Cyclophilin B, RCAS1, S2, L10a, Telomerase rt peptide, cdc27, fodrin, p120ctn, PRAME, GA733/EoCam, NY-BR-1, NY-BR-2, NY-BR-3, NY-BR-4, NY-BR-S, NY-BR-6, NY-BR-7, NY-ESO-1, L19H1, MAZ, PINCH, PRAME, Prp1p/Zer1p, WT1, APC, PHF3, LAGE-1, SART3, SCP-1, SSX-1, SSX-2, SSX-4, TAG-72, TRAG-3, MBTAA, a Smad tumor antigen, Imp1, HPV-16 E7, c-erbB-2, EBNA-1, HSVtk, L552S, TGF beta RII frame shift mutation, BAX frame shift mutation, or an immunogenic fragment thereof).

Immune cells that interact with a tumor cell include lymphocytes, plasma cells, B-cells, e.g., expressing an antibody against TAA, NK cells, LAK cells, and macrophages. Cells can be autologous (e.g., derived from a subject, treated, and readministered to the same subject) or allogeneic to a subject to be treated. Immune cells that enhance or stimulate an immune response against a TAA (e.g., dendritic cells or antigen presenting cells) are considered immune enhancing. In addition, a mammalian or non-mammalian cell that expresses an antibody (e.g., plasma cell, B-cell, or a mammalian or non-mammalian cell transfected with a nucleic acid encoding the antibody) that specifically binds to a TAA can be used in accordance with the invention. An immune cell that targets a tumor cell can be used in accordance with the invention. For example, adoptive immunotherapy, in which tumor-infiltrating or peripheral blood lymphocytes can be infused into a tumor patient, following optional stimulation with a cytokine.

In various aspects, the cell is selected from a T cell, NK cell, LAK cell, monocyte, or macrophage. In an additional aspect, the cell has been pre-selected to bind to an antigen (e.g., a TAA) expressed by the tumor (e.g., T lymphocytes selected for strong avidity to TAA as presented on HLA molecules, Dudley et al. (2002). Science 298:850; Yee et al. (2002). PNAS 99:16168).

Immune cells expressing chimeric immune receptors (e.g., chimeric T cell receptors) can also be used in the methods of the invention. Chimeric T cells receptors can contain, e.g., in a single chimeric species, the intracellular domain of CD3 zeta-chain, a signaling region from a costimulatory protein such as CD28, and a binding element that specifically interacts with a selected target.

The binding element can be, e.g., an extracellular domain able to specifically bind to a tumor (e.g., a TAA). The binding element can be the extracellular domain of a receptor that, in its native context, binds the particular extracellular marker of the tumor (e.g., a TAA). Alternatively, the extracellular domain can include any binding moiety specific for such an extracellular marker (e.g., a TAA), including, antibodies (e.g., single-chain Fv antibody fragments that are specific to a TAA and other antibodies and antibody analogs described below).

Specific chimeric immune receptors useful in the methods of the invention are disclosed, e.g., in U.S. Pat. Nos. 5,216,132; 5,502,167; 5,969,109; 6,083,751; 6,268,411; 6,734,013; 7,265,209; 7,446,179; 7,446,190; 7,446,191; 7,691,396; and 7,842,480; and U.S. Patent Application Nos. 2002/0006903; 2002/0086012; 2003/0148982; 2004/0043401; 2004/0204565; 2004/0249126; 2008/0160607; 2009/0304657; 2010/0105136; and 2011/0071273; each of which is incorporated by reference in its entirety.

The administration of immune cells can be combined with, e.g., lymphodepletion prior to administration of immune cells (e.g., T cells). Furthermore, treatment can also include the administration of one or more cytokines, e.g., IL-2, IL-7, and IL-15.

### Antigen-Binding Scaffolds

The invention also features the administration of an HSP90 inhibitor, OBAA, flunarizine, aphidicolin, damnacanthal, dantrolene, or an analog thereof, in combination with an antigen-binding scaffold, e.g., an antibody, soluble T cell receptor, or chimeric receptor.

Antibodies can be specific for any TAA (e.g., by being specific for a TAA selected from, e.g., Melan-A/MART-1, tyrosinase, gp100/pmel 17, TRP-1, TRP-2, an MITF, MITF-A, MITF-M, melanoma GP75, Annexin I, Annexin II, ADAbp, PGP 9.5, CRC-C017-1A/GA733, Ab2 BR3E4, CI17-1A/GA733, Hsp70, Hsp90, Hsp96, Hsp105, Hsp110, HSPPC-96, stress protein gp96, gp96-associated cellular peptide, G250, DPPIV, Mammaglobin, thyroglobulin, STn, CEA, CEA epitope CAP-I, CEA epitope CAP-2, etv6, amI1, PSA, PSA epitope PSA-1, PSA epitope PSA-2, PSA epitope PSA-3, Ad5-PSA, PSMA, PAP, PDEF, PTH-rP, EGFR, PLU1, OFA-iLR, MN/CA IX (CA9), HP59, Cytochrome oxidase 1, sp100, msa, Ran GTPase activating protein, a Rab-GAP protein, PARIS-I, T cell receptor/CD3-zeta chain, cTAGE-1, SCP-1, Glycolipid antigen-GM2, GD2 or GD3, GM3, FucosylGM1, Glycoprotein (mucin) antigens-Tn, Sialyl-Tn, TF, and Mucin-I, CA125 (MUC-16), a MAGE family antigen, GAGE-1,2, BAGE, RAGE, LAGE-1, GnT-V, EP-CAM/KSA, CDK4, a MUC family antigen, HER2/neu, ErbB-2/neu, p21ras, RCAS1, α-fetoprotein, E-cadherin, α-catenin, β-catenin, NeuGcGM3, Fos related antigen, Cyclophilin B, RCAS1, S2, L10a, Telomerase rt peptide, cdc27, fodrin, p120ctn, PRAME, GA733/EoCam, NY-BR-1, NY-BR-2, NY-BR-3, NY-BR-4, NY-BR-5, NY-BR-6, NY-BR-7, NY-ESO-1, L19H1, MAZ, PINCH, PRAME, Prp1p/Zer1p, WT1, APC, PHF3, LAGE-1, SART3, SCP-1, SSX-1, SSX-2, SSX-4, TAG-72, TRAG-3, MBTAA, a Smad tumor antigen, Imp1, HPV-16 E7, c-erbB-2, EBNA-1, HSVtk, L552S, TGF beta RII frame shift mutation, BAX frame shift mutation, any antigen listed in Table 6, or an immunogenic fragment thereof).

Antibodies include intact antibodies and antigen-binding fragments, e.g., the IgG, IgA, IgM, IgD, and IgE isotypes. Antibody fragments include separate variable heavy chains, variable light chains, Fab, Fab′, F(ab′)₂, Fabc, and scFv. Fragments can be produced by enzymatic or chemical separation of intact immunoglobulins. For example, a F(ab′)₂ fragment can be obtained from an IgG molecule by proteolytic digestion with pepsin at pH 3.0-3.5 using standard methods such as those described in Harlow and Lane, Antibodies: A Laboratory Manual, Cold Spring Harbor Pubs., New York, 1988. Fab fragments may be obtained from F(ab′)₂ fragments by limited reduction, or from whole antibody by digestion with papain in the presence of reducing agents. Fragments can also be produced by recombinant DNA techniques. Segments of nucleic acids encoding selected fragments are produced by digestion of full-length coding sequences with restriction enzymes, or by de novo synthesis. Often fragments are expressed in the form of phage-coat fusion proteins. This manner of expression is advantageous for affinity-sharpening of antibodies.

Methods of preparing chimeric and humanized antibodies and antibody fragments are described in, e.g., U.S. Pat. Nos. 4,816,567; 5,530,101; 5,622,701; 5,800,815; 5,874,540; 5,914,110; 5,928,904; 6,210,670; 6,677,436; and 7,067,313 and U.S. Patent Application Nos. 2002/0031508; 2004/0265311; and 2005/0226876. Preparation of antibody or antigen-binding fragments thereof is further described in, e.g., U.S. Pat. Nos. 6,331,415; 6,818,216; and 7,067,313.

Antigen-binding scaffolds also include, for example, soluble T cell receptors (as described, e.g., in Molloy et al., Curr. Opin. Pharmacol. 5:438-443, 2005) and chimeric receptors.

In addition, antigen-binding scaffolds include, e.g., antibody analogs specific for a TAA. Examples of such analogs are single domain antibodies (e.g., shark IgNAR and camelid VHH), protein frameworks including complementary determining regions (e.g., anticalins, affibodies, 4-helix bundle proteins, ankyrin repeat proteins, tetranectins, adnectins, A-domain proteins, lipocalins, immunity protein ImmE7, cytochrome b₅₆₂, amyloid β-protein precursor inhibitor, cellulose binding domain from cellobiohydrolase Cel7A, and carbohydrate binding module CBM4-2, C-type lectins), RNA and DNA aptamers, and molecularly imprinted nanoparticles, e.g., polymer nanoparticles.

The antigen-binding scaffolds can be conjugated to any known cytotoxic or therapeutic moiety to facilitate cancer therapy. Examples include but are not limited to antineoplastic agents such as: Acivicin; Aclarubicin; Acodazole Hydrochloride; Acronine; Adozelesin; Adriamycin; Aldesleukin; Altretamine; Ambomycin; A. metantrone Acetate; Aminoglutethimide; Amsacrine; Anastrozole; Anthramycin; Asparaginase; Asperlin; Azacitidine; Azetepa; Azotomycin; Batimastat; Benzodepa; Bicalutamide; Bisantrene Hydrochloride; Bisnafide Dimesylate; Bizelesin; Bleomycin Sulfate; Brequinar Sodium; Bropirimine; Busulfan; Cactinomycin; Calusterone; Camptothecin; Caracemide; Carbetimer; Carboplatin; Carmustine; Carubicin Hydrochloride; Carzelesin; Cedefingol; Chlorambucil; Cirolemycin; Cisplatin; Cladribine; Combretestatin A-4; Crisnatol Mesylate; Cyclophosphamide; Cytarabine; Dacarbazine; DACA (N-[2-(Dimethyl-amino)ethyl]acridine-4-carboxamide); Dactinomycin; Daunorubicin Hydrochloride; Daunomycin; Decitabine; Dexormaplatin; Dezaguanine; Dezaguanine Mesylate; Diaziquone; Docetaxel; Dolasatins; Doxorubicin; Doxorubicin Hydrochloride; Droloxifene; Droloxifene Citrate; Dromostanolone Propionate; Duazomycin; Edatrexate; Eflornithine Hydrochloride; Ellipticine; Elsamitrucin; Enloplatin; Enpromate; Epipropidine; Epirubicin Hydrochloride; Erbulozole; Esorubicin Hydrochloride; Estramustine; Estramustine Phosphate Sodium; Etanidazole; Ethiodized Oil I 131; Etoposide; Etoposide Phosphate; Etoprine; Fadrozole Hydrochloride; Fazarabine; Fenretinide; Floxuridine; Fludarabine Phosphate; Fluorouracil; 5-FdUMP; Flurocitabine; Fosquidone; Fostriecin Sodium; Gemcitabine; Gemcitabine Hydrochloride; Gold Au 198; Homocamptothecin; Hydroxyurea; Idarubicin Hydrochloride; Ifosfamide; Ilmofosine; Interferon Alfa-2a; Interferon Alfa-2b; Interferon Alfa-n1; Interferon Alfa-n3; Interferon Beta-I a; Interferon Gamma-I b; Iproplatin; Irinotecan Hydrochloride; Lanreotide Acetate; Letrozole; Leuprolide Acetate; Liarozole Hydrochloride; Lometrexol Sodium; Lomustine; Losoxantrone Hydrochloride; Masoprocol; Maytansinc; Mechlorethamine Hydrochloride; Megestrol Acetate; Melengestrol Acetate; Melphalan; Menogaril; Mercaptopurine; Methotrexate; Methotrexate Sodium; Metoprine; Meturedepa; Mitindomide; Mitocarcin; Mitocromin; Mitogillin; Mitomalcin; Mitomycin; Mitosper; Mitotane; Mitoxantrone Hydrochloride; Mycophenolic Acid; Nocodazole; Nogalamycin; Ormaplatin; Oxisuran; Paclitaxel; Pegaspargase; Peliomycin; Pentamustine; PeploycinSulfate; Perfosfamide; Pipobroman; Piposulfan; Piroxantrone Hydrochloride; Plicamycin; Plomestane; Porfimer Sodium; Porfiromycin; Prednimustine; Procarbazine Hydrochloride; Puromycin; Puromycin Hydrochloride; Pyrazofurin; Rhizoxin; Rhizoxin D; Riboprine; Rogletimide; Safingol; Safingol Hydrochloride; Semustine; Simtrazene; Sparfosate Sodium; Sparsomycin; Spirogermanium Hydrochloride; Spiromustine; Spiroplatin; Streptonigrin; Streptozocin; Strontium Chloride Sr 89; Sulofenur; Talisomycin; Taxane; Taxoid; Tecogalan Sodium; Tegafur; Teloxantrone Hydrochloride; Temoporfin; Teniposide; Teroxirone; Testolactone; Thiamiprine; Thioguanine; Thiotepa; Thymitaq; Tiazofurin; Tirapazamine; Tomudex; TOP53; Topotecan Hydrochloride; Toremifene Citrate; Trestolone Acetate; Triciribine Phosphate; Trimetrexate; Trimetrexate Glucuronate; Triptorelin; Tubulozole Hydrochloride; Uracil Mustard; Uredepa; Vapreotide; Verteporfin; Vinblastine; Vinblastine Sulfate; Vincristine; Vincristine Sulfate; Vindesine; Vindesine Sulfate; Vinepidine Sulfate; Vinglycinate Sulfate; Vinleurosine Sulfate; Vinorelbine Tartrate; Vinrosidine Sulfate; Vinzolidine Sulfate; Vorozole; Zeniplatin; Zinostatin; Zorubicin Hydrochloride; 2-Chlorodeoxyadenosine; 2′ Deoxyformycin; 9-aminocamptothecin; raltitrexed; N-propargyl-5,8-dideazafolic acid; 2chloro-2′-arabino-fluoro-2′-deoxyadenosine; 2-chloro-2′-deoxyadenosine; anisomycin; trichostatin A; hPRL-G129R; CEP-751; linomide; sulfur mustard; nitrogen mustard (mechlor ethamine); cyclophosphamide; melphalan; chlorambucil; ifosfamide; busulfan; N-methyl-Nnitrosourea (MNU); N,N′-Bis(2-chloroethyl)-N-nitrosourea (BCNU); N-(2-chloroethyl)-N′ cyclohexyl-N-nitrosourea (CCNU); N-(2-chloroethyl)-N′-(trans-4-methylcyclohexyl-N-nitrosourea (MeCCNU); N-(2-chloroethyl)-N′-(diethyl)ethylphosphonate-N-nitrosourea(fotemustine); streptozotocin; diacarbazine (DTIC); mitozolomide; temozolomide; thiotepa; mitomycin C; AZQ; adozelesin; Cisplatin; Carboplatin; Ormaplatin; Oxaliplatin; C1-973; DWA 2114R; JM216; JM335; Bis (platinum); tomudex; azacitidine; cytarabine; gemcitabine; 6-Mercaptopurine; 6-Thioguanine; Hypoxanthine; teniposide 9-amino camptothecin; Topotecan; CPT-11; Doxorubicin; Daunomycin; Epirubicin; darubicin; mitoxantrone; losoxantrone; Dactinomycin (Actinomycin D); amsacrine; pyrazoloacridine; all-trans retinol; 14-hydroxy-retro-retinol; all-trans retinoic acid; N-(4-Hydroxyphenyl) retinamide; 13-cis retinoic acid; 3-Methyl TTNEB; 9-cis retinoic acid; fludarabine (2-F-ara-AMP); or 2-chlorodeoxyadenosine (2-Cda).

Other therapeutic compounds include, but are not limited to, 20-pi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; argininedeaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bleomycin A2; bleomycin B2; bropirimine; budotitane; buthionine sulfoximine; calcipotriol; calphostin C; camptothecin derivatives (e.g., 10-hydroxy-camptothecin); canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; 2′deoxycoformycin (DCF); deslorelin; dexifosfamide; dexrazoxane; dexverapamil; diaziquone; didemnin B; didox; diethylnorspermine; dihydro-5-azacytidine; dihydrotaxol, 9-; dioxamycin; diphenyl spiromustine; discodermolide; docosanol; dolasetron; doxifluridine; droloxifene; dronabinol; duocarmycin SA; ebselen; ecomustine; edelfosine; edrecolomab; eflornithine; elemene; emitefur; epirubicin; epothilones (A, R=H; B, R=Me); epithilones; epristeride; estramustine analogue; estrogen agonists; estrogen antagonists; etanidazole; ctoposide; etoposide 4′-phosphate (etopofos); exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; hepsulfam; heregulin; hexamethylene bisacetamide; homoharringtonine (HHT); hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-; irinotecan; iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maytansine; mannostatin A; marimastat; masoprocol; maspin; matrilysin inhibitors; matrix metalloproteinase inhibitors; menogaril; merbarone; meterelin; methioninase; metoclopramide; MIF inhibitor; ifepristone; miltefosine; mirimostim; mismatched double stranded RNA; mithracin; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nernorubicin; neridronic acid; neutral endopeptidase; nilutamide; nisamycin; nitric oxide modulators; nitroxide antioxidant; nitrullyn; 06-benzylguanine; octreotide; okicenone; oligonucleotides; onapristone; ondansetron; ondansetron; oracin; oral cytokine inducer; ormaplatin; osaterone; oxaliplatin; oxaunomycin; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; podophyllotoxin; porfimer sodium; porfiromycin; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene conjugate; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; single chain antigen binding protein; sizofiran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thalidomide; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene dichloride; topotecan; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; velaresol; veramine; verdins; verteporfin; vinorelbine; vinxaltine; vitaxin; vorozole; zanoterone; zeniplatin; zilascorb; and zinostatin stimalamer.

An antigen-binding scaffold can also be coupled to a lytic peptide. Such lytic peptides induce cell death and include, but are not limited to, streptolysin O; stoichactis toxin; phallolysin; staphylococcus alpha toxin; holothurin A; digitonin; melittin; lysolecithin; cardiotoxin; and cerebratulus A toxin. An antigen-binding scaffold can also be conjugated to a synthetic peptide that shares some sequence homology or chemical characteristics with any of the naturally occurring peptide lysins; such characteristics include, but are not limited to, linearity, positive charge, amphipathicity, and formation of alpha-helical structures in a hydrophobic environment.

An antigen-binding scaffold can also be coupled to a radioactive agent to form an agent that can be used for therapeutic applications. Radioactive agents that can be used include but are not limited to ¹⁸F; ¹²⁵I; ¹³¹I; ¹²³I; ¹⁹⁷Hg; ²⁰³Hg; ⁷⁵Se; and ^{99m}Tc.

### Additional Therapeutic Agents

In addition to the HSP90 inhibitor, OBAA, flunarizine, aphidicolin, damnacanthal, dantrolene, or an analog thereof, one or more of an IFN-β receptor agonist, an IFN-γ receptor agonist, an immune stimulating molecule, a chemotherapeutic agent, an analgesic, an angiogenesis inhibitor, or a steroid may be administered or contained in the compositions of the invention.

IFN-β Receptor Agonists

The invention includes the administration of an IFN-β receptor agonist (e.g., IFN-β (also referred to herein as “IFN-B” and “IFN-beta”), an IFN-β mimic, or IFN-β receptor antibody peptide and mimetics) (as described, e.g., in U.S. Patent Application Publication No. 2004/0253235, which is hereby incorporated by reference in its entirety).

Exemplary forms of IFN-β are IFN-β-1a and IFN-β-1b. IFN-β-1a is sold, e.g., under the name Avonex®, and has the following amino acid sequence (human, mature form, N terminus (NH₂) to C terminus (COOH)):

(SEQ ID NO: 1)
MSYNLLGFLQRSSNFQCQKLLWQLNGRLEYCLKDRMNFDIPEEIKQLQQ
FQKEDAALTIYEMLQNIFAIFRQDSSSTGWNETIVENLLANVYHQINHL
KTVLEEKLEKEDFTRGKLMSSLHLKRYYGRILHYLKAKEYSHCAWTIVR
VEILRNFYFINRLTGYLRN.

IFN-β-1b is sold, e.g., under the name Betaseron®, and has the following amino acid sequence (human variant, mature form, N terminus to C terminus):

(SEQ ID NO: 2)
SYNLLGFLQRSSNFQSQKLLWQLNGRLEYCLKDRMNFDIPEEIKQLQQF
QKEDAALTIYEMLQNIFAIFRQDSSSTGWNETIVENLLANVYHQINHLK
TVLEEKLEKEDFTRGKLMSSLHLKRYYGRILHYLKAKEYSHCAWTIVRV
EILRNFYFINRLTGYLRN.

IFN-β receptor agonists include peptides and mimetics, and modified (variant) forms, provided that the modified form retains at least partial activity or function of unmodified or reference peptide or mimetic. For example, a modified IFN-β peptide or mimetic will retain at least a part of a TAA inducing activity. Modified (variant) peptides can have one or more amino acid residues substituted with another residue, added to the sequence or deleted from the sequence. Specific examples include one or more amino acid substitutions, additions, or deletions (e.g., 1, 2, 3, 4, 5, 6, 7, 8, 9, 10, 11, 12, 13, 14, 15, 16, 17, 18, 19, 20, or more). A modified (variant) peptide can have a sequence with 50%, 60%, 70%, 75%, 80%, 85%, 90%, 95%, 96%, 97%, 98%, 99%, or more identity to a reference sequence (e.g., IFN-β). The crystal structure of recombinant IFN-β can also be employed to predict the effect of IFN-β modifications (Senda, et al., EMBO J. 11:3193-3201, 1992).

Mammalian IFN-β sequences such as human (Gray and Goeddel (1982). Nature, 298:859); rat (Yokoyama, et al., (1997). Biochem Biophys Res Commun., 232:698); canine (Iwata, et al., (1996). J Interferon Cytokine Res., 10:765); porcine (J Interferon Res., (1992). 12:153) are known in the art. An example of IFN-β receptor agonist is anti-IFN anti-idotypic antibody (Osheroff et al. (1985). J Immunol, 135:306). A specific example of a IFN-β mimetic is SYR6 (Sato and Sone, (2003). Biochem J., 371(Pt 2):603). Additional modified IFN-β sequences are described, for example, in U.S. Pat. No. 6,514,729-recombinant interferon-beta muteins; U.S. Pat. No. 4,793,995-modified (1-56) beta interferons; U.S. Pat. No. 4,753,795-modified (80-113) beta interferons; and U.S. Pat. No. 4,738,845-modified (115-145) beta interferons.

IFN-γ Receptor Agonists

The invention includes the administration of an IFN-γ receptor agonist (e.g., IFN-γ (also referred to herein as “IFN-G” and “IFN-gamma”), an IFN-γ mimic, or IFN-γ receptor antibody peptides and mimetics).

Exemplary forms of IFN-γ are human natural IFN-γ-1a, IFN-γ-1b, and IFN-γ-1c. Human natural IFN-γ is a dimer, wherein each subunit has the following amino acid sequence (mature form, N terminus to C terminus):

(SEQ ID NO: 3)
QDPYVKEAENLKKYFNAGHSDVADNGTLFLGILKNWKEESDRKIMQSQI
VSFYFKLFKNFKDDQSIQKSVETIKEDMNVKFFNSNKKKRDDFEKLTNY
SVTDLNVQRKAIHELIQVMAELPPAAETGKRKRSQMLFRGRRASQ.

IFN-γ-1a is sold, e.g., under the names Immuneron® and Polyferon®, and is a dimer, wherein each subunit has the following amino acid sequence (human variant, mature form, N terminus to C terminus):

(SEQ ID NO: 4)
CYCQDPYVKEAENLKKYFNAGHSDVADNGTLFLGILKNWKEESDRKIMQ
SQIVSFYFKLFKNFKDDQSIQKSVETIKEDMNVKFFNSNKKKRDDFEKL
TNYSVTDLNVQRKAIHELIQVMAELPPAAETGKRKRSQMLFRGRRASQ.

IFN-γ-1b is sold, e.g., under the names Actimmune® and Immukin®, and is a dimer, wherein each subunit has the following amino acid sequence (human variant, mature form, N terminus to C terminus):

(SEQ ID NO: 5)
MQDPYVKEAENLKKYFNAGHSDVADNGTLFLGILKNWKEESDRKIMQSQ
IVSFYFKLFKNFKDDQSIQKSVETIKEDMNVKFFNSNKKKRDDFEKLTN
YSVTDLNVQRKAIHELIQVMAELPPAAETGKRKRSQMLFRGR.

IFN-γ-1c is a dimer, wherein each subunit has the following amino acid sequence (human variant, mature form, N terminus to C terminus):

(SEQ ID NO: 6)
MQDPYVKEAENLKKYFNAGHSDVADNGTLFLGILKNWKEESDRKIMQSQ
IVSFYFKLFKNFKDDQSIQKSVETIKEDMNVKFFNSNKKKRDDFEKLTN
YSVTDLNVQRKAIHELIQVMAELPPAAETGKRKRSQMLFRGRRASQ.

IFN-γ receptor agonists include peptides and mimetics, and modified (variant) forms, provided that the modified form retains at least partial activity or function of unmodified or reference peptide or mimetic. For example, a modified IFN-γ peptide or mimetic will retain at least a part of an MHC Class II upregulation activity. Modified (variant) peptides can have one or more amino acid residues substituted with another residue, added to the sequence or deleted from the sequence. Specific examples include one or more amino acid substitutions, additions, or deletions (e.g., 1, 2, 3, 4, 5, 6, 7, 8, 9, 10, 11, 12, 13, 14, 15, 16, 17, 18, 19, 20, or more). A modified (variant) peptide can have a sequence with 50%, 60%, 70%, 75%, 80%, 85%, 90%, 95%, 96%, 97%, 98%, 99%, or more identity to a reference sequence (e.g., IFN-γ-1b). The crystal structure of recombinant IFN-γ can also be employed to predict the effect of IFN-γ modifications (Ealick et al., Science 252:698-702, 1991).

Additional exemplary IFN-γ receptor agonists are described, e.g., in U.S. Pat. No. 5,595,888 and in U.S. Pat. No. 6,046,034.

Other Therapeutic Agents

Non-limiting examples of chemotherapeutic agents are cyclophosphamide, mechlorethamine, chlorambucil, melphalan, daunorubicin, doxorubicin, idarubicin, mitoxantrone, valrubicin, paclitaxel, docetaxel, etoposide, teniposide, tafluposide, azacitidine, azathioprine, capecitabine, cytarabine, doxifluridine, fluorouracil, gemcitabine, mercaptopurine, methotrexate, tioguanine, bleomycin, carboplatin, cisplatin, oxaliplatin, all-trans retinoic acid, vinblastine, vincristine, vindesine, and vinorelbine.

Non-limiting examples of analgesics are acetaminophen, diclonfenac, diflunisal, etodolac, fenoprofen, flurbiprofen, ibuprofen, indomethacin, ketoprofen, ketorolac, meclofenamate, mefenamic acid, meloxicam, nabumetone, naproxen, oxaprozin, phenylbutazone, piroxicam, sulindac, tolmetin, buprenorphine, butorphanol, codeine, hydrocodone, hydromorphone, levorphanol, meperidine, methadone, morphine, nalbuphine, oxycodone, oxymorphone, pentazocine, propoxyphene, tramadol, capsaicin, benzocaine, dibucaine, lidocaine, and prilocaine.

Non-limiting examples of angiogenesis inhibitors are soluble VEGFR-1 and NRP-1, angiopoietin-2, TSP-1, TSP-2, angiostatin, endostatin, vasostatin, calreticulin, platelet factor-4, TIMP, CDAI, Meth-1, Meth-2, interferon-α, interferon-β, interferon-γ, CXCL10, IL-4, IL-12, IL-18, prothrombin, anthrombin III fragment, prolactin, VEGI, SPARC, osteopontin, maspin, canstatin, proliferin-related protein, restin, bevacizumab, carboxyamidotriazole, TNP-470, CM101, suramin, SU5416, thrombospondin, VEGFR antagonists, cartilage-derived angiogenesis inhibitor factor, matrix metalloproteinase inhibitors, 2-methoxyestradiol, tecogalan, prolactin, and linomide. Non-limiting examples of steroids include: cortisone, hydrocortisone, prednisone, methylprednisone, corticosterone, deoxycorticosterone, 11-deoxycortisol, 18-hydroxycorticosterone, 1α-hydroxycorticosterone, and aldosterone.

Several chemotherapeutic agents, analgesics, angiogenesis inhibitors, and steroids are commercially available. Standard doses for chemotherapeutic agents, analgesics, angiogenesis inhibitors, and steroids are known in the art and can range from 0.1 mg to 500 mg (e.g., 0.1 mg to 400 mg, 0.1 mg to 300 mg, 0.1 mg to 250 mg, 1.0 mg to 200 mg, 1.0 mg to 150 mg; 1.0 mg to 100 mg; and 0.1 and 50 mg) for each individual chemotherapeutic agent, analgesic, angiogenesis inhibitor, and steroid.

Any compound, agent, therapy, or treatment having an immune-stimulating or enhancing activity or effect can be used in combination with an HSP90 inhibitor, OBAA, flunarizine, aphidicolin, damnacanthal, dantrolene, or an analog thereof. An immune enhancing compound provides an increase, stimulation, induction, or promotion of an immune response, humoral or cell-mediated. Such therapies can enhance immune response generally, or enhance immune response to the specific tumor. Specific non-limiting examples of immune enhancing agents include monoclonal, polyclonal antibody, and mixtures thereof (e.g., that specifically bind to a TAA).

Immune stimulating molecules, such as Flt3 ligand and cytokines (e.g., cell growth, proliferation, chemotactic and survival factors) that enhance or stimulate immunogenicity of TAA are considered immune enhancing, and can also be administered prior to, substantially contemporaneously with, or following administration of an HSP90 inhibitor, OBAA, flunarizine, aphidicolin, damnacanthal, dantrolene, or an analog thereof. Specific non-limiting examples of cytokines include IL-2, IL-1α, IL-β, IL-3, IL-7, IL-21, granulocyte-macrophage-colony stimulating factor (GM-CSF), IFNγ, IL-12, and TNF-β. GM-CSF stimulates antigen-presenting cells and exhibits anti-tumor activity, including against leukemia, melanoma, breast carcinoma, prostate carcinoma, and renal cell carcinoma, can be used in accordance with the invention.

Other forms of immunotherapy useful in the compositions, methods, and kits of the invention include CTLA-4 blockade (e.g., through inhibitor antibodies including MDX-010 (i.e., ipilimumab)) and inhibition of related factors (e.g., through antibodies or antagonists to PD-1, PD-L1, PD-L2, B7-H3, B7x/B7-H4, BTLA, B7.1, B7.2, and ICOS-L). Other examples of such immunotherapy are agonists against CD137 (4-1BB), ICOS, OX40, Toll like receptors (e.g., TLR9), and glucocorticoid induced tumor necrosis factor receptor (GITR) (e.g., agonist antibodies). Each of these therapies can also be used in combination with any of the above cytokine therapies (e.g., IL-2 therapy).

Molecules that that down-regulate the effects of TH1 immune response inhibitors are also considered as “immune enhancing.” Specific non-limiting examples include antibodies to IL-10 or IL-10 receptor, IL-4, and IL-5, thereby up-regulating the TH1 immune response.

Kinase inhibitors that enhance or stimulate TAA expression include Gleevec (STI571) and inhibitors of protein kinases (e.g. AKT inhibitor, H-89, PD98059, PD184352, U0126, HAIO77, forskolin and Y27632). Such kinase inhibitors may synergize with other compounds (e.g., an HSP90 inhibitor, OBAA, flunarizine, aphidicolin, damnacanthal, dantrolene, or an analog thereof) that stimulate, enhance or increase TAA expression.

Adjuvants refer to a class of substances which when added to an antigen improve the immune response. Examples include compounds which promote uptake by accessory cells (e.g. macrophages and dendritic cells) which process antigen, such as alum (aluminum hydroxide), incomplete Freund's adjuvant, complete Freund's adjuvant, Ribi, Montanide ISATM 51, GERBU vaccine adjuvant, CAP vaccine adjuvant, SLN (solid lipid nanoparticles), CpG DNA, and RC529 adjuvant, and GM-CSF (including using Sipuleucel-T treatment).

### Therapy

The invention features methods for treating a subject having cancer or at risk of developing a cancer (e.g., an increased risk of at least 10%, 20%, 30%, 40%, 50%, 60%, 70%, 80%, 90%, 100%, or more). Treatment is achieved by administering an HSP90 inhibitor, OBAA, flunarizine, aphidicolin, damnacanthal, dantrolene, or an analog thereof. Treatment can also be achieved by administering an HSP90 inhibitor, OBAA, flunarizine, aphidicolin, damnacanthal, dantrolene, or an analog thereof, in combination with administration of a TAA, an antigen binding scaffold (e.g., an antibody, a soluble T cell receptor, or chimeric receptor), a cell, and/or an IFN-β receptor agonist or IFN-γ receptor agonist. While the examples describe an HSP90 inhibitor, OBAA, flunarizine, aphidicolin, damnacanthal, dantrolene, or an analog thereof, it is understood that the combination of multiple agents is often desirable.

A subject may be diagnosed with a cancer by a physician using methods known in the art. The clinical symptoms of a cancer depend upon the specific type of cancer and include, without limitation, abscesses, poorly healing sores, lumps, indigestion, difficulty swallowing, hoarseness, persistent cough, bleeding, discharge, wart changes, mole changes, pain, unexplained weight loss, unexplained weight gain, fatigue, and fever. Desirably, the treatment decreases the severity or duration of one or more (e.g., 2, 3, 4, or 5) symptoms of a cancer.

Subjects include those who have risk factors associated with tumor development. For example, subjects at risk for developing melanoma include fair skin, high numbers of naevi (dysplastic nevus), sun exposure (ultraviolet radiation), patient phenotype, family history, and history of a previous melanoma. Subjects at risk for developing cancer can be identified with genetic screens for tumor associated genes, gene deletions or gene mutations. Subjects at risk for developing breast cancer lack Brca1, for example. Subjects at risk for developing colon cancer have deleted or mutated tumor suppressor genes, such as adenomatous polyposis coli (APC), for example.

Non-limiting examples of cancers that may be treated using the methods of the invention are: acute lymphoblastic leukemia, acute myeloid leukemia, adrenocortical carcinoma, anal cancer, appendix cancer, astrocytoma, atypical teratoid/rhabdoid tumor, basal cell carcinoma, bile duct cancer, bladder cancer, bone cancer, brain stem glioma, brain tumor, breast cancer, bronchial tumor, Burkitt lymphoma, carcinoid tumor, cervical cancer, chordoma, chronic lymphocytic leukemia, chronic myeloproliferative disorder, colon cancer, colorectal cancer, craniopharyngioma, cutaneous T cell lymphoma, endometrial cancer, ependymoblastoma, ependymoma, esophageal cancer, Ewing sarcoma, extracranial germ cell tumor, extragonadal germ cell tumor, extrahepatic bile duct cancer, eye cancer, gallbladder cancer, gastric cancer, gastroesophageal cancer, gastrointestinal cancer, germ cell tumor, gestational trophoblastic tumor, glioma, hairy cell leukemia, head and neck cancer, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, leukemia, lip and oral cavity cancer, liver cancer, lung cancer, malignant teratoma, non-Hodgkin lymphoma, macroglobulinemia, osteosarcoma, medulloblastoma, melanoma, merkel cell carcinoma, mesothelioma, mouth cancer, mycosis fungiodes, myelodysplastic syndrome, multiple myeloma, nasal cavity and paranasal sinus cancer, nasopharyngeal cancer, non-small cell lung cancer, oral cancer, oropharyngeal cancer, osteosarcoma, ovarian cancer, ovarian epithelial cancer, pancreatic cancer, papillomatosis, parathyroid cancer, penile cancer, pharyngeal cancer, pituitary tumor, prostate cancer, rectal cancer, renal cell cancer, retinoblastoma, rhabdomycosarcoma, salivary gland cancer, sarcoma, skin cancer, small intestine cancer, soft tissue sarcoma, testicular cancer, throat cancer, thomoma, thymic carcinoma, thyroid cancer, urethral cancer, uterine cancer, vaginal cancer, and Wilms tumor. The cancer to be treated may also be a metastatic cancer. Desirably, the treatment increases (e.g., by at least 10%, 15%, 20%, 25%, 30%, 35%, 40%, 45%, 50%, 55%, 60%, 65%, 70%, 80%, 90%, or even 100%) the cell death of cancer cells.

The invention includes the treatment of any metastatic or non-metastatic tumor, cancer, malignancy, or neoplasia of any cell or tissue origin.

In particular aspects, the treatment reduces tumor volume, inhibits an increase in tumor volume, stimulates tumor cell lysis or apoptosis, reduces tumor metastasis, reduces the cell number or viability of cells within a mestastasis, or reduces the number of new metastases. In another aspect, the subject is treated with or administered a further anti-tumor therapy (e.g., surgical resection, radiotherapy, immunotherapy, or chemotherapy).

The treatment of carcinomas refer to malignancies of epithelial or endocrine tissue, and include respiratory system carcinomas, gastrointestinal system carcinomas, genitourinary system carcinomas, testicular carcinomas, breast carcinomas, prostatic carcinomas, endocrine system carcinomas, and melanomas. Melanoma refers to malignant tumors of melanocytes and other cells derived from pigment cell origin that may arise in the skin, the eye (including retina), or other regions of the body, including the cells derived from the neural crest that also gives rise to the melanocyte lineage. A pre-malignant form of melanoma, known as dysplastic nevus or dysplastic nevus syndrome, is associated with melanoma development.

Exemplary carcinomas are those forming from the uterine cervix, lung, prostate, breast, head and neck, colon, pancreas, testes, adrenal, kidney, esophagus, stomach, liver and ovary. The term also includes carcinosarcomas, e.g., which include malignant tumors composed of carcinomatous and sarcomatous tissues. Adenocarcinoma includes a carcinoma of a glandular tissue, or in which the tumor forms a gland like structure.

Sarcomas include malignant tumors of mesenchymal cell origin. Exemplary sarcomas include for example, lymphosarcoma, liposarcoma, osteosarcoma, chondrosarcoma, leiomyosarcoma, rhabdomyosarcoma, and fibrosarcoma.

Neural neoplasias include glioma, glioblastoma, meningioma, neuroblastoma, retinoblastoma, astrocytoma, and oligodendrocytoma.

Liquid tumors are neoplasias of the reticuloendothelial or haematopoetic system, such as a lymphoma, myeloma and leukemia, or neoplasia that is diffuse in nature, as they do not typically form a solid mass. Particular examples of leukemias include acute and chronic lymphoblastic, myeloblastic, and multiple myeloma. Typically, such diseases arise from poorly differentiated acute leukemias, e.g., erythroblastic leukemia and acute megakaryoblastic leukemia. Specific myeloid disorders include, but are not limited to, acute pro myeloid leukemia (APML), acute myelogenous leukemia (AML), and chronic myelogenous leukemia (CML); lymphoid malignancies include, but are not limited to, acute lymphoblastic leukemia (ALL), which includes B-lineage ALL and T-lineage ALL, chronic lymphocytic leukemia (CLL), prolymphocytic leukemia (PLL), hairy cell leukemia (HLL), and Waldenstrom's macroglobulinemia (WM).

Specific malignant lymphomas include non Hodgkin lymphoma and variants, peripheral T cell lymphomas, adult T cell leukemia/lymphoma (ArL), cutaneous T cell lymphoma (CTCL), large granular lymphocytic leukemia (LGF), Hodgkin's disease, and Reed-Sternberg disease.

Cells comprising a tumor may be aggregated in a cell mass or be dispersed. A solid tumor is a neoplasia or metastasis that typically aggregates together and forms a mass. Specific examples include visceral tumors such as melanomas, breast, pancreatic, uterine, and ovarian cancers, testicular cancer, including seminomas, gastric or colon cancer, hepatomas, adrenal, renal, and bladder carcinomas, lung, head and neck cancers, and brain tumors/cancers.

A subject to be treated using the methods of the invention may be identified as being at risk for the development of a cancer (e.g., having at least a 5%, 10%, 15%, 20%, 25%, 30%, 40%, 50%, 60%, 70%, 80%, 90%, 100%, or more, increased chance of developing a cancer) by genotypic analysis, hazardous environmental exposure, and analysis of the medical history of the subject's family.

The invention therefore also provides methods of treating a tumor, methods of treating a subject having or at risk of having a tumor, and methods of increasing effectiveness of an anti-tumor therapy. In respective embodiments, a method includes administering to a subject with a tumor an amount of an HSP90 inhibitor, OBAA, flunarizine, aphidicolin, damnacanthal, dantrolene, or an analog thereof, and an antigen-binding scaffold (e.g., an antibody, a soluble T cell receptor, or a chimeric receptor), or a cell that produces an antigen-binding scaffold that specifically binds to a tumor associated antigen (TAA) sufficient to treat the tumor; administering to the subject an HSP90 inhibitor, OBAA, flunarizine, aphidicolin, damnacanthal, dantrolene, or an analog thereof, and an antigen-binding scaffold or a cell that produces an antigen-binding scaffold that specifically binds to a tumor associated antigen (TAA) sufficient to treat the subject; and administering to a subject that is undergoing or has undergone tumor therapy, an amount of an HSP90 inhibitor, OBAA, flunarizine, aphidicolin, damnacanthal, dantrolene, or an analog thereof, and an antibody or a cell that produces an antibody that specifically binds to a tumor associated antigen (TAA) sufficient to increase effectiveness of the anti-tumor therapy. In various aspects, the cell producing an antigen-binding scaffold that specifically binds to a tumor associated antigen (TAA) is selected from a plasma cell, B-cell, or a mammalian or non-mammalian cell transfected with a nucleic acid encoding the antigen-binding scaffold.

Methods of the invention include providing a detectable or measurable therapeutic benefit to a subject. A therapeutic benefit is any objective or subjective transient or temporary, or longer term improvement in the condition. Thus, a satisfactory clinical endpoint is achieved when there is an incremental improvement in the subject's condition or a partial reduction in the severity or duration of one or more associated adverse symptoms or complications or inhibition or reversal of one or more of the physiological, biochemical or cellular manifestations or characteristics of the disease. A therapeutic benefit or improvement need not be complete ablation of the tumor or any or all adverse symptoms or complications associated with the tumor. For example, inhibiting an increase in tumor cell mass (stabilization of a disease) can increase the subjects lifespan (reduce mortality) even if only for a few days, weeks or months, even though complete ablation of the tumor has not resulted.

Particular examples of therapeutic benefit or improvement include a reduction in tumor volume (size or cell mass), inhibiting an increase in tumor volume, a slowing or inhibition of tumor worsening or progression, stimulating tumor cell lysis or apoptosis, reducing or inhibiting tumor metastasis, reduced mortality, and for prolonging lifespan. Adverse symptoms and complications associated with tumor, neoplasia, and cancer that can be reduced or decreased include, for example, nausea, lack of appetite, and lethargy. Thus, a reduction in the severity or frequency of symptoms, an improvement in the subjects' subjective feeling, such as increased energy, appetite, psychological well being, are examples of therapeutic benefit.

### Administration

In the methods of the invention, one or more TAAs, cells that interact with the TAAs, or antigen-binding scaffolds (e.g., an antibody, a soluble T cell receptor, or a chimeric receptor) specific for the TAAs, may be administered substantially contemporaneously with an HSP90 inhibitor, OBAA, flunarizine, aphidicolin, damnacanthal, dantrolene, or an analog thereof, and/or additional therapeutic agents (e.g., an IFN-β receptor agonist or IFN-γ receptor agonist, e.g., IFN-β or IFN-γ), or may be administered to a subject within one or more hours (e.g., 1, 2, 3, 4, 5, 6, 7, 8, 9, 10, 11, 12, 18, 24, 36, 48, or 72 hours, or any range therein, e.g., 1-3, 1-6, 1-12, 1-24, 3-6, 6-12, 12-24, 24-48, or 24-72 hours), days (e.g., 1, 2, 3, 4, 5, 6, 7, 8, 9, 10, 11, 12, 13, 14, 15, 16,17, 18, 19, 20, 21, 22, 23, 24, 25, 26, 27, 28, 29, 30, or any range therein, e.g., 1-3, 1-4, 2-4, 3-5, 3-7, 4-7, 5-7, 7-10, 10-14, or 14-30 days) or months (1, 2, 3, 4, 5, 6, or any range therein, e.g., 1-2, 1-3, 1-4, 1-5, 1-6, 2-3, 2-6, or 3-6) before or after an HSP90 inhibitor, OBAA, flunarizine, aphidicolin, damnacanthal, dantrolene, or an analog thereof, and/or additional therapeutic agent (e.g., IFN-β or IFN-γ). Accordingly, one or more TAAs can be administered prior to, substantially contemporaneous with, or following administration of an HSP90 inhibitor, OBAA, flunarizine, aphidicolin, damnacanthal, dantrolene, or an analog thereof, and/or additional therapeutic agent, in any order desired.

Furthermore, one or more IFN-β or IFN-γ receptor agonists (e.g., IFN-β (e.g., IFN-β-1a, SEQ ID NO: 1) or IFN-γ (e.g., IFN-γ-1b, SEQ ID NO: 5)) may be administered substantially contemporaneously with an HSP90 inhibitor, OBAA, flunarizine, aphidicolin, damnacanthal, dantrolene, or an analog thereof, or may be administered within one or more hours (e.g., 1, 2, 3, 4, 5, 6, 7, 8, 9, 10, 11, 12, 18, 24, 36, 48, or 72 hours, or any range therein, e.g., 1-3, 1-6, 1-12, 1-24, 3-6, 6-12, 12-24, 24-48, or 24-72 hours), days (e.g., 1, 2, 3, 4, 5, 6, 7, 8, 9, 10, 11, 12, 13, 14, 15, 16,17, 18, 19, 20, 21, 22, 23, 24, 25, 26, 27, 28, 29, 30, or any range therein, e.g., 1-3, 1-4, 2-4, 3-5, 3-7, 4-7, 5-7, 7-10, 10-14, or 14-30 days) or months (1, 2, 3, 4, 5, 6, or any range therein, e.g., 1-2, 1-3, 1-4, 1-5, 1-6, 2-3, 2-6, or 3-6) before or after an HSP90 inhibitor, OBAA, flunarizine, aphidicolin, damnacanthal, dantrolene, or an analog thereof, with or without administration of a TAA. Accordingly, one or more IFN-β or IFN-γ receptor agonists can be administered prior to, substantially contemporaneous with, or following administration of an HSP90 inhibitor, OBAA, flunarizine, aphidicolin, damnacanthal, dantrolene, or an analog thereof, in any order desired. Desirably, IFN-β or IFN-γ is administered between one and three days prior to an HSP90 inhibitor, OBAA, flunarizine, aphidicolin, damnacanthal, dantrolene, or an analog thereof. If a subject is first administered TAA (singly or multiple times), the subject may subsequently be administered an HSP90 inhibitor, OBAA, flunarizine, aphidicolin, damnacanthal, dantrolene, or an analog thereof, and/or an additional therapeutic agent (e.g., one or more IFN-β or IFN-γ receptor agonists) multiple times. Likewise, if a subject is first administered an HSP90 inhibitor, OBAA, flunarizine, aphidicolin, damnacanthal, dantrolene, or an analog thereof, singly or multiple times, the subject may be subsequently administered TAA multiple times, and/or an additional therapeutic agent (e.g., one or more IFN-β or IFN-γ receptor agonists).

The HSP90 inhibitor, OBAA, flunarizine, aphidicolin, damnacanthal, dantrolene, or an analog thereof, may be administered in a low or subtherapeutic dosage, a standard dosage, or in a high dosage.

Likewise, the additional therapeutic agent (e.g., one or more IFN-β or IFN-γ receptor agonists) may be administered in a low or subtherapeutic dosage, a standard dosage, or in a high dosage.

Therapy according to the invention may be performed alone or in conjunction with another therapy and may be provided at home, the doctor's office, a clinic, a hospital's outpatient department, or a hospital. Treatment optionally begins at a hospital so that the doctor can observe the therapy's effects closely and make any adjustments that are needed, or it may begin on an outpatient basis. The duration of the therapy depends on the type of cancer being treated, the age and condition of the patient, the stage and type of the patient's cancer, and how the patient responds to the treatment. Additionally, a person having a greater risk of developing a cancer (e.g., a person with a familial history of cancer or subject to a toxic environmental exposure) may receive treatment to inhibit or delay the onset of a cancer.

Routes of administration for the various embodiments include, but are not limited to, topical, transdermal, and systemic administration (such as, intravenous, intramuscular, subcutaneous, inhalation, rectal, buccal, vaginal, intraperitoneal, intraarticular, ophthalmic, or oral administration). As used herein, “systemic administration” refers to all nondermal routes of administration, and specifically excludes topical and transdermal routes of administration.

The dosage and frequency of administration of each component of the combination can be controlled independently. For example, one compound may be administered three times per day, while the second compound may be administered once per day. Combination therapy may be given in on-and-off cycles that include rest periods so that the patient's body has a chance to recover from any as yet unforeseen side effects. The compounds may also be formulated together such that one administration delivers the two or more compounds.

### Formulation of Pharmaceutical Compositions

The HSP90 inhibitor, OBAA, flunarizine, aphidicolin, damnacanthal, dantrolene, or an analog thereof, may be contained in any appropriate amount in any suitable carrier substance, and is generally present in an amount of 1-95% by weight of the total weight of the composition. Likewise, the additional therapeutic agent (e.g., one or more IFN-β or IFN-γ receptor agonists), if present, may be contained in any appropriate amount in any suitable carrier substance, and is generally present in an amount of 1-95% by weight of the total weight of the composition. The pharmaceutical compositions may be provided in a dosage form that is suitable for the oral, parenteral (e.g., intravenously, intramuscularly), rectal, cutaneous, nasal, vaginal, inhalant, skin (patch), or ocular administration route. Thus, the compositions may be in the form of, e.g., tablets, capsules, pills, powders, granulates, suspensions, emulsions, solutions, gels including hydrogels, pastes, ointments, creams, plasters, drenches, osmotic delivery devices, suppositories, enemas, injectables, implants, sprays, or aerosols. The pharmaceutical compositions may be formulated according to conventional pharmaceutical practice (see, e.g., Remington: The Science and Practice of Pharmacy, 20th edition, 2000, Ed. A. R. Gennaro, Lippincott Williams & Wilkins, Philadelphia, and Encyclopedia of Pharmaceutical Technology, Eds. J. Swarbrick and J. C. Boylan, 1988-1999, Marcel Dekker, New York).

### Dosages

The dosage of the HSP90 inhibitor, OBAA, flunarizine, aphidicolin, damnacanthal, dantrolene, or an analog thereof, administered to a subject may be 0.1 mg per day to 900 mg per day (depending on the compound), desirably about 1.0 mg per day to 800 mg per day, 1.0 mg per day to 700 mg per day, 1.0 mg per day to 600 mg per day, 1.0 mg per day to 500 mg per day, 1.0 mg per day to 400 mg per day, 1.0 mg per day to 350 mg per day, 1.0 mg per day to 300 mg per day, 1.0 mg per day to 250 mg per day, 1.0 mg per day to 200 mg per day, 1.0 mg per day to 150 mg per day, 1.0 mg per day to 100 mg per day, and 0.1 mg per day to 50 mg per day. Desirably, the HSP90 inhibitor, OBAA, flunarizine, aphidicolin, damnacanthal, dantrolene, or an analog thereof, is administered in a low or subtherapeutic dose to the subject (e.g., human) in order to reduce adverse side effects of treatment. A single dosage of HSP90 inhibitor, OBAA, flunarizine, aphidicolin, damnacanthal, dantrolene, or an analog thereof, or a combination of one of these agents with one or more second agents may contain 0.5 mg, 1.0 mg, 2.0 mg, 3.0 mg, 4.0 mg, 5.0 mg, 7.5 mg, 10 mg, 15 mg, 20 mg, 25 mg, 50 mg, 75 mg, 100 mg, 125 mg, 150 mg, 175 mg, 200 mg, 225 mg, 250 mg, 275 mg, 300 mg, 350 mg, 400 mg, 450 mg, 500 mg, 550 mg, 600 mg, 650 mg, 700 mg, 750 mg, 800 mg, 850 mg, or 900 mg of an HSP90 inhibitor, OBAA, flunarizine, aphidicolin, damnacanthal, dantrolene, or an analog thereof, alone or in combination with one or more second agent(s) may be formulated using any of the above-described formulations (e.g., oral, topical, transdermal, intravenous, intramuscular, subcutaneous, inhalation, rectal, buccal, vaginal, intraperitoneal, intraarticular, or ophthalmic administration). For administration of the HSP90 inhibitor, OBAA, flunarizine, aphidicolin, damnacanthal, dantrolene, or an analog thereof, by injection, the dosage is normally about 0.1 mg to 900 mg, desirably about 0.01 mg to 600 mg, and more desirably about 1.0 mg to 100 mg. Injections are desirably given one to four times daily.

The dosage of the IFN-β receptor agonist (e.g., IFN-β, e.g., IFN-β-1a or IFN-β-1b) administered to a subject may be, e.g., 0.1 μg per day to 5 mg per day (depending on the compound), desirably about 1.0 μg per day to 1.0 mg per day, 1.0 μg per day to 900 μg per day, 1.0 μg per day to 800 μg per day, 1.0 μg per day to 700 μg per day, 1.0 μg per day to 600 μg per day, 1.0 μg per day to 500 μg per day, 1.0 μg per day to 400 μg per day, 1.0 μg per day to 300 μg per day, 1.0 μg per day to 200 μg per day, 2.0 μg per day to 200 μg per day, 5.0 μg per day to 200 μg per day, 10.0 μg per day to 200 μg per day, 20.0 μg per day to 200 μg per day, 50.0 μg per day to 200 μg per day, 100 μg per day to 200 μg per day, 10.0 μg per day to 100 μg per day, 30.0 μg per day to 100 μg per day, 50.0 μg per day to 100 μg per day, 30.0 μg per day to 60 μg per day, 20.0 μg per day to 40 μg per day, or any other range between any two of the following amounts: 0.1 μg per day, 0.2 μg per day, 0.5 μg per day, 1.0 μg per day, 2.0 μg per day, 5.0 μg per day, 10.0 μg per day, 20.0 μg per day, 30.0 μg per day, 40.0 μg per day, 50.0 μg per day, 60.0 μg per day, 70.0 μg per day, 80.0 μg per day, 90.0 μg per day, 100 μg per day, 200 μg per day, 300 μg per day, 400 μg per day, 500 μg per day, 600 μg per day, 700 μg per day, 800 μg per day, 900 μg per day, 1 mg per day, 2 mg per day, or 5 mg per day. In some instances, 30.0 μg of IFN-β-1a corresponds to about 6 million international units of antiviral activity. Desirably, the IFN-β receptor agonist is administered in a low or subtherapeutic dose to the subject (e.g., human) in order to reduce adverse side effects of treatment. A single dosage of IFN-β receptor agonist, or a combination of IFN-β receptor agonists or other compounds, may contain, e.g., 0.1 μg, 0.2 μg, 0.5 μg, 1.0 μg, 2.0 μg, 3.0 μg, 4.0 μg, 5.0 μg, 10.0 μg, 20.0 μg, 30.0 μg, 40.0 μg, 50.0 μg, 60.0 μg, 70.0 μg, 80.0 μg, 90.0 μg, 100 μg, 200 μg, 300 μg, 400 μg, 500 μg, 600 μg, 700 μg, 800 μg, 900 μg, 1.0 mg, 2.0 mg, or 5.0 mg of IFN-β receptor agonist, e.g., IFN-β, alone or in combination with one or more additional compounds, which may be formulated using any of the above-described formulations (e.g., oral, topical, transdermal, intravenous, intramuscular, subcutaneous, inhalation, rectal, buccal, vaginal, intraperitoneal, intraarticular, or ophthalmic administration). Any of the dosages listed herein could be administered more or less frequently than daily, e.g., once every 1, 2, 3, 4, 5, 6, 7, 8, 9, 10, 11, 12, 18, 24, 36, 48, or 72 hours, or any range therein, e.g., 1-3, 1-6, 1-12, 1-24, 3-6, 6-12, 12-24, 24-48, or 24-72 hours, days (e.g., 1, 2, 3, 4, 5, 6, 7, 8, 9, 10, 11, 12, 13, 14, 15, 16,17, 18, 19, 20, 21, 22, 23, 24, 25, 26, 27, 28, 29, 30, or any range therein, e.g., 1-3, 1-4, 2-4, 3-5, 3-7, 4-7, 5-7, 7-10, 10-14, or 14-30 days) or months (1, 2, 3, 4, 5, 6, or any range therein, e.g., 1-2, 1-3, 1-4, 1-5, 1-6, 2-3, 2-6, or 3-6). A desirable dosage for IFN-β-1a is 30.0 μg, e.g., three times per week.

The dosage of the IFN-γ receptor agonist (e.g., IFN-γ, e.g., human natural IFN-γ, IFN-γ-1a, IFN-γ-1b, or IFN-γ-1c) administered to a subject may be, e.g., 0.1 μg per day to 5 mg per day (depending on the compound), desirably about 1.0 μg per day to 1.0 mg per day, 1.0 μg per day to 900 μg per day, 1.0 μg per day to 800 μg per day, 1.0 μg per day to 700 μg per day, 1.0 μg per day to 600 μg per day, 1.0 μg per day to 500 μg per day, 1.0 μg per day to 400 μg per day, 1.0 μg per day to 300 μg per day, 1.0 μg per day to 200 μg per day, 2.0 μg per day to 200 μg per day, 5.0 μg per day to 200 μg per day, 10.0 μg per day to 200 μg per day, 20.0 μg per day to 200 μg per day, 50.0 μg per day to 200 μg per day, 100 μg per day to 200 μg per day, 10.0 ng per day to 100 μg per day, 30.0 μg per day to 100 μg per day, 50.0 μg per day to 100 μg per day 30.0 μg per day to 60 μg per day, 20.0 μg per day to 40 μg per day, or any other range between any two of the following amounts: 0.1 μg per day, 0.2 μg per day, 0.5 μg per day, 1.0 μg per day, 2.0 μg per day, 5.0 μg per day, 10.0 μg per day, 20.0 μg per day, 30.0 μg per day, 40.0 μg per day, 50.0 μg per day, 60.0 μg per day, 70.0 μg per day, 80.0 μg per day, 90.0 μg per day, 100 μg per day, 200 μg per day, 300 μg per day, 400 μg per day, 500 μg per day, 600 μg per day, 700 μg per day, 800 μg per day, 900 μg per day, 1 mg per day, 2 mg per day, or 5 mg per day. In some instances, 50.0 μg of IFN-β-1a corresponds to about 1 million international units of antiviral activity. Desirably, the IFN-γ receptor agonist is administered in a low or subtherapeutic dose to the subject (e.g., human) in order to reduce adverse side effects of treatment. A single dosage of IFN-γ receptor agonist, or a combination of IFN-γ receptor agonists or other compounds, may contain, e.g., 0.1 μg, 0.2 μg, 0.5 μg, 1.0 μg, 2.0 μg, 3.0 μg, 4.0 μg, 5.0 μg, 10.0 μg, 20.0 μg, 30.0 μg, 40.0 μg, 50.0 μg, 60.0 μg, 70.0 μg, 80.0 μg, 90.0 μg, 100 μg, 200 μg, 300 μg, 400 μg, 500 μg, 600 μg, 700 μg, 800 μg, 900 μg, 1.0 mg, 2.0 mg, or 5.0 mg of IFN-γ receptor agonist, e.g., IFN-γ, alone or in combination with one or more additional compounds, which may be formulated using any of the above-described formulations (e.g., oral, topical, transdermal, intravenous, intramuscular, subcutaneous, inhalation, rectal, buccal, vaginal, intraperitoneal, intraarticular, or ophthalmic administration). Any of the dosages listed herein could be administered more or less frequently than daily, e.g., once every 1, 2, 3, 4, 5, 6, 7, 8, 9, 10, 11, 12, 18, 24, 36, 48, or 72 hours, or any range therein, e.g., 1-3, 1-6, 1-12, 1-24, 3-6, 6-12, 12-24, 24-48, or 24-72 hours, days (e.g., 1, 2, 3, 4, 5, 6, 7, 8, 9, 10, 11, 12, 13, 14, 15, 16,17, 18, 19, 20, 21, 22, 23, 24, 25, 26, 27, 28, 29, 30, or any range therein, e.g., 1-3, 1-4, 2-4, 3-5, 3-7, 4-7, 5-7, 7-10, 10-14, or 14-30 days) or months (1, 2, 3, 4, 5, 6, or any range therein, e.g., 1-2, 1-3, 1-4, 1-5, 1-6, 2-3, 2-6, or 3-6). A desirable dosage for IFN-γ-1b is 50.0-100.0 μg, e.g., three times per week, e.g., administered subcutaneously, or 100.0 μg per day, e.g., administered intravenously, e.g., for 14 days.

The dosage of any of the above compositions can be “about” the recited dosage, wherein a dosage “about” a particular range is within 10% of the recited range. Doses also considered sufficient are those that result in a reduction of the use of another therapeutic regimen or protocol. For example, an HSP90 inhibitor, OBAA, flunarizine, aphidicolin, damnacanthal, dantrolene, or an analog thereof, and one or more TAAs (or cells interacting with a TAA, or antigen-binding scaffold (e.g., an antibody, a soluble T cell receptor, or a chimeric receptor) specific for the TAA), and/or an additional therapeutic agent (e.g., one or more IFN-β receptor agonist or IFN-γ receptor agonist), are considered as having a therapeutic effect if administration results in less chemotherapeutic drug, radiation or immunotherapy being required for tumor treatment.

### EXAMPLES

The following examples are provided for the purpose of illustrating the invention and are not meant to limit the invention in any way.

### Example 1

### Screening Assays and Results

A summary of the assay used to screen for compounds described herein is summarized in Table 7. The assay relies on the use of an antigen-specific responder T cell line. To achieve this goal, we utilized a transduced TCR with specificity for a known Melan-A/MART-1 peptide in the context of a specific MHC molecule, with suitable binding affinity and demonstrated biological signaling functionality. The chosen TCR reacts specifically to a Melan-A/MART-1 decapeptide (₂₆EAAGIGILTV₃₅) presented by HLA-A2, with appropriate triggering of T cell activation. We then used a lentiviral vector system for transduction of this specific TCR into the J.RT3-T3.5 T cell line, a Jurkat cell derivative which fails to express endogenous surface TCR through a mutation in its beta chain. The functional specificity of this transduced Jurkat cell line (J-TCR-M1) was established by its stimulation (as measured by IL-2 ELISA) only by HLA-A2+ tumor cells presenting the correct Melan-A/MART-1 peptide.

The stimulator melanoma cell line for the assay was selected on the basis of its constitutive low, but perceptible, expression of Melan-A/MART-1 that can be consistently enhanced by treatment with IFN-β, a cytokine previously shown to upregulate Melan-A/MART-1 and MHC Class I. As the transduced TCR used in the responding J-TCR-M1 cells is restricted by HLA-A2, the tumor cells were of necessity HLA-A2+. We evaluated several candidate melanoma cell lines for the ability to stimulate the J-TCR-M1 cell line. Levels of Melan-A/MART-1 (determined by intracellular staining and flow cytometry) and corresponding levels of induced IL-2 (determined by coculture with J-TCR-M1 and IL-2 ELISA) were tested for different melanoma cell lines. A clear correlation was seen between Melan-A/MART-1 levels and IL-2 induction (Table 8). In most of the melanoma cell lines tested, IFN-β increased Melan-A/MART-1 level and IL-2 induction, with the greatest signal to noise shown by MU89, possibly due to its lower initial baseline expression level of Melan-A/MART-1. The MU89 cell line was chosen for use in the cell based assay because of these characteristics.

IFN-β was chosen as a positive control to include in the assay because of its favorable induction of IL-2 by the MU89 melanoma cell line. The robustness of a high-volume screening assay is generally assessed by calculating the Z′ factor, which assesses control variation, as well as the difference between untreated and positive controls. This factor is defined from the mean (μ) and standard deviations (σ) of the positive (+) and negative (−) controls in a screen by the equation

$$Z \tilde{O} = 1 - \frac{3 \times ( {σ}_{+} + {σ}_{-} )}{( {μ}_{+} - {μ}_{-} )} .$$

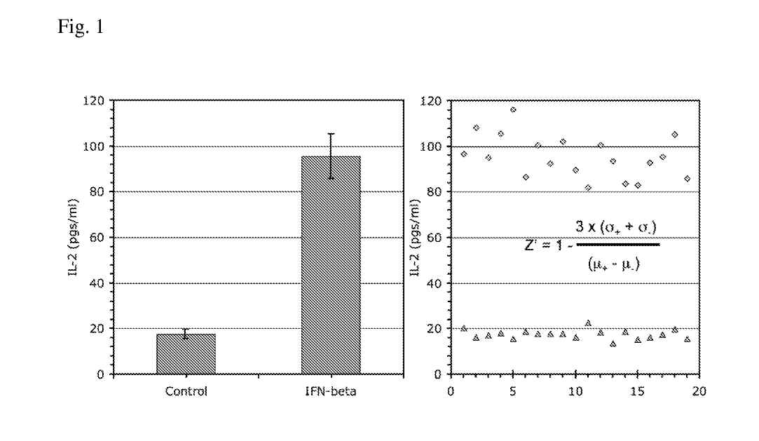

Our positive and negative control data was accordingly used to derive a Z′ value for the screening assay (FIG. 1). Since a satisfactory Z′-factor is above 0.5, the calculated Z′-factor level (0.55 from an n=16) indicated that our assay showed sufficient robustness for its continued development.

The use of IFN-β as a positive control has an important role in determining if a screened plate is of acceptable quality. Based on empirical testing, a screened plate generally is not considered valid unless the IFN-β positive control shows a 2-fold increase over the untreated control value. The IFN-β responses thus act as a quality control measure, flagging plates with poor performance in the assay that are thus necessarily rejected.

Previous experience has suggested that a three day treatment period would be suitable for evaluation of antigen-enhancing effects of either small molecules or protein mediators, and this time frame was also validated with the IL-2 assay co-culture assay. We chose to use a concentration of 10 μM for library screening. Although most clinically relevant compounds work at nanomolar concentrations, by the use of a higher concentration we aimed to avoid excluding compounds with lower activity, whose potencies could be improved by structure-activity relationship studies and analog syntheses during subsequent lead development.

In order to determine optimal cell numbers per well for the indicator melanoma cell line MU89, it is necessary to account for assay sensitivity limits, cell growth limitations for assay scale-up (10 to 20 plates at a time), and allowance for the three day culture period prior to the addition of the transduced Jurkat responder line J-TCR-M1. During this three day incubation, cell growth will occur and may even be stimulated by certain screened library molecules, but the assay must also accommodate reduction in cell numbers through cytostatic or cytotoxic compounds. With these restrictions in mind, we chose to use 5×10⁴ tumor cells per well of a 96 well plate for assay development.

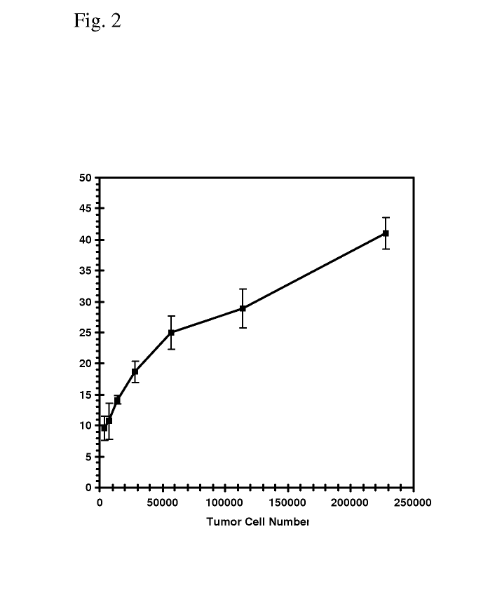

Given the inherent variation in stimulator melanoma cell numbers after the initial three day treatment, it was important to determine the optimal ratio of stimulator cells to responder transduced T cells. We chose a 2:1 stimulator to responder ratio as empirically giving reproducible results with tested positive controls. Since large excesses of stimulator cells alone (added at the beginning of the assay period) result in significant increases in IL-2 production from the responders (FIG. 2), in principle some test compounds might give positive signals through differential augmentation of melanoma stimulator cell growth over the T cell responders, rather than via enhancement of antigen expression per cell. In practice, there is no reason for suspecting such agents will be common, and culture conditions set physical limits to the maximal possible extent of stimulator cell proliferation. If such hypothetical agents were scored as primary hits, they will in any case be rapidly excluded through the first set of secondary screens, as detailed below.

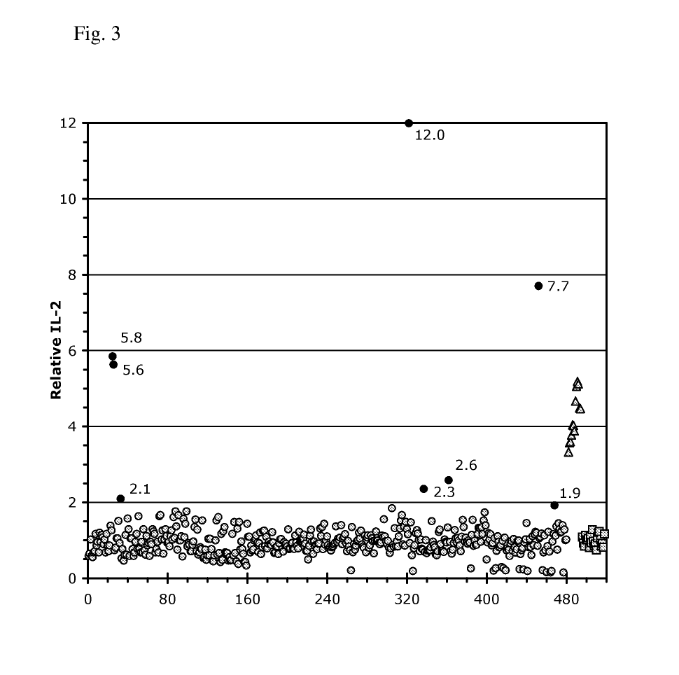

For evaluation of our cell based assay, we screened 480 known bioactive compounds, with results presented in FIG. 3. Using the definition of a hit as a compound inducing a 2-fold signal above the untreated control, we found eight hits in the primary screen (Table 9). A 2-fold relative increase of IL-2 above the untreated control level is equivalent to 6 standard deviations (of the controls) from the control mean, a highly significant factor.

Compounds which are highly toxic to either cell type involved in the assay will suppress the IL-2 read-out, sometimes to below baseline levels (as shown for some compounds plotted in FIG. 3). There is also the possibility that some molecules will block the signal transduction pathways involved in the activation of the T cell and subsequent IL-2 production. In turn, such toxic library members may fail to be scored as hits even if they possess inherent antigen up-regulation potential. This issue is underscored by the observation that certain compounds within the ICCB known bioactivcs library were independently identified as up-regulators of melanocyte gene expression including topoisomerase inhibitors (doxorubicin, etoposide, and camptothecin) and MEK inhibitors (U0126 and PD98059), that failed to be identified during our screening. In the case of daunorubicin (chemically very similar to doxorubicin), we have previously confirmed that this compound interferes with the direct co-culture assay, yet an enhanced IL-2 signal is elicited if this drug is removed by washing after tumor cell treatment, prior to exposure of responder T cells. This interference could be due to direct T cell toxicity. Both daunorubicin and doxorubicin are able to induce strong antigen up-regulation. Thus, doxorubicin is a false negative, actually able to up-regulate antigen expression, but not detected by our screen due to the ability of the compound to inhibit the assay read-out of T cell IL-2 production. The failure of the MEK inhibitors U0126 and PD98059 to induce signals in our assay is attributable to similar toxic effects or to inhibition of signal transduction.

The 480 compounds were screened in six plates, each containing 80 compounds and controls. The differences among the wells and plates assayed on the same day were minimal (FIG. 3). There is variability from day to day in absolute IL-2 induction, but after normalization, (relative to untreated controls), there is very low day to day variation in the assay (FIG. 3). Thus, we observed that the assay had good reproducibility yielding similar results among different plates and different assay days.

Secondary screens were carried out on the 8 primary hits, to further test performance robustness, specificity, and certain functional properties. Initially, for any previously-described compound, extensive literature searches must be performed to look for all relevant known bioactivities, especially towards melanocytic cells or T cells. Experimental secondary screens on primary hits involved (1) testing for non-specific T cell stimulation; (2) extensive dose-response testing in the co-culture assay over broad concentration ranges; (3) testing effects on a Melan-A/MART-1 promoter EGFP reporter cell line; and (4) performing intracellular staining testing multiple melanoma cell lines and using antibodies specific for an additional melanocyte-specific antigen. Testing for non-specific T cell stimulation will identify false positives, and compounds failing this test will not be further evaluated in any other secondary screens. Testing the compounds again in the IL-2 assay was implemented to confirm the primary hits and allow us to identify optimal doses for use in performing the other secondary screens. Because several of the compounds in the primary screen were cytostatic or cytotoxic to the tumor cells when testing over a wide dose range, the starting tumor cell number was increased to 5×10⁴ tumor cells to accommodate for cell loss during drug treatment. A corresponding number of T cells, 2.5×10⁴, were used to maintain a 2:1 tumor to T cell ratio.

Secondary screening with assays that are functionally orthogonal to the primary screen are useful since hits passing such evaluations are assigned a higher probability that their observed effects are not assay artifacts, and are biologically significant. The primary assay we employed measures a cell-based functional result dependent on multiple signaling, including processing, presentation, and activation effects. In principle a pharmacological agent can act at numerous different levels, any of which may contribute to an observed beneficial outcome. In such circumstances it may be expedient to use convenient orthogonal secondary assays which make certain assumptions regarding an agent's mode of action. While agents failing such a secondary screen may still be useful, agents passing both robust primary screening and secondary orthogonal assays can be confidently given higher priority in the downstream evaluation pipeline.

The EGFP reporter driven by the Melan-A/MART-1 promoter represents one such orthogonal assay because it evaluates directly the effect of the compounds on Melan-A/MART-1 promoter activity independent of Melan-A/MART-1 protein expression. Compounds passing the other secondary screens, but failing this one, may affect antigen presentation by other mechanisms such as increasing the efficiency of antigen processing, enhancing MHC Class I levels, or by stabilizing the antigen/MHC complex.

A further orthogonal secondary screen is the measurement of Melan-A/MART-1 and gp100 in additional melanoma cell lines and gliomas in response to the primary hit candidates. An observable increase in gp100 demonstrates that the compounds are able to positively modulate protein levels of distinct melanocyte specific antigens. If the compounds of interest increase antigen levels in multiple melanoma cell lines and two gliomas, the effects of the compounds are clearly not limited to a single melanoma cell type.

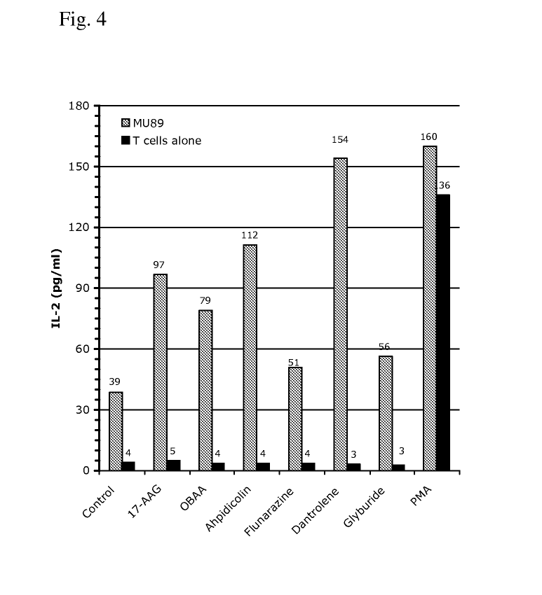

The published databases allowed us to identify one of the hits, phorbol 12-myristate 13-acetate (PMA), as a known T cell activator which is capable of directly inducing T cell IL-2 production. The results of the secondary screen for non-specific T cell activators is shown in FIG. 4, showing that only PMA caused T cells stimulation in the absence of tumor. This allowed us to exclude this agent as a “false positive.”

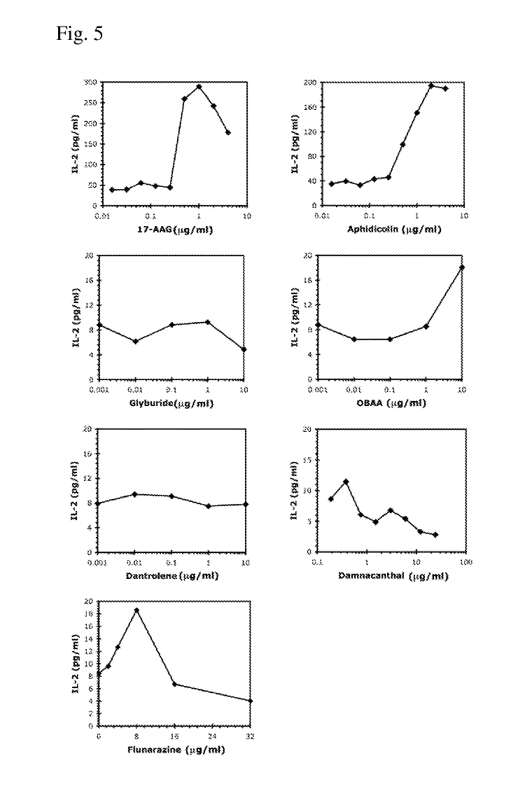

To confirm the activity of the hits from the primary screen, these eight hits were re-tested over a wider dose range. FIG. 5 shows IL-2 production induced by the various hits. We noted that 17-AAG and aphidicolin are compounds with large IL-2 stimulation effects, about 7-fold and 5-fold respectively. Two additional compounds, flunarizine and OBAA, gave more modest stimulation (1.5-2-fold) of IL-2 production by the responding T cells.

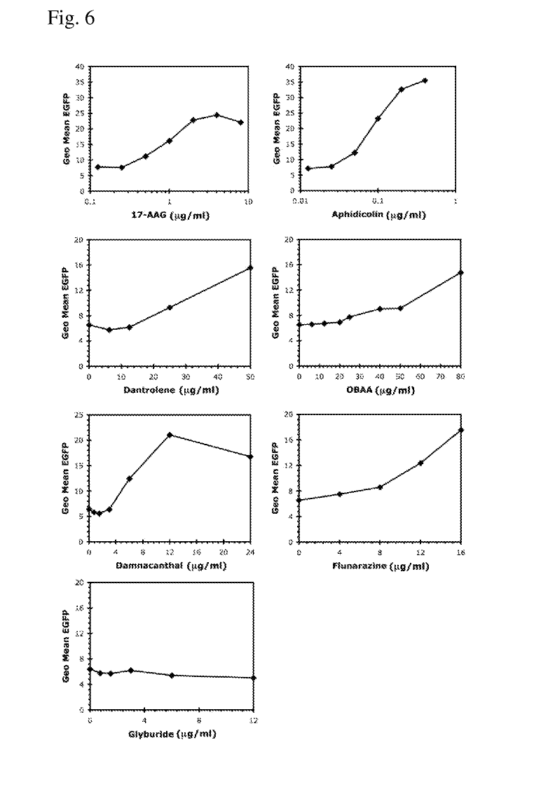

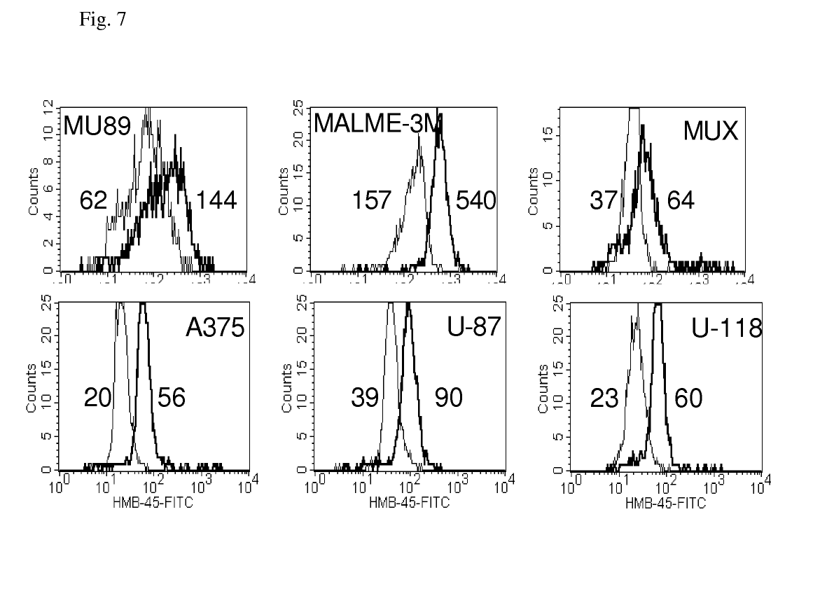

An orthogonal assay to demonstrate biological activity of the test compounds involves their ability to stimulate an EGFP reporter driven by the Melan-A/MART-1 promoter in a cell-based system. The seven hits (excluding PMA) from the primary screen were evaluated over a dose range as shown in FIG. 6. As for the primary screen, 17-AAG and aphidicolin, had the largest impact on EGFP stimulation, inducing 3-fold and 5-fold increases respectively. Four additional compounds: damnacanthal, flunarizine, OBAA, and dantrolene stimulated EGFP more than 2-fold. We then tested the ability of the hit compounds to induce antigen increases in a more diverse set of tumors including melanomas and gliomas. In addition, the gp100 antigen that is expressed on melanocytes and gliomas was added to the Melan-A/MART-1 antigen expression tested in the screening cellular assay. FIG. 7 illustrates examples of the flow histograms derived from the intracellular staining used to generate the data presented in Table 10. As can be seen by the concentrations of the compounds needed to reach this cut-off level, some hits clearly outperform others. Again 17-AAG and aphidicolin are the best performers in this assay, inducing high levels of protein increases at low concentrations. Damnacanthal, OBAA, and flunarizine induced significant responses (increasing protein >1.5-fold in most cell lines), but required much higher concentrations for the observed effect.

TABLE 7
Cell-based assay screening protocol.

| Step | Parameter | Description |
|---|---|---|
| 1 | Media | 50 μl DMEM containing 10% scrum plated in 384-well plate |
| | | Corning (cat no 3712) |
| 2 | Add Compounds | Use pin transfer to add 0.1 μl of test compounds (5 mg/ml) |
| | | Final concentration for most compounds about 10 μM |
| 3 | Plate tumor cells | 96-well plate, 1 × 10⁵ cells per well in 100 μl |
| | | Falcon (cat no 353072) tissue culture treated plates |
| 4 | Add compounds to | 80 wells contain test compounds. 8 wells are established to |
| | 96-well plate with | generate a standard curve of known IL-2 pg/ml |
| | tumor cells | 4 wells contain IFN-β, and four wells are untreated controls |
| 5 | Incubate | 72 hours in 37 degree tissue culture incubator containing 5% CO2. |
| 6 | Add T cells | 5 × 10⁴ cells in 50 μl per well |
| | | Spin plates gently to pellet cells and facilitate interaction |
| 7 | Incubate | 24 hours in 37 degree tissue culture incubator containing 5% CO₂. |
| 8 | Remove supernatant | 100 μl aliquots from each well |
| 9 | IL-2 ELISA | IL-2 ELISA kit from BD Biosciences (cat no 555190) Microtest |
| | | ELISA plates BD Bioscience (cat no 353279) |
| | | Read OD in plate reader BioRad 3550 450 nm-595 nm |
| 10 | Calculations | Use standard curve to calculate IL-2 pg/ml for each well. |
| | | Calculate fold over average of untreated control. |
| | | Establish efficacy of positive control (IFN-β) (>2-fold over |
| | | control) |

TABLE 8
Correlation of Melan-A/MART-1 intracellular staining and IL-2
secretion by T cells co-cultured with control vs. IFN-β treated
tumor cells.

| Cell Line^{a} | Treatment^{b} | MART-1^{c} | IL-2^{d} |
|---|---|---|---|
| MU89 | Control | 79 | 25 |
| | IFN-β | 153 | 96 |
| 453A | Control | 99 | 114 |
| | IFN-β | 113 | 159 |
| H59-44T | Control | 91 | 21 |
| | IFN-β | 103 | 23 |
| MALME-3M | Control | 117 | 186 |
| | IFN-β | 130 | 289 |
| MUX | Control | 13 | 10 |
| | IFN-β | 18 | 12 |
| A375 | Control | 9 | 15 |
| | IFN-β | 9 | 7 |

^{a}All cell lines are HLA-A2
^{b}IFN-β treatment with 5000 IU/ml for three days
^{c}Geometric Mean of intracellular staining using anti-MART-1
^{d}IL-2 (pg/ml) after coculture with MART-1 TCR Jurkat cells.

TABLE 9
Hits from pilot screen

| Compound | Fold increase | Known Inhibitory Targets |
|---|---|---|
| OBAA | 5.9 | Phospholipase A2 |
| Flunarizine | 11.9 | Ca²⁺-channel blocker |
| 17-AAG | 7.7 | HSP90 |
| Aphidicolin | 2.0 | DNA polymerase |
| Damnacanthal | 2.1 | p56lck |
| PMA | 1.9 | PKC activator |
| Dantrolene | 2.4 | Ca²⁺ release from sarcoplama reticulum |
| Glyburide | 2.6 | ATP dependent K channels |

OBAA = 3-(4-octadecyl)benzoylacrylic acid;
17-AAG = 17-allylaminogeldanamycin;
PMA = phorbol-12-myristate-13 acetate.

TABLE 10
Intracellular Staining for Melan-A/MART-1 and gp100 melanomas and gliomas exposed to hits.

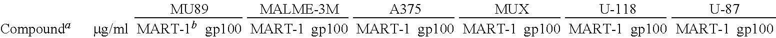

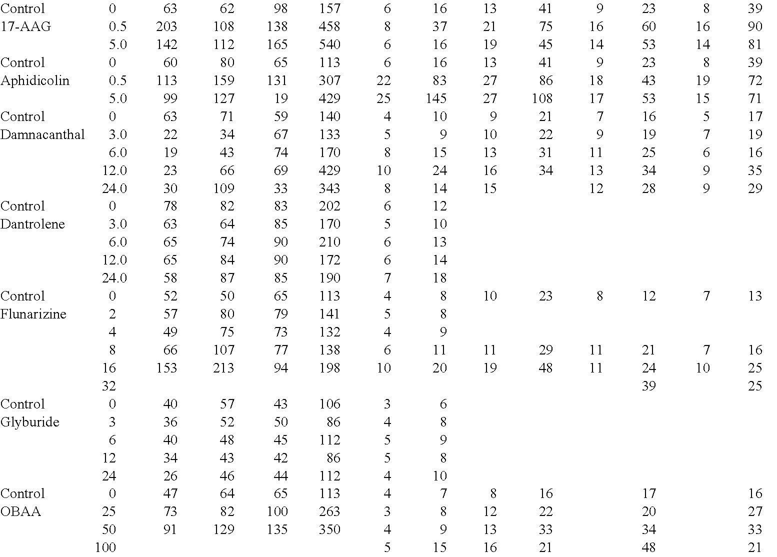

^{a}Treatment with indicated dose of compound was for three days.
^{b}Geometric Mean of intracellular staining using antibodies to MART-1 or gp100 as indicated.
Shaded squares indicate an increase of >=1.5 fold over control with compound treatment.

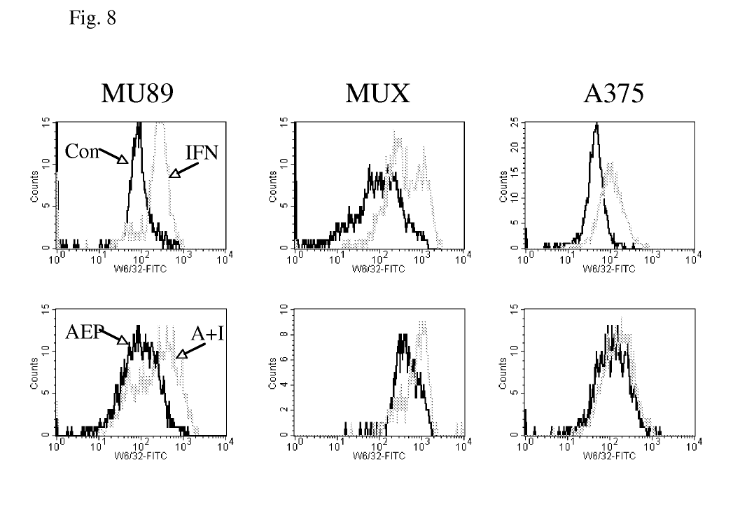

Based on the results with 17-AAG, we also tested additional inhibitors of HSP90. FIG. 8 shows the flow histograms from MHC Class I staining of treated cells with IFN-β and the HSP90 inhibitor 17-AEP individually and in combination. An increase in the fluorescence staining indicates an increase in MHC Class I. The geometric mean fluorescence intensities of these flow histograms were used to generate Table 11. Table 11 shows several melanoma cell lines treated with IFN-β and HS90 inhibitors individually and in combination. The level of MHC Class I expression increases between two to five fold with IFN-β treatment alone as expected. As shown previously the level of MHC Class I expression also increases between 2.5 to 4.5 fold with HSP90 inhibition.

This effect is observed with the use of three separate inhibitors of HSP90, 17-AEP a water soluble relative of 17-AAG which are relatives of the natural product geldanamycin, PU-H71, a purine derivative found to block HSP90 by competing with ATP binding, and CCT018159, an HSP90 inhibitor identified by drug screening. The combination treatments were carried using two methods. Both involve the pre-treatment of the melanoma tumor cells with IFN-β for four days, after which in one set of conditions the IFN-β is removed and the other set of conditions it is added again. Under both sets of conditions, a synergy between IFN-β and HSP90 inhibition is observed, incubated together higher levels of MHC Class I are observed than with either alone. Some of the increases seen were in excess of an order of magnitude (10-fold) more MHC Class I expression. The four day pretreatment without further IFN-β in combination with the HSP90 inhibitors was always lower than the four day pretreatment with addition of more IFN-β in combination with HSP90 inhibition. These results were seen with multiple cells lines and for multiple HSP90 inhibitors. The MU89 cell line was also more responsive to the combination of IFN-β and HSP90 inhibitors than the other melanoma cell lines MUX and A375.

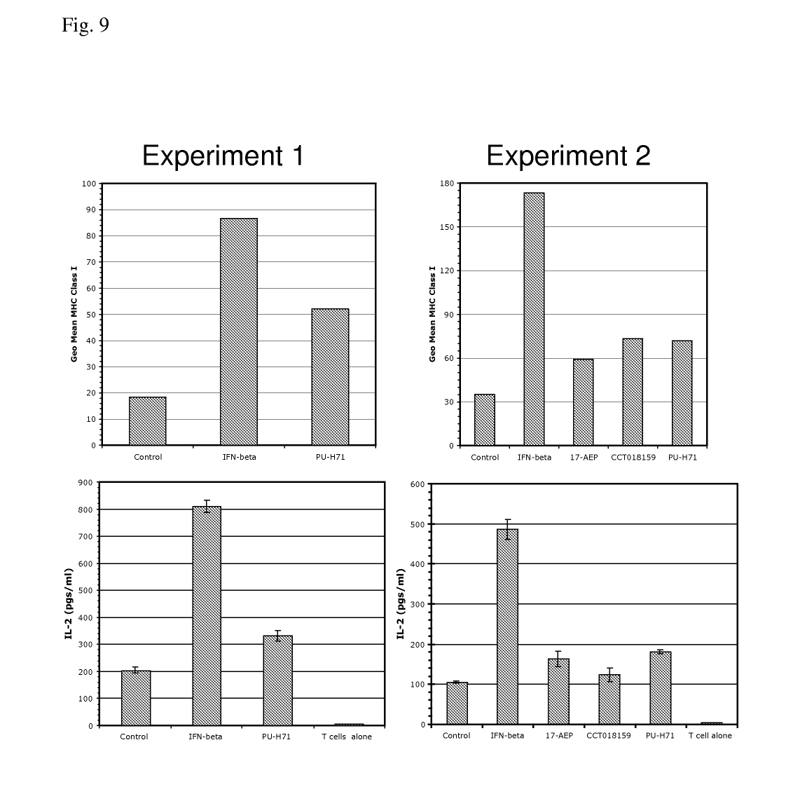

As described, we use a Jurkat T cell line solely expressing an HLA-2 restricted T cell receptor recognizing a MART-1 specific peptide. The melanoma tumor line MU89 expresses the MART-1 protein and will induce the Jurkat MART-1 TCR cell line to make and secrete a certain level of IL-2 upon co-culture of the two cell lines together. If the MU89 cell line is treated to up-regulate either MART-1 or MCH Class I protein levels then the level of IL-2 secretion will increase. We found synergy of IFN-β and HSP90 inhibitor treatment using this system. MU89 cells treated with IFN beta alone increased IL-2 levels by 2-fold. MU89 cells treated with HSP90 inhibitors increased IL-2 level 1.5 to 2-fold. Together the IL-2 levels increased by three fold for all three of the HSP90 inhibitors tested (FIG. 9). This increase was due to up-regulation of MHC Class I because, as shown in Tables 12-14, the combination of IFN-β and HSP90 inhibitors did not increase the level of the MART-1 protein only the level of MHC Class I. Table 15 shows class II expression.

TABLE 11
MHC Class I expression after treatment of melanoma
cell lines with HSP90 inhibitors.

| Cell Line | Treatment | Class I |
|---|---|---|
| MALME | Control | 44 |
| | IFN-β | 194 |
| | 17-AEP | 105 |
| | PU-H71 | 137 |
| | CCT | 72 |
| MU89 | Control | 18 |
| | IFN-β | 51 |
| | 17-AEP | 54 |
| | PU-H71 | 66 |
| | CCT | 75 |
| A375^{c} | Control | 36 |
| | IFN-β | 84 |
| | 17-AEP | 67 |
| | PU-H71 | 71 |
| | CCT | 70 |
| MUX | Control | 104 |
| | IFN-β | 158 |
| | 17-AEP | 219 |
| | PU-H71 | 212 |
| | CCT | 174 |

^{a}Cells were untreated (control) or treated with 5000 Units/ml of IFN-β, or with the HSP90 inhibitors 17-AEP (1 mg/ml), PU-H71 (0.3 mg/ml), CCT018159 (10 mg/ml) for three days.
^{b}Number represents geometric mean of surface staining with the MHC Class I antibody W6/32 of live gated cells.
^{c}Concentration of HSP90 inhibitors for the A375 cell line were: 17-AEP (0.5 mg/ml), PU-H71 (0.15 mg/ml), CCT018159 (2.5 mg/ml).

TABLE 12
MHC Class I expression after treatment of various
cancer cell lines with HSP90 inhibitors.

| Cell line | Tumor type | Treatment^{a} | Class I^{b} |
|---|---|---|---|
| U-118 | Glioma | Control | 48 |
| | | IFN-β | 62 |
| | | PU-H71 | 88 |
| U-87 | Glioma | Control | 62 |
| | | IFN-β | 138 |
| | | PU-H71 | 71 |
| HeLa | Cervical | Control | 113 |
| | carcinoma | IFN-β | 185 |
| | | PU-H71 | 182 |
| EBV-19 | B cell | Control | 100 |
| | lymphoma | IFN-β | 219 |
| | | PU-H71 | 119 |
| U2-OS | Osteo- | Control | 58 |
| | sarcoma | IFN-β | 180 |
| | | PU-H71 | 108 |
| MCF-7 | Breast | Control | 47 |
| | cancer | IFN-β | 62 |
| | | PU-H71 | 61 |
| Jurkat | T cell | Control | 28 |
| | lymphoma | IFN-β | 65 |
| | | PU-H71 | 41 |
| Raji | B cell | Control | 25 |
| | lymphoma | IFN-β | 28 |
| | | PU-H71 | 79 |

^{a}Cells were untreated (control) or treated with 5000 Units/ml of IFN-β, or with the HSP90 inhibitor PU-H71 (0.075-0.3 mg/ml) for three days.
^{b}Number represents geometric mean of surface staining with the MHC Class I antibody W6/32 of live gated cells.

TABLE 13
MHC Class I staining of Melanoma cell lines
treated with IFN-β and HSP90 inhibitors.

| | | |
|---|---|---|
| IFN-β | | |
| Treatment^{a} | HSP90 | MHC |

| Cell Line | 4 day | 3 day^{b} | Inhibitor^{c} | Class I^{d} |
|---|---|---|---|---|
| MU89 | None | None | None | 19 |
| | None | Yes | None | 83 |
| | None | None | 17-AEP | 84 |
| | None | None | PU-H71 | 75 |
| | None | None | CCT018159 | 73 |
| | Yes | None | None | 36 |
| | Yes | None | 17-AEP | 86 |
| | Yes | None | PU-H71 | 307 |
| | Yes | None | CCT018159 | 232 |
| | Yes | Yes | None | 115 |
| | Yes | Yes | 17-AEP | 227 |
| | Yes | Yes | PU-H71 | 390 |
| | Yes | Yes | CCT018159 | 281 |
| MUX | None | None | None | 76 |
| | None | Yes | None | 388 |
| | None | None | 17-AEP | 286 |
| | None | None | PU-H71 | 282 |
| | None | None | CCT018159 | 265 |
| | Yes | None | None | 207 |
| | Yes | None | 17-AEP | 496 |
| | Yes | None | PU-H71 | 477 |
| | Yes | None | CCT018159 | 397 |
| | Yes | Yes | None | 499 |
| | Yes | Yes | 17-AEP | 615 |
| | Yes | Yes | PU-H71 | 579 |
| | Yes | Yes | CCT018159 | 599 |
| A375 | None | None | None | 42 |
| | None | Yes | None | 87 |
| | None | None | 17-AEP | 109 |
| | None | None | PU-H71 | 131 |
| | None | None | CCT018159 | 119 |
| | Yes | None | None | 42 |
| | Yes | None | 17-AEP | 90 |
| | Yes | None | PU-H71 | 174 |
| | Yes | None | CCT018159 | 139 |
| | Yes | Yes | None | 99 |
| | Yes | Yes | 17-AEP | 144 |
| | Yes | Yes | PU-H71 | 226 |
| | Yes | Yes | CCT018159 | 181 |

^{a}Cells were treated with 5000 Units/ml of IFN-β in each instance.
^{b}After four day pretreatment with IFN-β, media was changed for continued three day treatment, thus fresh IFN-β was added if treatment was continued.
^{c}Concentration of HSP90 inhibitors for MU89 and MUX cell lines: 17-AEP (1 mg/ml), PU-H71 (0.3 mg/ml), CCT018159 (10 mg/ml). HSP90 inhibitors the A375 cell line: 17-AEP (0.5 mg/ml), PU-H71 (0.15 mg/ml), CCT018159 (2.5 mg/ml). All treatments with HSP90 inhibitors were for three days concurrent with IFN-β three day treatment.
^{d}Number represents geometric mean of surface staining with the MHC Class I antibody W6/32 of live gated cells.

TABLE 14
MHC Class I staining of other tumor cell lines treated
with IFN-β and HSP90 inhibitors.

| | | |
|---|---|---|
| IFN-β | | |
| Treatment^{a} | HSP90 | Class I |

| Cell Line | 4 day | 3 day^{b} | Inhibitor^{c} | MHC^{d} |
|---|---|---|---|---|
| U-118 | None | None | None | 53 |
| | None | Yes | None | 56 |
| | None | None | PU-H71 | 138 |
| | Yes | None | None | 88 |
| | Yes | None | PU-H71 | 224 |
| | Yes | Yes | None | 81 |
| | Yes | Yes | PU-H71 | 184 |
| Raji | None | None | None | 28 |
| | None | Yes | None | 102 |
| | None | None | PU-H71 | 157 |
| | Yes | None | None | 63 |
| | Yes | None | PU-H71 | 139 |
| | Yes | Yes | None | 61 |
| | Yes | Yes | PU-H71 | 129 |
| HeLa | None | None | None | 107 |
| | None | Yes | None | 126 |
| | None | None | PU-H71 | 165 |
| | Yes | None | None | 120 |
| | Yes | None | PU-H71 | 168 |
| | Yes | Yes | None | 107 |
| | Yes | Yes | PU-H71 | 137 |
| MCF-7 | None | None | None | 47 |
| | None | Yes | None | 62 |
| | None | None | PU-H71 | 61 |
| | Yes | None | None | 165 |
| | Yes | None | PU-H71 | 227 |
| | Yes | Yes | None | 208 |
| | Yes | Yes | PU-H71 | 213 |

^{a}Cells were treated with 5000 Units/ml of IFN-β in each instance.
^{b}After four day pretreatment with IFN-β media was changed for continued three day treatment, thus fresh IFN-β was added if treatment was continued.
^{c}Concentration of the HSP90 inhibitor PU-H71 was 0.3 mg/ml. All treatments with HSP90 inhibitors were for three days concurrent with IFN-β three day treatment.
^{d}Number represents geometric mean of surface staining with the MHC Class I antibody W6/32 of live gated cells.

TABLE 15
MHC Class II expression after treatment with a HSP90 inhibitor.

| | | MHC Class | |
|---|---|---|---|
| Cell Line | Treatment^{a} | I^{b} | MHC Class II |
| MU89 | Control | 52 | 9 |
| | IFN-β | 245 | 14 |
| | IFN-γ | 213 | 139 |
| | PU-H71 | 99 | 43 |
| EBV-19 | Control | 144 | 59 |
| | IFN-β | 314 | 27 |
| | IFN-γ | 277 | 118 |
| | PU-H71 | 124 | 114 |
| Raji | Control | 42 | 202 |
| | IFN-β | 81 | 278 |
| | IFN-γ | 117 | 142 |
| | PU-H71 | 139 | 160 |
| Giloma-118 | Control | 48 | 2 |
| | IFN-β | 62 | 3 |
| | IFN-γ | 137 | 40 |
| | PU-H71 | 88 | 4 |
| HeLa | Control | 181 | 8 |
| | IFN-β | 240 | 8 |
| | IFN-γ | 637 | 214 |
| | PU-H71 | 402 | 19 |
| U2-OS | Control | 52 | 4 |
| | IFN-β | 82 | 4 |
| | IFN-γ | 284 | 237 |
| | PU-H71 | 87 | 4 |
| Jurkat | Control | 28 | 1 |
| | IFN-β | 31 | 1 |
| | IFN-γ | 87 | 1 |
| | PU-H71 | 24 | 1 |

^{a}Cells were untreated (control) or treated with 5000 Units/ml of IFN-β, 100 ng/ml of IFN-γ, or with the HSP90 inhibitor PU-H71 (0.3 mg/ml) for three days.
^{b}Number represents geometric mean of surface staining with the MHC Class I antibody W6/32 or with the MHC Class II antibody L2443 of live gated cells.

Materials and Methods for Example 1

Cell Culture.

Melanoma cells were cultured in DMEM with 10% FBS. The T cell receptor (TCR)-negative Jurkat T cell line derivative J.RT3-T3.5 was obtained from ATCC and these T cells were cultured in RPMI with 10% FBS. IFN-β-1a (Avonex) was obtained from Biogen-Idec (Cambridge, Mass.) and reconstituted according to the manufacturer's recommendations.

Subcloning of the Melan-A/MART-1 Specific TCR.

A Melan-A/MART-1 specific TCR was produced and inserted into a lentiviral vector. In brief, a TCR specific for the MART-1 peptide EAAGIGILTV presented in HLA-A2 MHC Class I molecules (1D3) was synthesized by GeneArt (Burlingame, Calif.) and placed into a third generation lentiviral transfer vector. The full length insert (1820 bps) containing both chains of the TCR was then subcloned into the lentiviral vector using the restriction sites NheI and SalI. A silent mutation was made in the seventh amino acid (P139) of the alpha chain constant region of the TCR to generate an EagI restriction site. Two silent mutations were also made in the eighth (P141) and ninth amino acid (P142) of the beta chain constant region of the TCR to generate a BspeI restriction site. The construction of the vector with these additional restriction enzyme sites facilitates the subcloning of other TCRs by creating a way to readily synthesize and subclone 450 bp of the variable regions of each chain of the TCR.

Characteristics of the Melan-A/MART-1 Specific TCR.

The sequence of the alpha and beta chains of a TCR specific for the MART-1 peptide EAAGIGILTV presented in HLA-A2 MHC Class I molecules was codon-optimized to increase expression. The TCRα and TCRβ sequences were separated by a 2A sequence to facilitate stoichiometric coordinate expression under control of the EF-1α promoter. The alpha and beta chains each contained a mutation (T183C and S190C respectively), which added a cysteine to facilitate pairing and surface expression of the two chains of the TCR, by forming an additional inter-chain disulfide bond.

J-TCR-M1 Cell Line Derivation.

The TCR-minus cell line J.RT3-T3.5 was infected with lentiviral particles containing the Melan-A/MART-1 specific TCR as previously described. For transduction of the Melan-A/MART-1 specific TCR, 1×10⁵ J.RT3-T3.5 cells were incubated with lentiviral vector at a MOI of 10. The surface expression and peptide specificity of the transduced TCR was established using tetramer staining. The function of the transduced TCR was shown after co-culture with peptide pulsed tumor cells and cytokine ELISA. A pool of transduced J.RT3 cells stably expressing the exogenous Melan-A/MART-1 specific TCR at >95% efficiency was expanded for further use.

Chemicals.

A chemical library of 480 compounds was obtained from Enzo (Plymouth Meeting, Pa.) and is referred to as the “ICCB Known Bioactives Library.” The compounds within this library include a variety of receptor agonists, ion channel functional modulators, and kinase and enzyme inhibitors. Compounds for use in secondary screens were purchased and resuspended at 10 mg/ml following the manufactures recommendations. 17-AAG was purchased from Sigma Aldrich (St. Louis, Mo.). PMA, flunarizine, OBAA, dantrolene, damnacanthal, aphidicolin, and glyburide were obtained from Enzo Life Sciences (Plymouth Meeting, Pa.).

Cell-Based Screening Assay Procedure.

The 480 compounds were tested in six parallel 96 well plates. The first two columns (16 wells) of each plate were used for standards (8 wells), untreated controls (4 wells), and positive controls (4 wells). The other ten columns (80 wells) were used for testing compounds in separate wells. A100 μl aliquot containing 5×10⁴ MU89 cells was added to wells that contained 0.1 μl of each test drug diluted in 50 μl of RPMI media. The plates were incubated with drug for 72 hours at 37° C. in a humidified incubator with 5% CO₂. After 72 hours, each well received 2.5×10⁴J-TCR-M1 cells in 50 μl medium, and then incubated a further 24 hours. At the conclusion of the 24 hour co-culture period, each plate was centrifuged to pellet the cells and 100 μl supernatant fluid was collected from each well and plated in a parallel 96 well flat bottom well for IL-2 ELISA assay.

Confirmation Assays: Repeat Cellular Assay.

Melanoma cells were treated with hits from the primary screen for three days prior to counting and plating for the coculture assay (cells were washed and resuspended in fresh medium that no longer contained the treatment agents during the co-culture). A100 μl volume of 5×10⁵ cells/ml tumor cells were mixed with 50 μl of 2.5×10⁵ cells/ml T cells in a 96-well V-bottom plate and incubated overnight (16-24 hours). Plates were centrifuged to pellet the cells and 100 μl of supernatant fluid was removed from each well for cytokine assay. To assess antigen-specific T cell responses, a final concentration of 3.33 μg/ml Melan-A/MART-1 peptide (₂₆ELAGIGILTV₃₅ (A27L from wild type)) was added to the co-culture. MU89 tumor cells were also treated with IFN-β (5000 U/ml) for three days as a positive control.

Cytokine ELISA.

The protocol for evaluation of the cytokine IL-2 was performed using a the BD OptEAI kit, human IL-2 ELISA set from BD Biosciences (San Diego, Calif.) following the manufacturer's recommendations. The absorbance 450 nm was read in a BioRad 3550 plate reader. A standard curve using known concentrations of IL-2 (from 500 to 8 pg/ml) was included in each ELISA plate. IL-2 levels for experimental samples (pg/ml) were calculated from the standard curve.

EGFP Reporter Cell Line.

A1200 base pair human genomic DNA segment encompassing the Melan-A/MART-1 promoter was used to generate a construct driving expression of the EGFP reporter gene. Stable transfectants with this construct were generated in the low-antigen cell line A375 and the high-antigen line MM96L+. The EGFP expression response patterns of these cells to IFN-β and MAP kinase inhibitors recapitulates the antigen up-regulation of endogenous Melan-A/MART-1 induced by these agents.

Flow Cytometry.

Cells were fixed with 1% formaldehyde, permeabilized with 0.1% saponin, stained with monoclonal antibodies to melanocyte antigens. Antibodies were obtained with specificity for: MART-1 from Vector Labs (Burlingame, Calif.); gp100 HMB45 from DakoCytomation (Carpinteria, Calif.) and then visualized with goat anti-mouse FITC-conjugated secondary antibody from Invitrogen (Fredrick, Md.). The level of EGFP was determined by flow cytometry of unfixed cells that were washed and resuspended in 1×PBS.

### Example 2

### Assays with Additional Hsp90 Inhibitors and Tumor Cells

To further evaluate enhancement of immune recognition of cancer cells by inhibition of Hsp90 function, it was shown that a total of twelve different Hsp90 inhibitors were active in several molecular and cellular assays on a series of cell lines, including eleven human melanomas, the murine B16 melanoma, and two human gliomas. A group of Hsp90 inhibitors, including 17-AAG derivatives and structurally distinct compounds including PU-H71 and CCT018159, are active on a variety of melanomas with different levels of antigen expression, (MALME, MU-89, A375, MU-X), two gliomas (U-87MG and U-118MG), and a murine melanoma (B16). The melanomas all express varying levels of differentiation antigens Melan-A/MART-1, gp100 and TRP-2, as well as the MHC-Class I antigen (as evidenced by W6/32 antibody staining) that is required for T cell recognition of the tumor cells. Each of these antigens is enhanced by all of the Hsp90 inhibitors. The murine melanoma, B16, can be stained with the gp100 and TRP-2 antibodies (both are raised against human proteins, but cross-react with the murine counterpart), but the Melan-A/MART-1 antibody does not react with this mouse-derived melanoma. Similar to the human counterparts, the B16 mouse tumor also can be induced to express enhanced antigen levels upon 3-day exposure to each of the Hsp90 inhibitors (Table 16). The mouse H-2 Class I antigen is likewise induced by the Hsp90 inhibitors.

Both melanomas and gliomas are of neural crest origin, and gliomas are known to express gp100, but not most of the other melanocyte differentiation antigens. Thus, the gliomas were tested for induction of gp100 and MHC class I expression and noted that gliomas can be enhanced by Hsp90 inhibitor treatment (Table 16).

Several cell lines expressing varying native-sequences, (wild-type (WT) or mutant (M) alleles of these genes were evaluated), to determine if there is a relationship between levels of Hsp90 inhibitor-mediated antigen up-regulation and BRAF and NRAS mutational status. Mutant status of the NRAS and BRAF loci were determined previously. Among the cells tested, the highest responses were observed for cells heterozygous for BRAF mutation and wild-type for native sequence NRAS (Table 17; activating mutations in these genes appear to be mutually exclusive. Of note, the Hsp90 inhibitors affected both types of cell lines (those with mutant NRAS and wild-type native sequence BRAF, as well as cells expressing wild-type native NRAS and mutant BRAF), indicating that the effect of Hsp90 inhibition is not limited to the mutant status of either BRAF or NRAS genes.

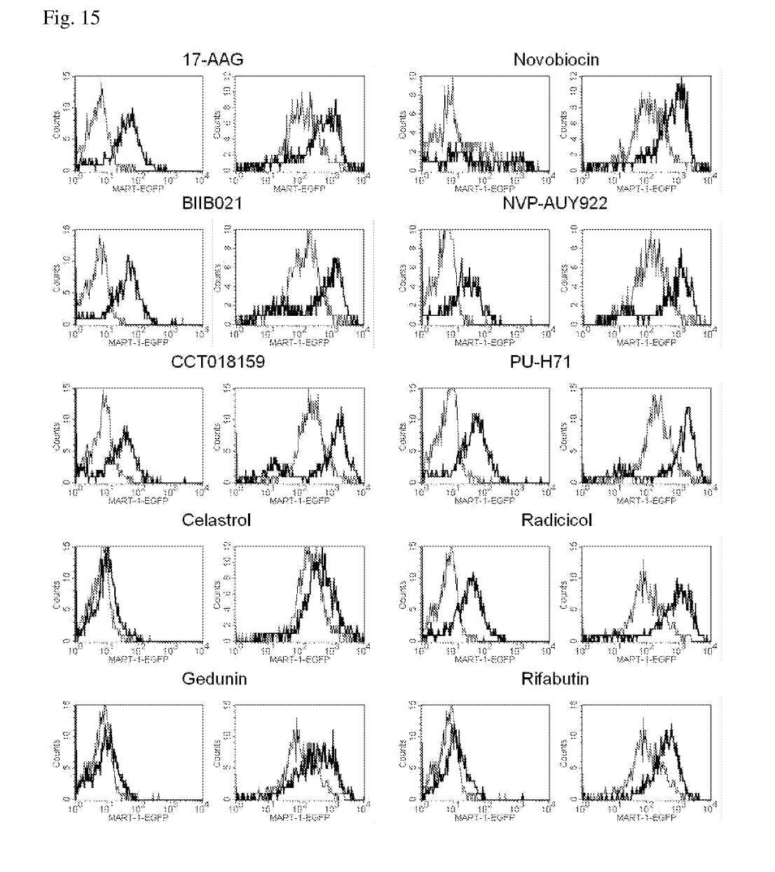

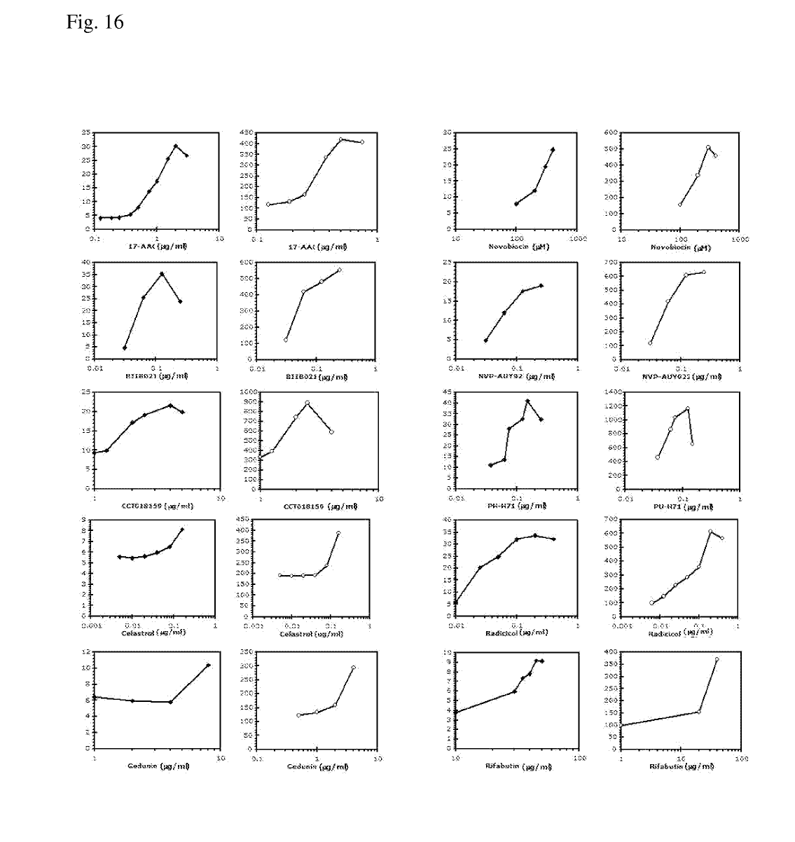

Using an EGFP-linked promoter assay, the activity of this class of drugs was tested with a larger panel of 12 different Hsp90 inhibitors, initially using wide dose ranges for each drug. At optimal doses, all of the Hsp90 inhibitors increased the level of EGFP, in a cell line with low levels of Melan-A/MART-1 (A375) and also in a cell line with higher levels of endogenous Melan-A/MART-1 (MM96L+) (Table 18). Examples of the flow histograms used to generate the data for dose response curves and for the data in Table 18 are provided in FIG. 15. Such optima were determined using dose response curves to identify the highest dose of inhibitor giving the maximum fold increase in EGFP (FIG. 16). These results are consistent with the hypothesis that the effect of Hsp90 inhibition on antigen levels operates through transcriptional up-regulation.

Similar augmentation of EGFP fluorescence was seen in response to the extended panel of Hsp90 inhibitors as observed for 17-AAG. The tested inhibitors included 7 compounds that bind to the amino-terminal ATP-binding region of Hsp90, while three of the inhibitors, (gedunin, celastrol, and novobiocin), do not bind to this ATP-binding site and manifest their activity via distinct mechanisms. Similar relative levels of EGFP induction were observed after treatment with either class of Hsp90 inhibitor (Table 18).

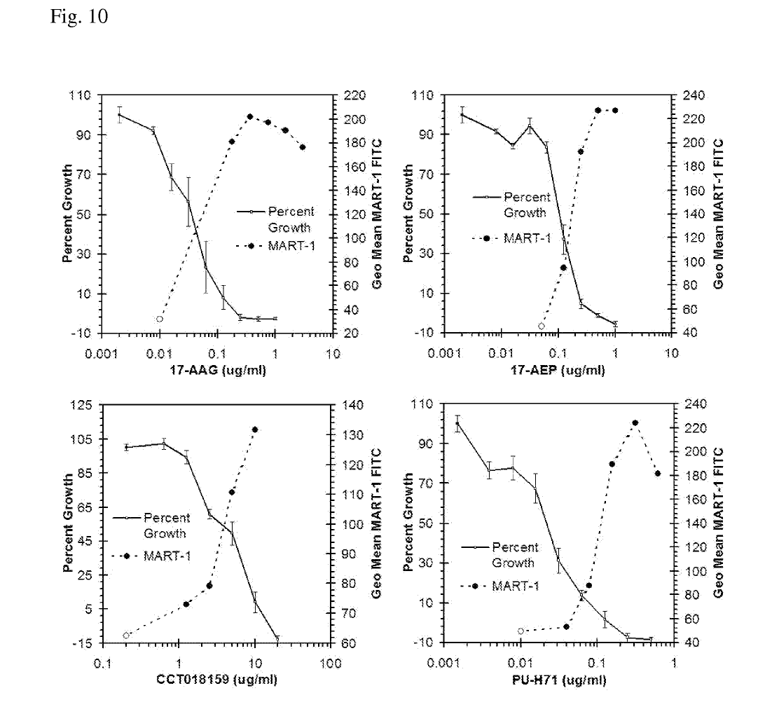

The Hsp90 inhibitors 17-AAG, 17-AEP, CCT018159, and PU-H71 were tested for effects on cell growth and the kinetics of melanocytic antigen upregulation. The toxicity of Hsp90 inhibitors to melanoma cells has been reported previously. The WST assay was used to assess the effect of Hsp90 inhibitor treatment on melanoma cell growth over a range of doses. A linear increase in growth inhibition was observed (FIG. 10). The Hsp90 inhibitors 17-AEP, CCT018159, and PU-H71 showed similar growth inhibitory effects as 17-AAG. Of note, as shown in FIG. 10, the increased levels of Melan-A/MART-1 antigen induction correlate with the decrease in cell growth. In fact, the doses required for total growth inhibition and maximal Melan-A/MART-1 induction correspond. Thus, the optimal dose for antigen expression occurs at doses of Hsp90 inhibitor that significantly inhibit growth of the cells.

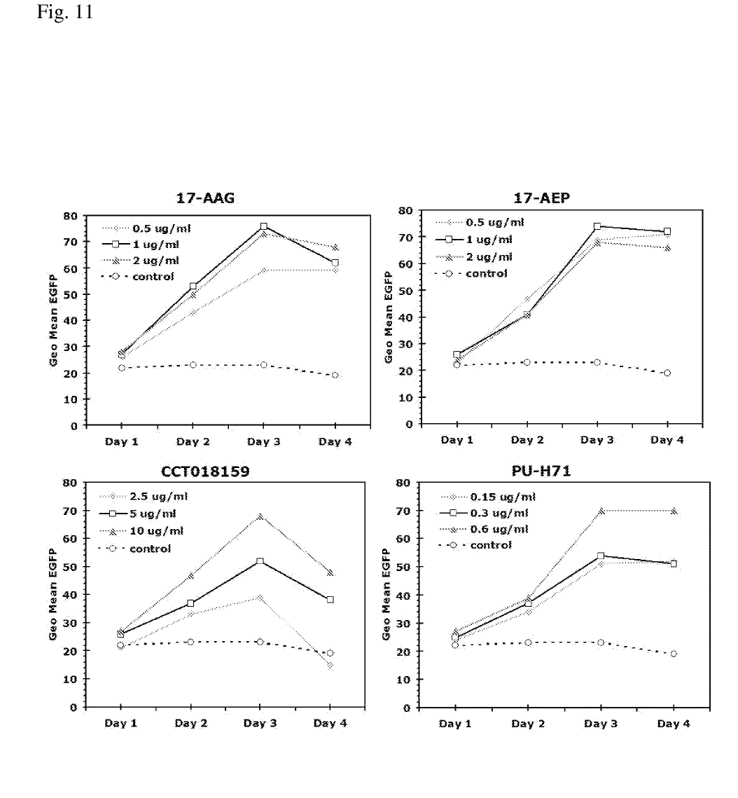

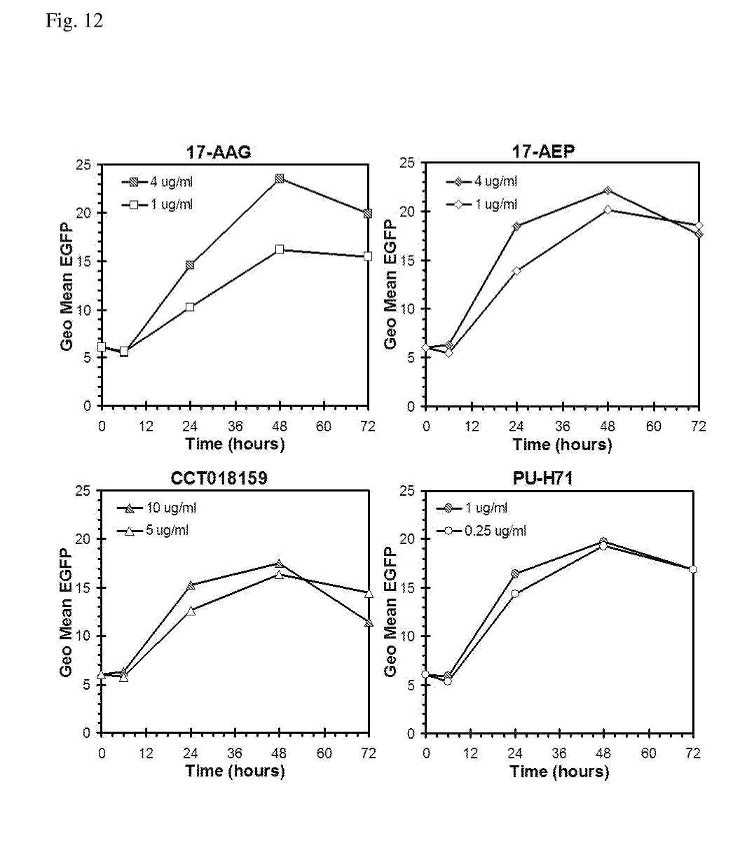

A kinetic analysis was performed to determine the effect of Hsp90 inhibition on Melan-A/MART-1 promoter activity over time. Using the Melan-A/MART-1 promoter EGFP system in the melanoma cell line MU89, cells treated with 4 separate Hsp90 inhibitors (17-AAG, 17-AEP, CCT018159, and PU-H71) significantly enhanced the fluorescent reporter signal as early as 2 days (FIG. 11). Reporter activity increases steadily for the first 72 hours of Hsp90 inhibition, and then plateaus. The need for continued presence of Hsp90 inhibitors in order for them to be effective (i.e. whether the drug can be removed after short exposure, or whether it must it be continually present) was assessed. Transient exposure of EGFP-expressing A375 cells to the IIsp90 inhibitors 17-AAG, 17-AEP, CCT018159, and PU-H71 requires a minimum of 24 hours of exposure in order to induce significant increases in Melan-A/MART-1 promoter EGFP reporter levels (FIG. 12). To achieve full signal enhancement, it is desirable to retain the drug for at least 48 hours, after which the effect remains constant or slightly decreases.

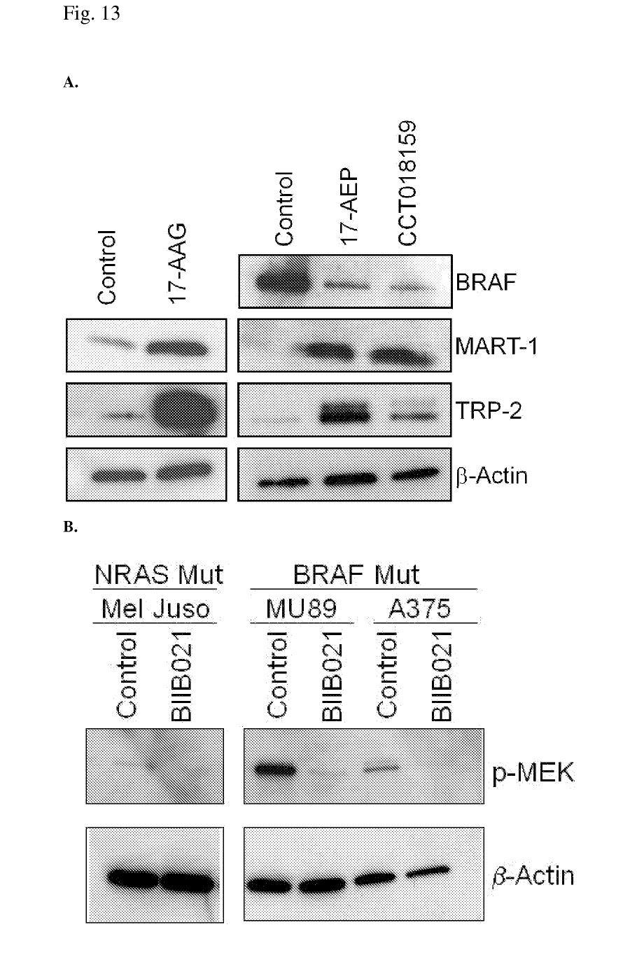

To address the effect of Hsp90 inhibition on the MAP kinase pathway, protein levels and signaling activity (protein phosphorylation levels) of the proteins in this pathway were analyzed directly. As client proteins of Hsp90, BRAF and NRAS will be destabilized and reduced in effective cellular concentrations when Hsp90 is inhibited. Western blots of the BRAF protein established that this protein was degraded after Hsp90 inhibitor treatment (FIG. 13A). In contrast to the decreased level of BRAF in Hsp90 inhibitor-treated cells, the level of melanocyte antigens increased (FIG. 13A). Both Melan-A/MART-1 and TRP-2 increased significantly after Hsp90 inhibitor treatment compared to untreated control samples. Western blotting for phosphorylated MEK showed that signaling was blocked by Hsp90 inhibitor treatment of both cells that are mutant for NRAS (and also wild-type BRAF), as well as wild-type NRAS cells that are also mutant for BRAF (FIG. 13B).

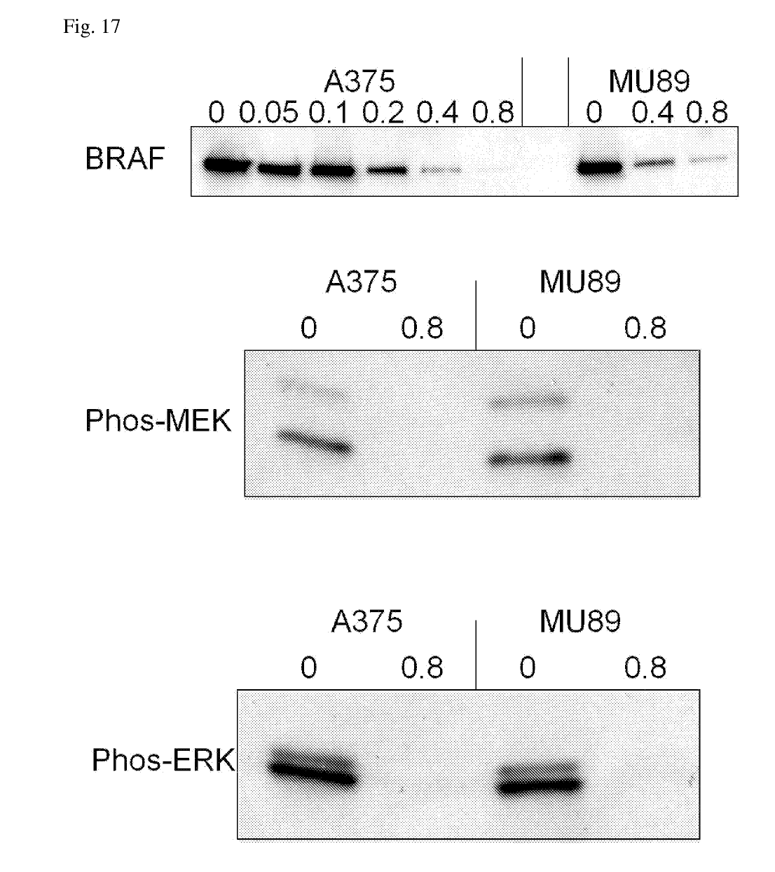

Furthermore, FIG. 17 demonstrates that for a dose of Hsp90 inhibitor that is effective at decreasing BRAF expression, there is a parallel decrease in the downstream pMEK and pERK that would normally be induced by activated BRAF, but are blocked by the Hsp90 inhibitor.

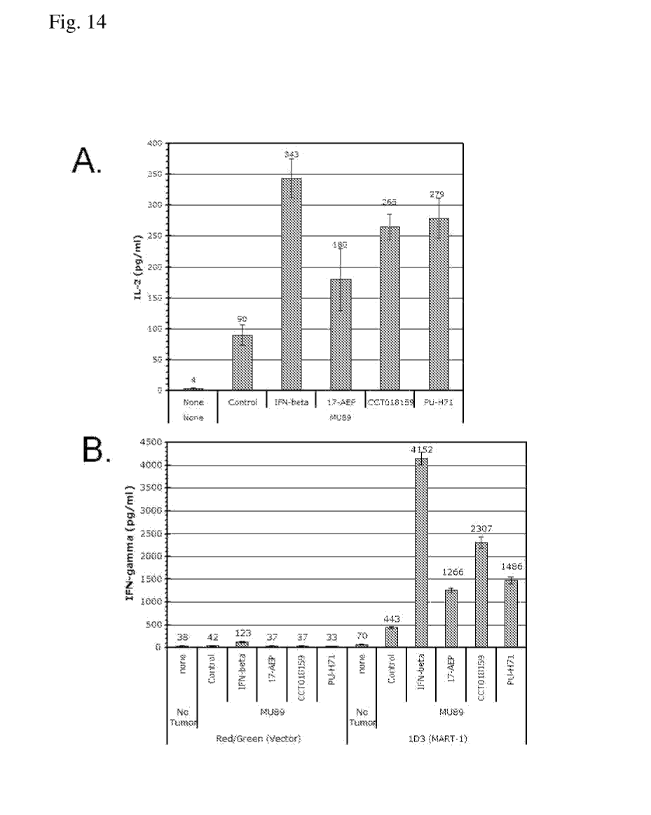

Using a cell-based assay to evaluate tumor recognition by T cells, 17-AAG was identified as a hit. To extend these results, an additional three Hsp90 inhibitors (17-AEP, CCT and PU-H71) were tested in the same cell-based assay. The results presented in FIGS. 14 A and B illustrate IL-2 levels of control untreated tumor cells versus cells treated with the Hsp90 inhibitors. Increased IL-2 secretion by Jurkat T cells is a manifestation of recognition of the tumor cells by the Melan-A/MART-1 specific TCR expressing T cells. As shown in FIG. 14A, co-culture of these TCR-transduced JURKAT cells with Hsp90 inhibitor-treated tumor cells results in increased IL-2 production as compared to control tumor cells, demonstrating that a 3 day treatment with Hsp90 inhibitors increases T cell recognition of the tumor.

The same anti-Melan-A/MART-1 specific TCR used in the JURKAT cells was also transduced into normal CD8+ Peripheral Blood Leukocyte (PBL)-derived T cells. As shown in FIG. 14B, increased IFN-gamma levels are produced after treatment with 17-AEP, CCT or PU-H71. Increases between 3 and 5-fold over untreated control are routinely seen when the transduced PBL are co-cultured with tumor cells that have been treated with the Hsp90 inhibitors.

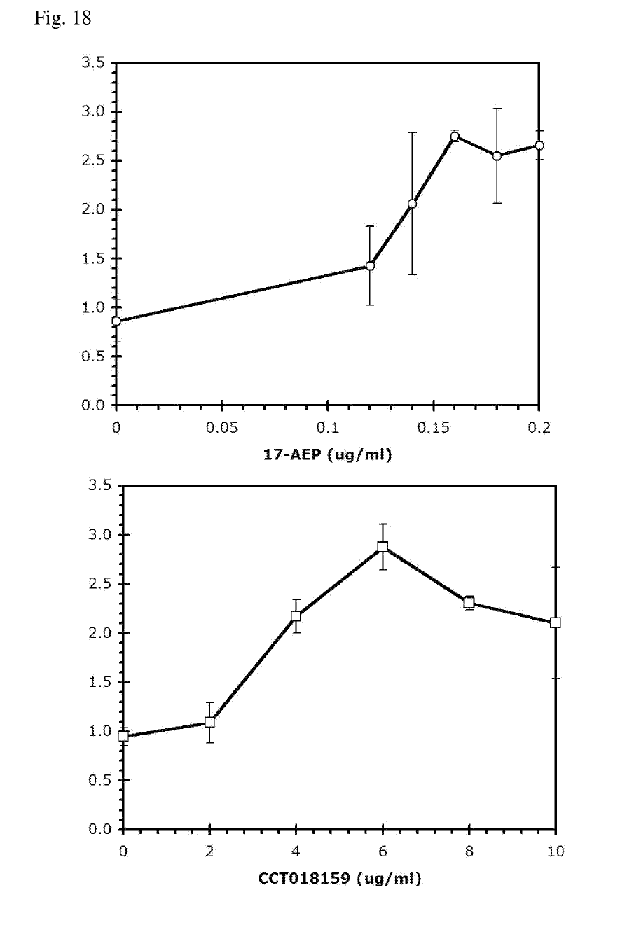

As a further indication that the increased protein levels detected with the antibodies, and the increased T cell recognition, are truly a reflection of turning on of the appropriate genes, we used a luciferase reporter assay to demonstrate that the MART-1 promoter is turned on by Hsp90 treatment of the cells (FIG. 18).

Together, these data demonstrate that Hsp90 client proteins, such as BRAF, are diminished in the treated tumor cells, but the antigens recognized by the T cells are not Hsp90 client proteins, and in contrast to the decrease in BRAF, there is enhanced gene promoter activity, increased protein synthesis and increased antibody staining that all demonstrate that the antigen induction is a true reflection of the biological response in the presence of Hsp90 inhibitors.

TABLE 16
Effect of Hsp90 Inhibitors on differentiation antigens and MHC Class I.

| Cell Line | Treatment^{a} | μg/ml^{b} | MART-1^{c} | gp100^{c} | TRP-2^{c} | Class I^{d} |
|---|---|---|---|---|---|---|
| MALME- | Control | | 117 | 227 | 106 | 44 |
| 3M | IFN-beta | | 130 (1.1) | 534 (2.4) | 179 (1.7) | 194 (4.4) |
| (+) | 17-AEP | 1.0 | 205 (1.8) | 839 (3.7) | 256 (2.4) | 105 (2.4) |
| | PU-H71 | 0.3 | 226 (1.9) | 511 (2.3) | 279 (2.6) | 137 (3.1) |
| | CCT018159 | 10.0 | 291 (2.5) | 823 (3.6) | 222 (2.1) | 72 (1.6) |
| MU89 | Control | | 33 | 123 | 92 | 18 |
| (+) | IFN-beta | | 59 (1.8) | 140 (1.1) | 150 (1.6) | 51 (2.8) |
| | 17-AEP | 1.0 | 130 (3.9) | 342 (2.8) | 144 (1.6) | 54 (3.0) |
| | PU-H71 | 0.3 | 147 (4.5) | 381 (3.1) | 214 (2.3) | 66 (3.7) |
| | CCT018159 | 10.0 | 99 (3.0) | 273 (2.2) | 252 (2.7) | 75 (4.2) |
| A375 | Control | | 6 | 15 | 16 | 36 |
| (−) | IFN-beta | | 8 (1.3) | 17 (1.1) | 24 (1.5) | 84 (2.3) |
| | 17-AEP | 0.5 | 11 (1.8) | 22 (1.5) | 31 (1.9) | 67 (1.9) |
| | PU-H71 | 0.15 | 11 (1.8) | 24 (1.6) | 26 (1.6) | 71 (2.0) |
| | CCT018159 | 2.5 | 13 (2.2) | 32 (2.1) | 37 (2.3) | 70 (1.9) |
| MUX | Control | | 13 | 26 | 41 | 104 |
| (−) | IFN-beta | | 18 (1.4) | 46 (1.8) | 55 (1.3) | 158 (1.5) |
| | 17-AEP | 1.0 | 19 (1.5) | 34 (1.3) | 48 (1.2) | 219 (2.1) |
| | PU-H71 | 0.3 | 19 (1.5) | 32 (1.2) | 41 (1.0) | 212 (2.0) |
| | CCT018159 | 10.0 | 22 (1.7) | 40 (1.5) | 40 (1.0) | 174 (1.7) |
| U-118 MG | Control | | n.a. | 45 | n.d. | 50 |
| (glioma) | IFN-beta | | n.a. | 49 (1.1) | n.d. | 132 (2.6) |
| | 17-AEP | 1.0 | n.a. | 61 (1.4) | n.d. | 117 (2.3) |
| | PU-H71 | 0.3 | n.a. | 63 (1.9) | n.d. | 132 (2.6) |
| | CCT018159 | 10.0 | n.a. | 83 (1.4) | n.d. | 101 (2.0) |
| U-87 MG | Control | | n.a. | 26 | n.d. | 27 |
| (glioma) | IFN-beta | | n.a. | 36 (1.4) | n.d. | 73 (2.7) |
| | 17-AEP | 0.5 | n.a. | 51 (2.0) | n.d. | 66 (2.4) |
| | PU-H71 | 0.3 | n.a. | 66 (2.7) | n.d. | 53 (2.0) |
| | CCT018159 | 5.0 | n.a. | 71 (2.5) | n.d. | 28 (1.0) |
| B16 | Control | | n.a. | 80 | 94 | 2 |
| (murine | 17-AEP | 0.5 | n.a. | 420 (5.3) | 288 (3.1) | 13 (5.4) |
| melanoma) | PU-H71 | 0.15 | n.a. | 595 (7.4) | 360 (3.8) | 14 (6.0) |
| | CCT018159 | 2.5 | n.a. | 414 (5.2) | 364 (3.9) | 16 (6.8) |

^{a}Cells were untreated (control) or treated with 5000 Units/ml of IFN-beta, or with the Hsp90 inhibitors as indicated for 3 days.
^{b}Dose indicated is optimal dose for antigen increase
^{c}Number represents geometric mean of intracellular staining with an antibody to Melan-A/MART-1, gp100 or TRP-2 of live gated cells. Number is parenthesis is fold increase relative to untreated control.
^{d}Number represents geometric mean of surface staining with the MHC Class I antibody W6/32 (or H2kb for B16) of live gated cells.
Antigen status of human melanoma cell lines: (+) = Melan-A/MART-land gp100 high (−) = Melan-A/MART-land gP100 low.
n.a. = not applicable, glioma do not express Melan-A/MART-1, and human Melan-A/MART-lantibody did not cross react with murine Melan-A/MART-1
n.d. = not determined these cells were not stained with the TRP-2 antibody.

TABLE 17
Effect of Hsp90 inhibitors on melanocyte differentiation antigens.

| BRAF^{a} | NRAS | Cell Line | Treatment^{b} | μg/ml^{c} | MART-1^{d} | HMB45^{d} |
|---|---|---|---|---|---|---|
| WT | M/M | Mel-Juso | 17-AEP | 0.50 | 1.75 | 1.81 |
| | | (−/+) | CCT018159 | 5.00 | 1.17 | 1.12 |
| | | | PU-H71 | 0.15 | 1.41 | 1.99 |
| | | Roth | 17-AEP | 0.50 | nd | 1.89 |
| | | (−/+) | CCT018159 | 2.50 | nd | 1.95 |
| | | | PU-H71 | 0.25 | nd | 2.97 |
| | | H59-44T | 17-AEP | 0.50 | 2.09 | 1.91 |
| | | (+) | CCT018159 | 5.00 | 3.90 | 3.77 |
| | | | PU-H71 | 0.15 | 3.30 | 3.59 |
| M/WT | WT | MU89 | 17-AEP | 0.50 | 3.61 | 3.26 |
| | | (+) | CCT018159 | 10.00 | 3.22 | 3.92 |
| | | | PU-H71 | 0.30 | 3.42 | 3.57 |
| | | MALME-3M | 17-AEP | 0.25 | 2.34 | 2.25 |
| | | (+) | CCT018159 | 5.00 | 2.48 | 3.63 |
| | | | PU-H71 | 0.30 | 1.83 | 2.25 |
| | | 453A | 17-AEP | 1.00 | 2.29 | 3.78 |
| | | (+) | CCT018159 | 10.00 | 3.29 | 3.38 |
| | | | PU-H71 | 0.30 | 3.22 | 3.09 |
| | | MM96L+ | 17-AEP | 1.00 | 1.79 | 0.92 |
| | | (+) | CCT018159 | 5.00 | 3.04 | 1.81 |
| | | | PU-H71 | 0.30 | 2.59 | 1.78 |
| | | MM455 | 17-AEP | 0.50 | nd | 1.39 |
| | | (−/+) | CCT018159 | 2.50 | nd | 1.16 |
| | | | PU-H71 | 0.25 | nd | 1.50 |
| M/M | WT | MUX | 17-AEP | 0.50 | 1.55 | 1.75 |
| | | (−) | CCT018159 | 5.00 | 1.64 | 1.89 |
| | | | PU-H71 | 0.30 | 1.54 | 1.59 |
| | | MM96L− | 17-AEP | | nd | nd |
| | | (−) | CCT018159 | 5.00 | 1.28 | nd |
| | | | PU-H71 | 0.15 | 1.82 | Nd |
| | | A375 | 17-AEP | 0.50 | 1.46 | 1.47 |
| | | (−) | CCT018159 | 2.50 | 2.22 | 2.22 |
| | | | PU-H71 | 0.15 | 1.80 | 1.86 |

^{a}WT = homozygous WT, M/WT = heterozygous WT, M/M = homozygous mutant.
^{b}Cells were treated with the indicated concentration of Hsp90 inhibitor for 3 days before intracellular staining.
^{c}Dose indicated is optimal dose for antigen increase
^{d}Level of induction of indicated melanocyte proteins relative to untreated control cells.
nd = not determined.
(+) = antigen-positive;
(+/−) = gp100 positive/Melan-A/MART-1 negative;
(−) = antigen-negative

TABLE 18
Effect of Hsp90 inhibitors on Melan-A/MART-1
promoter driven EGFP reporter.

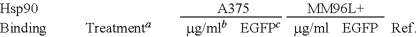

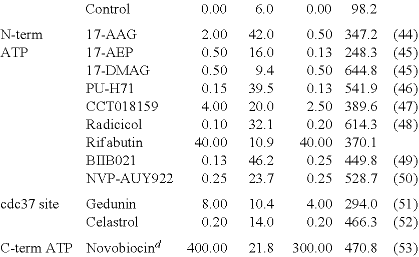

^{a}Cell were treated for 3 days.
^{b}Dose indicated is optimal dose for antigen increase
^{c}Geometric mean of EGFP flow histogram.
^{d}Concentration of Novobiocin in μM
gray shading indicates similar chemical structure.

Materials and Methods for Example 2

Cell Culture, and Hsp90 Inhibitors.

General culture conditions for cell propagation and the origins of most of the melanoma cell lines have been previously described. Melanoma cells were cultured in DMEM with 10% FBS. The glioma cell lines U87 MG and U118 MG were obtained from the American Type Culture Collection (ATCC). The B16 murine melanoma cell line was provided by Dr. Andrew Hurwitz and has been previously described. The T cell receptor (TCR)-negative Jurkat T cell line derivative J.RT3-T3.5 was obtained from ATCC. The construction of the J.RT3-T3.5 cell line expressing a Melan-A/MART-1 specific TCR has been described previously. T cells were cultured in RPMI with 10% FBS. IFN-beta-1a (Avonex) was obtained from Biogen-Idec (Cambridge, Mass.) and reconstituted according to the manufacturer's recommendations. The Hsp90 inhibitor radicicol was purchased from A.G. Scientific (San Diego, Calif.). Novobiocin was ordered from BioMol (Plymouth Meeting, Pa.). 17-DMAG was obtained from LC laboratories (Woburn, Mass.). 17-AEP-AP was purchased from InVivoGen (San Diego, Calif.). The Hsp90 inhibitors rifabutin, PU-H71, and 17-AAG were purchased from Sigma (St. Louis, Mass.). Gedunin, CCT018159, and celastrol were purchased from Tocris (Ellisville, Mo.). Selleck Chemicals (Houston, Tex.) provided NVP-AUY922 and BIIB021.

EGFP Reporter Cell Line.

The generation and application of EGFP reporter cells has been previously described. Briefly, a 1200 base pair human genomic DNA segment encompassing the Melan-A/MART-1 promoter was used to generate a construct driving expression of the EGFP reporter gene. Stable transfectants with this construct were generated in the low-antigen cell line A375 and the high-antigen line MM96L+ and MU89. The EGFP expression response patterns of these cells to IFN-beta and MAP kinase inhibitors recapitulates the antigen up-regulation of endogenous Melan-A/MART-1 induced by these agents.

Measurement of Cell Growth.

Cell growth was measured using the WST reagent system from Roche (Indianapolis, Ind.). 2000 cells were plated in a 96-well culture plate. The time zero absorbance (To) of the cells was measured at 450 nm 24 hours after plating, and inhibitors were added at this time. Cells were allowed to grow for an additional 72 hrs. WST was added to each well and after 1 hr the hydrolysis of the WST was read (Tx) in a plate reader at 450 nm. Untreated control cells were measured (Con) to establish 100 percent growth. Wells were normalized by subtracting a 655 nm background reading. Triplicates for each sample were determined. A control for medium only was subtracted from each reading. % Growth=100×(To−Tx)/(Con−Tx).

Treatment of Cells with Hsp90 Inhibitors.

Cells were plated at a density of 1×10⁵ in 1 ml of medium in a 24-well plate and cultured for varying times as indicated in results. Typically, 3 days of culture in the presence of Hsp90 inhibitors was optimal for the functional studies performed. After incubation, cells were collected by trypsinization and then evaluated by flow cytometry.

Flow Cytometry.

Intracellular staining and flow cytometric analyses of cytoplasmic Melan-A/MART-1, and gp100 expression were performed as described previously. Cells were fixed with 1% formaldehyde, permeabilized with 0.1% saponin, stained with monoclonal antibodies to melanocyte antigens. Antibodies were obtained with specificity for: Melan-A/MART-1 from Vector Labs (Burlingame, Calif.); gp100/HMB45 from DakoCytomation (Carpinteria, Calif.) and then visualized with goat anti-mouse FITC-conjugated secondary antibody from Invitrogen (Frederick, Md.). The level of EGFP was determined by flow cytometry of unfixed cells washed and resuspended in 1×PBS. Surface staining of MHC Class I was carried out using the antibody W6/32 on cells harvested with Cell Stripper from MediaTech Inc. (Manassas, Va.) and carried out on ice.

Western Blot Analysis.

Cell lysates were prepared using RIPA buffer from Santa Cruz (Santa Cruz, Calif.) containing protease and phosphatase inhibitors. Protein concentrations were determined using Bradford assay from BioRad (Hercules, Calif.). Equal amounts of protein were loaded in each well for PAGE analysis. Proteins were transferred to PVDF membranes from Pierce Biotechnology (Rockford, Ill.). Blocking and both primary and secondary antibody incubations were performed using Starting Block from Pierce (Rockford, Ill.). Blots were washed with 1×TBS with 0.5% tween 20. Primary antibodies used BRAF H-145 and TRP-2 from Santa Cruz (Santa Cruz, Calif.), and Phos ERK from Cell Signaling Technologies (Danvers, Mass.) beta actin from Sigma (St. Louis, Mo.). Goat anti-rabbit HRP conjugated secondary antibody was purchased from Pierce (Rockford, Ill.). Chemiluminesce was performed using the Femto kit form Pierce (Rockford, Ill.). Membranes were used to expose films, which were then developed for visualization of antibody detection of protein bands. Sizes of proteins were determined by comparison to the Broad prestained protein standard ladder from BioRad (Hercules, Calif.).

Assays of CD8+ T Lymphocytes.

Primary CD8+ T lymphocytes were obtained using heparin-treated blood incubated with a negative selection cocktail RosettaSep from STEMCELL Technologies (Vancouver, BC, Canada) to generate 95% pure CD8 T cells. These cells were stimulated for 24 hours with CD3/CD28 beads (InVitrogen Dynal AS, Oslo, Norway), and cultured with 200 IU/ml recombinant IL-2 (Proleukin) from Cetus (Emeryville, Calif.) supplemented media. For transduction of the Melan-A/MART-1 specific TCR, 1×10⁵ stimulated primary CD8 cells were incubated with lentiviral vector at a MOI of 10. Cells were grown for two days after lentiviral infection and then stained with tetramer as described previously. The CD8+ T cells were further propagated for 2 weeks in medium containing 200 IU/ml recombinant IL-2 prior to use in cellular assays described below.

Assay for T Cell Activation by Tumor Cells.

Melanoma cells were treated with antigen-modulating agents, (Hsp90 inhibitors or IFN-β), for three days prior to counting and plating for the co-culture assay (cells were washed and resuspended in fresh medium that no longer contained the treatment agents during the co-culture). 100 μl of 5×10⁵ tumor cells/ml were mixed with 50 μl of T cells (at 5×10⁵ cells/ml) in a 96-well V-bottom plate and incubated overnight (16-24 hours). Plates were centrifuged to pellet the cells and 120 μl of supernatant fluid was removed from each well for cytokine assay. To assess antigen-specific T cell responses, 3.33 μg/ml Melan-A/MART-1 peptide was added to the co-culture. The sequence of the Melan-A/MART-1 peptide was ₂₆ELAGIGILTV₃₅ (A27L from wild type) and the NY-ESO-1 peptide ₁₅₇SLLMWITQV₁₆₅ (C165V from wild type).

Cytokine ELISA.

The protocols for evaluation of either IL-2 or IFN-gamma were similar, following the manufacturer's recommendations. A standard curve using known concentrations of each cytokine was included in each ELISA experiment. ELISA plates were coated with capture antibody and then washed with 1×PBS 0.5% Tween-20. Wells were blocked with 1×PBS 1% FBS for 1 hour and rewashed. Supernatants and standards were added to the wells and incubated for 2 hr at room temperature and wells were washed again. The detection antibody and HRP secondary were added together and incubated for 1 hr. The HRP color reaction proceeded for 15 min before being stopped with 2N H₂SO₄. The absorbance 450 nm was read in a BioRad 3550 plate reader. IL-2 levels for experimental samples (pg/ml) were calculated from the standard curve.

### Example 3

### MHC Class I and II Enhancement by IFN Treatment

MHC Class I enhancement by iHSP is not limited to melanomas but can be demonstrated on several tumor cell lines of different origin including a glioma, an osteosarcoma, a B cell lymphoma and a cervical carcinoma. In addition, HLA Class II expression can also be induced or enhanced by Hsp90 inhibition on otherwise negative tumors cells. Pre-treatments with IFN-beta for 3-7 days further enhanced MHC Class I up-regulation by Hsp90 inhibitors, beyond the levels achieved with either IFN-beta or Hsp90 inhibitors alone. These increases were also seen on a variety of tumor cell lines. In contrast, the ability of Hsp90 inhibitors to enhance IFN-gamma induction of MHC Class II antigen expression was dependent on the timing of treatment with these agents. If iHsp90s and IFN-gamma are added at the same time, the induction of Class II antigen is ablated. However, if IFN-gamma is first allowed to induce Class II expression, then the subsequent addition of iHsp90s can synergistically increase the levels of Class II MHC expression achieved.

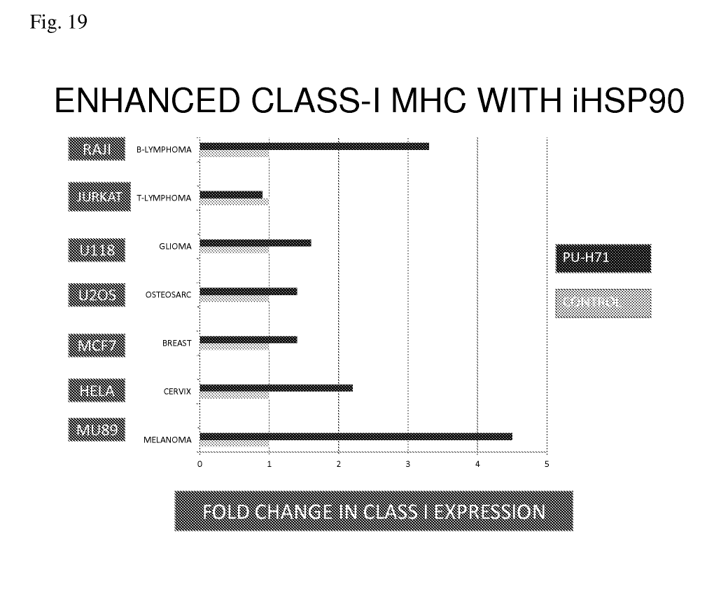

Hsp90 inhibition causes an increase in MHC Class I expression in tumor cell lines. The Hsp90 inhibitor, PU-H71 enhances Class I MHC on a variety of tumor types (FIG. 19). The greatest levels of MHC induction are seen on the melanoma (MU89), cervical carcinoma (HeLa) and B cell lymphoma (RAJI), while lower levels of induction are seen on the Breast carcinoma (MCF7), osteosarcoma (U2OS) and glioma (U118). The T cell lymphoma (Jurkat) did not show any increase in its very low level of MHC Class I antigen expression in response to PU-H71.

As shown in Table 19, both Inteferon-beta and Inteferon-gamma induce significant increases in Class I MHC expression in all the tumors except the JURKAT cells that only responded to IFN-gamma with respect to Class I enhancement. In contrast to MHC Class I expression, there was no induction of Class II MHC expression on any of the tumors by PU-H71. Among the tumors tested, only the RAJI B cell lymphoma expresses Class II antigens without any treatment, but even this tumor did not show any Class II induction by PU-H71. Interferon-beta also failed to induce Class II expression on any of the tumors, while IFN-gamma induced significant Class II on all of the tumors with the exception of the JURKAT cells, that remained Class II negative with all treatments. These data are also demonstrated in the bar graph in FIG. 19, in which the control level of MHC antigen is normalized for each tumor type to make side-by-side comparison of the MHC antigen induction for each of the cells. Again, the lack of Class II induction by HSp90 inhibitors is evident.

Combination of Hsp90 inhibition and IFN-beta treatment in melanoma cell lines were considered. We wanted to determine if the MHC induction we observed would impact the levels of MHC expression induced with the drugs individually or in combination.

Table 20 demonstrates that the effects on Class I induction is very different than what is observed for Class II induction. For example, in the MU-89 tumor cells, the Hsp90 inhibitor PU-H71 and IFN-beta not only stimulate significant Class I expression by themselves, but in combination, there is an additive effect of the two drugs when they are used together. In contrast, the IFN-gamma induction of Class I is not further enhanced by PU-H71, and while IFN-gamma is the only stimulant of Class II induction on the MU-89 cells, the combination of TFN-gamma with PIT-H71 results in ablation of the IFN-gamma induction of Class II antigen.

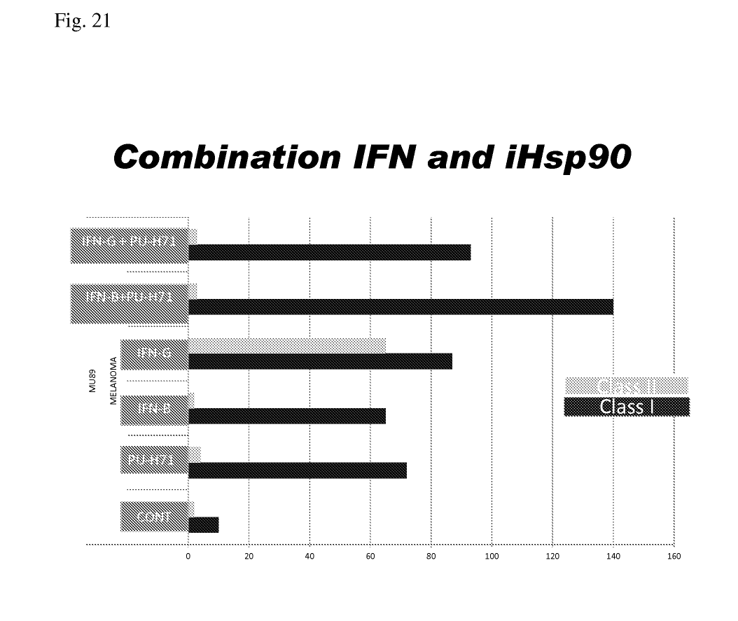

These data are also presented in FIG. 21, in which it is evident that only IFN-gamma is effective at Class II induction, but this induction is ablated by the addition of Hsp90 inhibitor. In contrast, both of the interferons and Hsp90 inhibitor induce increased Class I expression, and there is an additive effect on the melanoma tumor cells when IFN-beta and PU-H71 are added at the same time to tumor cells that will be cultured a further three days prior to staining assay.

The results on the HeLa cells do not show the synergy between PU-H-71 and IFN-beta on Class I, but both drugs are highly stimulatory on their own, and the level of Class I achieved with the combination of interferon and PU-H71 is comparable to that achieved with the drugs individually. Again, the ability of IFN-gamma to induce Class I expression on HeLa cells is ablated by the Hsp90 inhibitor.

The pattern of responses seen for HeLa cells is also seen in the U118 gliomas, although it is noteworthy here that the combination of interferon-gamma and Hsp90 inhibitor is better than either drug alone with respect to Class I induction, while the Class II induction is again prevented by co-treatment with both IFN-gamma and PU-H71. Likewise, the osteosarcoma U2-OS cells are strongly induced to Class I induction by each of the drugs, and the highest levels are achieved in combination with both interferons and PU-H71. However, as with the other tumor cells, the induction of Class II antigen seen with IFN-gamma is prevented by PU-H71.

The induction of Class I antigen by PU-H71 in the MCF-7 breast tumor line is less than that seen in the other tumors in this combination treatment, and the strong stimulation induced with either IFN-beta or IFN-gamma is diminished by the addition of PU-H71. Again, the induction of Class II on the MCF-7 is inhibited in the combination of PU-H71 and IFN-gamma.

While the combination of interferon-gamma and iHsp90 is inhibitory with respect of Class II expression (FIG. 21), the interplay of the agents is more difficult to interpret with respect to Class I as each of the agents is stimulatory on its own, and the combination may actually improve Class I expression, in contrast to what is observed with Class II expression that is clearly dependent on IFN-gamma induction in order to achieve significant levels of Class II on these tumor cells that otherwise do not express Class II antigens.

The discrepant induction of Class I and II antigens by the combination of iHsp90 and interferons led us to test the hypothesis that the iHsp90 induction we observed was dependent on pre-existing expression of MHC antigen, as is the case for Class I antigen on all of the tumors in Table 20, while Class II was only observed after IFN-gamma stimulation. Furthermore, the almost complete ablation of Class II induction by iHsp90 seen in combination treatments was not seen for Class I in all of the tumors as it was for Class II.

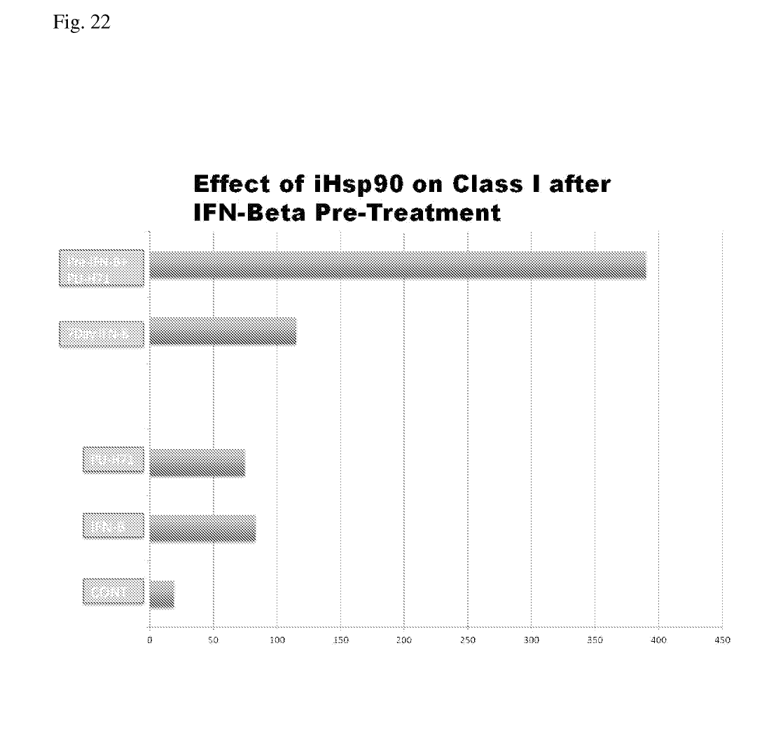

FIG. 22 shows that the 3 day-pre-treatment of melanoma MU89 with IFN-beta is further synergistically enhanced by the later addition of PU-H71. Thus, the addition of Hsp90 inhibitor for 3 days following the initial addition of IFN-beta shows the highest levels of Class I antigen induction that were achieved for any of the cells tested with agents individually, or in simultaneous culture.

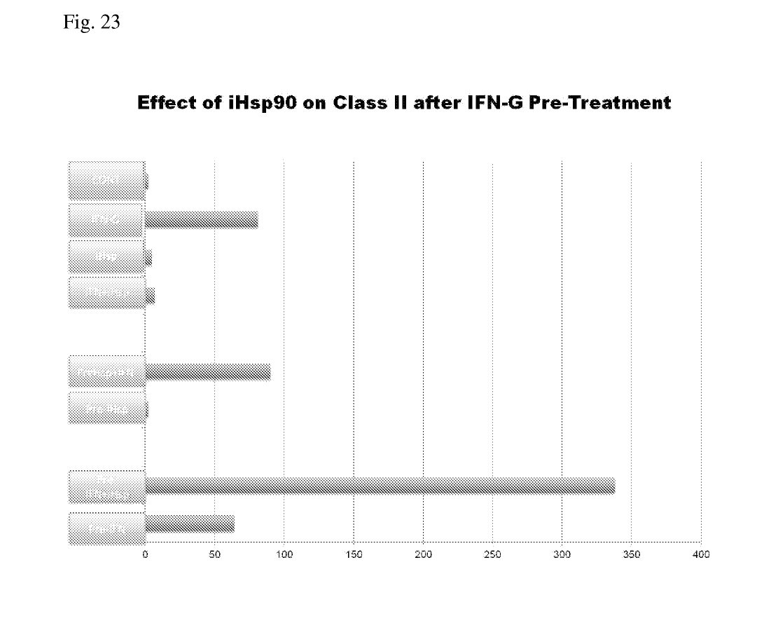

The data in Table 21 and in FIG. 23 (for melanoma MU89) demonstrate that pre-treatment with interferon-gamma changes the pattern of MHC expression induced by iHsp90. The data for both MU89 and HeLa tumor cells shows that Class II expression is consistently inhibited by co-treatment with IFN-gamma and iHsp90, but if the tumor cells are first exposed to IFN-gamma for 3 days to induce Class II expression, and the cells are subsequently treated with PU-H71, the level of Class II antigen achieved was significantly greater than that achieved with IFN-gamma alone. In contrast to iHsp90 alone, or the inhibitory effects of iHsp90 on initial induction of Class II antigen, the sequential treatment with these drugs leads to the highest levels of Class II expression.

Importantly, if cells are pre-treated for 3 days with PU-H71, the inhibitory effect is largely lost if the iHsp90 is removed and the cells are subsequently stimulated with IFN-gamma, in which case Class II induction is observed, while the simultaneous addition of both drugs in co-culture leads to loss of IFN-gamma induced Class II antigen. These results indicate that iHsp90 synergistically enhances Class II MHC expression after IFN-gamma has begun the induction process, while by itself, PU-H71 does not induce Class II, and it is inhibitory to initial IFN-gamma signaling. Significantly, after IFN-gamma pre-treatment the subsequent addition of PU-H71 achieves even higher levels of Class II expression (FIG. 23).

We also tested the ability of pre-treatment Interferons to enhance Class I expression when the cells were subsequently treated with PU-H71. If tumor cells are treated first for 4 days with IFN-beta, and then Hsp90 inhibitor (PU-H71) is added, the resultant levels of Class I antigen expression far exceeded what was achieved with either drug alone, or in simultaneous culture (FIG. 22).

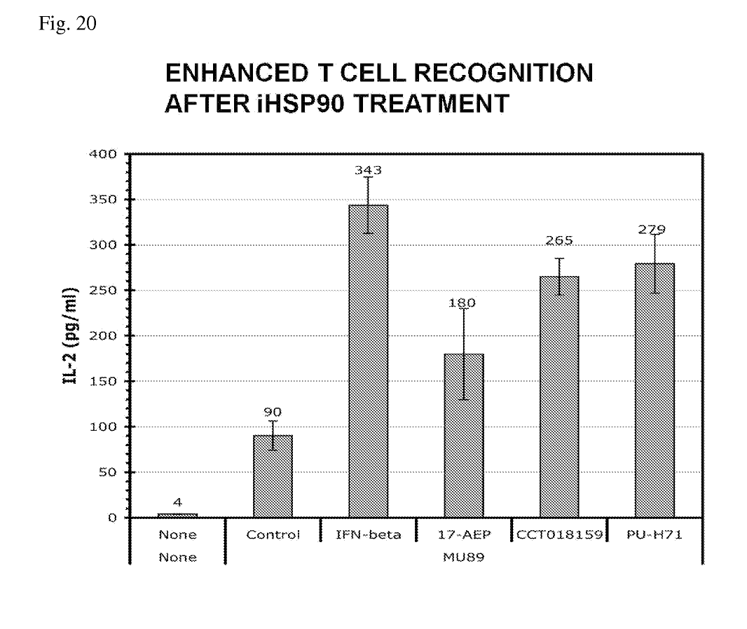

We utilized an in vitro cell line system we have developed to study recognition of tumor cells by T cells after MHC Class I up-regulation. We have engineered a Jurkat T cell line whose sole expressed TCR recognizes HLA-A2 regardless of the presented peptide, such that any cell line expressing surface HLA-A2 will be recognized by means of the binding specificity of this TCR. Depending on the level of Class I MHC on target HLA-A2 tumor cells, the Jurkat HLA-A2 specific TCR cell line will be activated to make and secrete IL-2 upon co-culture of the two cell lines together. Levels of IL-2 secretion increase concomitantly with MHC Class I up-regulation. We used this system to demonstrate the effect of IFN-beta and Hsp90 inhibitor treatment on HLA levels. MU89 cells treated with IFN beta alone increased IL-2 levels by 4-fold in two separate experiments. MU89 cells treated with Hsp90 inhibitors increased IL-2 levels 1.5-fold in two separate experiments (FIG. 20).

TABLE 19
Class I is induced on many tumors by Hsp90, but not Class II

| SAMPLE | TREATMENT | W6/32 | L243 |
|---|---|---|---|
| HELA | — | 130.7 | 2.1 |
| HELA | PU-H71 | 286.6 | 3.5 |
| HELA | IFN-BETA | 370.8 | 5.2 |
| HELA | IFN-GAMMA | 464.8 | 86.0 |
| MCF-7 | — | 80.5 | 3.0 |
| MCF-7 | PU-H71 | 113.0 | 4.7 |
| MCF-7 | IFN-BETA | 368.2 | 10.3 |
| MCF-7 | IFN-GAMMA | 478.5 | 59.1 |
| U2-OS | — | 73.3 | 1.8 |
| U2-OS | PU-H71 | 100.8 | 1.8 |
| U2-OS | IFN-BETA | 290.3 | 1.9 |
| U2-OS | IFN-GAMMA | 293.2 | 87.6 |
| MU-89 | — | 25.9 | 1.6 |
| MU-89 | PU-H71 | 118.5 | 4.5 |
| MU-89 | IFN-Beta | 151.3 | 2.8 |
| MU-89 | INF-Gamma | 138.0 | 57.6 |
| U118 | — | 58.5 | 1.9 |
| U118 | PU-H71 | 77.9 | 4.1 |
| U118 | IFN-Beta | 299.2 | 2.7 |
| U118 | IFN-Gamma | 250.5 | 65.3 |
| Jurkat | — | 27.5 | 1.2 |
| Jurkat | PU-H71 | 23.7 | 1.2 |
| Jurkat | IFN-BETA | 31.1 | 1.0 |
| Jurkat | IFN-GAMMA | 86.5 | 1.1 |
| Raji | — | 42 | 117.9 |
| Raji | PU-H71 | 139 | 79.0 |
| Raji | IFN-BETA | 81 | 129.8 |
| Raji | IFN-GAMMA | 117 | 339.3 |

TABLE 20
Class I following combination PU-H71 and Interferons for Class I, and
class II. Class II induction is blocked, but not Class I induction.

| SAMPLE | TREATMENT | W6/32 | L243 |
|---|---|---|---|
| MU89 | — | 9.9 | 1.5 |
| MU89 | IFN-B | 65.1 | 1.8 |
| MU89 | IFN-G | 86.7 | 64.3 |
| MU89 | PU-H71 | 72.1 | 4.1 |
| MU89 | IFN-B + PU-H71 | 140.1 | 3.2 |
| MU89 | IFN-G + PU-H71 | 93.2 | 3.1 |
| HeLa | — | 130.7 | 2.1 |
| HeLa | IFN-B | 370.8 | 5.2 |
| HeLa | IFN-G | 464.8 | 86.0 |
| HeLa | PU-H71 | 286.6 | 3.8 |
| HeLa | IFN-B + PU-H71 | 375.4 | 3.8 |
| HeLa | IFN-G + PU-H71 | 357.1 | 3.9 |
| U118 | — | 86.7 | 4.5 |
| U118 | IFN-B | 299.2 | 2.7 |
| U118 | IFN-G | 232.8 | 53.6 |
| U118 | PU-H71 | 135.7 | 6.3 |
| U118 | IFN-B + PU-H71 | 269.5 | 11.6 |
| U118 | IFN-G + PU-H71 | 342.4 | 13.2 |
| MCF-7 | — | 80.5 | 3.0 |
| MCF-7 | IFN-B | 368.2 | 10.3 |
| MCF-7 | IFN-G | 478.5 | 59.1 |
| MCF-7 | PU-H71 | 113.0 | 4.7 |
| MCF-7 | IFN-B + PU-H71 | 205.7 | 5.1 |
| MCF-7 | IFN-G + PU-H71 | 149.2 | 5.5 |
| U2-OS | — | 66.7 | 1.5 |
| U2-OS | IFN-B | 416.6 | 3.2 |
| U2-OS | IFN-G | 397.1 | 142.8 |
| U2-OS | PU-H71 | 109.0 | 1.9 |
| U2-OS | IFN-B + PU-H71 | 480.1 | 3.3 |
| U2-OS | IFN-G + PU-H71 | 498.7 | 2.1 |

TABLE 21
Pre-Treatment with IFN-gamma allows Synergy with follow-up Hsp90

| SAMPLE | PRE-TREATMENT | TREATMENT | L243 |
|---|---|---|---|
| MU89 | — | — | 2 |
| MU89 | — | IFN-G | 81 |
| MU89 | — | PU-H71 | 5 |
| MU89 | — | IFN-G + PU-H71 | 7 |
| MU89 | IFN-G | — | 64 |
| MU89 | IFN-G | PU-H71 | 338 |
| MU89 | PU-H71 | — | 2 |
| MU89 | PU-H71 | IFN-G | 90 |
| HeLa | — | — | 3 |
| HeLa | — | IFN-G | 97 |
| HeLa | — | PU-H71 | 4 |
| HeLa | — | IFN-G + PU-H71 | 7 |
| HeLa | IFN-G | — | 76 |
| HeLa | IFN-G | PU-H71 | 185 |
| HeLa | PU-H71 | — | 6 |
| HeLa | PU-H71 | IFN-G | 33 |

Materials and Methods for Example 3

Cell Lines and Cell Culture.

Melanoma cell lines MU89, MUX, and A375 were grown in DMEM with 10% FBS. A Jurkat cell line expressing a HLA-A2 specific TCR was constructed using a previously described TCR. Briefly, a lentivirus encoding the alpha and beta chain of the HLA-A2 specific TCR was used to transduce the Jurkat TCR-minus cell line J.RT3. Expression of the correct TCR in transduced cells was confirmed by CD3 and tetramer surface staining. Specificity of the TCR was shown by reaction of transduced Jurkat cells with the different HLA-typed tumors and with different peptides. 17-AEP (InvivoGen, San Diego, Calif.) was resuspended at 1 mg/ml in water. PU-H71 (Sigma Aldrich, St. Louis, Mo.) and CCT018159 (Tocris, Ellisville, Mo.) were resuspended in 100% DMSO at 10 mg/ml, with final dilutions in the tissue culture medium used in the cellular assays. IFN-beta (Avonex, Biogen) was resuspended in 1×PBS at 5×10⁵ Units/ml. For IFN-gamma, 100 μg/ml stock solution of IFN-γ-1b was prepared in PBS and stored at −20° C. Just prior to use, an aliquot was removed and a net 1000× dilution (1 mcl/ml net) was used for in vitro stimulation assays.

Drug Treatments.

Cell were seeded at 1×10⁶ cells in a T25 flask with 10 ml of media for the 4 day IFN-beta pre-treatments. Cells were then harvested and plated in 24 well plate at 1×10⁵ cells per well in 1 ml of media for 3 day treatments.

Antibody Staining for Flow Cytometry.

For surface staining of HLA Class I and II antigens, cells were harvested using CellStripper™ from Mediatech (Manassas, Va.). All staining steps were performed on ice. Following a first incubation with antibody to Class I (W6/32) or Class II (L243) for 30 minutes. Stained cells were washed twice with cold PBS and then stained with a goat anti-mouse FITC secondary antibody to allow for quantitative analysis by flow cytometry. For evaluation of cytoplasmic antigens, including Melan-A/MART-1 and gp100, cells were first fixed in paraformaldehyde and permeabilized with saponin as previously described. Staining for cytoplasmic antigens was carried out at room temperature (22° C.).

T Cell Recognition of Treated Tumor Cells.

In order to evaluate T cell recognition of tumor cells, a TCR-transduced Jurkat cell line was used as previously described. In brief, a Jurkat cell transcued to express a TCR specific for HLA-A2 was co-cultured with tumors cells, and after 24 hours of co-culture, the supernatants were collected for measurement of IL-2 produced by the responding Jurkat T cells. Tumor cells were treated with either iHsp90 or interferons in combination, as described in the text, and the treated tumor cells were collected and counted after 3 to 6 days of treatment. 5×104 tumor cells were incubated with 2×105 TCR-transduced Jurkat T cells for 24 hours. The supernatants were then assayed by ELISA for levels of IL-2 (BD Bioscience, San Diego, Calif.). The level of IL-2 is calculated by comparison to a standard curve of known quantities of IL-2.

### Other Embodiments

All publications and patents cited in this specification are incorporated herein by reference as if each individual publication or patent were specifically and individually indicated to be incorporated by reference. Although the foregoing invention has been described in some detail by way of illustration and example for purposes of clarity of understanding, it will be readily apparent to those of ordinary skill in the art in light of the teachings of this invention that certain changes and modifications may be made thereto without departing from the spirit or scope of the appended claims.

What is claimed is:

1. A method of treating a cancer in a subject, said method comprising administering to said subject a first composition comprising a compound selected from the group consisting of an HSP90 inhibitor, 3-(4-octadecyl)benzoylacrylic acid (OBAA), flunarizine, aphidicolin, damnacanthal, dantrolene, and an analog thereof, and administering a second composition comprising a cell, thereby treating the cancer; wherein
said first composition up-regulates expression of one or more tumor associated antigens (TAAs) on a cancer cell; and
the cell of said second composition interacts with said TAA.

2. The method of claim 1, wherein said cell is a white blood cell selected from the group consisting of a T cell, a NK cell, a LAK cell, monocyte, and a macrophage.

3. (canceled)

4. The method of claim 1, wherein said cell is engineered to express a receptor specific for at least one of said TAAs.

5. The method of claim 4, wherein said receptor specific for at least one of said TAAs is a chimeric T cell receptor.

6. The method of claim 5, wherein said chimeric T cell receptor comprises an antibody fragment specific for said TAA.

7. The method of claim 1, wherein said cell is autologous or allogeneic to said subject.

8. A method of treating a cancer in a subject, said method comprising administering to said subject a first composition comprising a compound selected from the group consisting of an HSP90 inhibitor, OBAA, flunarizine, aphidicolin, damnacanthal, dantrolene, and an analog thereof, and administering a second composition comprising a TAA.

9. The method of claim 8, wherein said second composition is administered singly or multiple times to the subject before or after administering the first composition; or wherein the first composition is administered singly or multiple times.

10. The method of claim 9, wherein said second composition is administered singly or multiple times to the subject 1 to 14 days before or after administering the first composition; wherein said second composition is administered singly or multiple times to the subject 14 to 30 days before or after administering the first composition; or wherein said second composition is administered singly or multiple times to the subject 1 to 6 months before or after administering the first composition.

11-13. (canceled)

14. The method of claim 8, further comprising administering a cell that interacts with a cell of the cancer.

15. The method of claim 14, wherein said cell being administered is a white blood cell selected from the group consisting of a T cell, a NK cell, a LAK cell, monocyte, and a macrophage.

16. (canceled)

17. The method of claim 14, wherein said cell being administered is engineered to express a receptor specific for at least one of said TAAs; or wherein said cell being administered is autologous orallogeneic to said subject.

18. The method of claim 17, wherein said receptor specific for at least one of said TAAs is a chimeric T cell receptor.

19. The method of claim 18, wherein said chimeric T cell receptor comprises an antibody fragment specific for said TAA.

20. (canceled)

21. A method of treating a cancer in a subject, said method comprising administering to said subject a first composition comprising a compound selected from the group consisting of an HSP90 inhibitor, OBAA, flunarizine, aphidicolin, damnacanthal, dantrolene, and an analog thereof, and administering a second composition comprising an antigen-binding scaffold specific for a TAA.

22. The method of claim 21, wherein the antigen-binding scaffold is an antibody, a soluble T cell receptor, or a chimeric receptor.

23. The method of claim 1, further comprising administering a third composition comprising an IFN-β receptor agonist, an IFN-γ receptor agonist, or a CTLA-4 antagonist.

24. The method of claim 23, wherein said IFN-β receptor agonist comprises IFN-β, an IFN-β mimic, an IFN-β receptor antibody, or a fragment thereof; or wherein said IFN-β receptor agonist comprises a polypeptide comprising the amino acid sequence of SEQ ID NO: 1 or SEQ ID NO: 2.

25. (canceled)

26. The method of claim 24, wherein the amino acid sequence of said polypeptide consists of the amino acid sequence of SEQ ID NO: 1; or wherein the amino acid sequence of said polypeptide consists of the amino acid sequence of SEQ ID NO:2.

27. (canceled)

28. The method of claim 23, wherein said IFN-γ receptor agonist comprises IFN-γ, an IFN-γ mimic, an IFN-γ receptor antibody, or a fragment thereof; or wherein said IFN-γ receptor agonist comprises a polypeptide comprising the amino acid sequence of SEQ ID NO: 3, SEQ ID NO: 4, SEQ ID NO: 5, or SEQ ID NO: 6.

29. (canceled)

30. The method of claim 28, wherein the amino acid sequence of said polypeptide consists of the amino acid sequence of SEQ ID NO: 3; wherein the amino acid sequence of said polypeptide consists of the amino acid sequence of SEQ ID NO: 4, wherein the amino acid sequence of said polypeptide consists of the amino acid sequence of SEQ ID NO: 5; or wherein the amino acid sequence of said polypeptide consists of the amino acid sequence of SEQ ID NO: 6.

31-33. (canceled)

34. The method of claim 23, wherein said third composition comprises IFN-β or IFN-γ, and wherein said first composition comprises an HSP90 inhibitor.

35. The method of claim 34, wherein said HSP90 inhibitor is selected from the group consisting of 17-AAG-nab; 17-AAG; 17-AEP; 17-DMAG; Alvespimycin; Autolytimycin; AUY13387; AT13387; BIIB028; BIIB021; BX-2819; CCT018159; Celastrol; CUDC-305; CUDC-305; Curvularin; Debio 0932; DS-2248; Flavopiridol; Geldamycin; Gedunin; Herbimycin A; Herbimycin B; Herbimycin C; HSP990; IPI-493; IPI-504; KW 2478; Lebstatin; L-783,277; LL-Z1640-2; Macbecin I; Maytansine; MPC-3100; MPC-6827; Mycograb; NCS-683664; NXD30001; NVP-AUY922; NVP-HSP990; Novobiocin; PF-04929113; Pochonin D; PU-H71; PU24FC1; PU-3; Radicicol; Reblastatin; Redicicol; Rifabutin; SNX-2112; SNX-5422; SNX-7081; STA-1474; STA-9090; Tanespimycin; VER49009; Xestodecalactone; XL888; and Zearalenone.

36. (canceled)

37. The method of claim 23, wherein said third composition and said first composition are administered within 14 days of each other.

38. The method of claim 37, wherein said third composition is administered between one and seven days prior to said administration of said first composition; wherein said third composition is administered between one and three days prior to said administration of said first composition; wherein said third composition is administered between one and 24 hours prior to said administration of said first composition; wherein said third composition is administered between one and seven days following said administration of said first composition; wherein said third composition is administered between one and three days following said administration of said first composition; or wherein said third composition is administered between one and 24 hours following said administration of said first composition.

39-43. (canceled)

44. The method of claim 1, wherein said cancer is selected from the group consisting of acute lymphoblastic leukemia, acute myeloid leukemia, adrenocortical carcinoma, anal cancer, appendix cancer, astrocytoma, atypical teratoid/rhabdoid tumor, basal cell carcinoma, bile duct cancer, bladder cancer, bone cancer, brain stem glioma, brain tumor, breast cancer, bronchial tumor, Burkitt lymphoma, carcinoid tumor, cervical cancer, chordoma, chronic lymphocytic leukemia, chronic myeloproliferative disorder, colon cancer, colorectal cancer, craniopharyngioma, cutaneous T cell lymphoma, endometrial cancer, ependymoblastoma, ependymoma, esophageal cancer, Ewing sarcoma, extracranial germ cell tumor, extragonadal germ cell tumor, extrahepatic bile duct cancer, eye cancer, gallbladder cancer, gastric cancer, gastroesophageal cancer, gastrointestinal cancer, germ cell tumor, gestational trophoblastic tumor, glioma, hairy cell leukemia, head and neck cancer, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, leukemia, lip and oral cavity cancer, liver cancer, lung cancer, malignant teratoma, non-Hodgkin lymphoma, macroglobulinemia, osteosarcoma, medulloblastoma, melanoma, merkel cell carcinoma, mesothelioma, mouth cancer, mycosis fungiodes, myelodysplastic syndrome, multiple myeloma, nasal cavity and paranasal sinus cancer, nasopharyngeal cancer, non-small cell lung cancer, oral cancer, oropharyngeal cancer, osteosarcoma, ovarian cancer, ovarian epithelial cancer, pancreatic cancer, papillomatosis, parathyroid cancer, penile cancer, pharyngeal cancer, pituitary tumor, prostate cancer, rectal cancer, renal cell cancer, retinoblastoma, rhabdomycosarcoma, salivary gland cancer, sarcoma, skin cancer, small intestine cancer, soft tissue sarcoma, testicular cancer, throat cancer, thomoma, thymic carcinoma, thyroid cancer, urethral cancer, uterine cancer, vaginal cancer, and Wilms tumor.

45-48. (canceled)

49. The method of claim 1, wherein said TAA is selected from the group consisting of Melan-A/MART-1, tyrosinase, gp100/pmel 17, TRP-1, TRP-2, an MITF, MITF-A, MITF-M, melanoma GP75, Annexin I, Annexin II, adenosine deaminase-binding protein (ADAbp), PGP 9.5, Colorectal associated antigen (CRC)-C017-1A/GA733, Ab2 BR3E4, CI17-1A/GA733, Hsp70, Hsp90, Hsp96, Hsp105, Hsp110, HSPPC-96, stress protein gp96, gp96-associated cellular peptide, G250, Dipeptidyl peptidase IV (DPPIV), Mammaglobin, thyroglobulin, STn, Carcinoembryonic Antigen (CEA), CEA epitope CAP-I, CEA epitope CAP-2, etv6, amI1, Prostate Specific Antigen (PSA), PSA epitope PSA-1, PSA epitope PSA-2, PSA epitope PSA-3, Ad5-PSA, prostate-specific membrane antigen (PSMA), Prostatic Acid Phosphatase (PAP), Prostate epithelium-derived Ets transcription factor (PDEF), Parathyroid-hormone-related protein (PTH-rP), EGFR, PLU1, Oncofetal antigen-immature laminin receptor (OFA-iLR), MN/CA IX (CA9), HP59, Cytochrome oxidase 1, sp100, msa, Ran GTPase activating protein, a Rab-GAP (Rab GTPase-activating) protein, PARIS-I, T cell receptor/CD3-zeta chain, cTAGE-1, SCP-1, Glycolipid antigen-GM2, GD2 or GD3, GM3, FucosylGM1, Glycoprotein (mucin) antigens-Tn, Sialyl-Tn, TF, and Mucin-I, CA125 (MUC-16), a MAGE family antigen, GAGE-1,2, BAGE, RAGE, LAGE-1, GnT-V, EP-CAM/KSA, CDK4, a MUC family antigen, HER2/neu, ErbB-2/neu, p21 ras, RCAS1, α-fetoprotein, E-cadherin, α-catenin, β-catenin, NeuGcGM3, Fos related antigen, Cyclophilin B, RCAS1, S2, L10a, Telomerase rt peptide, cdc27, fodrin, p120ctn, PRAME, GA733/EoCam, NY-BR-I, NY-BR-2, NY-BR-3, NY-BR-4, NY-BR-5, NY-BR-6, NY-BR-7, NY-ESO-1, L19H1, MAZ, PINCH, PRAME, Prp1p/Zer1p, WT1, adenomatous polyposis coli protein (APC), PHF3, LAGE-1, SART3, SCP-1, SSX-1, SSX-2, SSX-4, TAG-72, TRAG-3, MBTAA, a Smad tumor antigen, Imp1, HPV-16 E7, c-erbB-2, EBV-encoded nuclear antigen (EBNA)-1, Herpes simplex thymidine kinase (HSVtk), alternatively spliced isoform of XAGE-1 (L552S), TGF beta RII frame shift mutation, BAX frame shift mutation, and an immunogenic fragment thereof.

50. The method of claim 1, wherein said TAA is selected from Table 6 or an immunogenic fragment of any of the TAAs listed in Table 6.

51. The method of claim 1, wherein said treating reduces tumor volume, inhibits an increase in tumor volume, stimulates tumor cell lysis or apoptosis, reduces tumor metastasis, reduces the cell number or viability of cells within a mestastasis, or reduces the number of new metastases.

52. The method of claim 1, further comprising administering an anti-tumor therapy.

53. The method of claim 52, wherein the anti-tumor therapy comprises surgical resection, radiotherapy, or chemotherapy.

54. The method of claim 1, wherein said HSP90 inhibitor is selected from the group consisting of 17-AAG-nab; 17-AAG; 17-AEP; 17-DMAG; Alvespimycin; Autolytimycin; AUY13387; AT13387; BIIB028; BIIB021; BX-2819; CCT018159; Celastrol; CUDC-305; CUDC-305; Curvularin; Debio 0932; DS-2248; Flavopiridol; Geldamycin; Gedunin; Herbimycin A; Herbimycin B; Herbimycin C; HSP990; IPI-493; IPI-504; KW 2478; Lebstatin; L-783,277; LL-Z1640-2; Macbecin I; Maytansine; MPC-3100; MPC-6827; Mycograb; NCS-683664; NXD30001; NVP-AUY922; NVP-HSP990; Novobiocin; PF-04929113; Pochonin D; PU-H71; PU24FC1; PU-3; Radicicol; Reblastatin; Redicicol; Rifabutin; SNX-2112; SNX-5422; SNX-7081; STA-1474; STA-9090; Tanespimycin; VER49009; Xestodecalactone; XL888; and Zearalenone.

55. (canceled)

56. The method of claim 1, wherein said HSP90 inhibitor is selected from the compounds in Table 1.

57. The method of claim 1, wherein said flunarizine analog is cinnarizine.

58. The method of claim 1, wherein said compound of said first composition is selected from a compound listed in Tables 2-5.

59. A method of treating a cancer in a subject, said method comprising administering to said subject a first composition comprising a compound selected from the group consisting of an HSP90 inhibitor, OBAA, flunarizine, aphidicolin, damnacanthal, dantrolene, and an analog thereof, and administering a second composition comprising an IFN-β receptor agonist or an IFN-γ receptor agonist, thereby treating the cancer.

60. The method of claim 59, wherein said IFN-β receptor agonist comprises IFN-β, an IFN-β mimic, an IFN-β receptor antibody, or a fragment thereof; or wherein said IFN-β receptor agonist comprises a polypeptide comprising or consisting of the amino acid sequence of SEQ ID NO: 1 or SEQ ID NO: 2.

61. (canceled)

62. The method of claim 59, wherein said IFN-γ receptor agonist comprises IFN-γ, an IFN-γ mimic, an IFN-γ receptor antibody, or a fragment thereof; or wherein said IFN-γ receptor agonist comprises a polypeptide comprising or consisting of the amino acid sequence of SEQ ID NO: 3, SEQ ID NO: 4, SEQ ID NO: 5, or SEQ ID NO: 6.

63. (canceled)

64. The method of claim 59, wherein said second composition comprises IFN-β or IFN-γ, and wherein said first composition comprises an HSP90 inhibitor.

65. The method of claim 64, wherein said HSP90 inhibitor is selected from the group consisting of 17-AAG-nab; 17-AAG; 17-AEP; 17-DMAG; Alvespimycin; Autolytimycin; AUY13387; AT13387; BIIB028; BIIB021; BX-2819; CCT018159; Celastrol; CUDC-305; CUDC-305; Curvularin; Debio 0932; DS-2248; Flavopiridol; Geldamycin; Gedunin; Herbimycin A; Herbimycin B; Herbimycin C; HSP990; IPI-493; IPI-504; KW 2478; Lebstatin; L-783,277; LL-Z1640-2; Macbecin I; Maytansine; MPC-3100; MPC-6827; Mycograb; NCS-683664; NXD30001; NVP-AUY922; NVP-HSP990; Novobiocin; PF-04929113; Pochonin D; PU-H71; PU24FC1; PU-3; Radicicol; Reblastatin; Redicicol; Rifabutin; SNX-2112; SNX-5422; SNX-7081; STA-1474; STA-9090; Tanespimycin; VER49009; Xestodecalactone; XL888; and Zearalenone.

66. (canceled)

67. The method of claim 59, wherein said third composition and said first composition are administered within 14 days of each other.

68. The method of claim 67, wherein said third composition is administered between one and seven days prior to said administration of said first composition; wherein said third composition is administered between one and three days prior to said administration of said first composition; wherein said third composition is administered between one and 24 hours prior to said administration of said first composition; wherein said third composition is administered between one and seven days following said administration of said first composition; wherein said third composition is administered between one and three days following said administration of said first composition; or wherein said third composition is administered between one and 24 hours following said administration of said first composition.

69-73. (canceled)

74. The method of claim 59, wherein said cancer is selected from the group consisting of acute lymphoblastic leukemia, acute myeloid leukemia, adrenocortical carcinoma, anal cancer, appendix cancer, astrocytoma, atypical teratoid/rhabdoid tumor, basal cell carcinoma, bile duct cancer, bladder cancer, bone cancer, brain stem glioma, brain tumor, breast cancer, bronchial tumor, Burkitt lymphoma, carcinoid tumor, cervical cancer, chordoma, chronic lymphocytic leukemia, chronic myeloproliferative disorder, colon cancer, colorectal cancer, craniopharyngioma, cutaneous T cell lymphoma, endometrial cancer, ependymoblastoma, ependymoma, esophageal cancer, Ewing sarcoma, extracranial germ cell tumor, extragonadal germ cell tumor, extrahepatic bile duct cancer, eye cancer, gallbladder cancer, gastric cancer, gastroesophageal cancer, gastrointestinal cancer, germ cell tumor, gestational trophoblastic tumor, glioma, hairy cell leukemia, head and neck cancer, hepatocellular cancer, histiocytosis, Hodgkin lymphoma, hypopharyngeal cancer, intraocular melanoma, islet cell tumor, Kaposi sarcoma, kidney cancer, Langerhans cell histiocytosis, laryngeal cancer, leukemia, lip and oral cavity cancer, liver cancer, lung cancer, malignant teratoma, non-Hodgkin lymphoma, macroglobulinemia, osteosarcoma, medulloblastoma, melanoma, merkel cell carcinoma, mesothelioma, mouth cancer, mycosis fungiodes, myelodysplastic syndrome, multiple myeloma, nasal cavity and paranasal sinus cancer, nasopharyngeal cancer, non-small cell lung cancer, oral cancer, oropharyngeal cancer, osteosarcoma, ovarian cancer, ovarian epithelial cancer, pancreatic cancer, papillomatosis, parathyroid cancer, penile cancer, pharyngeal cancer, pituitary tumor, prostate cancer, rectal cancer, renal cell cancer, retinoblastoma, rhabdomycosarcoma, salivary gland cancer, sarcoma, skin cancer, small intestine cancer, soft tissue sarcoma, testicular cancer, throat cancer, thomoma, thymic carcinoma, thyroid cancer, urethral cancer, uterine cancer, vaginal cancer, and Wilms tumor.

75. The method of claim 59, wherein said TAA is selected from the group consisting of Melan-A/MART-1, tyrosinase, gp100/pmel 17, TRP-1, TRP-2, an MITF, MITF-A, MITF-M, melanoma GP75, Annexin I, Annexin II, adenosine deaminase-binding protein (ADAbp), PGP 9.5, Colorectal associated antigen (CRC)-C017-1A/GA733, Ab2 BR3E4, CI17-1A/GA733, Hsp70, Hsp90, Hsp96, Hsp105, Hsp110, HSPPC-96, stress protein gp96, gp96-associated cellular peptide, G250, Dipeptidyl peptidase IV (DPPIV), Mammaglobin, thyroglobulin, STn, Carcinoembryonic Antigen (CEA), CEA epitope CAP-I, CEA epitope CAP-2, etv6, amI1, Prostate Specific Antigen (PSA), PSA epitope PSA-1, PSA epitope PSA-2, PSA epitope PSA-3, Ad5-PSA, prostate-specific membrane antigen (PSMA), Prostatic Acid Phosphatase (PAP), Prostate epithelium-derived Ets transcription factor (PDEF), Parathyroid-hormone-related protein (PTH-rP), EGFR, PLU1, Oncofetal antigen-immature laminin receptor (OFA-iLR), MN/CA IX (CA9), HP59, Cytochrome oxidase 1, sp100, msa, Ran GTPase activating protein, a Rab-GAP (Rab GTPase-activating) protein, PARIS-I, T cell receptor/CD3-zeta chain, cTAGE-1, SCP-1, Glycolipid antigen-GM2, GD2 or GD3, GM3, FucosylGM1, Glycoprotein (mucin) antigens-Tn, Sialyl-Tn, TF, and Mucin-I, CA125 (MUC-16), a MAGE family antigen, GAGE-1,2, BAGE, RAGE, LAGE-1, GnT-V, EP-CAM/KSA, CDK4, a MUC family antigen, HER2/neu, ErbB-2/neu, p21 ras, RCAS1, α-fetoprotein, E-cadherin, α-catenin, β-catenin, NeuGcGM3, Fos related antigen, Cyclophilin B, RCAS1, S2, L10a, Telomerase rt peptide, cdc27, fodrin, p120ctn, PRAME, GA733/EoCam, NY-BR-I, NY-BR-2, NY-BR-3, NY-BR-4, NY-BR-5, NY-BR-6, NY-BR-7, NY-ESO-1, L19H1, MAZ, PINCH, PRAME, Prp1p/Zer1p, WT1, adenomatous polyposis coli protein (APC), PHF3, LAGE-1, SART3, SCP-1, SSX-1, SSX-2, SSX-4, TAG-72, TRAG-3, MBTAA, a Smad tumor antigen, Imp1, HPV-16 E7, c-erbB-2, EBV-encoded nuclear antigen (EBNA)-1, Herpes simplex thymidine kinase (HSVtk), alternatively spliced isoform of XAGE-1 (L552S), TGF beta RII frame shift mutation, BAX frame shift mutation, and an immunogenic fragment thereof.

76. The method of claim 59, wherein said TAA is selected from Table 6 or an immunogenic fragment of any of the TAAs listed in Table 6.

77. The method of claim 59, wherein said HSP90 inhibitor is selected from the group consisting of 17-AAG-nab; 17-AAG; 17-AEP; 17-DMAG; Alvespimycin; Autolytimycin; AUY13387; AT13387; BIIB028; BIIB021; BX-2819; CCT018159; Celastrol; CUDC-305; CUDC-305; Curvularin; Debio 0932; DS-2248; Flavopiridol; Geldamycin; Gedunin; Herbimycin A; Herbimycin B; Herbimycin C; HSP990; IPI-493; IPI-504; KW 2478; Lebstatin; L-783,277; LL-Z1640-2; Macbecin I; Maytansine; MPC-3100; MPC-6827; Mycograb; NCS-683664; NXD30001; NVP-AUY922; NVP-HSP990; Novobiocin; PF-04929113; Pochonin D; PU-H71; PU24FC1; PU-3; Radicicol; Reblastatin; Redicicol; Rifabutin; SNX-2112; SNX-5422; SNX-7081; STA-1474; STA-9090; Tanespimycin; VER49009; Xestodecalactone; XL888; and Zearalenone.

78. (canceled)

79. A kit comprising:
(i) an HSP90 inhibitor, OBAA, flunarizine, aphidicolin, damnacanthal, dantrolene, or an analog thereof;
(ii) a TAA; and
(iii) instructions for the administration of the HSP90 inhibitor, OBAA, flunarizine, aphidicolin, damnacanthal, dantrolene, or an analog thereof and the TAA to a subject having cancer or having an increased risk of developing a cancer; or a kit comprising
(i) a composition comprising a TAA and an HSP90 inhibitor, OBAA, flunarizine, aphidicolin, damnacanthal, dantrolene, or an analog thereof; and
(ii) instructions for the administration of said composition to a subject having cancer or having an increased risk of developing a cancer; or a kit comprising
(i) an HSP90 inhibitor, OBAA, flunarizine, aphidicolin, damnacanthal, dantrolene, or an analog thereof;
(ii) a composition comprising an IFN-β receptor agonist or IFN-γ receptor agonist; and
(iii) instructions for the administration of the HSP90 inhibitor, OBAA, flunarizine, aphidicolin, damnacanthal, dantrolene, or an analog thereof and the IFN-β receptor agonist or IFN-γ receptor agonist to a subject having cancer or having an increased risk of developing a cancer.

80. (canceled)

81. The kit of claim 79, wherein said TAA is selected from the group consisting of Melan-A/MART-1, tyrosinase, gp100/pmel 17, TRP-1, TRP-2, an MITF, MITF-A, MITF-M, melanoma GP75, Annexin I, Annexin II, ADAbp, PGP 9.5, CRC-C017-1A/GA733, Ab2 BR3E4, CI17-1A/GA733, Hsp70, Hsp90, Hsp96, Hsp105, Hsp110, HSPPC-96, stress protein gp96, gp96-associated cellular peptide, G250, DPPIV, Mammaglobin, thyroglobulin, STn, CEA, CEA epitope CAP-I, CEA epitope CAP-2, etv6, amI1, PSA, PSA epitope PSA-1, PSA epitope PSA-2, PSA epitope PSA-3, Ad5-PSA, PSMA, PAP, PDEF, PTH-rP, EGFR, PLU1, OFA-iLR, MN/CA IX (CA9), HP59, Cytochrome oxidase 1, sp100, msa, Ran GTPase activating protein, a Rab-GAP protein, PARIS-I, T cell receptor/CD3-zeta chain, cTAGE-1, SCP-1, Glycolipid antigen-GM2, GD2 or GD3, GM3, FucosylGM1, Glycoprotein (mucin) antigens-Tn, Sialyl-Tn, TF, and Mucin-I, CA125 (MUC-16), a MAGE family antigen, GAGE-1,2, BAGE, RAGE, LAGE-1, GnT-V, EP-CAM/KSA, CDK4, a MUC family antigen, HER2/neu, ErbB-2/neu, p21 ras, RCAS1, α-fetoprotein, E-cadherin, α-catenin, β-catenin, NeuGcGM3, Fos related antigen, Cyclophilin B, RCAS1, S2, L10a, Telomerase rt peptide, cdc27, fodrin, p120ctn, PRAME, GA733/EoCam, NY-BR-I, NY-BR-2, NY-BR-3, NY-BR-4, NY-BR-5, NY-BR-6, NY-BR-7, NY-ESO-1, L19H1, MAZ, PINCH, PRAME, Prp1p/Zer1p, WT1, APC, PHF3, LAGE-1, SART3, SCP-1, SSX-1, SSX-2, SSX-4, TAG-72, TRAG-3, MBTAA, a Smad tumor antigen, Imp1, HPV-16 E7, c-erbB-2, EBNA-1, HSVtk, L552S, TGF beta RII frame shift mutation, BAX frame shift mutation, and an immunogenic fragment thereof.

82. (canceled)

83. A composition comprising (i) a compound selected from the group consisting of an HSP90 inhibitor, OBAA, flunarizine, aphidicolin, damnacanthal, dantrolene, and an analog thereof, and (ii) a TAA; or a composition comprising (i) a compound selected from the group consisting of an HSP90 inhibitor, OBAA, flunarizine, aphidicolin, damnacanthal, dantrolene, and an analog thereof, and (ii) an IFN-β receptor agonist or IFN-γ receptor agonist.

84. The composition of claim 83, wherein said TAA is selected from the group consisting of Melan-A/MART-1, tyrosinase, gp100/pmel 17, TRP-1, TRP-2, an MITF, MITF-A, MITF-M, melanoma GP75, Annexin I, Annexin II, ADAbp, PGP 9.5, CRC-C017-1A/GA733, Ab2 BR3E4, CI17-1A/GA733, Hsp70, Hsp90, Hsp96, Hsp105, Hsp110, HSPPC-96, stress protein gp96, gp96-associated cellular peptide, G250, DPPIV, Mammaglobin, thyroglobulin, STn, CEA, CEA epitope CAP-I, CEA epitope CAP-2, etv6, amI1, PSA, PSA epitope PSA-1, PSA epitope PSA-2, PSA epitope PSA-3, Ad5-PSA, PSMA, PAP, PDEF, PTH-rP, EGFR, PLU1, OFA-iLR, MN/CA IX (CA9), HP59, Cytochrome oxidase 1, sp100, msa, Ran GTPase activating protein, a Rab-GAP protein, PARIS-I, T cell receptor/CD3-zeta chain, cTAGE-1, SCP-1, Glycolipid antigen-GM2, GD2 or GD3, GM3, FucosylGM1, Glycoprotein (mucin) antigens-Tn, Sialyl-Tn, TF and Mucin-I, CA125 (MUC-16), a MAGE family antigen, GAGE-1,2, BAGE, RAGE, LAGE-1, GnT-V, EP-CAM/KSA, CDK4, a MUC family antigen, HER2/neu, ErbB-2/neu, p21 ras, RCAS1, α-fetoprotein, E-cadherin, α-catenin, β-catenin, NeuGcGM3, Fos related antigen, Cyclophilin B, RCAS1, S2, L10a, Telomerase rt peptide, cdc27, fodrin, p120ctn, PRAME, GA733/EoCam, NY-BR-I, NY-BR-2, NY-BR-3, NY-BR-4, NY-BR-5, NY-BR-6, NY-BR-7, NY-ESO-1, L19H1, MAZ, PINCH, PRAME, Prp1p/Zer1p, WT1, APC, PHF3, LAGE-1, SART3, SCP-1, SSX-1, SSX-2, SSX-4, TAG-72, TRAG-3, MBTAA, a Smad tumor antigen, Imp1, HPV-16 E7, c-erbB-2, EBNA-1, HSVtk, L552S, TGF beta RII frame shift mutation, BAX frame shift mutation, and an immunogenic fragment thereof.

85. (canceled)

86. The composition of claim 83, wherein the IFN-β receptor agonist or IFN-γ receptor agonist is IFN-β-1a or IFN-γ-1b.

87. The composition of claim 83, wherein said HSP 90 inhibitor is selected from the group consisting of 17-AAG, 17-AEP, 17-DMAG, BIIB021, CCT018159, Celastrol, Gedunin, NVP-AUY922, PU-H71, and Radicicol.


SEQUENCE LISTING (6 sequences)

SEQ ID NO: 1 - protein, 166 aa, Homo sapiens
  MISC_FEATURE (1)..(166): IFN-beta-1a
MSYNLLGFLQRSSNFQCQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 2 - protein, 165 aa, Artificial Sequence
  IFN-beta-1b
SYNLLGFLQRSSNFQSQKLLWQLNGRLEYCLKDRMNFDIPEEIKQLQQFQKEDAALTIYEMLQNIFAIFRQDSSSTGWNETIVENLLANVYHQINHLKTVLEEKLEKEDFTRGKLMSSLHLKRYYGRILHYLKAKEYSHCAWTIVRVEILRNFYFINRLTGYLRN

SEQ ID NO: 3 - protein, 143 aa, Homo sapiens
  MISC_FEATURE (1)..(143): IFN-gamma
QDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELPPAAETGKRKRSQMLFRGRRASQ

SEQ ID NO: 4 - protein, 146 aa, Artificial Sequence
  IFN-gamma-1a
CYCQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELPPAAETGKRKRSQMLFRGRRASQ

SEQ ID NO: 5 - protein, 140 aa, Artificial Sequence
  IFN-gamma-1b
MQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELPPAAETGKRKRSQMLFRGR

SEQ ID NO: 6 - protein, 144 aa, Artificial Sequence
  IFN-gamma-1c
MQDPYVKEAENLKKYFNAGHSDVADNGTLFLGILKNWKEESDRKIMQSQIVSFYFKLFKNFKDDQSIQKSVETIKEDMNVKFFNSNKKKRDDFEKLTNYSVTDLNVQRKAIHELIQVMAELPPAAETGKRKRSQMLFRGRRASQ